(19) United States
(12) Patent Application Publication
(10) Pub. No.: US 2024/0327490 A1
(43) Pub. Date: Oct. 3, 2024

(54) SARS-COV-2-SPECIFIC T CELL RECEPTORS AND RELATED MATERIALS AND METHODS OF USE

(71) Applicant: The Johns Hopkins University, Baltimore, MD (US)

(72) Inventors: Kellie Smith, Dundalk, MD (US); Arbor Dykema, Baltimore, MD (US); Andrew Pardoll, Brookeville, MD (US); Andrea Lynn Cox, Baltimore, MD (US); Joel Blankson, Baltimore, MD (US); Franco D'Alessio, Baltimore, MD (US)

(21) Appl. No.: 18/271,615

(22) PCT Filed: Jan. 7, 2022
(86) PCT No.: PCT/US22/11650

§ 371 Date: Jul. 10, 2023

Related U.S. Application Data
(60) Provisional application No. 63/135,534, filed on Jan. 8, 2021.

(51) Int. Cl. C07K 14/725; A61K 38/00; A61K 45/06

(52) U.S. Cl. CPC C07K 14/7051; A61K 45/06; A61K 38/00

(57) ABSTRACT

Provided herein are, inter alia, methods, compositions and kits for treating, preventing and diagnosing coronaviruses, specifically COVID-19. Also included herein are kits for treating, preventing and diagnosing coronaviruses, specifically COVID-19.

### CROSS-REFERENCE TO RELATED APPLICATIONS

This application is a 35 U.S.C. § 371 national phase entry of International Patent Application No. PCT/US22/11650 filed Jan. 7, 2022, which claims the benefit of priority under 35 U.S.C. 119 from U.S. provisional application No. 63/135,534, filed Jan. 8, 2021, the disclosures of which are incorporated herein by reference in their entireties.

### INCORPORATION BY REFERENCE OF SEQUENCE LISTING

The contents of the sequence listing text file named “Sequence_Listing_048317_617001WO_122348945_1.txt”, which was created on Jul. 10, 2023 and is 257,824 bytes in size, are incorporated herein by reference in its entirety.

### FIELD

New compositions and methods for treating coronaviruses, specifically severe acute respiratory syndrome coronavirus 2 (SARS-CoV-2 or COVID-19).

### BACKGROUND

Coronaviruses are a family of RNA viruses which may cause illness in animals or humans. In humans, several coronaviruses are known to cause respiratory infections ranging from the common cold to more severe diseases such as Middle East Respiratory Syndrome (MERS) and Severe Acute Respiratory Syndrome (SARS).

The 2019 SARS-CoV-2 outbreak and ensuing global pandemic has resulted in significant global morbidity and mortality. Coronavirus disease 19 (COVID-19) symptom severity ranges from mild, or even asymptomatic, to the development of acute respiratory distress syndrome (ARDS), hospitalization, and death (1, 2).

It would be desirable have new therapies for treating against SARS-CoV-2 (COVID-19).

### BRIEF SUMMARY

Provided herein are, inter alia, methods, compositions and kits for treating and preventing severe acute respiratory syndrome coronavirus 2 (SARS-CoV-2 or COVID-19).

In aspects, provided herein is a T cell receptor (TCR) having an alpha and/or beta chain. In embodiments, the alpha and/or beta chain has at least one complementarity-determining region (CDR3) having at least one sequence of SEQ IDs 1-547.

In embodiments, the TCR described herein has an alpha chain, where the alpha chain has at least one sequence of SEQ ID NO: 1-13 or 545-547.

In other embodiments, the TCR has a peptide, where the peptide is cross-reactive (for example, cross-reactive may mean present in multiple patients and shares sequence homology). The cross-reactive peptide for example has at least one sequence of CAWSVQGNYGYTF, CAWSVGGNYGYTF, CAWSVSSSYGYTF, CAWSVQANYGYTF, CAWSVQQSYGYTF, CAWSVQQNYGYTF or CAWSVSQNYGYTF.

In other embodiments, the TCR has at least one mono-reactive peptide (e.g., for example the mono-reactive peptide is against coronavirus spike protein and homology between multiple patients. For example, the mono-reactive peptide has at least one sequence of CSARDVTGNYGYTF, CSARGTGTNYGYTF, CSVENGGNYGYTF, CASREGQGGYGYTF, CSAREWGGGYGYTF, CSARGTGNYGYTF, CSARGEGNYGYTF, CSARGWGNYGYTF, CSARGQGNYGYTF, CSGRGQGNYGYTF, CAIGGDSNYGYTF, CAWGWFNYGYTF, CASSLLSANYGYTF, CASSLGPFYGYTF, CASSLGWQLYGYTF, CASSPLGTGRYGYTF, CASSLGDGRSYGYTF, CASSFGGSRYGYTF, CASSYRGAQGYTF, CASSQRGAHGYTF, CASSLRGADGYTF, CASSLRGANGYTF, CASSLRGGNGYTF, CASSLAGGHGYTF, CASSRRGGDGYTF, or CASSERTGGEGYTF.

In embodiments, the TCR described herein further includes a variable domain, a constant domain, or fragments thereof.

In embodiments, the TCR also includes the ability to bind to a tumor antigen/HLA complex.

In aspects, provided herein is a nucleic acid molecule encoding the TCR described herein, e.g, a T cell receptor (TCR) having an alpha and/or beta chain. In embodiments, the alpha and/or beta chain has at least one complementarity-determining region (CDR3) having at least one sequence of SEQ IDs 1-547 (SEQ IDs 1-547 set forth in Table 1 below).

In other aspects, provided herein is a vector including a nucleic acid molecule encoding the TCR described herein, e.g, a T cell receptor (TCR) having an alpha and/or beta chain. In embodiments, the alpha and/or beta chain has at least one complementarity-determining region (CDR3) having at least one sequence of SEQ IDs 1-547 (SEQ IDs 1-547 set forth in Table 1 below).

In further aspects, provided herein is a T cell containing (e.g., expressing) a TCR having an alpha and/or beta chain. In embodiments, the alpha and/or beta chain has at least one complementarity-determining region (CDR3) having at least one sequence of SEQ IDs 1-547 (SEQ IDs 1-547 set forth in Table 1 below). For example, the T cell may be a human T cell or may be a non-human T cell.

In aspects, provided herein is a composition for treating a coronavirus, e.g., COVID19, where the composition has a T cell receptor (TCR) with an alpha and/or beta chain comprising SEQ IDs 1-547, or a TCR having an alpha chain with the sequence of SEQ ID NO: 1-13 or 545-547. SEQ IDs 1-547 set forth in Table 1 below. In embodiments, the treatment of a coronavirus (e.g., COVID 19) further includes antiviral therapy or immunotherapy.

In certain aspects, a T cell receptor (TCR) comprises an alpha and beta chain, wherein the alpha and beta chain SEQ ID NOS: 1-13 or 545-547. In certain aspects, aspects, the TCR further comprises a hinge domain, a transmembrane domain, an intracellular domain, a co-stimulatory domain, and/or a CD3ζ signaling domain.

In certain aspects, provided herein is an engineered cell expressing a T cell receptor (TCR) comprising an alpha and beta chain, wherein the alpha and beta chain comprises SEQ ID NOS: 1-13 or 545-547. In certain aspects, aspects, the TCR further comprises a hinge domain, a transmembrane domain, an intracellular domain, a co-stimulatory domain, and/or a CD3ζ signaling domain. In certain aspects, the co-stimulatory domain comprises CD28, 41BB or fragments thereof. In certain embodiments, the cell comprises an immune cell, wherein the immune cell comprises natural killer (NK) cells or T cells.

In certain aspects, a T cell receptor (TCR) alpha chain comprises amino acid sequences of: CAVRGSGGSNYKLTF, CAFMKPRGSQGNLIF, CALSEGNYGGSQGNLIF, CAGRQGAQKLVF, CAVSSSGGSYIPTF, CAVTKPSGTALIF, CIESNTGKLIF, CAVSLTYKYIF, CAMTVNSGYSTLTF, CAENSGTYKYIF, CAENGSRDTGRRALTF, CIVRDNAGNMLTF, CVVKSRGGGGKLIF, CAVRTYGQNFVF, CAGFNYGGSQGNLIF or CAGRVYNNNDMRF.

In certain aspects, a T cell receptor (TCR) comprises an alpha chain comprising an amino acid sequence of: CAVRGSGGSNYKLTF, CAFMKPRGSQGNLIF, CALSEGNYGGSQGNLIF, CAGRQGAQKLVF, CAVSSSGGSYIPTF, CAVTKPSGTALIF, CIESNTGKLIF, CAVSLTYKYIF, CAMTVNSGYSTLTF, CAENSGTYKYIF, CAENGSRDTGRRALTF, CIVRDNAGNMLTF, CVVKSRGGGGKLIF, CAVRTYGQNFVF, CAGFNYGGSQGNLIF or CAGRVYNNNDMRF and a beta chain comprising SEQ ID NOS: 1-546 or 547.

In other aspects, the invention includes a composition including at least one sequence of SEQ ID NOs: 1-547 to diagnose, treat or monitor COVID-19 disease.

In further aspects, methods for treating a subject having a COVID-19 infection, exhibiting symptoms of a COVID-19 infection, or having suspected exposure to COVID-19 are provided. The methods in general comprise administering an effective of a composition disclosed herein to a subject in needed thereof, such as a human subject that has tested positive for COVID-19, or is suspected to having been exposed to COVID-19.

In further aspects, methods are provided for treating cells infected by or exposed to COVID-19. These methods in general comprise administering to the infected or exposed cells (such as mammalian cells, particularly human cells) an effective amount of a composition as disclosed herein.

Other aspects of the invention are disclosed infra.

### DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1H shows data of the detection of memory CD4⁺ T cell clonotypes that cross-recognize SARS-CoV-2 and CCC spike proteins. The ViraFEST assay detected cross-reactivity to the S protein from SARS-CoV-2 and at least one other CCC (HCoV-NL63, HCoV-OC43, HCoV-229E, and HCoV-HKU1) in 8 out of 12 CCPs tested (FIGS. 1A-1H). Peptide co-culture was done in triplicate unless otherwise noted. Data are shown as the frequency (%) after culture (y-axis) of antigen-specific CD4+ T cell clonotypes (z-axis) for all peptide pools tested (x-axis). The top five mono-reactive TCR Vβ CDR3 amino acid clonotypes for each CCP are shown. Solid green bars represent significant (FDR<0.05) clonotypic expansion in response to the indicated antigenic peptide pool(s), whereas translucent green color indicates the clonotype was present at low frequency in the well, but did not significantly expand. Grey indicates the relevant TCR clonotype was not detected in that well. An identical, shared TCR Vβ CDR3 amino acid clonotype corresponding to cross-reactive memory CD4+ T cells in CCP4, CCP5, and CCP6 is shown in red (FIG. 1B, FIG. 1C, and FIG. 1D). Single cell TCR sequencing was performed on stimulated CD4+ T cells from CCP4 to identify the cognate TCR a chain for TCR β chains of interest (*).

FIGS. 2A-2D are data showing the functional validation and avidity of SARS-CoV-2 and HCoV-NL63 S-reactive TCRs. Single cell TCR sequencing was performed to identify the cognate TCRα of SARS-CoV-2/HCoV-NL63 cross-reactive (FIGS. 2A and 2B) and SARS-CoV-2 mono-reactive (FIGS. 2C and 2D) TCRβ clonotypes. TCRs were cloned and transfected into a Jurkat NFAT-luciferase reporter cell line. Jurkat cells expressing TCRs of interest were co-cultured with patient lymphoblastic cell lines (LCL) and SARS-CoV-2 S or HCoV-NL63 S peptide pools in titrating concentrations of S protein peptide pools (FIG. 2A and FIG. 2C) or the mapped 17-mer (FIG. 2B and FIG. 2D). Data are shown as relative luminescence units (RLU) at each pool or peptide concentration. Functional avidity for the specific 17-mer epitope was determined by calculating an EC50 (concentration at which response was 1/2 of maximum RLU, black x).

FIGS. 3A-3C are data showing the functional activity of SARS-CoV-2 S cross-reactive and mono-reactive CD4+ T cell responses. A heatmap was generated to visualize the fold change of SARS-CoV-2/CCC cross-reactive (red) and SARS-CoV-2 mono-reactive (blue) T cell clonotypes relative to the negative control (FIG. 3A). Each row represents a single TCR Vβ CDR3 clonotype and each column represents a peptide pool. To account for clones that were only present in one condition, a pseudocount of I was added to all clonotype counts and frequency was recalculated. Fold change was then computed by dividing frequency in response to a peptide of interest (i.e., SARS-CoV-2 S or CCC S) over the negative control (HIV). Red represents high fold change and blue represents low fold change. The average fold change was calculated to compare overall functional avidity of cross-reactive clones for SARS-CoV-2 S relative to CCC S (n=51), where the highest fold change across any recognized CCC was used for each clonotype (FIG. 3B), and to compare relative functional activity for the SARS-CoV-2 pool between cross-reactive (n=51) and mono-reactive clones (n=40); FIG. 3C). Each dot represents a SARS-CoV-2 S-reactive clonotype from a sample with grey line indicating a pair. One-sided Wilcoxon signed-rank test was performed to compare the fold change of cross-reactive clones in response to CCC S vs. SARS-CoV-2 S. One-sided Mann-Whitney U test was used to compare the fold change of SARS-CoV-2 S mono-reactive vs. cross-reactive clones. *, P<0.05; **, P≤0.01; ***, P<0.001; ****: P≤0.0001.

FIGS. 4A-4C are data showing the detection of recall SARS-CoV-2/CCC cross-reactive CD4⁺ T cells in unexposed donors. The ViraFEST assay was used to probe peripheral blood CD4⁺ T cells obtained prior to the COVID-19 pandemic (2011-2018) for reactivity to SARS-CoV-2 S and N as well as CCCs (HCoV-NL63, HCoV-OC43, HCoV-229E, and HCoV-HKU1) as cell number allowed. The assay was performed in triplicate unless otherwise noted. Responses were detected in PC3 (FIG. 4A) and JH014 (FIG. 4B). ViraFEST was also performed on cells obtained from JH014 during the pandemic, in July 2020, and SARS-CoV-2/CCC cross-reactive responses were again detected (FIG. 4C). Data are shown as the frequency (%) after culture (y-axis) of antigen-specific CD4+T cell clonotypes (z-axis) for all peptide pools tested (x-axis). Solid green bars represent significant (FDR<0.05) clonotypic expansion in response to the indicated antigenic peptide pool(s), whereas translucent green color indicates the clonotype was present at low frequency in the well but did not significantly expand. Grey indicates the relevant TCR clonotype was not detected in that well. An identical, shared TCR Vβ CDR3 amino acid clonotype corresponding to cross-reactive memory CD4+ T cells, previously identified in CCP4, CCP5, and CCP6, is shown in red.

FIG. 5 is a heatmap showing shared sequence homology of SARS-CoV-2/CCC cross-reactive TCRs within and among patients. A heatmap was generated to visualize sequence homology of cross-reactive clones within and between the patients in our study. Each row/column represents a cross-reactive clonotype in a sample. The color represents the levels of sequence homology using Levenshtein distance, with red indicating high degree of sequence homology and blue for less sequence similarity. Different colors were used to distinguish patient samples in the color sidebar. The black box highlights an area of high sequence homology across multiple patients with Levenshtein distance ≤3.

FIGS. 6A and 6B are data showing the prevalence of SARS-CoV-2/CCC cross-reactive TCR clonotypes. The TCR Vβ CDR3 clonotypes were used and identified in the ViraFEST assay to assess the distribution of percentage of patients having cross-reactive clones in pre-COVID (orange) and COVID-era (red) datasets (FIG. 6A). Additionally, the change in productive frequency in TCR-seq samples was queried of patients pre-COVID (orange) and COVID-era (red) (FIG. 6B). The productive frequency was plotted on a log 10 scale. Comparison of the productive frequency of each TCR Vb clonotype between pre-COVID and COVID populations was performed using the Mann-Whitney U test with multiple testing correction. *, FDR≤0.05; **, FDR≤0.01; ***, FDR≤0.001; ** FDR≤0.0001.

FIGS. 7A-7L are data showing SARS-CoV-2 and CCC memory CD4+ T cell responses in COVID-19 convalescent patients. The ViraFEST assay was performed to test for reactivity to SARS-CoV-2 and CCC (HCoV-NL63, HCoV-OC43, HCoV-229E, and HCoV-HKU1) spike glycoprotein pools in COVID-19 convalescent patients (CCPs; FIGS. 7A-7L). Peptide co-culture was done in triplicate unless otherwise noted. Data are shown as the frequency (%) after culture (y-axis) of antigen-specific CD4+ T cell clonotypes (z-axis) for all peptide pools tested (x-axis). The top five mono-reactive TCR Vβ CDR3 amino acid clonotypes for each CCP are shown. Solid color represents significant (FDR<0.05) clonotypic expansion in response to the indicated antigenic peptide pool(s), whereas translucent color indicates the clonotype was present at low frequency in the well, but did not significantly expand. Grey indicates the relevant TCR clonotype was not detected in that well.

FIGS. 8A-8E are data showing mono-reactive responses to SARS-CoV-2 and CCC in pre-COVID specimens. The ViraFEST assay was used to query CD4+ T cells obtained from 4 donors (FIGS. 8A-8D) prior to the COVID-19 pandemic for reactivity to SARS-CoV-2 and CCC. Peptide stimulations were performed in triplicate unless otherwise noted. Data are shown as the frequency (%) after culture (y-axis) of antigen-specific CD4+ T cell clonotypes (z-axis) for all peptide pools tested (x-axis). Solid color represents significant (FDR<0.05) clonotypic expansion in response to the indicated antigenic peptide pool(s), whereas translucent color indicates the clonotype was present at low frequency in the well, but did not significantly expand. Grey indicates the relevant TCR clonotype was not detected in that well. Monoreactive responses are also shown in CD4+ T cells obtained from JH014 in July 2020, during the COVID-19 pandemic (FIG. 8E).

FIGS. 9A-9J are data showing cross-reactive CD4+ T cell responses in CCPs. CD4+ T cell responses cross-reactive for CCC are shown for CCP6 (FIG. 9A), CCP7 (FIG. 9B), CCP9 (FIG. 9C), CCP10 (FIG. 9D), CCP11 (FIG. 9E), CCP12 (FIG. 9F), JH014 during the COVID-19 pandemic (FIG. 9G), and the pre-treatment specimens obtained from JH014 (FIG. 9H), PC2 (FIG. 9I), and PC3 (FIG. 9J). Peptide stimulations were performed in triplicate unless otherwise noted. Data are shown as the frequency (%) after culture (y-axis) of antigen-specific CD4+ T cell clonotypes (z-axis) for all peptide pools tested (x-axis). Solid color represents significant (FDR<0.05) clonotypic expansion in response to the indicated antigenic peptide pool(s), whereas translucent color indicates the clonotype was present at low frequency in the well, but did not significantly expand. Grey indicates the relevant TCR clonotype was not detected in that well.

FIGS. 10A-10E are data showing 17-mer peptide was defined for five cross-reactive (FIGS. 10A-10C) and three mono-reactive (FIG. 10D and FIG. 10E) TCRs identified in CCP4. Single cell TCRseq was performed on cultured cells from CCP4 to enumerate the cognate alpha chain corresponding to the mono-reactive TCR beta chain. TCRs were cloned into a jurkat NFAT luciferase reporter system. TCR-transfected CD4+ jurkat cells were screened for reactivity to mini-pools of 10-peptides each (FIG. 10A, FIG. 10B, FIG. 10D). After the positive mini pool was identified, TCR-transfected jurkats were screened for reactivity to each individual peptide within the positive mini pool (Table 2) to determine the exact stimulating 17-mer (FIG. 10C and FIG. 10E).

FIGS. 11A-11B are data showing stimulating epitope regions in SARS-CoV-2 spike glycoprotein. The position in the SARSCoV-2 proteome is shown for regions eliciting CD4+ T cell cross-reactivity (FIG. 11A) and mono-reactivity (FIG. 11B). Nterminal-domain (NTD), structural domain signal peptide (SP), receptor-binding domain (RBD), subdomain (SD), cleavage loop (CL), upstream helix (UH), fusion peptide (FP), connecting region (CR), heptad repeat (HR), central helix (CH), β-hairpin (BH), transmembrane region (TM), and cytoplasmic part (CP).

FIG. 12 are data showing the validation of SARS-CoV-2 mono-reactive TCRs. High concentration peptide titrations were used validate the mono-reactivity of the SARS-CoV-2-reactive TCRs validated in FIGS. 10A-10E. Peptide concentrations ranging from 50 ug/ml-12.5 ug/ml of SARS-CoV-2 S and CCC S peptide pools for NL63, OC4S, and 229E.

FIG. 13 are data showing shared sequence homology of CCC-reactive TCRs between patients. An unrooted phylogenetic tree was generated for all cross-reactive clonotypes from the patients in our study. Each leaf represents a cross-reactive clonotype in a sample and different colors were used to distinguish patient samples. The black box highlights an area of high sequence homology across multiple patients as shown in FIG. 5.

FIG. 14 is a heatmap showing shared sequence homology of SARS-CoV-2 S mono-reactive TCRs within and among patients. A heatmap was generated to visualize sequence homology for SARS-CoV-2 S mono-reactive clones. Each row/column represents a SARS-CoV-2 S mono-reactive clonotype in a sample. The color represents the levels of sequence homology using levenshtein distance, with red indicating a high degree of sequence homology and blue for less sequence similarity. Different colors were used to distinguish patient samples in the color sidebar. Area of high sequence homology within patients were amplified in bottom left and area of interest showing high sequence homology across patients were amplified in bottom right.

FIG. 15 is a heatmap showing TCRs specific for CMV, EBV, and flu share sequence homology between patients. A heatmap was generated to visualize sequence homology of CEF-specific clones. Each row/column represents a CEFreactive clonotype in a sample. The color represents the levels of sequence homology using levenshtein distance, with red indicating high degree of sequence homology and blue for less sequence similarity. Different colors were used to distinguish patient samples in the color sidebar. Area of high sequence homology across patients were amplified in the bottom.

FIGS. 16A-16B are data showing the prevalence of SARS-CoV-2 S mono-reactive TCR clonotypes. The TCR Vβ CDR3 clonotypes were used and identified in the ViraFEST assay to assess the distribution of percentage of patients having SARSCoV-2 S mono-reactive clones in pre-COVID (orange) and COVID-era (red) datasets (FIG. 16A). Additionally, the change in productive frequency was queried in TCRseq samples of patients pre-COVID (orange) and COVIDera (red) (FIG. 16B). The productive frequency was plotted on a log 10 scale. Comparison of productive frequency between pre-COVID and COVID-era for each clonotype were evaluated using the Mann-Whitney U test with multiple testing correction. *, FDR<=0.05; **, FDR<=0.01; ***, FDR<=0.001; ****: FDR<=0.0001.

FIGS. 17A-17B are data showing the prevalence of SARS-CoV-2 N mono-reactive TCR clonotypes. The TCR Vβ CDR3clonotypes were used and identified in the ViraFEST assay to assess the distribution of percentage of patients having SARSCoV-2 N mono-reactive clones in pre-COVID (orange) and COVID-era (red) datasets (FIG. 17A). Additionally, the change in productive frequency was queried in TCR-seq samples of patients pre-COVID (orange) and COVIDera (red) (FIG. 17B). The productive frequency was plotted on a log 10 scale. Comparison of productive frequency between pre-COVID and COVID-era for each clonotype were evaluated using the Mann-Whitney U test with multiple testing correction. *, FDR<=0.05; **, FDR<=0.01; ***, FDR<=0.001; ****: FDR<=0.0001.

FIGS. 18A-18C are graphs showing the comparison of prevalence between pre-COVID and COVID for cross-reactive, SARS-CoV-2 S mono-reactive and SARS-CoV-2 N mono-reactive clones. The TCR Vβ CDR3 clonotypes identified were used in the ViraFEST assay to assess the distribution of percentage of patients in three categories: crossreactive (FIG. 18A), SARS-CoV-2 S mono-reactive (FIG. 18B) and SARS-CoV-2 N mono-reactive (FIG. 18C), in pre-COVID (orange) and COVID-era (red) datasets. Each dot represents a queried clonotype in each category and grey line indicates a pair (i.e., identical clonotype). Comparison of prevalence between pre-COVID and post-COVID for each category were evaluated using the Wilcoxon signed-rank test with multiple testing correction. ****: P<=0.0001.

### DETAILED DESCRIPTION

Provided herein are, inter alia, methods, compositions and kits for treating and preventing coronaviruses, specifically COVID-19.

The present invention identifies T cell receptors (e.g., TCRs; SEQ IDs 1-547) recognizing the SARS-CoV-2 spike(S) and nucleocapsid (N) proteins, many of which are public (shared among many unrelated patients). The present invention covers an isolated nucleic acid molecule encoding these TCRs, a T cell expressing these TCRs, a therapeutic for use in the treatment of cells expressing or infected with SARS CoV-2 S and/or N proteins, and diagnosis and monitoring of SARS-CoV-2 infection. The TCRs can be used, for example, to treat patients infected with SARS-CoV-2, to diagnose disease based on clonal dynamics of SARS-CoV-2-specific TCRs, or to monitor vaccine efficacy.

T cell receptor-based therapy, diagnostics, and biomarkers are not currently used for the management of COVID-19. This is owing to the paucity of data on the clonal identity of SARS-CoV-2-specific T cells. That is, the identity of TCRs corresponding to SARS-CoV-2-reactive T cells have not been identified. The present invention addresses this knowledge gap by discovering such TCRs.

The TCRs corresponding to SEQ IDs 1-547 may be used to predict disease severity and clinical outcome, may be used to inform clinical decision making, may be used to test for recent SARS-CoV-2 infection or exposure, could be used to stratify patients for experimental therapies, and could be used to monitor and predict SARS-CoV-2 vaccine efficacy.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

The term “disease” refers to any deviation from the normal health of a mammal and includes a state when disease symptoms are present, as well as conditions in which a deviation (e.g., SARS-CoV-2) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B; ” “one or more of A and B; ” and “A and/or B” are each intended to mean “A alone, B alone, or A and B together.” A similar interpretation is also intended for lists including three or more items. For example, the phrases “at least one of A, B, and C; ” “one or more of A, B, and C; ” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg. 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (RNA or DNA) is free of the genes or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

A “control” sample or value refers to a sample that serves as a reference, usually a known reference, for comparison to a test sample. For example, a test sample can be taken from a test subject, e.g., a subject with SARS-CoV-2, and compared to samples from known conditions, e.g., a subject (or subjects) that does not have SARS-CoV-2 (a negative or normal control), or a subject (or subjects) who does have SARS-CoV-2 (positive control). A control can also represent an average value gathered from a number of tests or results. One of skill in the art will recognize that controls can be designed for assessment of any number of parameters. One of skill in the art will understand which controls are valuable in a given situation and be able to analyze data based on comparisons to control values. Controls are also valuable for determining the significance of data. For example, if values for a given parameter are variable in controls, variation in test samples will not be considered as significant.

The term, “normal amount” with respect to a compound (e.g., a protein or mRNA) refers to a normal amount of the compound in an individual who does not have SARS-CoV-2 in a healthy or general population. The amount of a compound can be measured in a test sample and compared to the “normal control” level, utilizing techniques such as reference limits, discrimination limits, or risk defining thresholds to define cutoff points and abnormal values (e.g., for SARS-CoV-2 or a symptom thereof). The normal control level means the level of one or more compounds or combined compounds typically found in a subject known not suffering from SARS-CoV-2. Such normal control levels and cutoff points may vary based on whether a compounds is used alone or in a formula combining with other compounds into an index. Alternatively, the normal control level can be a database of compounds patterns from previously tested subjects who did not develop SARS-CoV-2 or a particular symptom thereof (e.g., in the event the SARS-CoV-2 develops or a subject already having SARS-CoV-2 is tested) over a clinically relevant time horizon.

The level that is determined may be the same as a control level or a cut off level or a threshold level, or may be increased or decreased relative to a control level or a cut off level or a threshold level. In some aspects, the control subject is a matched control of the same species, gender, ethnicity, age group, smoking status, body mass index (BMI), current therapeutic regimen status, medical history, or a combination thereof, but differs from the subject being diagnosed in that the control does not suffer from the disease (or a symptom thereof) in question or is not at risk for the disease.

Relative to a control level, the level that is determined may an increased level. As used herein, the term “increased” with respect to level (e.g., protein or mRNA level) refers to any % increase above a control level. In various embodiments, the increased level may be at least or about a 5% increase, at least or about a 10% increase, at least or about a 15% increase, at least or about a 20% increase, at least or about a 25% increase, at least or about a 30% increase, at least or about a 35% increase, at least or about a 40% increase, at least or about a 45% increase, at least or about a 50% increase, at least or about a 55% increase, at least or about a 60% increase, at least or about a 65% increase, at least or about a 70% increase, at least or about a 75% increase, at least or about a 80% increase, at least or about a 85% increase, at least or about a 90% increase, at least or about a 95% increase, relative to a control level.

Relative to a control level, the level that is determined may a decreased level. As used herein, the term “decreased” with respect to level (e.g., protein or mRNA level) refers to any % decrease below a control level. In various embodiments, the decreased level may be at least or about a 5% decrease, at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, relative to a control level.

The terms “polypeptide,” “peptide” and “protein” are used interchangeably herein to refer to a polymer of amino acid residues, wherein the polymer may in embodiments be conjugated to a moiety that does not consist of amino acids. The terms also apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed or chemically synthesized as a single moiety.

“Polypeptide fragment” refers to a polypeptide that has an amino-terminal and/or carboxy-terminal deletion, in which the remaining amino acid sequence is usually identical to the corresponding positions in the naturally-occurring sequence. Fragments typically are at least 5, 6, 8 or 10 amino acids long, at least 14 amino acids long, at least 20 amino acids long, at least 50 amino acids long, or at least 70 amino acids long.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. In embodiments, the percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. In embodiments, two sequences are 100% identical. In embodiments, two sequences are 100% identical over the entire length of one of the sequences (e.g., the shorter of the two sequences where the sequences have different lengths). In embodiments, identity may refer to the complement of a test sequence. In embodiments, the identity exists over a region that is at least about 10 to about 100, about 20 to about 75, about 30 to about 50 amino acids or nucleotides in length. In embodiments, the identity exists over a region that is at least about 50 amino acids or nucleotides in length, or more preferably over a region that is 100 to 500, 100 to 200, 150 to 200, 175 to 200, 175 to 225, 175 to 250, 200 to 225, 200 to 250 or more amino acids or nucleotides in length.

For sequence comparison, typically one sequence acts as a reference sequence, to which test sequences are compared. In embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In embodiments, a comparison window is the entire length of one or both of two aligned sequences. In embodiments, two sequences being compared comprise different lengths, and the comparison window is the entire length of the longer or the shorter of the two sequences. In embodiments relating to two sequences of different lengths, the comparison window includes the entire length of the shorter of the two sequences. In embodiments relating to two sequences of different lengths, the comparison window includes the entire length of the longer of the two sequences.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Non-limiting examples of algorithms that are suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information (NCBI), as is known in the art. An exemplary BLAST algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0)) and N (penalty score for mismatching residues; always <0)). For amino acid sequences, a scoring matrix is used to calculate the cumulative score. Extension of the word hits in each direction are halted when: the cumulative alignment score falls off by the quantity X from its maximum achieved value; the cumulative score goes to zero or below; due to the accumulation of one or more negative-scoring residue alignments; or the end of either sequence is reached. The BLAST algorithm parameters W, T, and X determine the sensitivity and speed of the alignment. In embodiments, the NCBI BLASTN or BLASTP program is used to align sequences. In embodiments, the BLASTN or BLASTP program uses the defaults used by the NCBI. In embodiments, the BLASTN program (for nucleotide sequences) uses as defaults: a word size (W) of 28; an expectation threshold (E) of 10; max matches in a query range set to 0; match/mismatch scores of 1,-2; linear gap costs; the filter for low complexity regions used; and mask for lookup table only used. In embodiments, the BLASTP program (for amino acid sequences) uses as defaults: a word size (W) of 3; an expectation threshold (E) of 10; max matches in a query range set to 0; the BLOSUM62 matrix (see Henikoff & Henikoff, Proc. Natl. Acad. Sci. USA 89:10915 (1992)); gap costs of existence: 11 and extension: 1; and conditional compositional score matrix adjustment.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

“Nucleic acid” refers to nucleotides (e.g., deoxyribonucleotides, ribonucleotides, and 2′-modified nucleotides) and polymers thereof in either single-, double- or multiple-stranded form, or complements thereof. The terms “polynucleotide,” “oligonucleotide,” “oligo” or the like refer, in the usual and customary sense, to a linear sequence of nucleotides. The term “nucleotide” refers, in the usual and customary sense, to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g. mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent, or other interaction.

The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580), CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

“Operably linked” refers to a juxtaposition wherein the components so described are in a relationship permitting them to function in their intended manner. A control sequence “operably linked” to a coding sequence is ligated in such a way that expression of the coding sequence is achieved under conditions compatible with the control sequences

As may be used herein, the terms “nucleic acid,” “nucleic acid molecule,” “nucleic acid oligomer,” “oligonucleotide,” “nucleic acid sequence,” “nucleic acid fragment” and “polynucleotide” are used interchangeably and are intended to include, but are not limited to, a polymeric form of nucleotides covalently linked together that may have various lengths, either deoxyribonucleotides and/or ribonucleotides, and/or analogs, derivatives or modifications thereof. Different polynucleotides may have different three-dimensional structures, and may perform various functions, known or unknown. Non-limiting examples of polynucleotides include genomic DNA, a genome, mitochondrial DNA, a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may comprise natural nucleic acid sequences and variants thereof, artificial nucleic acid sequences, or a combination of such sequences.

The term “amino acid residue,” as used herein, encompasses both naturally-occurring amino acids and non-naturally-occurring amino acids. Examples of non-naturally occurring amino acids include, but are not limited to, D-amino acids (i.e. an amino acid of an opposite chirality to the naturally-occurring form), N-α-methyl amino acids, C-α-methyl amino acids, β-methyl amino acids and D- or L-β-amino acids. Other non-naturally occurring amino acids include, for example, β-alanine (β-Ala), norleucine (Nle), norvaline (Nva), homoarginine (Har), 4-aminobutyric acid (γ-Abu), 2-aminoisobutyric acid (Aib), 6-aminohexanoic acid (8-Ahx), ornithine (orn), sarcosine, α-amino isobutyric acid, 3-aminopropionic acid, 2,3-diaminopropionic acid (2,3-diaP), D- or L-phenylglycine, D-(trifluoromethyl)-phenylalanine, and D-p-fluorophenylalanine.

As used herein, “peptide bond” can be a naturally-occurring peptide bond or a non-naturally occurring (i.e. modified) peptide bond. Examples of suitable modified peptide bonds are well known in the art and include, but are not limited to, —CH₂NH—, —CH₂S—, —CH₂CH₂—, —CH═CH— (cis or trans), —COCH₂—, —CH(OH)CH₂—, —CH₂SO—, —CS—NH— and —NH—CO— (i.e. a reversed peptide bond) (see, for example, Spatola, Vega Data Vol. 1, Issue 3, (1983); Spatola, in Chemistry and Biochemistry of Amino Acids Peptides and Proteins, Weinstein, ed., Marcel Dekker, New York, p. 267 (1983); Morley, J. S., Trends Pharm. Sci. pp. 463-468 (1980); Hudson et al., Int. J. Pept. Prot. Res. 14:177-185 (1979); Spatola et al., Life Sci. 38:1243-1249 (1986); Hann, J. Chem. Soc. Perkin Trans. 1 307-314 (1982); Almquist et al., J. Med. Chem. 23:1392-1398 (1980); Jennings-White et al., Tetrahedron Lett. 23:2533 (1982); Szelke et al., EP 45665 (1982); Holladay et al., Tetrahedron Lett. 24:4401-4404 (1983); and Hruby, Life Sci. 31:189-199 (1982))

A polynucleotide is typically composed of a specific sequence of four nucleotide bases: adenine (A); cytosine (C); guanine (G); and thymine (T) (uracil (U) for thymine (T) when the polynucleotide is RNA). Thus, the term “polynucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Polynucleotides may optionally include one or more non-standard nucleotide(s), nucleotide analog(s) and/or modified nucleotides.

An antibody described herein may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice, or human), e.g., the antibody comprises a monoclonal antibody.

An “antagonist antibody” or a “blocking antibody” is one that inhibits or reduces a biological activity of the antigen it binds to. In some embodiments, blocking antibodies or antagonist antibodies substantially or completely inhibit the biological activity of the antigen. Alternatively, an “agonist” or activating antibody is one that enhances or initiates signaling by the antigen to which it binds. In some embodiments, agonist antibodies cause or activate signaling without the presence of the natural ligand

An “isolated antibody,” as used herein, is intended to refer to an antibody that is substantially free of other antibodies having different antigenic specificities. Moreover, an isolated antibody may be substantially free of other cellular material and/or chemicals.

The antibody of the present invention may be a polyclonal antisera or monoclonal antibody. The term antibody may include any of the various classes or sub-classes of immunoglobulin (e.g., IgG, IgA, IgM, IgD, or IgE derived from any animal, e.g., any of the animals conventionally used, e.g., sheep, rabbits, goats, or mice). Preferably, the antibody comprises a monoclonal antibody.

The terms “monoclonal antibody” or “monoclonal antibody composition” as used herein refer to a preparation of antibody molecules of single molecular composition. A monoclonal antibody composition displays a single binding specificity and affinity for a particular epitope.

An “antibody fragment” comprises a portion of an intact antibody, preferably the antigen binding and/or the variable region of the intact antibody. Non-limiting examples of antibody fragments include Fab, Fab*, F (ab′)2 and Fv fragments; diabodies; linear antibodies; single-chain antibody molecules and multispecific antibodies formed from antibody fragments

The invention may further comprise a humanized antibody, wherein the antibody is from a non-human species, whose protein sequence has been modified to increase their similarity to antibody variants produced naturally in humans. Generally, a humanized antibody has one or more amino acid residues introduced into it from a source which is non-human. These non-human amino acid residues are referred to herein as “import” residues. which are typically taken from an “import” antibody domain, particularly a variable domain.

The term “recombinant human antibody,” as used herein, includes all human antibodies that are prepared, expressed, created or isolated by recombinant means, such as (a) antibodies isolated from animals (e.g., sheep, rabbits, goats, or mice) that are transgenic or transchromosomal for human immunoglobulin genes, (b) antibodies isolated from a host cell transformed to express the human antibody. (c) antibodies isolated from a recombinant, combinatorial human antibody library, and (d) antibodies prepared, expressed, created or isolated by any other means that involve splicing of human immunoglobulin gene sequences to other DNA sequences.

An “antibody fragment” comprises a portion of an intact antibody, preferably the antigen binding and/or the variable region of the intact antibody. Examples of antibody fragments include Fab, Fab*, F (ab′)2 and Fv fragments; diabodies; linear antibodies; single-chain antibody molecules and multispecific antibodies formed from antibody fragments. Papain digestion of antibodies produced two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F (ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F (ab′)2 antibody fragments originally were produced as pairs of Fab′ fragments which have hinge cysteines between them. Other chemical couplings of antibody fragments are also known.

By “antigen” is defined as a molecule that provokes an immune response. This immune response may involve either antibody production, or the activation of specific immunologically-competent cells, or both. For example, any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein

### T Cells

A T cell or T lymphocyte is a type of lymphocyte (a subtype of white blood cell) that plays a central role in cell-mediated immunity. T cells are distinguished from other lymphocytes, such as B cells and natural killer cells, by the presence of a T-cell receptor on the cell surface. T cells are so named because they mature in the thymus from thymocytes (although some also mature in the tonsils).

The several subsets of T cells each have a distinct function. There are many types of T cells including effector T cells, T helper cells, cytotoxic (killer) T cells, memory T cells, regulatory T cells, natural killer cells, gamma delta T cells, and mucosal associated invariant T cells. The majority of human T cells rearrange their alpha and beta chains on the cell receptor and are termed alpha beta T cells (.alpha.beta. T cells) and are part of the adaptive immune system. Specialized gamma delta T cells, a small minority of T cells in the human body, more frequent in ruminants, have invariant T cell receptors with limited diversity that can effectively present antigens to other T cells and are considered to be part of the innate immune system.

A unique feature of T cells is their ability to discriminate between healthy and abnormal (e.g. infected or cancerous) cells in the body. Healthy cells typically express a large number of self derived peptide-loaded major histocombatibility complex (pMHC) on their cell surface and although the T cell antigen receptor can interact with at least a subset of these self pMHC, the T cell generally ignores these healthy cells. However, when these very same cells contain even minute quantities of pathogen derived pMHC, T cells are able to become activated and initiate immune responses. The ability of T cells to ignore healthy cells, but respond when these same cells contain pathogen (or cancer) derived pMHC is known as antigen discrimination.

### T Cell Receptor

The T-cell receptor, or TCR, is a molecule found on the surface of T cells, or T lymphocytes, that is responsible for recognizing fragments of antigen as peptides bound to MHC molecules. The binding between TCR and antigen peptides is of relatively low affinity and is degenerate: that is, many TCRs recognize the same antigen peptide and many antigen peptides are recognized by the same TCR. The TCR is composed of two different protein chains (that is, it is a heterodimer). In humans, in 95% of T cells the TCR consists of an alpha (.alpha.) and beta (.beta.) chain, whereas in 5% of T cells the TCR consists of gamma and delta (.gamma./.delta.) chains. This ratio changes during ontogeny and in diseased states as well as in different species.

When the TCR engages with antigenic peptide and MHC (peptide/MHC), the T lymphocyte is activated through signal transduction, that is, a series of biochemical events mediated by associated enzymes, co-receptors, specialized adaptor molecules, and activated or released transcription factors.

The TCR is a disulfide-linked membrane-anchored heterodimeric protein normally consisting of the highly variable alpha (.alpha.) and beta (.beta.) chains expressed as part of a complex with the invariant CD3 chain molecules. T cells expressing this receptor are referred to as .alpha.:.beta. (or .alpha..beta.) T cells, though a minority of T cells express an alternate receptor, formed by variable gamma (.gamma.) and delta (.delta.) chains, referred as .gamma.delta. T cells. Each chain is composed of two extracellular domains: Variable (V) region and a Constant (C) region, both of Immunoglobulin superfamily (IgSF) domain forming antiparallel .beta.-sheets. The constant region is proximal to the cell membrane, followed by a transmembrane region and a short cytoplasmic tail, while the Variable region binds to the peptide/MHC complex. The constant domain of the TCR domain consists of short connecting sequences in which a cysteine residue forms disulfide bonds, which forms a link between the two chains.

The variable domain of both the TCR .alpha.-chain and .beta.-chain each have three hypervariable or complementarity determining regions (CDRs), whereas the variable region of the .beta.-chain has an additional area of hypervariability (HV4) that does not normally contact antigen and, therefore, is not considered a CDR. The residues are located in two regions of the TCR, at the interface of the .alpha.- and .beta.-chains and in the .beta.-chain framework region that is thought to be in proximity to the CD3 signal-transduction complex. CDR3 is the main CDR responsible for recognizing processed antigen, although CDR1 of the alpha chain has also been shown to interact with the N-terminal part of the antigenic peptide, whereas CDR1 of the n-chain interacts with the C-terminal part of the peptide. CDR2 recognizes the MHC. CDR4 of the 1-chain does not participate in antigen recognition, but has been shown to interact with superantigens.

Processes for the generation of TCR diversity are based mainly on genetic recombination of the DNA encoded segments in individual somatic T cells—either somatic V(D)J recombination using recombinant activating gene 1 (RAG1) and RAG2 recombinases or gene conversion using cytidine deaminases (AID). Each recombined TCR possess unique antigen specificity, determined by the structure of the antigen-binding site formed by the .alpha. and .beta. chains in case of .alpha..beta. T cells or .gamma. and .delta. chains on case of .gamma..delta. T cells. The TCR alpha chain is generated by VJ recombination, whereas the beta chain is generated by VDJ recombination (both involving a somewhat random joining of gene segments to generate the complete TCR chain). Likewise, generation of the TCR gamma chain involves VJ recombination, whereas generation of the TCR delta chain occurs by VDJ recombination. The intersection of these specific regions (V and J for the alpha or gamma chain; V, D, and J for the beta or delta chain) corresponds to the CDR3 region that is important for peptide/MHC recognition.

### Methods for Diagnosing SARS-CoV-2

Included herein is a method of diagnosing a coronavirus, e.g., COVID19 in a subject in need thereof. In further embodiments, the method comprises administering to the subject an effective amount of the composition comprising a composition having a T cell receptor (TCR) with an alpha and/or beta chain comprising of SEQ IDs 1-547, or a TCR having an alpha chain with the sequence of SEQ ID NO: 1-13 or 545-547, or a TCR having a beta chain. For example, methods diagnosing coronavirus, e.g., COVID19 include administering a composition comprising a composition having a T cell receptor (TCR) with an alpha and/or beta chain comprising of SEQ IDs 1-547.

### Methods for Treating SARS-CoV-2

Included herein is a method of preventing or treating a coronavirus, e.g., COVID19 in a subject in need thereof. In further embodiments, the method comprises administering to the subject an effective amount of the composition comprising a composition having a T cell receptor (TCR) with an alpha and/or beta chain comprising of SEQ IDs 1-547, or a TCR having an alpha chain with the sequence of SEQ ID NO: 1-13 or 545-547. For example, methods for preventing or treating coronavirus, e.g., COVID19 include administering a composition comprising a composition having a T cell receptor (TCR) with an alpha and/or beta chain comprising of SEQ IDs 1-547, or a TCR having an alpha chain with the sequence of SEQ ID NO: 1-13 or 545-547.

In other embodiments, the methods for treating coronavirus, e.g., COVID19 comprise administering to a subject a composition comprising a composition having a T cell receptor (TCR) with an alpha and/or beta chain comprising of SEQ IDs 1-547, or a TCR having an alpha chain with the sequence of SEQ ID NO: 1-13 or 545-547 produced according to the methods described herein, in combination with methods for controlling the outset of symptoms. In particular, the combination treatment can include administering readily known treatments. Additionally, combination therapy may include antiviral or immunotherapy treatment (therapy). In embodiments, the combination therapy may include administration of an antiviral agent, or immunotherapy compositions, e.g., engineered CART cells.

The described composition can be administered as a pharmaceutically or physiologically acceptable preparation or composition containing a physiologically acceptable carrier, excipient, or diluent, and administered to the tissues of the recipient organism of interest, including humans and non-human animals.

The composition comprising a composition having a T cell receptor (TCR) with an alpha and/or beta chain comprising of SEQ IDs 1-547, or a TCR having an alpha chain with the sequence of SEQ ID NO: 1-13 or 545-547 can be prepared by re-suspending in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising a T cell receptor (TCR) with an alpha and/or beta chain comprising of SEQ IDs 1-547, or a TCR having an alpha chain with the sequence of SEQ ID NO: 1-13 or 545-547) is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

In embodiments, a therapeutically effective amount of the composition (e.g., a composition comprising a T cell receptor (TCR) with an alpha and/or beta chain comprising of SEQ IDs 1-547, or a TCR having an alpha chain with the sequence of SEQ ID NO: 1-13 or 545-547) in humans can be any therapeutically effective amount. In one embodiment, the composition (e.g., a composition comprising a T cell receptor (TCR) with an alpha and/or beta chain comprising of SEQ IDs 1-547, or a TCR having an alpha chain with the sequence of SEQ ID NO: 1-13 or 545-547) is administered thrice daily, twice daily, once daily, fourteen day's on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising a T cell receptor (TCR) with an alpha and/or beta chain comprising of SEQ IDs 1-547, or a TCR having an alpha chain with the sequence of SEQ ID NO: 1-13 or 545-547) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

Additional advantages of the methods described herein include that T cell receptor (TCR) with an alpha and/or beta chain comprising of SEQ IDs 1-547, or a TCR having an alpha chain with the sequence of SEQ ID NO: 1-13 or 545-547 can be injected systemically, as opposed to local delivery. Additional advantages include that patients requiring treatment typically require at least 1 local injections, and the injections are about 7 days apart. The compositions and methods described herein provide that patients require about 1 injection(s), systemically. In some examples, the injections can be every week.

### Pharmaceutical Compositions and Formulations

The present invention provides pharmaceutical compositions comprising an effective amount of a composition (e.g., a composition comprising a T cell receptor (TCR) having an alpha and/or beta chain comprising of SEQ IDs 1-547, or a TCR having an alpha chain with the sequence of SEQ ID NO: 1-13 or 545-547) and at least one pharmaceutically acceptable excipient or carrier, wherein the effective amount is as described above in connection with the methods of the invention.

In one embodiment, the composition (e.g., a composition comprising a composition having a T cell receptor (TCR) with an alpha and/or beta chain comprising of SEQ IDs 1-547, or a TCR having an alpha chain with the sequence of SEQ ID NO: 1-13 or 545-547) is further combined with at least one additional therapeutic agent in a single dosage form. In one embodiment, the at least one additional therapeutic agent comprises an antiviral agent.

Non-limiting examples of anti-viral agents that may be used in combination with a TCR as described herein include Remdesivir, Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In one embodiment, the composition (e.g., a composition comprising a composition having a T cell receptor (TCR) with an alpha and/or beta chain comprising of SEQ IDs 1-547, or a TCR having an alpha chain with the sequence of SEQ ID NO: 1-13 or 545-547) is further combined with at least one additional therapeutic agent in a single dosage form. In one embodiment, the at least one additional therapeutic agent comprises monoclonal antibody therapy.

Non-limiting examples of monoclonal antibody therapy that may be used in combination with a TCR as described herein include bamlanivimab, casirivimab, imdevimab, The monoclonal antibodies may be administered in combination or individually, e.g., by intravenous injection (IV).

In one embodiment, the composition (e.g., a composition comprising a composition having a T cell receptor (TCR) with an alpha and/or beta chain comprising of SEQ IDs 1-547, or a TCR having an alpha chain with the sequence of SEQ ID NO: 1-13 or 545-547) is further combined with at least one additional therapeutic agent in a single dosage form. In one embodiment, the at least one additional therapeutic agent comprises immunomodulators, interleukin 6 inhibitors, or kinase inhibitors. Non-limiting examples of immunomodulators include corticosteroids (e.g., dexamethasone), interferon α (FINα), interferon β (FINβ), interleukin inhibitors (e.g., anakinra). Non-limiting examples of interleukin 6 inhibitors (IL-6) include sarilumab, siltuximab, or tocilizumab. Non-limiting examples of kinase inhibitors include acalabrutinib, ibrutinib, or zanubrutinib, Janus kinase inhibitors (baricitinib, ruxolitinib, or tofacitinib).

In particular asepcts, agents that may be used in combination with a TCR as described hereinmay include one or more of hyperimmune globulins, remdesivir, oseltamivir, Galidesivir (BCX4430, Immucillin-A), 3-Deazaneplanocin A (DZNep, C-c3Ado), Favipiravir (T-705, Avigan), lopinavir; ritonavir, lopinavir/ritonavir (e.g. KALETRA), ribavirin, lopinavir/ritonavir/ribavirin, Recombinant human interferon α1β, Huaier (including Huaier Granule), Eculizumab (Soliris), Recombinant human angiotensin-converting enzyme 2 (rhACE2), Carrimycin, Umifenivir (Arbidol), chloroquine phosphate, T89 (Dantonic), Fingolimod (including Fingolimod 0.5 mg), N-acetylcysteine, N-acetylcysteine+ Fuzheng Huayu Tablet, YinHu QingWen Decoction, LV-SMENP-DC vaccine and/or antigen-specific CTLs; steroids such as dexamethasone; antibodies including monoclonal antibodies such as amlanivimab (LY-CoV555; Eli Lilly) and REGN-COV2 (Regeneron) and AZD7442 (antibody combination, AstraZenca); and/or immunosuppressive agent including IL-6 receptor agonists such as tocilizumab (IL-6 receptor agonist, Roche) and sarilumab (Sanofi).

In one embodiment, the composition (e.g., a composition comprising a composition having a T cell receptor (TCR) with an alpha and/or beta chain comprising of SEQ IDs 1-547, or a TCR having an alpha chain with the sequence of SEQ ID NO: 1-13 SEQ ID NO: 1-13 or 545-547) is further combined or unused in conjunction with monoclonal antibody therapy which may include administration of bamlanivimab.

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g. liquids, aerosols, solutions, inhalants, mists, sprays; or solids, powders, ointments, pastes, creams, lotions, gels, patches and the like) for administration by any desired route (e.g. pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value. All percentages and ratios used herein, unless otherwise indicated, are by weight.

Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits Comprising the TCR

In aspects, a kit for producing a T cell receptor (TCR) with an alpha and/or beta chain comprising of SEQ IDs 1-547, or a TCR having an alpha chain with the sequence of SEQ ID NO: 1-13 or 545-547is provided. In embodiments, the kit comprises the a T cell receptor (TCR) with an alpha and/or beta chain comprising of SEQ IDs 1-547, or a TCR having an alpha chain with the sequence of SEQ ID NO: 1-13 or 545-547 and reagents.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a coronavirus, e.g., COVID19), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: CD4+ T Cells From COVID-19 Convalescent Patients are Cross-Reactive for SARS-CoV-2 and CCC Spike Proteins

The central hypothesis provided that unique, individual CD4⁺ T cell clonotypes recognize both CCC and SARS-CoV-2 S proteins. This is supported by prior studies demonstrating that some SARS-CoV-2-unexposed donors have reactivity to this antigen (12-14, 16, 17). Bona fide cross-reactivity mediated by a single T cell clonotype expressing a unique T cell receptor (TCR) heterodimer has not yet been shown for T cells that target SARS-CoV-2. The functional expansion of specific T cells (FEST) assay identifies canonical antigen-specific memory T cell responses and the cognate TCR(s) contributing to this response via a 10-day T cell culture with relevant antigen followed by TCR Vj3 complementarity determining region 3 (CDR3) sequencing (24-28). This assay has also been used to identify TCRs cross-reactive for related viral epitope variants (ViraFEST) (29). The ViraFEST assay was used to probe peripheral blood CD4⁺ T cells from 12 convalescent COVID-19 patients (CCPs; Table 1, below) for cross-recognition of the S proteins from SARS-CoV-2 and four known CCC: HCoV-NL63-S, HCoV-OC43-S, HCoV-229E-S, and HCoV-HKU1-S.

SEQ IDs. 1-547 referred to herein are set forth in the following Table 1:

TABLE 1

| | | | | | | | | | | CoV2 | NL63 |
|---|---|---|---|---|---|---|---|---|---|---|---|
| | TCR | | | Vβ | Dβ | Jβ | Vα | Jα | | Peptide | Peptide |
| SEQ ID | Chain | CDR3 β | CDR3 α | Gene Usage | Gene Usage | Gene Usage | Gene Usage | Gene Usage | Antigen reactivity | Reactivity | Reactivity |
| 1 | Full | CATSVVSGVRTG | CAVRGSGGSNY | TRBV6-5 | | TRBJ2-2 | TRAV12-2 | TRAJ53 | SARS-SARS-COV-2/HCoV- | SKRSFIEDLLFNKV | IAGRSALEDLLFSK |
| | | ELFF | KLTF | | | | | | NL63 S cross reactive | TLA | VVT |
| 2 | Full | CASSRPGTGGAA | CAFMKPRGSQG | TRBV5-6 | | TRBJ1-2 | TRAV38-1 | TRAJ42 | SARS-SARS-COV-2/HCoV- | NA | NA |
| | | YGYTF | NLIF | | | | | | NL63 S cross reactive | | |
| 3 | Full | CASRTYPAARYN | CALSEGNYGGS | TRBV5-8 | | TRBJ2-1 | TRAV9-2 | TRAJ42 | SARS-SARS-COV-2/HCoV- | NA | NA |
| | | EQFF | QGNLIF | | | | | | NL63 S cross reactive | | |
| 4 | Full | CASRDTGRLETQ | CAGRQGAQKLV | TRBV28 | | TRBJ2-5 | TRAV35* | TRAJ54 | SARS-SARS-COV-2/HCoV- | SKRSFIEDLLFNKV | IAGRSALEDLLFSK |
| | | YF | F | | | | 02 | | NL63 S cross reactive | TLA | VVT |
| 5 | Full | CAWSVQQNYGYT | CAVSSSGGSYI | TRBV30 | | TRBJ1-2 | TRAV8- | TRAJ6 | SARS-SARS-COV-2/HCoV- | SKRSFIEDLLFNKV | LAGRSALEDLLFSK |
| | | F | PTF | | | | 6*02 | | NL63 S cross reactive | TLA | VVT |
| 6 | Full | CAWSVGGNYGYT | CAVTKPSGTAL | TRBV30 | | TRBJ1-2 | TRAV8- | TRAJ15 | SARS-SARS-COV-2/HCoV- | SKRSFIEDLLFNKV | LAGRSALEDLLFSK |
| | | F | IF | | | | 6*02 | | NL63 S cross reactive | TLA | VVT |
| 7 | Full | CASSKKRTGPGA | CIESNTGKLIF | TRBV6-5 | | TRBJ2-6 | TRAV26-1 | TRAJ37* | SARS-SARS-COV-2/HCoV- | NA | NA |
| | | NVLTF | | | | | | 02 | NL63 S cross reactive | | |
| 8 | Full | CAWSVQGNYGYT | CAVSLTYKYIF | TRBV30 | | TRBJ1-2 | TRAV8- | TRAJ40 | SARS-SARS-COV-2/HCoV- | SKRSFIEDLLFNKV | LAGRSALEDLLFSK |
| | | F | | | | | 6*02 | | NL63 S cross reactive | TLA | VVT |
| 9 | Full | CSVELDRGRRGY | CAMTVNSGYST | TRBV29-1 | | TRBJ1-2 | TRAV12-3 | TRAJ11 | SARS-COV-2 S | GINITRFQTLLALH | NA |
| | | TF | LTF | | | | | | | RSY | |
| 10 | Full | CASSQLGRTGEL | CAENSGTYKYI | TRBV7- | | TRBJ2-2 | TRAV25 | TRAJ40 | SARS-COV-2 S | NA | NA |
| | | FF | F | 9*03 | | | | | | | |
| 11 | Full | CASSLSVGDTPY | CAENGSRDTGR | TRBV5-1 | | TRBJ2-7 | TRAV13-2 | TRAJ5 | SARS-COV-2 S | GINITRFQTLLALH | NA |
| | | EQYF | RALTF | | | | | | | RSY | |
| 12 | Full | CSAGRATDGHLE | CIVRDNAGNML | TRBV20-1 | | TRBJ2-7 | TRAV26-1 | TRAJ39 | SARS-COV-2 S | NA | NA |
| | | QYF | TF | | | | | | | | |
| 13 | Full | CASSEQGYGEQF | CVVKSRGGGGK | TRBV10-1 | | TRBJ2-1 | TRAV12-1 | TRAJ23 | SARS-COV-2 S | QFCNDPFLGVYHKN | NA |
| | | F | LIF | | | | | | | NK | |
| 14 | Beta | CAIGGDSNYGYT | | TRBV10-3 | TRBD1 | TRBJ1-2 | NA | NA | SARS-COV-2 S | NA | NA |
| | | F | | | | | | | | | |
| 15 | Beta | CASKRDSINGYT | | TRBV6-1 | TRBD1 | TRBJ1-2 | NA | NA | SARS-COV-2 S | NA | NA |
| | | F | | | | | | | | | |
| 16 | Beta | CASSDAYYGYTF | | TRBV10-1 | | TRBJ1-2 | NA | NA | SARS-COV-2 S | NA | NA |
| 17 | Beta | CASSRGGFDTEA | | TRBV5-1 | TRBD1 | TRBJ1-1 | NA | NA | SARS-COV-2 S | NA | NA |
| | | FF | | | | | | | | | |
| 18 | Beta | CASTETPGAFRG | | TRBV6-1 | TRBD1 | TRBJ1-2 | NA | NA | SARS-COV-2 S | NA | NA |
| | | YTF | | | | | | | | | |
| 19 | Beta | CSARATGTSVYN | | TRBV20-1 | TRBD1 | TRBJ2-1 | NA | NA | SARS-COV-2 S | NA | NA |
| | | EQFF | | | | | | | | | |
| 20 | Beta | CSARSTDRAGGY | | TRBV20-1 | TRBD1 | TRBJ1-2 | NA | NA | SARS-COV-2 S | NA | NA |
| | | TF | | | | | | | | | |
| 21 | Beta | CSVTGSGEKLFF | | TRBV20-1 | TRBD1 | TRBJ1-4 | NA | NA | SARS-COV-2 S | NA | NA |
| 22 | Beta | CASSERETQYF | | TRBV2 | | TRBJ2-5 | NA | NA | SARS-COV-2 S | NA | NA |
| 23 | Beta | CASSLRRGDRGS | | TRBV11-2 | TRBD1 | TRBJ1-2 | NA | NA | SARS-COV-2 S | NA | NA |
| | | YGYTF | | | | | | | | | |
| 24 | Beta | CASSLVGGGAET | | TRBV7-2, | TRBD1, | TRBJ2-5 | NA | NA | SARS-COV-2 S | NA | NA |
| | | QYF | | TRBV7-8 | TRBD2 | | | | | | |
| 25 | Beta | CASSPLRGQKAF | | TRBV28 | TRBD1 | TRBJ1-1 | NA | NA | SARS-COV-2 S | NA | NA |
| | | F | | | | | | | | | |
| 26 | Beta | CASSSRQGALEG | | TRBV14 | TRBD1 | TRBJ1-2 | NA | NA | SARS-COV-2 S | NA | NA |
| | | YTF | | | | | | | | | |
| 27 | Beta | CATSSRTGGGNT | | TRBV15 | TRBD1 | TRBJ1-3 | NA | NA | SARS-COV-2 S | NA | NA |
| | | IYF | | | | | | | | | |
| 28 | Beta | CSVENGGNYGYT | | TRBV29-1 | TRBD2 | TRBJ1-2 | NA | NA | SARS-COV-2 S | NA | NA |
| | | F | | | | | | | | | |
| 29 | Beta | CAISEGQGSTDT | | TRBV10-3 | TRBD1, | TRBJ2-3 | NA | NA | SARS-COV-2 S | NA | NA |
| | | QYF | | | TRBD2 | | | | | | |
| 30 | Beta | CASRGTREGLTD | | TRBV6-1, | TRBD2 | TRBJ2-3 | NA | NA | SARS-COV-2 S | NA | NA |
| | | TQYF | | TRBV6-5, | | | | | | | |
| | | | | TRBV6-6, | | | | | | | |
| | | | | TRBV6-8 | | | | | | | |
| 31 | Beta | CASRSGQESSYN | | TRBV7-2, | TRBD1 | TRBJ1-6 | NA | NA | SARS-COV-2 S | NA | NA |
| | | SPLHF | | TRBV7-8 | | | | | | | |
| 32 | Beta | CASRTSGQPQHF | | TRBV6-2, | | TRBJ1-5 | NA | NA | SARS-COV-2 S | NA | NA |
| | | | | TRBV6-3 | | | | | | | |
| 33 | Beta | CASSFGGSGADT | | TRBV7-2, | TRBD2 | TRBJ2-3 | NA | NA | SARS-COV-2 S | NA | NA |
| | | QYF | | TRBV7-8 | | | | | | | |
| 34 | Beta | CASSLYVRWAHN | | TRBV7-2, | | TRBJ2-1 | NA | NA | SARS-COV-2 S | NA | NA |
| | | EQFF | | TRBV7-8 | | | | | | | |
| 35 | Beta | CASSSEGRNYGY | | TRBV7-6 | TRBD2 | TRBJ1-2 | NA | NA | SARS-COV-2 S | NA | NA |
| | | TF | | | | | | | | | |
| 36 | Beta | CASSSFGGGTDT | | TRBV7-2, | TRBD2 | TRBJ2-3 | NA | NA | SARS-COV-2 S | NA | NA |
| | | QYF | | TRBV7-8 | | | | | | | |
| 37 | Beta | CASSYALERADT | | TRBV6-2, | | TRBJ2-3 | NA | NA | SARS-COV-2 S | NA | NA |
| | | QYF | | TRBV6-3, | | | | | | | |
| | | | | TRBV6-5, | | | | | | | |
| | | | | TRBV6-6 | | | | | | | |
| 38 | Beta | CASSYGWTPGGV | | TRBV6-1, | TRBD1, | TRBJ1-1 | NA | NA | SARS-COV-2 S | NA | NA |
| | | NTEAFF | | TRBV6-3, | TRBD2 | | | | | | |
| | | | | TRBV6-5, | | | | | | | |
| | | | | TRBV6-6, | | | | | | | |
| | | | | TRBV6-8 | | | | | | | |
| 39 | Beta | CASSYRAGHLPD | | TRBV6-1, | TRBD1, | TRBJ2-2 | NA | NA | SARS-COV-2 S | NA | NA |
| | | TGELFF | | TRBV6-3, | TRBD2 | | | | | | |
| | | | | TRBV6-5, | | | | | | | |
| | | | | TRBV6-6, | | | | | | | |
| | | | | TRBV6-8 | | | | | | | |
| 40 | Beta | CSAGRGGGNNEQ | | TRBV20-1 | TRBD1, | TRBJ2-1 | NA | NA | SARS-COV-2 S | NA | NA |
| | | FF | | | TRBD2 | | | | | | |
| 41 | Beta | CSAHGPWTEQET | | TRBV20-1 | TRBD2 | TRBJ2-5 | NA | NA | SARS-COV-2 S | NA | NA |
| | | QYF | | | | | | | | | |
| 42 | Beta | CSASPTTDTQYF | | TRBV20-1 | | TRBJ2-3 | NA | NA | SARS-COV-2 S | NA | NA |
| 43 | Beta | CSVPFRGGGDIN | | TRBV20-1 | TRBD1 | TRBJ1-5 | NA | NA | SARS-COV-2 S | NA | NA |
| | | QPQHF | | | | | | | | | |
| 44 | Beta | CASGDRGAFF | | TRBV6-1, | TRBD1 | TRBJ1-1 | NA | NA | SARS-COV-2 S | NA | NA |
| | | | | TRBV6-5, | | | | | | | |
| | | | | TRBV6-6, | | | | | | | |
| | | | | TRBV6-8 | | | | | | | |
| 45 | Beta | CASKEGVKGSNQ | | TRBV19 | TRBD1 | TRBJ1-5 | NA | NA | SARS-COV-2 S | NA | NA |
| | | PQHF | | | | | | | | | |
| 46 | Beta | CASLLSAGGGRY | | TRBV7-2, | TRBD1, | TRBJ1-2 | NA | NA | SARS-COV-2 S | NA | NA |
| | | GYTF | | TRBV7-8 | TRBD2 | | | | | | |
| 47 | Beta | CASRGLAGARDT | | TRBV7-2, | TRBD2 | TRBJ2-3 | NA | NA | SARS-COV-2 S | NA | NA |
| | | QYF | | TRBV7-8 | | | | | | | |
| 48 | Beta | CASRPRGGNQPQ | | TRBV6-1, | TRBD1 | TRBJ1-5 | NA | NA | SARS-COV-2 S | NA | NA |
| | | HF | | TRBV6-5, | | | | | | | |
| | | | | TRBV6-6, | | | | | | | |
| | | | | TRBV6-8 | | | | | | | |
| 49 | Beta | CASSDRDRGYYE | | TRBV6-1, | TRBD1 | TRBJ2-7 | NA | NA | SARS-COV-2 S | NA | NA |
| | | QYF | | TRBV6-5, | | | | | | | |
| | | | | TRBV6-6 | | | | | | | |
| 50 | Beta | CASSDSKFSTDT | | TRBV6-4 | | TRBJ1-1 | NA | NA | SARS-COV-2 S | NA | NA |
| | | EAFF | | | | | | | | | |
| 51 | Beta | CASSDSRSNQPQ | | TRBV25-1 | | TRBJ1-5 | NA | NA | SARS-COV-2 S | NA | NA |
| | | HF | | | | | | | | | |
| 52 | Beta | CASSEKASGYNE | | TRBV10-1 | TRBD2 | TRBJ2-1 | NA | NA | SARS-COV-2 S | NA | NA |
| | | QFF | | | | | | | | | |
| 53 | Beta | CASSFGGSRYGY | | TRBV28 | TRBD1 | TRBJ1-2 | NA | NA | SARS-COV-2 S | NA | NA |
| | | TF | | | | | | | | | |
| 54 | Beta | CASSFGLAGFYE | | TRBV6-1, | TRBD2 | TRBJ2-7 | NA | NA | SARS-COV-2 S | NA | NA |
| | | QYF | | TRBV6-5, | | | | | | | |
| | | | | TRBV6-6, | | | | | | | |
| | | | | TRBV6-9 | | | | | | | |
| 55 | Beta | CASSGGLAGATY | | TRBV25-1 | TRBD2 | TRBJ2-7 | NA | NA | SARS-COV-2 S | NA | NA |
| | | EQYF | | | | | | | | | |
| 56 | Beta | CASSIGIDRPYG | | TRBV19 | TRBD1 | TRBJ1-2 | NA | NA | SARS-COV-2 S | NA | NA |
| | | YTF | | | | | | | | | |
| 57 | Beta | CASSIGTDRPYG | | TRBV19 | TRBD1 | TRBJ1-2 | NA | NA | SARS-COV-2 S | NA | NA |
| | | YTF | | | | | | | | | |
| 58 | Beta | CASSIKGSSNQP | | TRBV19 | | TRBJ1-5 | NA | NA | SARS-COV-2 S | NA | NA |
| | | QHF | | | | | | | | | |
| 59 | Beta | CASSIQGLQQTQ | | TRBV28 | TRBD1 | TRBJ2-5 | NA | NA | SARS-COV-2 S | NA | NA |
| | | YF | | | | | | | | | |
| 60 | Beta | CASSLAPGTGFG | | TRBV5-4, | TRBD1 | TRBJ1-2 | NA | NA | SARS-COV-2 S | NA | NA |
| | | YGYTF | | TRBV5-8 | | | | | | | |
| 61 | Beta | CASSLGKARIYE | | TRBV7-2 | | TRBJ2-7 | NA | NA | SARS-COV-2 S | NA | NA |
| | | QYF | | | | | | | | | |
| 62 | Beta | CASSLGLAAFYE | | TRBV6-1, | TRBD2 | TRBJ2-7 | NA | NA | SARS-COV-2 S | NA | NA |
| | | QYF | | TRBV6-5, | | | | | | | |
| | | | | TRBV6-6, | | | | | | | |
| | | | | TRBV6-8 | | | | | | | |
| 63 | Beta | CASSLGSQLYGY | | TRBV7-2, | | TRBJ1-2 | NA | NA | SARS-COV-2 S | NA | NA |
| | | TF | | TRBV7-8 | | | | | | | |
| 64 | Beta | CASSPGDRGSGQ | | TRBV27 | TRBD1 | TRBJ1-5 | NA | NA | SARS-COV-2 S | NA | NA |
| | | PQHF | | | | | | | | | |
| 65 | Beta | CASSPLGTGRYG | | TRBV7-2, | TRBD1 | TRBJ1-2 | NA | NA | SARS-COV-2 S | NA | NA |
| | | YTF | | TRBV7-8 | | | | | | | |
| 66 | Beta | CASSPSGNTIYF | | TRBV25-1 | | TRBJ1-3 | NA | NA | SARS-COV-2 S | NA | NA |
| 67 | Beta | CASSPTDRGNQP | | TRBV6-1 | TRBD1 | TRBJ1-5 | NA | NA | SARS-COV-2 S | NA | NA |
| | | QHF | | TRBV6-5, | | | | | | | |
| | | | | TRBV6-6, | | | | | | | |
| | | | | TRBV6-8 | | | | | | | |
| 68 | Beta | CASSQGLAGFYE | | TRBV6-1, | TRBD2 | TRBJ2-7 | NA | NA | SARS-COV-2 S | NA | NA |
| | | QYF | | TRBV6-5, | | | | | | | |
| | | | | TRBV6-6, | | | | | | | |
| | | | | TRBV6-8 | | | | | | | |
| 69 | Beta | CASSRLTGREAF | | TRBV6-1, | TRBD1 | TRBJ1-1 | NA | NA | SARS-COV-2 S | NA | NA |
| | | F | | TRBV6-2, | | | | | | | |
| | | | | TRBV6-3, | | | | | | | |
| | | | | TRBV6-5, | | | | | | | |
| | | | | TRBV6-6 | | | | | | | |
| 70 | Beta | CASSSQFWEQYF | | TRBV11-2 | | TRBJ2-3 | NA | NA | SARS-COV-2 S | NA | NA |
| 71 | Beta | CASSTDGRQPQH | | TRBV2 | | TRBJ1-5 | NA | NA | SARS-COV-2 S | NA | NA |
| | | F | | | | | | | | | |
| 72 | Beta | CASSWGKRGRAQ | | TRBV28 | TRBD1 | TRBJ1-5 | NA | NA | SARS-COV-2 S | NA | NA |
| | | PQHF | | | | | | | | | |
| 73 | Beta | CATRDSSYNEQF | | TRBV24-1 | TRBD1, | TRBJ2-1 | NA | NA | SARS-COV-2 S | NA | NA |
| | | F | | | TRBD2 | | | | | | |
| 74 | Beta | CATSDGDLPNNQ | | TRBV24-1 | TRBD2 | TRBJ1-5 | NA | NA | SARS-COV-2 S | NA | NA |
| | | PQHF | | | | | | | | | |
| 75 | Beta | CATSDPLGPTDT | | TRBV24-1 | TRBD1, | TRBJ2-3 | NA | NA | SARS-COV-2 S | NA | NA |
| | | QYF | | | TRBD2 | | | | | | |
| 76 | Beta | CATSDPRQGMKA | | TRBV24-1 | TRBD1 | TRBJ1-1 | NA | NA | SARS-COV-2 S | NA | NA |
| | | FF | | | | | | | | | |
| 77 | Beta | CATSEKGEQYF | | TRBV24-1 | | TRBJ2-7 | NA | NA | SARS-COV-2 S | NA | NA |
| 78 | Beta | CSAIQEGRNSNQ | | TRBV20-1 | TRBD1, | TRBJ1-5 | NA | NA | SARS-COV-2 S | NA | NA |
| | | PQHF | | | TRBD2 | | | | | | |
| 79 | Beta | CSALDRRSGNTI | | TRBV20-1 | TRBD1 | TRBJ1-3 | NA | NA | SARS-COV-2 S | NA | NA |
| | | YF | | | | | | | | | |
| 80 | Beta | CSAPARGYSPLH | | TRBV20-1 | TRBD1, | TRBJ1-6 | NA | NA | SARS-COV-2 S | NA | NA |
| | | F | | | TRBD2 | | | | | | |
| 81 | Beta | CSARLPGRAQPQ | | TRBV20-1 | TRBD1, | TRBJ1-5 | NA | NA | SARS-COV-2 S | NA | NA |
| | | HF | | | TRBD2 | | | | | | |
| 82 | Beta | CSARPGLDNQPQ | | TRBV20-1 | TRBD1, | TRBJ1-5 | NA | NA | SARS-COV-2 S | NA | NA |
| | | HF | | | TRBD2 | | | | | | |
| 83 | Beta | CSARVGQGGSYE | | TRBV20-1 | TRBD1 | TRBJ2-7 | NA | NA | SARS-COV-2 S | NA | NA |
| | | QYF | | | | | | | | | |
| 84 | Beta | CSASIGRDSSYE | | TRBV20-1 | TRBD1, | TRBJ2-7 | NA | NA | SARS-COV-2 S | NA | NA |
| | | QYF | | | TRBD2 | | | | | | |
| 85 | Beta | CASGPHGTENSP | | TRBV11-2 | TRBD1 | TRBJ1-6 | NA | NA | SARS-COV-2 S | NA | NA |
| | | LHF | | | | | | | | | |
| 86 | Beta | CASRPRQSMNTE | | TRBV6-2 | TRBD1 | TRBJ1-1 | NA | NA | SARS-COV-2 S | NA | NA |
| | | AFF | | | | | | | | | |
| 87 | Beta | CASSLAGGTYNE | | TRBV5-1 | TRBD2 | TRBJ2-1 | NA | NA | SARS-COV-2 S | NA | NA |
| | | QFF | | | | | | | | | |
| 88 | Beta | CASSLAGPDTQY | | TRBV11-2 | TRBD1 | TRBJ2-3 | NA | NA | SARS-COV-2 S | NA | NA |
| | | F | | | | | | | | | |
| 89 | Beta | CASSLGVGGLAN | | TRBV4-3 | TRBD2 | TRBJ1-2 | NA | NA | SARS-COV-2 S | NA | NA |
| | | YGYTF | | | | | | | | | |
| 90 | Beta | CASSRRGGDGYT | | TRBV6-1 | TRBD1 | TRBJ1-2 | NA | NA | SARS-COV-2 S | NA | NA |
| | | F | | | | | | | | | |
| 91 | Beta | CASSSTYEQYF | | TRBV11-2 | | TRBJ2-7 | NA | NA | SARS-COV-2 S | NA | NA |
| 92 | Beta | CSARGQGNYGYT | | TRBV20-1 | TRBD1 | TRBJ1-2 | NA | NA | SARS-COV-2 S | NA | NA |
| | | F | | | | | | | | | |
| 93 | Beta | CSGRGQGNYGYT | | TRBV20-1 | TRBD1 | TRBJ1-2 | NA | NA | SARS-COV-2 S | NA | NA |
| | | F | | | | | | | | | |
| 94 | Beta | CASREGQGGYGY | | TRBV6-1 | TRBD1 | TRBJ1-2 | NA | NA | SARS-COV-2 S | NA | NA |
| | | TF | | | | | | | | | |
| 95 | Beta | CASRLTSGSENI | | TRBV6-4 | TRBD2 | TRBJ2-4 | NA | NA | SARS-COV-2 S | NA | NA |
| | | QYF | | | | | | | | | |
| 96 | Beta | CASSFRDRGQGG | | TRBV11-2 | TRBD1 | TRBJ1-2 | NA | NA | SARS-COV-2 S | NA | NA |
| | | YTF | | | | | | | | | |
| 97 | Beta | CASSFRNTGELF | | TRBV11-3 | | TRBJ2-2 | NA | NA | SARS-COV-2 S | NA | NA |
| | | F | | | | | | | | | |
| 98 | Beta | CASSLARKGYTF | | TRBV7-2 | | TRBJ1-2 | NA | NA | SARS-COV-2 S | NA | NA |
| 99 | Beta | CASSQGTANTGE | | TRBV6-9 | TRBD1 | TRBJ2-2 | NA | NA | SARS-COV-2 S | NA | NA |
| | | LFF | | | | | | | | | |
| 100 | Beta | CASSSGRDTQYF | | TRBV7-3 | TRBD2 | TRBJ2-3 | NA | NA | SARS-COV-2 S | NA | NA |
| 101 | Beta | CASSSSLLTGSG | | TRBV7-2 | TRBD1 | TRBJ1-3 | NA | NA | SARS-COV-2 S | NA | NA |
| | | NTIYF | | | | | | | | | |
| 102 | Beta | CAGRAGEQYTQY | | TRBV2 | TRBD1 | TRBJ2-3 | NA | NA | SARS-COV-2 S | NA | NA |
| | | F | | | | | | | | | |
| 103 | Beta | CAISESPSGGPT | | TRBV10-3 | TRBD2 | TRBJ2-1 | NA | NA | SARS-COV-2 S | NA | NA |
| | | NEQFF | | | | | | | | | |
| 104 | Beta | CARTELKTSGEN | | TRBV5-3 | TRBD2 | TRBJ2-1 | NA | NA | SARS-COV-2 S | NA | NA |
| | | NEQFF | | | | | | | | | |
| 105 | Beta | CASALAGEAKNI | | TRBV6-2 | TRBD2 | TRBJ2-4 | NA | NA | SARS-COV-2 S | NA | NA |
| | | QYF | | | | | | | | | |
| 106 | Beta | CASRTGNQPQHF | | TRBV19 | | TRBJ1-5 | NA | NA | SARS-COV-2 S | NA | NA |
| 107 | Beta | CASSDIGLAGDT | | TRBV10-1 | TRBD2 | TRBJ2-3 | NA | NA | SARS-COV-2 S | NA | NA |
| | | DTQYF | | | | | | | | | |
| 108 | Beta | CASSDRGGQGPD | | TRBV25-1 | TRBD1 | TRBJ2-3 | NA | NA | SARS-COV-2 S | NA | NA |
| | | TQYF | | | | | | | | | |
| 109 | Beta | CASSERTGGEGY | | TRBV6-1 | TRBD1 | TRBJ1-2 | NA | NA | SARS-COV-2 S | NA | NA |
| | | TF | | | | | | | | | |
| 110 | Beta | CASSESIGYTF | | TRBV6-1 | | TRBJ1-2 | NA | NA | SARS-COV-2 S | NA | NA |
| 111 | Beta | CASSETGSDGYT | | TRBV10-2 | TRBD1 | TRBJ1-2 | NA | NA | SARS-COV-2 S | NA | NA |
| | | F | | | | | | | | | |
| 112 | Beta | CASSFRDRGLTE | | TRBV5-6 | TRBD1 | TRBJ1-1 | NA | NA | SARS-COV-2 S | NA | NA |
| | | AFF | | | | | | | | | |
| 113 | Beta | CASSGSGALSPL | | TRBV12-3 | TRBD2 | TRBJ1-6 | NA | NA | SARS-COV-2 S | NA | NA |
| | | HF | | | | | | | | | |
| 114 | Beta | CASSGSGNLGAQ | | TRBV12-3 | TRBD1 | TRBJ2-5 | NA | NA | SARS-COV-2 S | NA | NA |
| | | YF | | | | | | | | | |
| 115 | Beta | CASSLARQGNTG | | TRBV7-6 | TRBD1 | TRBJ2-2 | NA | NA | SARS-COV-2 S | NA | NA |
| | | ELFF | | | | | | | | | |
| 116 | Beta | CASSLERGGQSP | | TRBV7-2 | TRBD1 | TRBJ1-6 | NA | NA | SARS-COV-2 S | NA | NA |
| | | LHF | | | | | | | | | |
| 117 | Beta | CASSLGDGRSYG | | TRBV5-1 | TRBD2 | TRBJ1-2 | NA | NA | SARS-COV-2 S | NA | NA |
| | | YTF | | | | | | | | | |
| 118 | Beta | CASSLLSANYGY | | TRBV7-8 | TRBD1 | TRBJ1-2 | NA | NA | SARS-COV-2 S | NA | NA |
| | | TF | | | | | | | | | |
| 119 | Beta | CASSLPGTGVTE | | TRBV5-4 | TRBD1 | TRBJ1-1 | NA | NA | SARS-COV-2 S | NA | NA |
| | | AFF | | | | | | | | | |
| 120 | Beta | CASSLRGADGYT | | TRBV7-2 | TRBD1 | TRBJ1-2 | NA | NA | SARS-COV-2 S | NA | NA |
| | | F | | | | | | | | | |
| 121 | Beta | CASSLRGANGYT | | TRBV25-1 | TRBD1 | TRBJ1-2 | NA | NA | SARS-COV-2 S | NA | NA |
| | | F | | | | | | | | | |
| 122 | Beta | CASSLRGGNGYT | | TRBV27 | TRBD1 | TRBJ1-2 | NA | NA | SARS-COV-2 S | NA | NA |
| | | F | | | | | | | | | |
| 123 | Beta | CASSLRGQAALG | | TRBV7-9 | TRBD1 | TRBJ2-2 | NA | NA | SARS-COV-2 S | NA | NA |
| | | ELFF | | | | | | | | | |
| 124 | Beta | CASSLVGGTRST | | TRBV7-8 | TRBD1 | TRBJ2-3 | NA | NA | SARS-COV-2 S | NA | NA |
| | | DTQYF | | | | | | | | | |
| 125 | Beta | CASSQLGRTGEL | | TRBV7-9 | TRBD1 | TRBJ2-2 | NA | NA | SARS-COV-2 S | NA | NA |
| | | FF | | | | | | | | | |
| 126 | Beta | CASSQTGQGVEG | | TRBV4-3 | TRBD1 | TRBJ1-2 | NA | NA | SARS-COV-2 S | NA | NA |
| | | YTF | | | | | | | | | |
| 127 | Beta | CASSSETSGSIT | | TRBV6-2 | TRBD2 | TRBJ2-5 | NA | NA | SARS-COV-2 S | NA | NA |
| | | GTQYF | | | | | | | | | |
| 128 | Beta | CASSSGKPQHF | | TRBV28 | | TRBJ1-5 | NA | NA | SARS-COV-2 S | NA | NA |
| 129 | Beta | CASSSGKTQYF | | TRBV28 | | TRBJ2-5 | NA | NA | SARS-COV-2 S | NA | NA |
| 130 | Beta | CASSSLGGLALN | | TRBV7-9 | TRBD2 | TRBJ2-1 | NA | NA | SARS-COV-2 S | NA | NA |
| | | EQFF | | | | | | | | | |
| 131 | Beta | CASSYSTTSTDT | | TRBV6-5 | | TRBJ2-3 | NA | NA | SARS-COV-2 S | NA | NA |
| | | QYF | | | | | | | | | |
| 132 | Beta | CASTDRGLFAGE | | TRBV6-2 | TRBD1 | TRBJ2-2 | NA | NA | SARS-COV-2 S | NA | NA |
| | | LFF | | | | | | | | | |
| 133 | Beta | CATSDFKGGYTF | | TRBV24-1 | | TRBJ1-2 | NA | NA | SARS-COV-2 S | NA | NA |
| 134 | Beta | CATSSRTGSIYG | | TRBV15 | TRBD1 | TRBJ1-2 | NA | NA | SARS-COV-2 S | NA | NA |
| | | YTF | | | | | | | | | |
| 135 | Beta | CSAPGRDRQPQH | | TRBV20-1 | TRBD1 | TRBJ1-5 | NA | NA | SARS-COV-2 S | NA | NA |
| | | F | | | | | | | | | |
| 136 | Beta | CSARDARGGNTE | | TRBV20-1 | TRBD1 | TRBJ1-1 | NA | NA | SARS-COV-2 S | NA | NA |
| | | AFF | | | | | | | | | |
| 137 | Beta | CSARNGPSNQPQ | | TRBV20-1 | TRBD1 | TRBJ1-5 | NA | NA | SARS-COV-2 S | NA | NA |
| | | HF | | | | | | | | | |
| 138 | Beta | CSASQPRGNTDT | | TRBV20-1 | TRBD1 | TRBJ2-3 | NA | NA | SARS-COV-2 S | NA | NA |
| | | QYF | | | | | | | | | |
| 139 | Beta | CSATDRGRAGEL | | TRBV20-1 | TRBD1 | TRBJ2-2 | NA | NA | SARS-COV-2 S | NA | NA |
| | | FF | | | | | | | | | |
| 140 | Beta | CSGSTVGEQYF | | TRBV29-1 | TRBD1 | TRBJ2-7 | NA | NA | SARS-COV-2 S | NA | NA |
| 141 | Beta | CSVELDRGRRGY | | TRBV29-1 | TRBD1 | TRBJ1-2 | NA | NA | SARS-COV-2 S | NA | NA |
| | | TF | | | | | | | | | |
| 142 | Beta | CSVRRGRDTEAF | | TRBV29-1 | TRBD1 | TRBJ1-1 | NA | NA | SARS-COV-2 S | NA | NA |
| | | F | | | | | | | | | |
| 143 | Beta | CASNQGQGSKTQ | | TRBV6-6 | TRBD1 | TRBJ2-5 | NA | NA | SARS-COV-2 S | NA | NA |
| | | YF | | | | | | | | | |
| 144 | Beta | CASSLLGIRGTD | | TRBV7-2 | TRBD1 | TRBJ2-3 | NA | NA | SARS-COV-2 S | NA | NA |
| | | TQYF | | | | | | | | | |
| 145 | Beta | CASSLVTGIRYE | | TRBV5-8 | TRBD1 | TRBJ2-7 | NA | NA | SARS-COV-2 S | NA | NA |
| | | QYF | | | | | | | | | |
| 146 | Beta | CASSRTGARSSY | | TRBV12-3 | TRBD1 | TRBJ1-6 | NA | NA | SARS-COV-2 S | NA | NA |
| | | NSPLHF | | | | | | | | | |
| 147 | Beta | CASSRTGFGSSY | | TRBV12-3 | TRBD1 | TRBJ1-6 | NA | NA | SARS-COV-2 S | NA | NA |
| | | NSPLHF | | | | | | | | | |
| 148 | Beta | CASSRTGFRSSY | | TRBV12-3 | TRBD1 | TRBJ1-6 | NA | NA | SARS-COV-2 S | NA | NA |
| | | NSPLHF | | | | | | | | | |
| 149 | Beta | CASSRTGGGSSY | | TRBV12-4 | TRBD2 | TRBJ1-6 | NA | NA | SARS-COV-2 S | NA | NA |
| | | NSPLHF | | | | | | | | | |
| 150 | Beta | CASSRTGGRSSY | | TRBV12-3 | TRBD1 | TRBJ1-6 | NA | NA | SARS-COV-2 S | NA | NA |
| | | NSPLHF | | | | | | | | | |
| 151 | Beta | CASSRTGIASSY | | TRBV12-3 | TRBD1 | TRBJ1-6 | NA | NA | SARS-COV-2 S | NA | NA |
| | | NSPLHF | | | | | | | | | |
| 152 | Beta | CASSRTGIGSSY | | TRBV12-3 | TRBD1 | TRBJ1-6 | NA | NA | SARS-COV-2 S | NA | NA |
| | | NSPLHF | | | | | | | | | |
| 153 | Beta | CASSRTGKGSSY | | TRBV12-3 | TRBD1 | TRBJ1-6 | NA | NA | SARS-COV-2 S | NA | NA |
| | | NSPLHF | | | | | | | | | |
| 154 | Beta | CASSRTGLGSSY | | TRBV12-3 | TRBD1 | TRBJ1-6 | NA | NA | SARS-COV-2 S | NA | NA |
| | | NSPLHF | | | | | | | | | |
| 155 | Beta | CASSRTGLKSSY | | TRBV12-3 | TRBD1 | TRBJ1-6 | NA | NA | SARS-COV-2 S | NA | NA |
| | | NSPLHF | | | | | | | | | |
| 156 | Beta | CASSRTGLRSSY | | TRBV12-4 | TRBD1 | TRBJ1-6 | NA | NA | SARS-COV-2 S | NA | NA |
| | | NSPLHF | | | | | | | | | |
| 157 | Beta | CASSRTGQGSSY | | TRBV12-3 | TRBD1 | TRBJ1-6 | NA | NA | SARS-COV-2 S | NA | NA |
| | | NSPLHF | | | | | | | | | |
| 158 | Beta | CASSRTGQRSSY | | TRBV12-4 | | TRBJ1-6 | NA | NA | SARS-COV-2 S | NA | NA |
| | | NSPLHF | | | | | | | | | |
| 159 | Beta | CASSRTGRGSSY | | TRBV12-3 | TRBD1 | TRBJ1-6 | NA | NA | SARS-COV-2 S | NA | NA |
| | | NSPLHF | | | | | | | | | |
| 160 | Beta | CASSRTGTGSSY | | TRBV12-3 | TRBD1 | TRBJ1-6 | NA | NA | SARS-COV-2 S | NA | NA |
| | | NSPLHF | | | | | | | | | |
| 161 | Beta | CASSRTGVGSSY | | TRBV12-3 | TRBD1 | TRBJ1-6 | NA | NA | SARS-COV-2 S | NA | NA |
| | | NSPLHF | | | | | | | | | |
| 162 | Beta | CASSRTGVKSSY | | TRBV12-3 | TRBD1 | TRBJ1-6 | NA | NA | SARS-COV-2 S | NA | NA |
| | | NSPLHF | | | | | | | | | |
| 163 | Beta | CASSRTGVRSSY | | TRBV12-4 | TRBD1 | TRBJ1-6 | NA | NA | SARS-COV-2 S | NA | NA |
| | | NSPLHF | | | | | | | | | |
| 164 | Beta | CASSSPATGRNQ | | TRBV7-2 | TRBD1 | TRBJ1-5 | NA | NA | SARS-COV-2 S | NA | NA |
| | | PQHF | | | | | | | | | |
| 165 | Beta | CSALRYGPGQGM | | TRBV20-1 | TRBD1 | TRBJ1-1 | NA | NA | SARS-COV-2 S | NA | NA |
| | | ENTEAFF | | | | | | | | | |
| 166 | Beta | CSARDVTGNYGY | | TRBV20-1 | | TRBJ1-2 | NA | NA | SARS-COV-2 S | NA | NA |
| | | TF | | | | | | | | | |
| 167 | Beta | CSARGTGNYGYT | | TRBV20-1 | TRBD1 | TRBJ1-2 | NA | NA | SARS-COV-2 S | NA | NA |
| | | F | | | | | | | | | |
| 168 | Beta | CAGSDGLLSPLH | | TRBV10-3 | | TRBJ1-6 | NA | NA | SARS-COV-2 S | NA | NA |
| | | F | | | | | | | | | |
| 169 | Beta | CAISEIVFVGSY | | TRBV10-3 | | TRBJ2-1 | NA | NA | SARS-COV-2 S | NA | NA |
| | | NEQFF | | | | | | | | | |
| 170 | Beta | CASNPGGAVYNE | | TRBV19 | TRBD1, | TRBJ2-1 | NA | NA | SARS-COV-2 S | NA | NA |
| | | QFF | | | TRBD2 | | | | | | |
| 171 | Beta | CASRLAGGSYEQ | | TRBV27 | TRBD2 | TRBJ2-7 | NA | NA | SARS-COV-2 S | NA | NA |
| | | YF | | | | | | | | | |
| 172 | Beta | CASSERWTGVTE | | TRBV2 | TRBD1 | TRBJ1-1 | NA | NA | SARS-COV-2 S | NA | NA |
| | | AFF | | | | | | | | | |
| 173 | Beta | CASSFRTGSLDY | | TRBV6-1, | TRBD1 | TRBJ1-2 | NA | NA | SARS-COV-2 S | NA | NA |
| | | GYTF | | TRBV6-3, | | | | | | | |
| | | | | TRBV6-5, | | | | | | | |
| | | | | TRBV6-6, | | | | | | | |
| | | | | TRBV6-8 | | | | | | | |
| 174 | Beta | CASSLAGEVNQP | | TRBV6-1, | TRBD1, | TRBJ1-5 | NA | NA | SARS-COV-2 S | NA | NA |
| | | QHF | | TRBV6-2, | TRBD2 | | | | | | |
| | | | | TRBV6-3, | | | | | | | |
| | | | | TRBV6-5, | | | | | | | |
| | | | | TRBV6-6 | | | | | | | |
| 175 | Beta | CASSLRGDTQYF | | TRBV11-1, | | TRBJ2-3 | NA | NA | SARS-COV-2 S | NA | NA |
| | | | | TRBV11-3 | | | | | | | |
| 176 | Beta | CASSLRLAGDGT | | TRBV7-6 | TRBD2 | TRBJ2-2 | NA | NA | SARS-COV-2 S | NA | NA |
| | | GELFF | | | | | | | | | |
| 177 | Beta | CASSLRTSGVYN | | TRBV7-3 | TRBD2 | TRBJ2-1 | NA | NA | SARS-COV-2 S | NA | NA |
| | | EQFF | | | | | | | | | |
| 178 | Beta | CASSPARSANTG | | TRBV6-1, | TRBD2 | TRBJ2-2 | NA | NA | SARS-COV-2 S | NA | NA |
| | | ELFF | | TRBV6-3, | | | | | | | |
| | | | | TRBV6-5, | | | | | | | |
| | | | | TRBV6-6, | | | | | | | |
| | | | | TRBV6-8 | | | | | | | |
| 179 | Beta | CASSPDLHSNEQ | | TRBV25-1 | | TRBJ2-1 | NA | NA | SARS-COV-2 S | NA | NA |
| | | FF | | | | | | | | | |
| 180 | Beta | CASSPGLAGAET | | TRBV12-3, | TRBD2 | TRBJ2-5 | NA | NA | SARS-COV-2 S | NA | NA |
| | | QYF | | TRBV12-4 | | | | | | | |
| 181 | Beta | CASSPGLAGAGR | | TRBV12-3, | TRBD2 | TRBJ2-7 | NA | NA | SARS-COV-2 S | NA | NA |
| | | QYF | | TRBV12-4 | | | | | | | |
| 182 | Beta | CASSPPAASDEQ | | TRBV18 | | TRBJ2-7 | NA | NA | SARS-COV-2 S | NA | NA |
| | | YF | | | | | | | | | |
| 183 | Beta | CASSVSGILGEQ | | TRBV12-3, | | TRBJ2-7 | NA | NA | SARS-COV-2 S | NA | NA |
| | | YF | | TRBV12-4 | | | | | | | |
| 184 | Beta | CASSYMTSGGTD | | TRBV6-2, | TRBD2 | TRBJ2-3 | NA | NA | SARS-COV-2 S | NA | NA |
| | | TQYF | | TRBV6-3, | | | | | | | |
| | | | | TRBV6-5, | | | | | | | |
| | | | | TRBV6-6 | | | | | | | |
| 185 | Beta | CASSYRGAQGYT | | TRBV6-2, | TRBD1 | TRBJ1-2 | NA | NA | SARS-COV-2 S | NA | NA |
| | | F | | TRBV6-3, | | | | | | | |
| | | | | TRBV6-5, | | | | | | | |
| | | | | TRBV6-6 | | | | | | | |
| 186 | Beta | CASSYSSGGIYG | | TRBV6-2, | TRBD1 | TRBJ1-2 | NA | NA | SARS-COV-2 S | NA | NA |
| | | YTF | | TRBV6-3, | | | | | | | |
| | | | | TRBV6-5, | | | | | | | |
| | | | | TRBV6-6, | | | | | | | |
| | | | | TRBV6-8 | | | | | | | |
| 187 | Beta | CASSYSSGGSYE | | TRBV6-2, | TRBD1 | TRBJ2-7 | NA | NA | SARS-COV-2 S | NA | NA |
| | | QYF | | TRBV6-3, | | | | | | | |
| | | | | TRBV6-5, | | | | | | | |
| | | | | TRBV6-6, | | | | | | | |
| | | | | TRBV6-8 | | | | | | | |
| 188 | Beta | CASSYSTGGIYE | | TRBV6-2, | TRBD1 | TRBJ2-7 | NA | NA | SARS-COV-2 S | NA | NA |
| | | QYF | | TRBV6-3, | | | | | | | |
| | | | | TRBV6-5, | | | | | | | |
| | | | | TRBV6-6, | | | | | | | |
| | | | | TRBV6-8 | | | | | | | |
| 189 | Beta | CASTESTGHGTF | | TRBV5-6, | TRBD1 | TRBJ1-2 | NA | NA | SARS-COV-2 S | NA | NA |
| | | | | TRBV5-7 | | | | | | | |
| 190 | Beta | CASTTGQGEGYT | | TRBV5-1 | TRBD1 | TRBJ1-2 | NA | NA | SARS-COV-2 S | NA | NA |
| | | F | | | | | | | | | |
| 191 | Beta | CASVQREGYTGE | | TRBV6-1, | TRBD2 | TRBJ2-2 | NA | NA | SARS-COV-2 S | NA | NA |
| | | LFF | | TRBV6-5, | | | | | | | |
| | | | | TRBV6-6, | | | | | | | |
| | | | | TRBV6-8 | | | | | | | |
| 192 | Beta | CATGTGLGYTF | | TRBV6-1, | TRBD1 | TRBJ1-2 | NA | NA | SARS-COV-2 S | NA | NA |
| | | | | TRBV6-5, | | | | | | | |
| | | | | TRBV6-6, | | | | | | | |
| | | | | TRBV6-8 | | | | | | | |
| 193 | Beta | CSVGATGLGGYT | | TRBV29-1 | TRBD1 | TRBJ1-2 | NA | NA | SARS-COV-2 S | NA | NA |
| | | F | | | | | | | | | |
| 194 | Beta | CASSEFRMSGDT | | TRBV25-1 | TRBD2 | TRBJ2-3 | NA | NA | SARS-COV-2 S | NA | NA |
| | | QYF | | | | | | | | | |
| 195 | Beta | CASSEGSWDIRS | | TRBV6-1, | | TRBJ1-6 | NA | NA | SARS-COV-2 S | NA | NA |
| | | PLHF | | TRBV6-5, | | | | | | | |
| | | | | TRBV6-6 | | | | | | | |
| 196 | Beta | CASSLANTYEQY | | TRBV5-1 | | TRBJ2-7 | NA | NA | SARS-COV-2 S | NA | NA |
| | | F | | | | | | | | | |
| 197 | Beta | CASSLATGAATD | | TRBV7-2, | TRBD1 | TRBJ2-3 | NA | NA | SARS-COV-2 S | NA | NA |
| | | TQYF | | TRBV7-8 | | | | | | | |
| 198 | Beta | CASSLDRLNQPQ | | TRBV7-2, | TRBD2 | TRBJ1-5 | NA | NA | SARS-COV-2 S | NA | NA |
| | | HF | | TRBV7-8 | | | | | | | |
| 199 | Beta | CASSLGPFYGYT | | TRBV11-2 | TRBD1, | TRBJ1-2 | NA | NA | SARS-COV-2 S | NA | NA |
| | | F | | | TRBD2 | | | | | | |
| 200 | Beta | CASSLSGPQETQ | | TRBV27 | TRBD2 | TRBJ2-5 | NA | NA | SARS-COV-2 S | NA | NA |
| | | YF | | | | | | | | | |
| 201 | Beta | CASSLTGNYGYT | | TRBV11-2 | TRBD1 | TRBJ1-2 | NA | NA | SARS-COV-2 S | NA | NA |
| | | F | | | | | | | | | |
| 202 | Beta | CASSPPANTDTQ | | TRBV19 | TRBD2 | TRBJ2-3 | NA | NA | SARS-COV-2 S | NA | NA |
| | | YF | | | | | | | | | |
| 203 | Beta | CASSPSATQQET | | TRBV28 | | TRBJ2-5 | NA | NA | SARS-COV-2 S | NA | NA |
| | | QYF | | | | | | | | | |
| 204 | Beta | CASSQRGAHGYT | | TRBV14 | TRBD1 | TRBJ1-2 | NA | NA | SARS-COV-2 S | NA | NA |
| | | F | | | | | | | | | |
| 205 | Beta | CASSSGTAPSFY | | TRBV27 | TRBD1 | TRBJ1-2 | NA | NA | SARS-COV-2 S | NA | NA |
| | | GYTF | | | | | | | | | |
| 206 | Beta | CASSTGGPYGYT | | TRBV5-1 | TRBD1 | TRBJ1-2 | NA | NA | SARS-COV-2 S | NA | NA |
| | | F | | | | | | | | | |
| 207 | Beta | CASSTLDRRGGP | | TRBV6-1, | TRBD1, | TRBJ1-6 | NA | NA | SARS-COV-2 S | NA | NA |
| | | LHF | | TRBV6-5, | TRBD2 | | | | | | |
| | | | | TRBV6-6, | | | | | | | |
| | | | | TRBV6-8, | | | | | | | |
| | | | | TRBV6-9 | | | | | | | |
| 208 | Beta | CASSYDRVGQPQ | | TRBV7-2, | TRBD1 | TRBJ1-5 | NA | NA | SARS-COV-2 S | NA | NA |
| | | HF | | TRBV7-8 | | | | | | | |
| 209 | Beta | CAWSVDRYEQYF | | TRBV30 | | TRBJ2-7 | NA | NA | SARS-COV-2 S | NA | NA |
| 210 | Beta | CSAPGVGSYEQY | | TRBV20-1 | TRBD1, | TRBJ2-7 | NA | NA | SARS-COV-2 S | NA | NA |
| | | F | | | TRBD2 | | | | | | |
| 211 | Beta | CSARGEGNYGYT | | TRBV20-1 | TRBD2 | TRBJ1-2 | NA | NA | SARS-COV-2 S | NA | NA |
| | | F | | | | | | | | | |
| 212 | Beta | CAISESITGGGR | | TRBV10-3 | TRBD2 | TRBJ1-1 | NA | NA | SARS-COV-2 S | NA | NA |
| | | AFF | | | | | | | | | |
| 213 | Beta | CASMGGLANTEA | | TRBV6-1, | TRBD2 | TRBJ1-1 | NA | NA | SARS-COV-2 S | NA | NA |
| | | FF | | TRBV6-5, | | | | | | | |
| | | | | TRBV6-6, | | | | | | | |
| | | | | TRBV6-8 | | | | | | | |
| 214 | Beta | CASRDNGQGVMW | | TRBV2 | TRBD1 | TRBJ1-2 | NA | NA | SARS-COV-2 S | NA | NA |
| | | TF | | | | | | | | | |
| 215 | Beta | CASRDRENYGYT | | TRBV6-1, | TRBD1 | TRBJ1-2 | NA | NA | SARS-COV-2 S | NA | NA |
| | | F | | TRBV6-3, | | | | | | | |
| | | | | TRBV6-5, | | | | | | | |
| | | | | TRBV6-6, | | | | | | | |
| | | | | TRBV6-8 | | | | | | | |
| 216 | Beta | CASRGQGNQPQH | | TRBV12-3, | TRBD1 | TRBJ1-5 | NA | NA | SARS-COV-2 S | NA | NA |
| | | F | | TRBV12-4 | | | | | | | |
| 217 | Beta | CASSEGQGAGGY | | TRBV2 | TRBD1 | TRBJ1-2 | NA | NA | SARS-COV-2 S | NA | NA |
| | | TF | | | | | | | | | |
| 218 | Beta | CASSKPRTGDGY | | TRBV21-1 | TRBD1 | TRBJ1-2 | NA | NA | SARS-COV-2 S | NA | NA |
| | | TF | | | | | | | | | |
| 219 | Beta | CASSLAGGHGYT | | TRBV7-9 | TRBD1 | TRBJ1-2 | NA | NA | SARS-COV-2 S | NA | NA |
| | | F | | | | | | | | | |
| 220 | Beta | CASSLAGGPVNG | | TRBV27 | TRBD1, | TRBJ2-2 | NA | NA | SARS-COV-2 S | NA | NA |
| | | GELFF | | | TRBD2 | | | | | | |
| 221 | Beta | CASSLGTVHTF | | TRBV7-2, | | TRBJ1-2 | NA | NA | SARS-COV-2 S | NA | NA |
| | | | | TRBV7-8 | | | | | | | |
| 222 | Beta | CASSLLQSRQGH | | TRBV6-1, | TRBD1 | TRBJ1-2 | NA | NA | SARS-COV-2 S | NA | NA |
| | | GYTF | | TRBV6-2, | | | | | | | |
| | | | | TRBV6-3, | | | | | | | |
| | | | | TRBV6-5, | | | | | | | |
| | | | | TRBV6-6 | | | | | | | |
| 223 | Beta | CASSLVVQDSLH | | TRBV7-9 | TRBD1 | TRBJ1-6 | NA | NA | SARS-COV-2 S | NA | NA |
| | | F | | | | | | | | | |
| 224 | Beta | CASSRDSSDGQP | | TRBV2 | TRBD1 | TRBJ1-5 | NA | NA | SARS-COV-2 S | NA | NA |
| | | QHF | | | | | | | | | |
| 225 | Beta | CASSSWDRGINS | | TRBV27 | TRBD1 | TRBJ1-6 | NA | NA | SARS-COV-2 S | NA | NA |
| | | PLHF | | | | | | | | | |
| 226 | Beta | CATSRAGGSADT | | TRBV24-1 | TRBD1 | TRBJ2-3 | NA | NA | SARS-COV-2 S | NA | NA |
| | | QYF | | | | | | | | | |
| 227 | Beta | CATSSDRVGSPL | | TRBV15 | TRBD1 | TRBJ1-6 | NA | NA | SARS-COV-2 S | NA | NA |
| | | HF | | | | | | | | | |
| 228 | Beta | CSAEAPRPGGGG | | TRBV20-1 | TRBD2 | TRBJ2-2 | NA | NA | SARS-COV-2 S | NA | NA |
| | | ELFF | | | | | | | | | |
| 229 | Beta | CSAILAGGRNEQ | | TRBV20-1 | TRBD2 | TRBJ2-1 | NA | NA | SARS-COV-2 S | NA | NA |
| | | FF | | | | | | | | | |
| 230 | Beta | CSAPLQGGGTGN | | TRBV20-1 | TRBD1 | TRBJ2-1 | NA | NA | SARS-COV-2 S | NA | NA |
| | | EQFF | | | | | | | | | |
| 231 | Beta | CSARGTGTNYGY | | TRBV20-1 | TRBD2 | TRBJ1-2 | NA | NA | SARS-COV-2 S | NA | NA |
| | | TF | | | | | | | | | |
| 232 | Beta | CSARPGLHYYEQ | | TRBV20-1 | TRBD1, | TRBJ2-7 | NA | NA | SARS-COV-2 S | NA | NA |
| | | YF | | | TRBD2 | | | | | | |
| 233 | Beta | CSARTGADGYTF | | TRBV20-1 | TRBD1 | TRBJ1-2 | NA | NA | SARS-COV-2 S | NA | NA |
| 234 | Beta | CASRPAQGPRGY | | TRBV27 | TRBD2 | TRBJ1-2 | NA | NA | SARS-COV-2 S | NA | NA |
| | | TF | | | | | | | | | |
| 235 | Beta | CASSGTGVWEQY | | TRBV19 | TRBD1 | TRBJ2-7 | NA | NA | SARS-COV-2 S | NA | NA |
| | | F | | | | | | | | | |
| 236 | Beta | CASSLGNWAGSN | | TRBV7-6 | TRBD1 | TRBJ1-1 | NA | NA | SARS-COV-2 S | NA | NA |
| | | TEAFF | | | | | | | | | |
| 237 | Beta | CASSLQDATEAF | | TRBV5-1 | | TRBJ1-1 | NA | NA | SARS-COV-2 S | NA | NA |
| | | F | | | | | | | | | |
| 238 | Beta | CASSLVAGTGIG | | TRBV7-2, | TRBD1 | TRBJ1-5 | NA | NA | SARS-COV-2 S | NA | NA |
| | | FQPQHF | | TRBV7-8 | | | | | | | |
| 239 | Beta | CASSPLGQGRDL | | TRBV18 | TRBD1, | TRBJ1-2 | NA | NA | SARS-COV-2 S | NA | NA |
| | | YGYTF | | | TRBD2 | | | | | | |
| 240 | Beta | CASSPSAFAGVS | | TRBV27 | TRBD2 | TRBJ2-5 | NA | NA | SARS-COV-2 S | NA | NA |
| | | LETQYF | | | | | | | | | |
| 241 | Beta | CASSSQTSGGQE | | TRBV7-2, | TRBD2 | TRBJ2-5 | NA | NA | SARS-COV-2 S | NA | NA |
| | | TQYF | | TRBV7-8 | | | | | | | |
| 242 | Beta | CASSVLAGGEGT | | TRBV6-1, | TRBD2 | TRBJ2-3 | NA | NA | SARS-COV-2 S | NA | NA |
| | | QYF | | TRBV6-5, | | | | | | | |
| | | | | TRBV6-6 | | | | | | | |
| 243 | Beta | CATSRGPLEKLF | | TRBV15 | TRBD2 | TRBJ1-4 | NA | NA | SARS-COV-2 S | NA | NA |
| | | F | | | | | | | | | |
| 244 | Beta | CATYPGGEAAEQ | | TRBV15 | TRBD1 | TRBJ2-7 | NA | NA | SARS-COV-2 S | NA | NA |
| | | YF | | | | | | | | | |
| 245 | Beta | CAWSPDGKAYEQ | | TRBV30 | TRBD1, | TRBJ2-7 | NA | NA | SARS-COV-2 S | NA | NA |
| | | YF | | | TRBD2 | | | | | | |
| 246 | Beta | CSARAATDTQYF | | TRBV20-1 | | TRBJ2-3 | NA | NA | SARS-COV-2 S | NA | NA |
| 247 | Beta | CSARDHGTDTQY | | TRBV20-1 | | TRBJ2-3 | NA | NA | SARS-COV-2 S | NA | NA |
| | | F | | | | | | | | | |
| 248 | Beta | CSARGQVGLAIE | | TRBV20-1 | TRBD2 | TRBJ2-3 | NA | NA | SARS-COV-2 S | NA | NA |
| | | YTQYF | | | | | | | | | |
| 249 | Beta | CSARRENGGSYN | | TRBV20-1 | TRBD2 | TRBJ2-1 | NA | NA | SARS-COV-2 S | NA | NA |
| | | EQFF | | | | | | | | | |
| 250 | Beta | CSARRMTGQAF | | TRBV20-1 | TRBD1 | TRBJ1-2 | NA | NA | SARS-COV-2 S | NA | NA |
| 251 | Beta | CSARVPVGRNTE | | TRBV20-1 | TRBD2 | TRBJ1-1 | NA | NA | SARS-COV-2 S | NA | NA |
| | | AFF | | | | | | | | | |
| 252 | Beta | CASGYPPGPNTG | | TRBV6-2 | TRBD1 | TRBJ2-2 | NA | NA | SARS-COV-2 S | NA | NA |
| | | ELFF | | | | | | | | | |
| 253 | Beta | CASSATGGSETQ | | TRBV9 | TRBD1 | TRBJ2-5 | NA | NA | SARS-COV-2 S | NA | NA |
| | | YF | | | | | | | | | |
| 254 | Beta | CASSEARVSEAT | | TRBV2 | TRBD1 | TRBJ2-2 | NA | NA | SARS-COV-2 S | NA | NA |
| | | GELFF | | | | | | | | | |
| 255 | Beta | CASSEGSGRPSR | | TRBV10-1 | TRBD2 | TRBJ2-1 | NA | NA | SARS-COV-2 S | NA | NA |
| | | DEQFF | | | | | | | | | |
| 256 | Beta | CASSETRGEQET | | TRBV2 | TRBD1 | TRBJ2-5 | NA | NA | SARS-COV-2 S | NA | NA |
| | | QYF | | | | | | | | | |
| 257 | Beta | CASSQLPQLSSY | | TRBV7-2 | TRBD1 | TRBJ2-1 | NA | NA | SARS-COV-2 S | NA | NA |
| | | NEQFF | | | | | | | | | |
| 258 | Beta | CASSSTGGWETQ | | TRBV7-3 | TRBD1 | TRBJ2-5 | NA | NA | SARS-COV-2 S | NA | NA |
| | | YF | | | | | | | | | |
| 259 | Beta | CSARSPSGSSYN | | TRBV20-1 | TRBD2 | TRBJ2-1 | NA | NA | SARS-COV-2 S | NA | NA |
| | | EQFF | | | | | | | | | |
| 260 | Beta | CASSRAGDTEAF | | TRBV18 | | TRBJ1-1 | NA | NA | SARS-COV-2 S | NA | NA |
| | | F | | | | | | | | | |
| 261 | Beta | CAWGQFNYGYTF | | TRBV30 | TRBD1 | TRBJ1-2 | NA | NA | SARS-COV-2 S | NA | NA |
| 262 | Beta | CASNLGQGGWYF | | TRBV6-5 | TRBD1 | TRBJ2-3 | NA | NA | SARS-COV-2 S | NA | NA |
| 263 | Beta | CASSWETSGSFP | | TRBV5-1 | TRBD2 | TRBJ2-7 | NA | NA | SARS-COV-2 S | NA | NA |
| | | SEQYF | | | | | | | | | |
| 264 | Beta | CSAREWGGGYGY | | TRBV20-1 | TRBD2 | TRBJ1-2 | NA | NA | SARS-COV-2 S | NA | NA |
| | | TF | | | | | | | | | |
| 265 | Beta | CAISVGTMGTSG | | TRBV10-3 | TRBD2 | TRBJ2-5 | NA | NA | S cross reactive | NA | NA |
| | | KETQYF | | | | | | | | | |
| 266 | Beta | CASSHPYGSTEA | | TRBV19 | TRBD2 | TRBJ1-1 | NA | NA | S cross reactive | NA | NA |
| | | FF | | | | | | | | | |
| 267 | Beta | CASSLTLRTASP | | TRBV5-1 | TRBD1 | TRBJ2-7 | NA | NA | S cross reactive | NA | NA |
| | | YEQYF | | | | | | | | | |
| 268 | Beta | CASSSRTGPRYG | | TRBV5-6, | TRBD1 | TRBJ1-2 | NA | NA | S cross reactive | NA | NA |
| | | YTF | | TRBV5-7 | | | | | | | |
| 269 | Beta | CASSTDTRGGEQ | | TRBV6-1, | TRBD1 | TRBJ2-7 | NA | NA | S cross reactive | NA | NA |
| | | YF | | TRBV6-3, | | | | | | | |
| | | | | TRBV6-5, | | | | | | | |
| | | | | TRBV6-6, | | | | | | | |
| | | | | TRBV6-8 | | | | | | | |
| 270 | Beta | CASSVALGQSYG | | TRBV9 | TRBD1 | TRBJ1-2 | NA | NA | S cross reactive | NA | NA |
| | | YTF | | | | | | | | | |
| 271 | Beta | CSAGPDRGRTGY | | TRBV20-1 | TRBD1 | TRBJ1-2 | NA | NA | S cross reactive | NA | NA |
| | | TF | | | | | | | | | |
| 272 | Beta | CAGRQGGIGNTF | | TRBV30 | TRBD1 | TRBJ1-2 | NA | NA | S cross reactive | NA | NA |
| 273 | Beta | CARSLRGTGGLN | | TRBV5-3 | TRBD1 | TRBJ1-5 | NA | NA | S cross reactive | NA | NA |
| | | QPQHF | | | | | | | | | |
| 274 | Beta | CASCPEGDTGEL | | TRBV12-3 | TRBD1 | TRBJ2-2 | NA | NA | S cross reactive | NA | NA |
| | | FF | | | | | | | | | |
| 275 | Beta | CASKGQGAAIRN | | TRBV19 | TRBD1 | TRBJ1-6 | NA | NA | S cross reactive | NA | NA |
| | | NSPLHF | | | | | | | | | |
| 276 | Beta | CASRDTGRLETQ | | TRBV28 | TRBD1 | TRBJ2-5 | NA | NA | S cross reactive | NA | NA |
| | | YF | | | | | | | | | |
| 277 | Beta | CASRTYPAARYN | | TRBV5-8 | TRBD2 | TRBJ2-1 | NA | NA | S cross reactive | NA | NA |
| | | EQFF | | | | | | | | | |
| 278 | Beta | CASSFSQGTGNS | | TRBV28 | TRBD1 | TRBJ1-6 | NA | NA | S cross reactive | NA | NA |
| | | PLHF | | | | | | | | | |
| 279 | Beta | CASSKKRTGPGA | | TRBV6-5 | TRBD1 | TRBJ2-6 | NA | NA | S cross reactive | NA | NA |
| | | NVLTF | | | | | | | | | |
| 280 | Beta | CASSLNRKNYGY | | TRBV12-3 | TRBD1 | TRBJ1-2 | NA | NA | S cross reactive | NA | NA |
| | | TF | | | | | | | | | |
| 281 | Beta | CASSRPGTGGAA | | TRBV5-6 | TRBD1 | TRBJ1-2 | NA | NA | S cross reactive | NA | NA |
| | | YGYTF | | | | | | | | | |
| 282 | Beta | CASSSTGGLYNE | | TRBV7-6 | TRBD1 | TRBJ2-1 | NA | NA | S cross reactive | NA | NA |
| | | QFF | | | | | | | | | |
| 283 | Beta | CASSYTRSGVGG | | TRBV6-5 | TRBD1 | TRBJ2-4 | NA | NA | S cross reactive | NA | NA |
| | | KNIQYF | | | | | | | | | |
| 284 | Beta | CATSDLRGSTDT | | TRBV24-1 | TRBD1 | TRBJ2-3 | NA | NA | S cross reactive | NA | NA |
| | | QYF | | | | | | | | | |
| 285 | Beta | CATSDLRTGGDT | | TRBV24-1 | TRBD1 | TRBJ2-3 | NA | NA | S cross reactive | NA | NA |
| | | QYF | | | | | | | | | |
| 286 | Beta | CATSRGRAGVGS | | TRBV15 | TRBD2 | TRBJ2-6 | NA | NA | S cross reactive | NA | NA |
| | | GANVLTF | | | | | | | | | |
| 287 | Beta | CATSVVSGVRTG | | TRBV6-5 | TRBD2 | TRBJ2-2 | NA | NA | S cross reactive | NA | NA |
| | | ELFF | | | | | | | | | |
| 288 | Beta | CAWSPGSQPQHF | | TRBV30 | TRBD1 | TRBJ1-5 | NA | NA | S cross reactive | NA | NA |
| 289 | Beta | CAWSVGGNYGYT | | TRBV30 | TRBD1 | TRBJ1-2 | NA | NA | S cross reactive | NA | NA |
| | | F | | | | | | | | | |
| 290 | Beta | CAWSVQGNYGYT | | TRBV30 | | TRBJ1-2 | NA | NA | S cross reactive | NA | NA |
| | | F | | | | | | | | | |
| 291 | Beta | CAWSVQQNYGYT | | TRBV30 | | TRBJ1-2 | NA | NA | S cross reactive | NA | NA |
| | | F | | | | | | | | | |
| 292 | Beta | CSALDRGSLYVY | | TRBV20-1 | TRBD1 | TRBJ1-2 | NA | NA | S cross reactive | NA | NA |
| | | GYTF | | | | | | | | | |
| 293 | Beta | CASSPTGAVQPQ | | TRBV18 | TRBD1 | TRBJ1-5 | NA | NA | S cross reactive | NA | NA |
| | | HF | | | | | | | | | |
| 294 | Beta | CAWSADRGVDTQ | | TRBV30 | TRBD1 | TRBJ2-3 | NA | NA | S cross reactive | NA | NA |
| | | YF | | | | | | | | | |
| 295 | Beta | CAWSAGQGARET | | TRBV30 | TRBD1 | TRBJ2-5 | NA | NA | S cross reactive | NA | NA |
| | | QYF | | | | | | | | | |
| 296 | Beta | CAWSVQQSYGYT | | TRBV30 | | TRBJ1-2 | NA | NA | S cross reactive | NA | NA |
| | | F | | | | | | | | | |
| 297 | Beta | CSARDQGRRQFF | | TRBV20-1 | TRBD1 | TRBJ2-1 | NA | NA | S cross reactive | NA | NA |
| 298 | Beta | CSARKLTGTQRY | | TRBV20-1 | TRBD1 | TRBJ1-2 | NA | NA | S cross reactive | NA | NA |
| | | GYTF | | | | | | | | | |
| 299 | Beta | CASSDPKFLGAL | | TRBV6-4 | TRBD1 | TRBJ2-6 | NA | NA | S cross reactive | NA | NA |
| | | RGANVLTF | | | | | | | | | |
| 300 | Beta | CASSSTGFSSYE | | TRBV6-1, | TRBD1 | TRBJ2-7 | NA | NA | S cross reactive | NA | NA |
| | | QYF | | TRBV6-3, | | | | | | | |
| | | | | TRBV6-5, | | | | | | | |
| | | | | TRBV6-6, | | | | | | | |
| | | | | TRBV6-8 | | | | | | | |
| 301 | Beta | CAWSGSGLGYGY | | TRBV30 | TRBD2 | TRBJ1-2 | NA | NA | S cross reactive | NA | NA |
| | | TF | | | | | | | | | |
| 302 | Beta | CAWSRDSNTEAF | | TRBV30 | TRBD1 | TRBJ1-1 | NA | NA | S cross reactive | NA | NA |
| | | F | | | | | | | | | |
| 303 | Beta | CAWSRDTSAYNE | | TRBV30 | TRBD1, | TRBJ2-1 | NA | NA | S cross reactive | NA | NA |
| | | QFF | | | TRBD2 | | | | | | |
| 304 | Beta | CAWSVSQNYGYT | | TRBV30 | | TRBJ1-2 | NA | NA | S cross reactive | NA | NA |
| | | F | | | | | | | | | |
| 305 | Beta | CAWSVSSSYGYT | | TRBV30 | | TRBJ1-2 | NA | NA | S cross reactive | NA | NA |
| | | F | | | | | | | | | |
| 306 | Beta | CASINAGSSSYE | | TRBV6-1, | | TRBJ2-7 | NA | NA | S cross reactive | NA | NA |
| | | QYF | | TRBV6-5, | | | | | | | |
| | | | | TRBV6-6, | | | | | | | |
| | | | | TRBV6-8, | | | | | | | |
| | | | | TRBV6-9 | | | | | | | |
| 307 | Beta | CASRDTSSRSEA | | TRBV11-1, | TRBD1, | TRBJ1-1 | NA | NA | S cross reactive | NA | NA |
| | | FF | | TRBV11-3 | TRBD2 | | | | | | |
| 308 | Beta | CASSAGLSGANV | | TRBV28 | TRBD2 | TRBJ2-6 | NA | NA | S cross reactive | NA | NA |
| | | LTF | | | | | | | | | |
| 309 | Beta | CASSLLTGLYGY | | TRBV28 | TRBD1 | TRBJ1-2 | NA | NA | S cross reactive | NA | NA |
| | | TF | | | | | | | | | |
| 310 | Beta | CASSQDRYEQYF | | TRBV19 | TRBD1 | TRBJ2-7 | NA | NA | S cross reactive | NA | NA |
| 311 | Beta | CSARGPGSRINE | | TRBV20-1 | TRBD1, | TRBJ2-7 | NA | NA | S cross reactive | NA | NA |
| | | QYF | | | TRBD2 | | | | | | |
| 312 | Beta | CASSIGTESYNE | | TRBV19 | TRBD1, | TRBJ2-1 | NA | NA | S cross reactive | NA | NA |
| | | QFF | | | TRBD2 | | | | | | |
| 313 | Beta | CASSPGSEVAKN | | TRBV7-9 | TRBD1 | TRBJ2-4 | NA | NA | S cross reactive | NA | NA |
| | | IQYF | | | | | | | | | |
| 314 | Beta | CAWIRDGQGAQA | | TRBV30 | TRBD1 | TRBJ1-1 | NA | NA | S cross reactive | NA | NA |
| | | FF | | | | | | | | | |
| 315 | Beta | CAWSGGGRRDTQ | | TRBV30 | TRBD1, | TRBJ2-3 | NA | NA | S cross reactive | NA | NA |
| | | YF | | | TRBD2 | | | | | | |
| 316 | Beta | CAWSLGTARDTQ | | TRBV30 | TRBD1 | TRBJ2-3 | NA | NA | S cross reactive | NA | NA |
| | | YF | | | | | | | | | |
| 317 | Beta | CASQGGPTEAFF | | TRBV30 | TRBD2 | TRBJ1-1 | NA | NA | S cross reactive | NA | NA |
| 318 | Beta | CASSEARGAGTQ | | TRBV2 | TRBD1 | TRBJ2-5 | NA | NA | S cross reactive | NA | NA |
| | | YF | | | | | | | | | |
| 319 | Beta | CASSETRGEQET | | TRBV2 | TRBD1 | TRBJ2-5 | NA | NA | S cross reactive | NA | NA |
| | | QYF | | | | | | | | | |
| 320 | Beta | CASSSLLAGVSY | | TRBV6-2 | TRBD2 | TRBJ2-7 | NA | NA | S cross reactive | NA | NA |
| | | EQYF | | | | | | | | | |
| 321 | Beta | CASSVATGPYGY | | TRBV28 | TRBD1 | TRBJ1-2 | NA | NA | S cross reactive | NA | NA |
| | | TF | | | | | | | | | |
| 322 | Beta | CAGFTGGMAGYT | | TRBV30 | TRBD1 | TRBJ1-2 | NA | NA | S cross reactive | NA | NA |
| | | F | | | | | | | | | |
| 323 | Beta | CASANGQAPYGY | | TRBV6-1, | TRBD1 | TRBJ1-2 | NA | NA | S cross reactive | NA | NA |
| | | TF | | TRBV6-3, | | | | | | | |
| | | | | TRBV6-5, | | | | | | | |
| | | | | TRBV6-6, | | | | | | | |
| | | | | TRBV6-8 | | | | | | | |
| 324 | Beta | CASGAGGTSSYE | | TRBV6-1, | TRBD1 | TRBJ2-7 | NA | NA | S cross reactive | NA | NA |
| | | QYF | | TRBV6-3, | | | | | | | |
| | | | | TRBV6-5, | | | | | | | |
| | | | | TRBV6-6, | | | | | | | |
| | | | | TRBV6-8 | | | | | | | |
| 325 | Beta | CASSYDRAPYGY | | TRBV6-1, | TRBD1 | TRBJ1-2 | NA | NA | S cross reactive | NA | NA |
| | | TF | | TRBV6-3, | | | | | | | |
| | | | | TRBV6-5, | | | | | | | |
| | | | | TRBV6-6, | | | | | | | |
| | | | | TRBV6-8 | | | | | | | |
| 326 | Beta | CAWSSTGGRRDT | | TRBV30 | TRBD1 | TRBJ2-3 | NA | NA | S cross reactive | NA | NA |
| | | QYF | | | | | | | | | |
| 327 | Beta | CAWSVQANYGYT | | TRBV30 | | TRBJ1-2 | NA | NA | S cross reactive | NA | NA |
| | | F | | | | | | | | | |
| 328 | Beta | CAWSVQPRGYTF | | TRBV30 | | TRBJ1-2 | NA | NA | S cross reactive | NA | NA |
| 329 | Beta | CSALDRKQYF | | TRBV20-1 | TRBD1 | TRBJ2-3 | NA | NA | S cross reactive | NA | NA |
| 330 | Beta | CASRGQVQETQY | | TRBV5-4 | TRBD1 | TRBJ2-5 | NA | NA | SARS-COV-2 N | NA | NA |
| | | F | | | | | | | | | |
| 331 | Beta | CASSLEFQAGEL | | TRBV5-4 | | TRBJ2-2 | NA | NA | SARS-COV-2 N | NA | NA |
| | | FF | | | | | | | | | |
| 332 | Beta | CASSLRQDSTSP | | TRBV5-4 | TRBD1 | TRBJ1-6 | NA | NA | SARS-COV-2 N | NA | NA |
| | | LHF | | | | | | | | | |
| 333 | Beta | CASSLVSGVGFY | | TRBV7-2 | TRBD1 | TRBJ1-2 | NA | NA | SARS-COV-2 N | NA | NA |
| | | GYTF | | | | | | | | | |
| 334 | Beta | CASSPTGTRTDT | | TRBV2 | TRBD1 | TRBJ2-3 | NA | NA | SARS-COV-2 N | NA | NA |
| | | QYF | | | | | | | | | |
| 335 | Beta | CATSTGTGRVDT | | TRBV24-1 | TRBD1 | TRBJ2-3 | NA | NA | SARS-COV-2 N | NA | NA |
| | | QYF | | | | | | | | | |
| 336 | Beta | CAIRDKSTDTQY | | TRBV10-3 | TRBD1 | TRBJ2-3 | NA | NA | SARS-COV-2 N | NA | NA |
| | | F | | | | | | | | | |
| 337 | Beta | CAISDISDEQYF | | TRBV10-3 | | TRBJ2-7 | NA | NA | SARS-COV-2 N | NA | NA |
| 338 | Beta | CAISEQALSNQP | | TRBV10-3 | TRBD2 | TRBJ1-5 | NA | NA | SARS-COV-2 N | NA | NA |
| | | QHF | | | | | | | | | |
| 339 | Beta | CAISGTSGGDTQ | | TRBV10-3 | TRBD2 | TRBJ2-3 | NA | NA | SARS-COV-2 N | NA | NA |
| | | YF | | | | | | | | | |
| 340 | Beta | CAISPANYGYTF | | TRBV10-3 | | TRBJ1-2 | NA | NA | SARS-COV-2 N | NA | NA |
| 341 | Beta | CASRLKPRSTGE | | TRBV27 | TRBD1, | TRBJ2-2 | NA | NA | SARS-COV-2 N | NA | NA |
| | | LFF | | | TRBD2 | | | | | | |
| 342 | Beta | CASSESLGGTDT | | TRBV10-1 | | TRBJ2-3 | NA | NA | SARS-COV-2 N | NA | NA |
| | | QYF | | | | | | | | | |
| 343 | Beta | CASSGQAWSTEA | | TRBV12-3, | TRBD1 | TRBJ1-1 | NA | NA | SARS-COV-2 N | NA | NA |
| | | FF | | TRBV12-4 | | | | | | | |
| 344 | Beta | CASSLALMNTEA | | TRBV5-1 | | TRBJ1-1 | NA | NA | SARS-COV-2 N | NA | NA |
| | | FF | | | | | | | | | |
| 345 | Beta | CASSLPMEPLEG | | TRBV11-1, | | TRBJ1-1 | NA | NA | SARS-COV-2 N | NA | NA |
| | | DTEAFF | | TRBV11-3 | | | | | | | |
| 346 | Beta | CASSLRGAVRTG | | TRBV11-2 | TRBD1 | TRBJ2-2 | NA | NA | SARS-COV-2 N | NA | NA |
| | | ELFF | | | | | | | | | |
| 347 | Beta | CASSPFTYEQYF | | TRBV18 | | TRBJ2-7 | NA | NA | SARS-COV-2 N | NA | NA |
| 348 | Beta | CASSPQGGSLAY | | TRBV19 | TRBD1 | TRBJ2-7 | NA | NA | SARS-COV-2 N | NA | NA |
| | | EQYF | | | | | | | | | |
| 349 | Beta | CASSPTTVKYGY | | TRBV7-2, | | TRBJ1-2 | NA | NA | SARS-COV-2 N | NA | NA |
| | | TF | | TRBV7-8 | | | | | | | |
| 350 | Beta | CASSQDETGDQE | | TRBV3-1, | TRBD1 | TRBJ2-5 | NA | NA | SARS-COV-2 N | NA | NA |
| | | TQYF | | TRBV3-2 | | | | | | | |
| 351 | Beta | CASSVDGSNQPQ | | TRBV5-1 | | TRBJ1-5 | NA | NA | SARS-COV-2 N | NA | NA |
| | | HF | | | | | | | | | |
| 352 | Beta | CASSYPEGQSLA | | TRBV6-1, | TRBD1 | TRBJ1-1 | NA | NA | SARS-COV-2 N | NA | NA |
| | | FF | | TRBV6-3, | | | | | | | |
| | | | | TRBV6-5, | | | | | | | |
| | | | | TRBV6-6, | | | | | | | |
| | | | | TRBV6-8 | | | | | | | |
| 353 | Beta | CASTLSGYNTGE | | TRBV5-4, | TRBD2 | TRBJ2-2 | NA | NA | SARS-COV-2 N | NA | NA |
| | | LFF | | TRBV5-8 | | | | | | | |
| 354 | Beta | CASTMGQGNTDT | | TRBV18 | TRBD1 | TRBJ2-3 | NA | NA | SARS-COV-2 N | NA | NA |
| | | QYF | | | | | | | | | |
| 355 | Beta | CASTTGLAGGQE | | TRBV12-3, | TRBD2 | TRBJ2-5 | NA | NA | SARS-COV-2 N | NA | NA |
| | | TQYF | | TRBV12-4 | | | | | | | |
| 356 | Beta | CAWSVRSSGALN | | TRBV30 | TRBD2 | TRBJ2-1 | NA | NA | SARS-COV-2 N | NA | NA |
| | | NEQFF | | | | | | | | | |
| 357 | Beta | CAWWDRGHGYTF | | TRBV30 | TRBD1 | TRBJ1-2 | NA | NA | SARS-COV-2 N | NA | NA |
| 358 | Beta | CSARKGQGNYGY | | TRBV20-1 | TRBD1 | TRBJ1-2 | NA | NA | SARS-COV-2 N | NA | NA |
| | | TF | | | | | | | | | |
| 359 | Beta | CSASGLAHSQYF | | TRBV20-1 | TRBD2 | TRBJ2-3 | NA | NA | SARS-COV-2 N | NA | NA |
| 360 | Beta | CSVDYGGGTDTQ | | TRBV29-1 | TRBD1 | TRBJ2-3 | NA | NA | SARS-COV-2 N | NA | NA |
| | | YF | | | | | | | | | |
| 361 | Beta | CAISESVGNYNE | | TRBV10-3 | TRBD1, | TRBJ2-1 | NA | NA | SARS-COV-2 N | NA | NA |
| | | QFF | | | TRBD2 | | | | | | |
| 362 | Beta | CAISPDMDGYTF | | TRBV10-3 | TRBD1 | TRBJ1-2 | NA | NA | SARS-COV-2 N | NA | NA |
| 363 | Beta | CASAWRTSGELF | | TRBV19 | TRBD1 | TRBJ2-2 | NA | NA | SARS-COV-2 N | NA | NA |
| | | F | | | | | | | | | |
| 364 | Beta | CASGTRHEQYF | | TRBV6-1, | TRBD2 | TRBJ2-7 | NA | NA | SARS-COV-2 N | NA | NA |
| | | | | TRBV6-3, | | | | | | | |
| | | | | TRBV6-5, | | | | | | | |
| | | | | TRBV6-6, | | | | | | | |
| | | | | TRBV6-8 | | | | | | | |
| 365 | Beta | CASKGPNTEAFF | | TRBV28 | | TRBJ1-1 | NA | NA | SARS-COV-2 N | NA | NA |
| 366 | Beta | CASLPGLGFGLQ | | TRBV7-9 | TRBD1, | TRBJ2-5 | NA | NA | SARS-COV-2 N | NA | NA |
| | | ETQYF | | | TRBD2 | | | | | | |
| 367 | Beta | CASSDYGAGTGL | | TRBV7-9 | TRBD1 | TRBJ1-2 | NA | NA | SARS-COV-2 N | NA | NA |
| | | KGYTF | | | | | | | | | |
| 368 | Beta | CASSEVRSNQPQ | | TRBV2 | | TRBJ1-5 | NA | NA | SARS-COV-2 N | NA | NA |
| | | HF | | | | | | | | | |
| 369 | Beta | CASSFRRENTIY | | TRBV7-2, | TRBD2 | TRBJ1-3 | NA | NA | SARS-COV-2 N | NA | NA |
| | | F | | TRBV7-8 | | | | | | | |
| 370 | Beta | CASSIDLRQGAT | | TRBV19 | TRBD1, | TRBJ2-3 | NA | NA | SARS-COV-2 N | NA | NA |
| | | QYF | | | TRBD2 | | | | | | |
| 371 | Beta | CASSLTGEQYF | | TRBV12-3, | TRBD1 | TRBJ2-7 | NA | NA | SARS-COV-2 N | NA | NA |
| | | | | TRBV12-4 | | | | | | | |
| 372 | Beta | CASSPRGNEQFF | | TRBV7-9 | TRBD1, | TRBJ2-1 | NA | NA | SARS-COV-2 N | NA | NA |
| | | | | | TRBD2 | | | | | | |
| 373 | Beta | CASSRDTDGYTF | | TRBV2 | TRBD1, | TRBJ1-2 | NA | NA | SARS-COV-2 N | NA | NA |
| | | | | | TRBD2 | | | | | | |
| 374 | Beta | CASSSAEGGNQP | | TRBV27 | TRBD2 | TRBJ1-5 | NA | NA | SARS-COV-2 N | NA | NA |
| | | QHF | | | | | | | | | |
| 375 | Beta | CASSSNGPSGYT | | TRBV7-9 | | TRBJ1-2 | NA | NA | SARS-COV-2 N | NA | NA |
| | | F | | | | | | | | | |
| 376 | Beta | CASKMDSRNQPQ | | TRBV6-2, | TRBD2 | TRBJ1-5 | NA | NA | SARS-COV-2 N | NA | NA |
| | | HF | | TRBV6-3 | | | | | | | |
| 377 | Beta | CASKPTGAAGAQ | | TRBV27 | TRBD1 | TRBJ1-5 | NA | NA | SARS-COV-2 N | NA | NA |
| | | HF | | | | | | | | | |
| 378 | Beta | CASSAGGAGANV | | TRBV10-1 | TRBD1 | TRBJ2-6 | NA | NA | SARS-COV-2 N | NA | NA |
| | | LTF | | | | | | | | | |
| 379 | Beta | CASSEATFGTSG | | TRBV6-1, | TRBD2 | TRBJ2-3 | NA | NA | SARS-COV-2 N | NA | NA |
| | | TDTQYF | | TRBV6-5 | | | | | | | |
| 380 | Beta | CASSEWSGAQET | | TRBV10-2 | TRBD1 | TRBJ2-5 | NA | NA | SARS-COV-2 N | NA | NA |
| | | QYF | | | | | | | | | |
| 381 | Beta | CASSFGVPGQVT | | TRBV7-2, | TRBD1 | TRBJ1-5 | NA | NA | SARS-COV-2 N | NA | NA |
| | | GQPQHF | | TRBV7-8 | | | | | | | |
| 382 | Beta | CASSSSNQPQHF | | TRBV7-6, | | TRBJ1-5 | NA | NA | SARS-COV-2 N | NA | NA |
| | | | | TRBV7-7 | | | | | | | |
| 383 | Beta | CASSTDRLGTYS | | TRBV6-1, | TRBD1, | TRBJ1-5 | NA | NA | SARS-COV-2 N | NA | NA |
| | | NQPQHF | | TRBV6-5, | TRBD2 | | | | | | |
| | | | | TRBV6-6, | | | | | | | |
| | | | | TRBV6-8 | | | | | | | |
| 384 | Beta | CASSWTVPPLHF | | TRBV12-3, | TRBD1 | TRBJ1-6 | NA | NA | SARS-COV-2 N | NA | NA |
| | | | | TRBV12-4 | | | | | | | |
| 385 | Beta | CATARAANTEAF | | TRBV6-1, | TRBD1, | TRBJ1-1 | NA | NA | SARS-COV-2 N | NA | NA |
| | | F | | TRBV6-5, | TRBD2 | | | | | | |
| | | | | TRBV6-6 | | | | | | | |
| 386 | Beta | CATSDPRQGASN | | TRBV24-1 | TRBD1 | TRBJ1-5 | NA | NA | SARS-COV-2 N | NA | NA |
| | | QPQHF | | | | | | | | | |
| 387 | Beta | CATSEQGAGNQP | | TRBV24-1 | TRBD1 | TRBJ1-5 | NA | NA | SARS-COV-2 N | NA | NA |
| | | QHF | | | | | | | | | |
| 388 | Beta | CATSRGERGGTE | | TRBV15 | TRBD1 | TRBJ1-1 | NA | NA | SARS-COV-2 N | NA | NA |
| | | AFF | | | | | | | | | |
| 389 | Beta | CRFSALGNTEAF | | TRBV20-1 | | TRBJ1-1 | NA | NA | SARS-COV-2 N | NA | NA |
| | | F | | | | | | | | | |
| 390 | Beta | CSAGRGDTEAFF | | TRBV20-1 | TRBD1, | TRBJ1-1 | NA | NA | SARS-COV-2 N | NA | NA |
| | | | | | TRBD2 | | | | | | |
| 391 | Beta | CSAGSRGGANTE | | TRBV20-1 | TRBD1, | TRBJ1-1 | NA | NA | SARS-COV-2 N | NA | NA |
| | | AFF | | | TRBD2 | | | | | | |
| 392 | Beta | CSAKGGVNTEAF | | TRBV20-1 | TRBD1 | TRBJ1-1 | NA | NA | SARS-COV-2 N | NA | NA |
| | | F | | | | | | | | | |
| 393 | Beta | CSALRGGNTIYF | | TRBV20-1 | TRBD1, | TRBJ1-3 | NA | NA | SARS-COV-2 N | NA | NA |
| | | | | | TRBD2 | | | | | | |
| 394 | Beta | CSAPPRGAPGTQ | | TRBV20-1 | TRBD1 | TRBJ2-5 | NA | NA | SARS-COV-2 N | NA | NA |
| | | YF | | | | | | | | | |
| 395 | Beta | CSAPQNGGGRET | | TRBV20-1 | TRBD1, | TRBJ2-5 | NA | NA | SARS-COV-2 N | NA | NA |
| | | QYF | | | TRBD2 | | | | | | |
| 396 | Beta | CSARNRLDGSYT | | TRBV20-1 | TRBD1, | TRBJ1-2 | NA | NA | SARS-COV-2 N | NA | NA |
| | | F | | | TRBD2 | | | | | | |
| 397 | Beta | CSARVYRAGASS | | TRBV20-1 | TRBD2 | TRBJ2-6 | NA | NA | SARS-COV-2 N | NA | NA |
| | | GANVLTF | | | | | | | | | |
| 398 | Beta | CSASNFRTSVSS | | TRBV20-1 | TRBD2 | TRBJ2-6 | NA | NA | SARS-COV-2 N | NA | NA |
| | | GANVLTF | | | | | | | | | |
| 399 | Beta | CSASSRHTGELF | | TRBV20-1 | | TRBJ2-2 | NA | NA | SARS-COV-2 N | NA | NA |
| | | F | | | | | | | | | |
| 400 | Beta | CSATWAFSNQPQ | | TRBV20-1 | TRBD2 | TRBJ1-5 | NA | NA | SARS-COV-2 N | NA | NA |
| | | HF | | | | | | | | | |
| 401 | Beta | CASSELQGAGYT | | TRBV2 | TRBD1 | TRBJ1-2 | NA | NA | SARS-COV-2 N | NA | NA |
| | | F | | | | | | | | | |
| 402 | Beta | CASSETGDRSPL | | TRBV2 | TRBD1 | TRBJ1-6 | NA | NA | SARS-COV-2 N | NA | NA |
| | | HF | | | | | | | | | |
| 403 | Beta | CASSIVSGDYGY | | TRBV19 | TRBD1 | TRBJ1-2 | NA | NA | SARS-COV-2 N | NA | NA |
| | | TF | | | | | | | | | |
| 404 | Beta | CASSPSPGESYE | | TRBV7-8 | TRBD2 | TRBJ2-7 | NA | NA | SARS-COV-2 N | NA | NA |
| | | QYF | | | | | | | | | |
| 405 | Beta | CASSQIQGAGYT | | TRBV14 | TRBD1 | TRBJ1-2 | NA | NA | SARS-COV-2 N | NA | NA |
| | | F | | | | | | | | | |
| 406 | Beta | CASSRSGTGSDT | | TRBV7-2 | TRBD1 | TRBJ2-3 | NA | NA | SARS-COV-2 N | NA | NA |
| | | QYF | | | | | | | | | |
| 407 | Beta | CASSTQPGLAGE | | TRBV19 | TRBD2 | TRBJ2-5 | NA | NA | SARS-COV-2 N | NA | NA |
| | | TQYF | | | | | | | | | |
| 408 | Beta | CASSYRGMSTDT | | TRBV6-2 | TRBD1 | TRBJ2-3 | NA | NA | SARS-COV-2 N | NA | NA |
| | | QYF | | | | | | | | | |
| 409 | Beta | CATMRGGSAEAF | | TRBV24-1 | TRBD1 | TRBJ1-1 | NA | NA | SARS-COV-2 N | NA | NA |
| | | F | | | | | | | | | |
| 410 | Beta | CAIRDYSADTQY | | TRBV10-3 | TRBD2 | TRBJ2-3 | NA | NA | SARS-COV-2 N | NA | NA |
| | | F | | | | | | | | | |
| 411 | Beta | CAISDRAGYGYT | | TRBV10-3 | TRBD1 | TRBJ1-2 | NA | NA | SARS-COV-2 N | NA | NA |
| | | F | | | | | | | | | |
| 412 | Beta | CAISEAGGFMNT | | TRBV10-3 | TRBD1 | TRBJ1-1 | NA | NA | SARS-COV-2 N | NA | NA |
| | | EAFF | | | | | | | | | |
| 413 | Beta | CAISGWDGHYGY | | TRBV10-3 | TRBD1 | TRBJ1-2 | NA | NA | SARS-COV-2 N | NA | NA |
| | | TF | | | | | | | | | |
| 414 | Beta | CASRAGQRYGYT | | TRBV27 | TRBD1 | TRBJ1-2 | NA | NA | SARS-COV-2 N | NA | NA |
| | | F | | | | | | | | | |
| 415 | Beta | CASSDRGRFYGY | | TRBV6-3 | TRBD1 | TRBJ1-2 | NA | NA | SARS-COV-2 N | NA | NA |
| | | TF | | | | | | | | | |
| 416 | Beta | CASSEGSHSTQH | | TRBV6-1 | | TRBJ1-5 | NA | NA | SARS-COV-2 N | NA | NA |
| | | F | | | | | | | | | |
| 417 | Beta | CASSLAGRGRSP | | TRBV7-2 | TRBD1 | TRBJ1-6 | NA | NA | SARS-COV-2 N | NA | NA |
| | | LHF | | | | | | | | | |
| 418 | Beta | CASSLDYSQIQH | | TRBV7-2 | | TRBJ1-5 | NA | NA | SARS-COV-2 N | NA | NA |
| | | F | | | | | | | | | |
| 419 | Beta | CASSLKGAGWTG | | TRBV27 | TRBD1 | TRBJ2-2 | NA | NA | SARS-COV-2 N | NA | NA |
| | | ELFF | | | | | | | | | |
| 420 | Beta | CASSMTDSYEQY | | TRBV7-8 | TRBD1 | TRBJ2-7 | NA | NA | SARS-COV-2 N | NA | NA |
| | | F | | | | | | | | | |
| 421 | Beta | CASSPVSNYGYT | | TRBV6-1 | TRBD1 | TRBJ1-2 | NA | NA | SARS-COV-2 N | NA | NA |
| | | F | | | | | | | | | |
| 422 | Beta | CASSTDRRKGGY | | TRBV7-2 | TRBD1 | TRBJ1-2 | NA | NA | SARS-COV-2 N | NA | NA |
| | | TF | | | | | | | | | |
| 423 | Beta | CATHIGQGSSNQ | | TRBV10-3 | TRBD1 | TRBJ1-5 | NA | NA | SARS-COV-2 N | NA | NA |
| | | PQHF | | | | | | | | | |
| 424 | Beta | CSAEQGAGELFF | | TRBV20-1 | TRBD1 | TRBJ2-2 | NA | NA | SARS-COV-2 N | NA | NA |
| 425 | Beta | CSAQRTYGYTF | | TRBV20-1 | TRBD2 | TRBJ1-2 | NA | NA | SARS-COV-2 N | NA | NA |
| 426 | Beta | CSARNRGLNNQP | | TRBV20-1 | TRBD2 | TRBJ1-5 | NA | NA | SARS-COV-2 N | NA | NA |
| | | QHF | | | | | | | | | |
| 427 | Beta | CASKRPGRMNTE | | TRBV19 | | TRBJ1-1 | NA | NA | SARS-COV-2 N | NA | NA |
| | | AFF | | | | | | | | | |
| 428 | Beta | CASRHREKTGGE | | TRBV6-5 | TRBD1 | TRBJ1-5 | NA | NA | SARS-COV-2 N | NA | NA |
| | | QNQPQHF | | | | | | | | | |
| 429 | Beta | CASRQREGATIY | | TRBV6-1 | TRBD2 | TRBJ1-2 | NA | NA | SARS-COV-2 N | NA | NA |
| | | GYTF | | | | | | | | | |
| 430 | Beta | CASSFGELFF | | TRBV28 | | TRBJ2-2 | NA | NA | SARS-COV-2 N | NA | NA |
| 431 | Beta | CASSFRIKFAGS | | TRBV27 | TRBD2 | TRBJ2-1 | NA | NA | SARS-COV-2 N | NA | NA |
| | | NNEQFF | | | | | | | | | |
| 432 | Beta | CASSFRIKLQGG | | TRBV6-5 | TRBD1 | TRBJ2-7 | NA | NA | SARS-COV-2 N | NA | NA |
| | | PAYEQYF | | | | | | | | | |
| 433 | Beta | CASSFRQGNSPL | | TRBV7-2 | TRBD1 | TRBJ1-6 | NA | NA | SARS-COV-2 N | NA | NA |
| | | HF | | | | | | | | | |
| 434 | Beta | CASSLKLDTEAF | | TRBV5-1 | | TRBJ1-1 | NA | NA | SARS-COV-2 N | NA | NA |
| | | F | | | | | | | | | |
| 435 | Beta | CASSLNGNQPQH | | TRBV5-1 | | TRBJ1-5 | NA | NA | SARS-COV-2 N | NA | NA |
| | | F | | | | | | | | | |
| 436 | Beta | CASSLPSGDSNE | | TRBV5-4 | TRBD2 | TRBJ2-1 | NA | NA | SARS-COV-2 N | NA | NA |
| | | QFF | | | | | | | | | |
| 437 | Beta | CASSLRAGGPTD | | TRBV5-4 | TRBD2 | TRBJ2-3 | NA | NA | SARS-COV-2 N | NA | NA |
| | | TQYF | | | | | | | | | |
| 438 | Beta | CASSSGQGTGEQ | | TRBV6-5 | TRBD1 | TRBJ2-7 | NA | NA | SARS-COV-2 N | NA | NA |
| | | YF | | | | | | | | | |
| 439 | Beta | CASSYPGIYNEQ | | TRBV11-3 | TRBD1 | TRBJ2-1 | NA | NA | SARS-COV-2 N | NA | NA |
| | | FF | | | | | | | | | |
| 440 | Beta | CASSYRVKYQGG | | TRBV6-5 | TRBD1 | TRBJ1-5 | NA | NA | SARS-COV-2 N | NA | NA |
| | | NQPQHF | | | | | | | | | |
| 441 | Beta | CSARAYQNYGYT | | TRBV20-1 | | TRBJ1-2 | NA | NA | SARS-COV-2 N | NA | NA |
| | | F | | | | | | | | | |
| 442 | Beta | CAEGGLAGREQY | | TRBV2 | TRBD2 | TRBJ2-7 | NA | NA | SARS-COV-2 N | NA | NA |
| | | F | | | | | | | | | |
| 443 | Beta | CASELAGVVGEL | | TRBV6-5 | TRBD2 | TRBJ2-2 | NA | NA | SARS-COV-2 N | NA | NA |
| | | FF | | | | | | | | | |
| 444 | Beta | CASSELGLNYGY | | TRBV2 | | TRBJ1-2 | NA | NA | SARS-COV-2 N | NA | NA |
| | | TF | | | | | | | | | |
| 445 | Beta | CASSERTEAFF | | TRBV2 | | TRBJ1-1 | NA | NA | SARS-COV-2 N | NA | NA |
| 446 | Beta | CASSESGNGADT | | TRBV2 | TRBD1 | TRBJ2-3 | NA | NA | SARS-COV-2 N | NA | NA |
| | | QYF | | | | | | | | | |
| 447 | Beta | CASSFWGNSNQP | | TRBV12-3 | TRBD1 | TRBJ1-5 | NA | NA | SARS-COV-2 N | NA | NA |
| | | QHF | | | | | | | | | |
| 448 | Beta | CASSGGTVGQYF | | TRBV6-1 | TRBD1 | TRBJ2-7 | NA | NA | SARS-COV-2 N | NA | NA |
| 449 | Beta | CASSIDGGSGEA | | TRBV19 | TRBD1 | TRBJ1-1 | NA | NA | SARS-COV-2 N | NA | NA |
| | | FF | | | | | | | | | |
| 450 | Beta | CASSKGPSLETQ | | TRBV2 | TRBD2 | TRBJ2-5 | NA | NA | SARS-COV-2 N | NA | NA |
| | | YF | | | | | | | | | |
| 451 | Beta | CASSLAGGGGYE | | TRBV7-2 | TRBD1 | TRBJ2-7 | NA | NA | SARS-COV-2 N | NA | NA |
| | | QYF | | | | | | | | | |
| 452 | Beta | CASSLARGYDTQ | | TRBV27 | TRBD1 | TRBJ2-3 | NA | NA | SARS-COV-2 N | NA | NA |
| | | YF | | | | | | | | | |
| 453 | Beta | CASSLDPGGGYG | | TRBV7-8 | TRBD1 | TRBJ1-2 | NA | NA | SARS-COV-2 N | NA | NA |
| | | YTF | | | | | | | | | |
| 454 | Beta | CASSLDRGAGGY | | TRBV7-9 | TRBD1 | TRBJ1-2 | NA | NA | SARS-COV-2 N | NA | NA |
| | | TF | | | | | | | | | |
| 455 | Beta | CASSLGVQGENT | | TRBV7-8 | TRBD1 | TRBJ1-1 | NA | NA | SARS-COV-2 N | NA | NA |
| | | EAFF | | | | | | | | | |
| 456 | Beta | CASSLNREYDEQ | | TRBV27 | TRBD1 | TRBJ2-7 | NA | NA | SARS-COV-2 N | NA | NA |
| | | YF | | | | | | | | | |
| 457 | Beta | CASSLNRGQDEQ | | TRBV6-5 | TRBD1 | TRBJ2-7 | NA | NA | SARS-COV-2 N | NA | NA |
| | | YF | | | | | | | | | |
| 458 | Beta | CASSLNRVRDTQ | | TRBV27 | TRBD1 | TRBJ2-3 | NA | NA | SARS-COV-2 N | NA | NA |
| | | YF | | | | | | | | | |
| 459 | Beta | CASSQDWGKVQP | | TRBV4-3 | TRBD1 | TRBJ1-5 | NA | NA | SARS-COV-2 N | NA | NA |
| | | QHF | | | | | | | | | |
| 460 | Beta | CASSSWGRGEKL | | TRBV21-1 | TRBD1 | TRBJ1-4 | NA | NA | SARS-COV-2 N | NA | NA |
| | | FF | | | | | | | | | |
| 461 | Beta | CASSVTSGGATS | | TRBV7-2 | TRBD2 | TRBJ2-3 | NA | NA | SARS-COV-2 N | NA | NA |
| | | STDTQYF | | | | | | | | | |
| 462 | Beta | CASSWGTDTQYF | | TRBV12-3 | TRBD1 | TRBJ2-3 | NA | NA | SARS-COV-2 N | NA | NA |
| 463 | Beta | CSAAPGQGLGYT | | TRBV20-1 | TRBD1 | TRBJ1-2 | NA | NA | SARS-COV-2 N | NA | NA |
| | | F | | | | | | | | | |
| 464 | Beta | CSARASTGTGGG | | TRBV20-1 | TRBD1 | TRBJ1-4 | NA | NA | SARS-COV-2 N | NA | NA |
| | | KLFF | | | | | | | | | |
| 465 | Beta | CSASHTGGVSDT | | TRBV20-1 | TRBD1 | TRBJ2-3 | NA | NA | SARS-COV-2 N | NA | NA |
| | | QYF | | | | | | | | | |
| 466 | Beta | CSNRASGFSPLH | | TRBV20-1 | TRBD1 | TRBJ1-6 | NA | NA | SARS-COV-2 N | NA | NA |
| | | F | | | | | | | | | |
| 467 | Beta | CASNRIDPSEQF | | TRBV2 | | TRBJ2-1 | NA | NA | SARS-COV-2 N | NA | NA |
| | | F | | | | | | | | | |
| 468 | Beta | CASRANRDLNYG | | TRBV2 | TRBD1 | TRBJ1-2 | NA | NA | SARS-COV-2 N | NA | NA |
| | | YTF | | | | | | | | | |
| 469 | Beta | CASSAAREWETQ | | TRBV2 | TRBD2 | TRBJ2-5 | NA | NA | SARS-COV-2 N | NA | NA |
| | | YF | | | | | | | | | |
| 470 | Beta | CASSAYTANYGY | | TRBV2 | | TRBJ1-2 | NA | NA | SARS-COV-2 N | NA | NA |
| | | TF | | | | | | | | | |
| 471 | Beta | CASSDRLATGEL | | TRBV7-3 | TRBD2 | TRBJ2-2 | NA | NA | SARS-COV-2 N | NA | NA |
| | | FF | | | | | | | | | |
| 472 | Beta | CASSENRDGHEQ | | TRBV2 | TRBD1, | TRBJ2-1 | NA | NA | SARS-COV-2 N | NA | NA |
| | | FF | | | TRBD2 | | | | | | |
| 473 | Beta | CASSENTEYGYT | | TRBV2 | | TRBJ1-2 | NA | NA | SARS-COV-2 N | NA | NA |
| | | F | | | | | | | | | |
| 474 | Beta | CASSEVASGERA | | TRBV2 | TRBD2 | TRBJ2-5 | NA | NA | SARS-COV-2 N | NA | NA |
| | | QYF | | | | | | | | | |
| 475 | Beta | CASSFGGTGAFF | | TRBV11-1, | TRBD1 | TRBJ1-1 | NA | NA | SARS-COV-2 N | NA | NA |
| | | | | TRBV11-3 | | | | | | | |
| 476 | Beta | CASSFLAGGNTG | | TRBV11-1, | TRBD1, | TRBJ2-2 | NA | NA | SARS-COV-2 N | NA | NA |
| | | ELFF | | TRBV11-3 | TRBD2 | | | | | | |
| 477 | Beta | CASSFVAYLNEQ | | TRBV11-1, | TRBD2 | TRBJ2-1 | NA | NA | SARS-COV-2 N | NA | NA |
| | | FF | | TRBV11-3 | | | | | | | |
| 478 | Beta | CASSLRAARGAY | | TRBV28 | TRBD1, | TRBJ1-2 | NA | NA | SARS-COV-2 N | NA | NA |
| | | GYTF | | | TRBD2 | | | | | | |
| 479 | Beta | CASSPVGDRAYE | | TRBV2 | TRBD1 | TRBJ2-7 | NA | NA | SARS-COV-2 N | NA | NA |
| | | QYF | | | | | | | | | |
| 480 | Beta | CASSRDGSSYNE | | TRBV5-4, | TRBD1, | TRBJ2-1 | NA | NA | SARS-COV-2 N | NA | NA |
| | | QFF | | TRBV5-8 | TRBD2 | | | | | | |
| 481 | Beta | CSARGIWTSGGD | | TRBV20-1 | TRBD2 | TRBJ2-7 | NA | NA | SARS-COV-2 N | NA | NA |
| | | SYEQYF | | | | | | | | | |
| 482 | Beta | CASRGDGRSPLH | | TRBV2 | TRBD1, | TRBJ1-6 | NA | NA | SARS-COV-2 N | NA | NA |
| | | F | | | TRBD2 | | | | | | |
| 483 | Beta | CASSAEKGRSGY | | TRBV2 | TRBD1, | TRBJ1-2 | NA | NA | SARS-COV-2 N | NA | NA |
| | | TF | | | TRBD2 | | | | | | |
| 484 | Beta | CASSAGTENIQY | | TRBV7-2, | TRBD1 | TRBJ2-4 | NA | NA | SARS-COV-2 N | NA | NA |
| | | F | | TRBV7-8 | | | | | | | |
| 485 | Beta | CASSESGRAMET | | TRBV2 | TRBD2 | TRBJ2-5 | NA | NA | SARS-COV-2 N | NA | NA |
| | | QYF | | | | | | | | | |
| 486 | Beta | CASSKVVITGPY | | TRBV21-1 | TRBD1, | TRBJ1-2 | NA | NA | SARS-COV-2 N | NA | NA |
| | | GYTF | | | TRBD2 | | | | | | |
| 487 | Beta | CASSLAGGLDGY | | TRBV7-2, | TRBD1 | TRBJ1-2 | NA | NA | SARS-COV-2 N | NA | NA |
| | | TF | | TRBV7-8 | | | | | | | |
| 488 | Beta | CASSLETGTGGD | | TRBV27 | TRBD1 | TRBJ1-5 | NA | NA | SARS-COV-2 N | NA | NA |
| | | NQPQHF | | | | | | | | | |
| 489 | Beta | CASSLGSSGEKD | | TRBV5-1 | TRBD2 | TRBJ2-3 | NA | NA | SARS-COV-2 N | NA | NA |
| | | TQYF | | | | | | | | | |
| 490 | Beta | CASSLIAGGHYE | | TRBV7-6, | TRBD2 | TRBJ2-7 | NA | NA | SARS-COV-2 N | NA | NA |
| | | QYF | | TRBV7-7 | | | | | | | |
| 491 | Beta | CASSLNPAGSTD | | TRBV7-3 | TRBD2 | TRBJ2-3 | NA | NA | SARS-COV-2 N | NA | NA |
| | | TQYF | | | | | | | | | |
| 492 | Beta | CASSLVPGQGSY | | TRBV7-2, | TRBD1, | TRBJ2-7 | NA | NA | SARS-COV-2 N | NA | NA |
| | | EQYF | | TRBV7-8 | TRBD2 | | | | | | |
| 493 | Beta | CASSLVPYEQYF | | TRBV28 | | TRBJ2-7 | NA | NA | SARS-COV-2 N | NA | NA |
| 494 | Beta | CASSPRVYEQFF | | TRBV2 | TRBD1 | TRBJ2-1 | NA | NA | SARS-COV-2 N | NA | NA |
| 495 | Beta | CASSQGDIGNQP | | TRBV4-2, | | TRBJ1-5 | NA | NA | SARS-COV-2 N | NA | NA |
| | | QHF | | TRBV4-3 | | | | | | | |
| 496 | Beta | CASSQGQGDSPL | | TRBV4-1, | TRBD1 | TRBJ1-6 | NA | NA | SARS-COV-2 N | NA | NA |
| | | HF | | TRBV4-2, | | | | | | | |
| | | | | TRBV4-3 | | | | | | | |
| 497 | Beta | CASSRGLAGGTD | | TRBV6-1, | TRBD2 | TRBJ2-3 | NA | NA | SARS-COV-2 N | NA | NA |
| | | TQYF | | TRBV6-5, | | | | | | | |
| | | | | TRBV6-6, | | | | | | | |
| | | | | TRBV6-8, | | | | | | | |
| | | | | TRBV6-9 | | | | | | | |
| 498 | Beta | CASSRRGGSSPL | | TRBV28 | TRBD1, | TRBJ1-6 | NA | NA | SARS-COV-2 N | NA | NA |
| | | HF | | | TRBD2 | | | | | | |
| 499 | Beta | CASSRWQGRTEA | | TRBV7-2, | TRBD1 | TRBJ1-1 | NA | NA | SARS-COV-2 N | NA | NA |
| | | FF | | TRBV7-8 | | | | | | | |
| 500 | Beta | CASSSRGVGTEA | | TRBV7-9 | TRBD1 | TRBJ1-1 | NA | NA | SARS-COV-2 N | NA | NA |
| | | FF | | | | | | | | | |
| 501 | Beta | CASSTTGQGSDE | | TRBV2 | TRBD1 | TRBJ2-1 | NA | NA | SARS-COV-2 N | NA | NA |
| | | QFF | | | | | | | | | |
| 502 | Beta | CASVSNQPQHF | | TRBV21-1 | | TRBJ1-5 | NA | NA | SARS-COV-2 N | NA | NA |
| 503 | Beta | CATSDLGEGETQ | | TRBV24-1 | | TRBJ2-5 | NA | NA | SARS-COV-2 N | NA | NA |
| | | YF | | | | | | | | | |
| 504 | Beta | CSARDLTGDGYT | | TRBV20-1 | TRBD1 | TRBJ1-2 | NA | NA | SARS-COV-2 N | NA | NA |
| | | F | | | | | | | | | |
| 505 | Beta | CSASDNRRNYGY | | TRBV20-1 | | TRBJ1-2 | NA | NA | SARS-COV-2 N | NA | NA |
| | | TF | | | | | | | | | |
| 506 | Beta | CAISDRGASEAF | | TRBV10-3 | | TRBJ1-1 | NA | NA | SARS-COV-2 N | NA | NA |
| | | F | | | | | | | | | |
| 507 | Beta | CAISERSGDYGY | | TRBV10-3 | | TRBJ1-2 | NA | NA | SARS-COV-2 N | NA | NA |
| | | TF | | | | | | | | | |
| 508 | Beta | CASSFESTDTQY | | TRBV7-9 | | TRBJ2-3 | NA | NA | SARS-COV-2 N | NA | NA |
| | | F | | | | | | | | | |
| 509 | Beta | CASSFKGQGFSP | | TRBV27 | TRBD1 | TRBJ1-6 | NA | NA | SARS-COV-2 N | NA | NA |
| | | LHF | | | | | | | | | |
| 510 | Beta | CASSGQGSTDTQ | | TRBV7-9 | TRBD1 | TRBJ2-3 | NA | NA | SARS-COV-2 N | NA | NA |
| | | YF | | | | | | | | | |
| 511 | Beta | CASSLEGSTDTQ | | TRBV7-9 | | TRBJ2-3 | NA | NA | SARS-COV-2 N | NA | NA |
| | | YF | | | | | | | | | |
| 512 | Beta | CASSTSNEYGYT | | TRBV27 | | TRBJ1-2 | NA | NA | SARS-COV-2 N | NA | NA |
| | | F | | | | | | | | | |
| 513 | Beta | CASSVTGTGGLG | | TRBV27 | TRBD1 | TRBJ1-6 | NA | NA | SARS-COV-2 N | NA | NA |
| | | NSPLHF | | | | | | | | | |
| 514 | Beta | CASSYPESNTEA | | TRBV6-1, | | TRBJ1-1 | NA | NA | SARS-COV-2 N | NA | NA |
| | | FF | | TRBV6-3, | | | | | | | |
| | | | | TRBV6-5, | | | | | | | |
| | | | | TRBV6-6, | | | | | | | |
| | | | | TRBV6-8 | | | | | | | |
| 515 | Beta | CASSYTGQGAGY | | TRBV6-1, | TRBD1 | TRBJ1-2 | NA | NA | SARS-COV-2 N | NA | NA |
| | | TF | | TRBV6-3, | | | | | | | |
| | | | | TRBV6-5, | | | | | | | |
| | | | | TRBV6-6, | | | | | | | |
| | | | | TRBV6-8 | | | | | | | |
| 516 | Beta | CASTLEYSTDTQ | | TRBV7-9 | TRBD2 | TRBJ2-3 | NA | NA | SARS-COV-2 N | NA | NA |
| | | YF | | | | | | | | | |
| 517 | Beta | CASTQTSGSDEQ | | TRBV6-1, | TRBD2 | TRBJ2-7 | NA | NA | SARS-COV-2 N | NA | NA |
| | | YF | | TRBV6-3, | | | | | | | |
| | | | | TRBV6-5, | | | | | | | |
| | | | | TRBV6-6, | | | | | | | |
| | | | | TRBV6-8 | | | | | | | |
| 518 | Beta | CATAAGGSYGYT | | TRBV15 | TRBD1 | TRBJ1-2 | NA | NA | SARS-COV-2 N | NA | NA |
| | | F | | | | | | | | | |
| 519 | Beta | CATSDGQGFADT | | TRBV24-1 | TRBD1 | TRBJ2-3 | NA | NA | SARS-COV-2 N | NA | NA |
| | | QYF | | | | | | | | | |
| 520 | Beta | CSARDISAYEQY | | TRBV20-1 | | TRBJ2-7 | NA | NA | SARS-COV-2 N | NA | NA |
| | | F | | | | | | | | | |
| 521 | Beta | CSARRQGALNQP | | TRBV20-1 | TRBD1 | TRBJ1-5 | NA | NA | SARS-COV-2 N | NA | NA |
| | | QHF | | | | | | | | | |
| 522 | Beta | CSARVNAGGGYT | | TRBV20-1 | TRBD1 | TRBJ1-2 | NA | NA | SARS-COV-2 N | NA | NA |
| | | F | | | | | | | | | |
| 523 | Beta | CSASIQGADYGY | | TRBV20-1 | TRBD1 | TRBJ1-2 | NA | NA | SARS-COV-2 N | NA | NA |
| | | TF | | | | | | | | | |
| 524 | Beta | CSASLARANTEA | | TRBV20-1 | TRBD1, | TRBJ1-1 | NA | NA | SARS-COV-2 N | NA | NA |
| | | FF | | | TRBD2 | | | | | | |
| 525 | Beta | CAIKTGSTNYGY | | TRBV10-3 | TRBD1 | TRBJ1-2 | NA | NA | SARS-COV-2 N | NA | NA |
| | | TF | | | | | | | | | |
| 526 | Beta | CAISERGLGYGY | | TRBV10-3 | TRBD1 | TRBJ1-2 | NA | NA | SARS-COV-2 N | NA | NA |
| | | TF | | | | | | | | | |
| 527 | Beta | CASSAGRNQPQH | | TRBV12-3, | TRBD2 | TRBJ1-5 | NA | NA | SARS-COV-2 N | NA | NA |
| | | F | | TRBV12-4 | | | | | | | |
| 528 | Beta | CASSFRREPYEQ | | TRBV5-4, | TRBD2 | TRBJ2-7 | NA | NA | SARS-COV-2 N | NA | NA |
| | | YF | | TRBV5-8 | | | | | | | |
| 529 | Beta | CASSIEVGETQY | | TRBV19 | | TRBJ2-5 | NA | NA | SARS-COV-2 N | NA | NA |
| | | F | | | | | | | | | |
| 530 | Beta | CASSLTGAVFGT | | TRBV7-9 | TRBD1 | TRBJ1-1 | NA | NA | SARS-COV-2 N | NA | NA |
| | | EAFF | | | | | | | | | |
| 531 | Beta | CASSLTGGRLGE | | TRBV7-9 | TRBD1 | TRBJ2-2 | NA | NA | SARS-COV-2 N | NA | NA |
| | | LFF | | | | | | | | | |
| 532 | Beta | CASSPGRITDTQ | | TRBV2 | TRBD1, | TRBJ2-3 | NA | NA | SARS-COV-2 N | NA | NA |
| | | YF | | | TRBD2 | | | | | | |
| 533 | Beta | CASSQEPGIMNT | | TRBV19 | | TRBJ1-1 | NA | NA | SARS-COV-2 N | NA | NA |
| | | EAFF | | | | | | | | | |
| 534 | Beta | CASSQWGINQPQ | | TRBV14 | TRBD1 | TRBJ1-5 | NA | NA | SARS-COV-2 N | NA | NA |
| | | HF | | | | | | | | | |
| 535 | Beta | CASSRRGGGASY | | TRBV6-1, | TRBD1, | TRBJ1-2 | NA | NA | SARS-COV-2 N | NA | NA |
| | | GYTF | | TRBV6-5, | TRBD2 | | | | | | |
| | | | | TRBV6-6, | | | | | | | |
| | | | | TRBV6-8 | | | | | | | |
| 536 | Beta | CSAGWGGGNAGG | | TRBV20-1 | TRBD1, | TRBJ1-2 | NA | NA | SARS-COV-2 N | NA | NA |
| | | TF | | | TRBD2 | | | | | | |
| 537 | Beta | CSAPKGQLLQET | | TRBV20-1 | TRBD1 | TRBJ2-5 | NA | NA | SARS-COV-2 N | NA | NA |
| | | QYF | | | | | | | | | |
| 538 | Beta | CSARDFRGTYGY | | TRBV20-1 | TRBD2 | TRBJ1-2 | NA | NA | SARS-COV-2 N | NA | NA |
| | | TF | | | | | | | | | |
| 539 | Beta | CSARDLHSNQPQ | | TRBV20-1 | | TRBJ1-5 | NA | NA | SARS-COV-2 N | NA | NA |
| | | HF | | | | | | | | | |
| 540 | Beta | CASSFSGANVLT | | TRBV28 | | TRBJ2-6 | NA | NA | SARS-COV-2 N | NA | NA |
| | | F | | | | | | | | | |
| 541 | Beta | CASSLGQNTIYF | | TRBV7-9 | | TRBJ1-3 | NA | NA | SARS-COV-2 N | NA | NA |
| 542 | Beta | CASSQIVTGGAF | | TRBV14 | TRBD2 | TRBJ1-1 | NA | NA | SARS-COV-2 N | NA | NA |
| | | F | | | | | | | | | |
| 543 | Beta | CASSQMGNTEAF | | TRBV28 | | TRBJ1-1 | NA | NA | SARS-COV-2 N | NA | NA |
| | | F | | | | | | | | | |
| 547 | Beta | CASSQVQSSGYT | | TRBV14 | | TRBJ1-2 | NA | NA | SARS-COV-2 N | NA | NA |
| | | F | | | | | | | | | |
| 545 | Full | CSARNGPSNQPQ | CAVRTYGQNFV | | | | | | | | |
| | | HF | F | | | | | | | | |
| 546 | Full | CASSLRGADGYT | CAGFNYGGSQG | | | | | | | | |
| | | F | NLIF | | | | | | | | |
| 547 | Full | CASSQTGQGVEG | CAGRVYNNND | | | | | | | | |
| | | YTF | MRF | | | | | | | | |

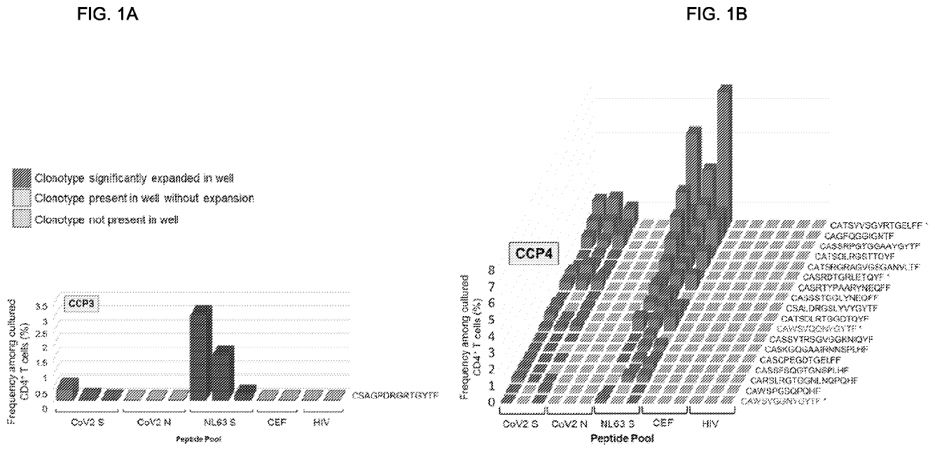

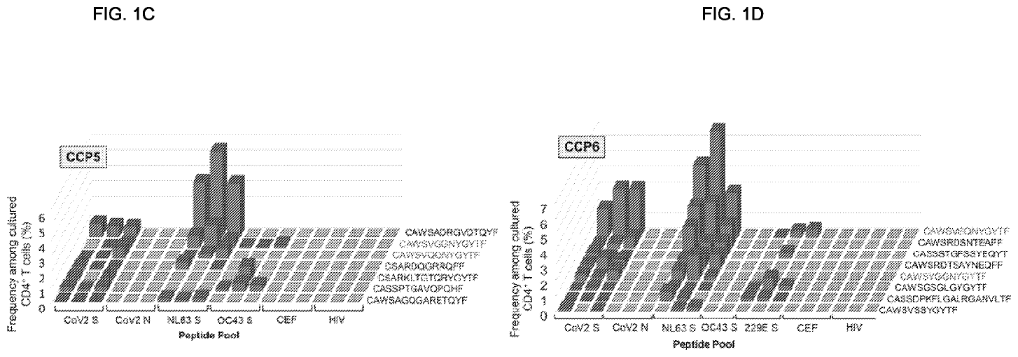

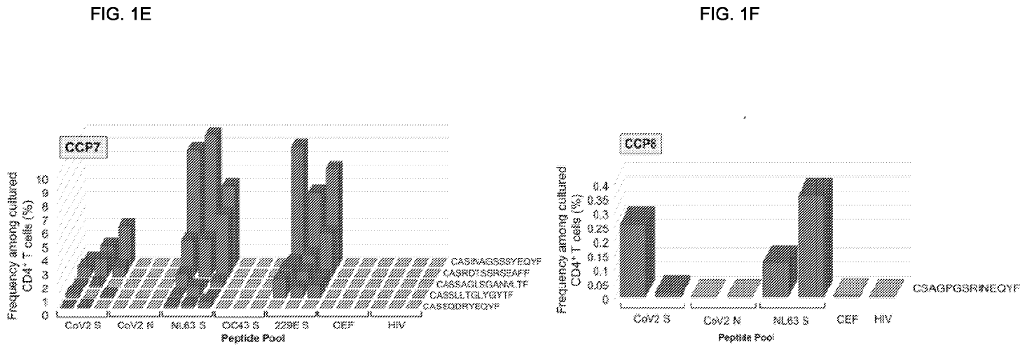

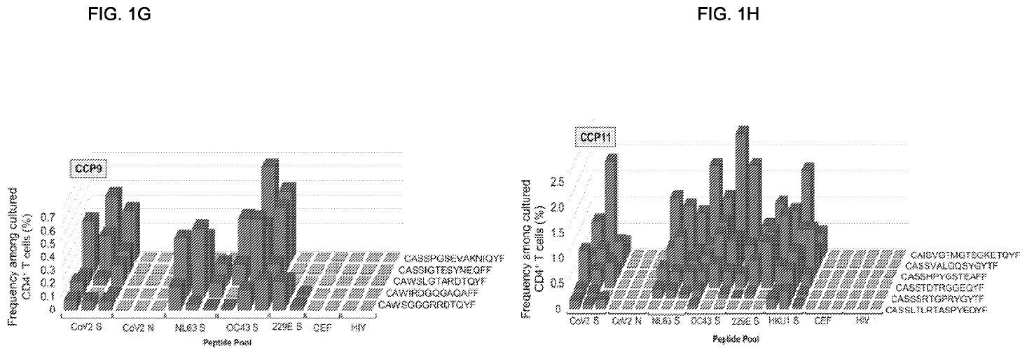

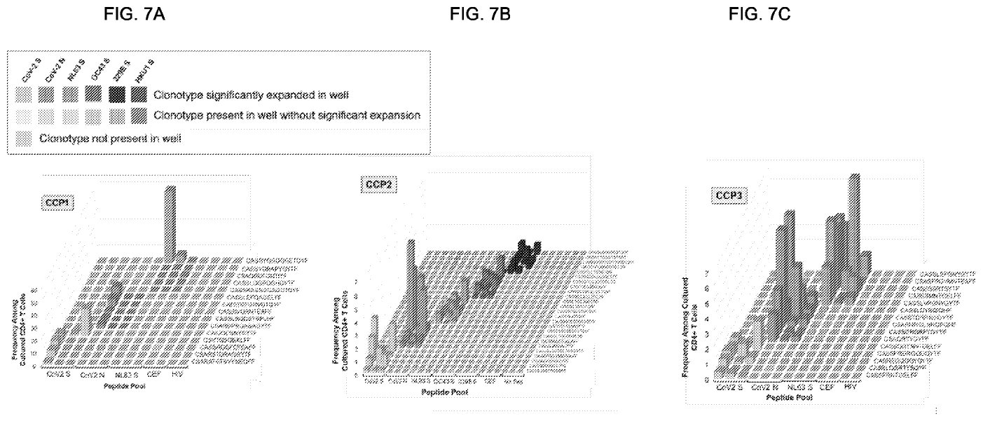

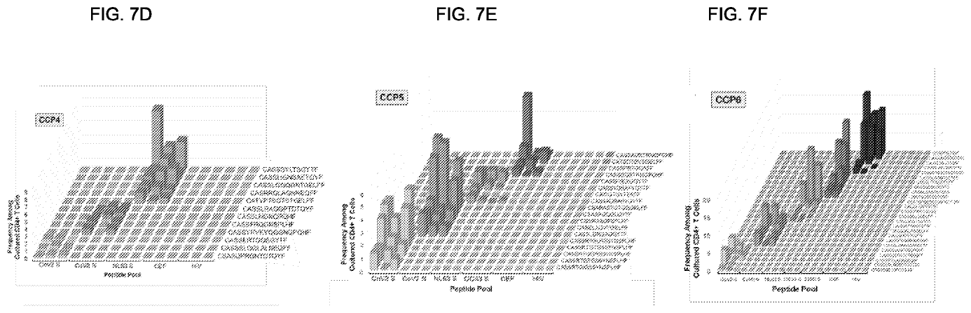

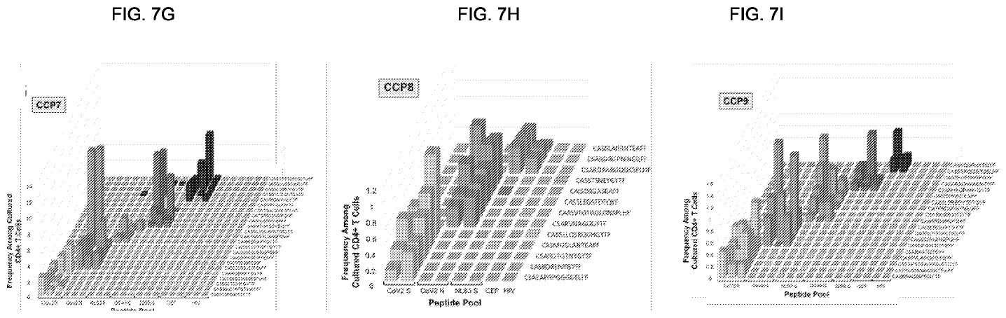

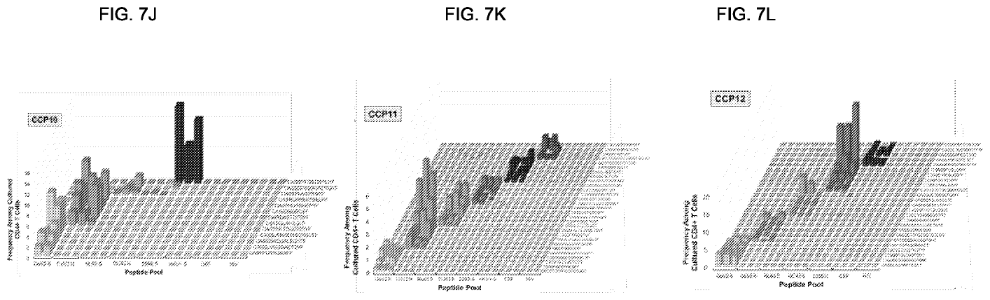

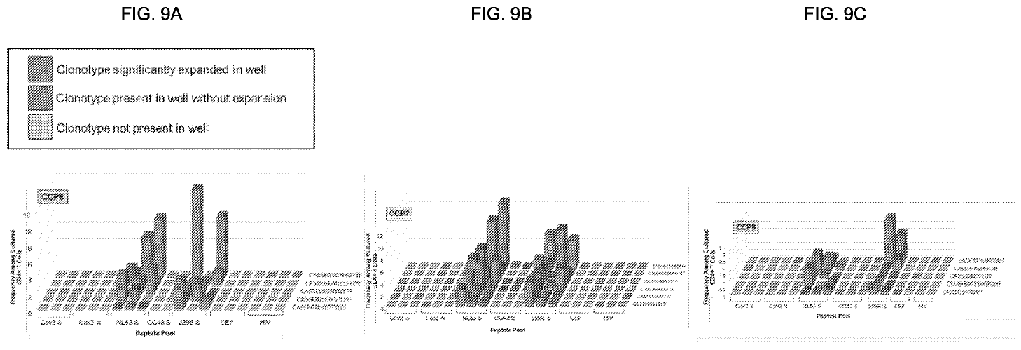

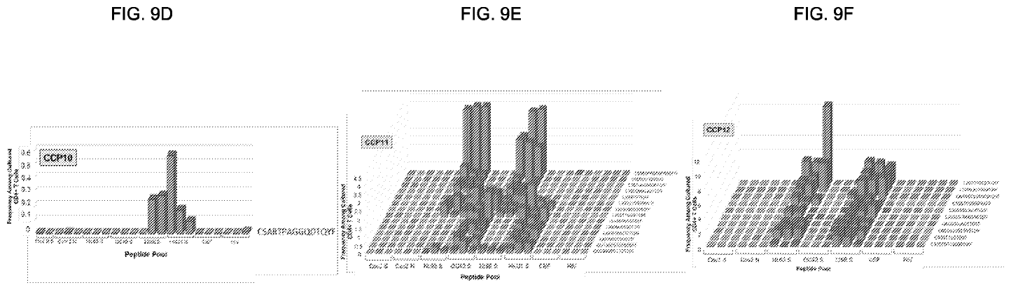

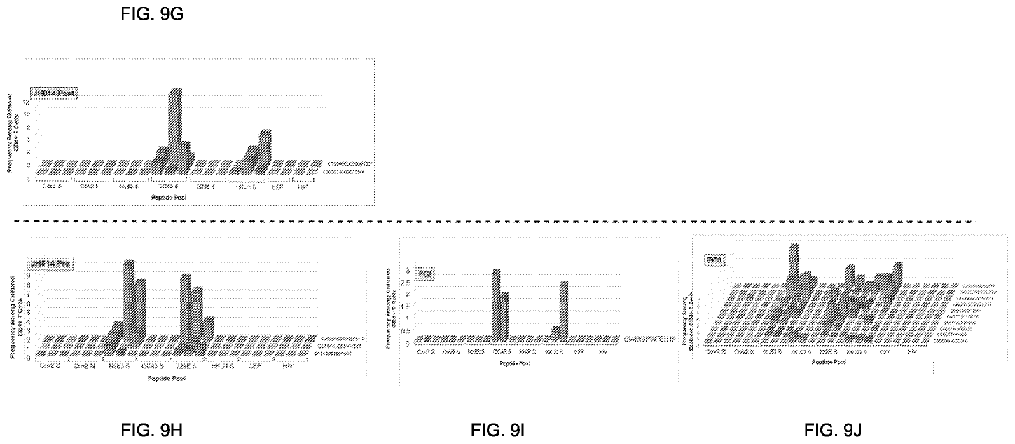

Consistent with other studies (11-13) SARS-CoV-2-specific memory CD4⁺ T cell responses were detected in 100% of the CCPs tested (n=12; FIG. 7A-7L). Strikingly, eight of these patients (67%) also had TCRs cross-reactive for the S protein from SARS-CoV-2 and at least one other CCC, as evidenced by clonal expansion of the same TCR Vj3 CDR3 clonotype in response to multiple S protein peptide pools (FIG. 1A-1H). While responses were also detected against the SARS-CoV-2 nucleocapsid (N) protein and the CMV, EBV, and flu (CEF) control, as expected, none of these TCRs were cross-reactive with any of the S proteins. Cross-reactivity to the S protein of two or more CCCs occurred in all CCP donors in which multiple CCC S peptide pools were tested (FIG. 9A-9J). Surprisingly, SARS-CoV-2/HCoV-NL63 cross-reactive memory responses by three highly homologous TCRβ CDR3 clonotypes (CAWSVQQNYGYTF (SEQ ID NO: 291 and SEQ ID NO: 5), CAWSVGGNYGYTF (SEQ ID NO: 289 and SEQ ID NO: 6), and CAWSVQGNYGYTF (SEQ ID NO: 290 and SEQ ID NO: 8)) were independently detected in more than one CCP (red clones, FIG. 1B-1D). Together, these data show that SARS-CoV-2/CCC cross-reactive memory CD4⁺ T cells are readily detected in the peripheral blood of CCPs, can functionally expand upon antigenic stimulation, and that a subset of TCR Vβ CDR3 clonotypes are shared among patients.

### Example 2: SARS-CoV-2-Specific TCR Avidity and Epitope Identification

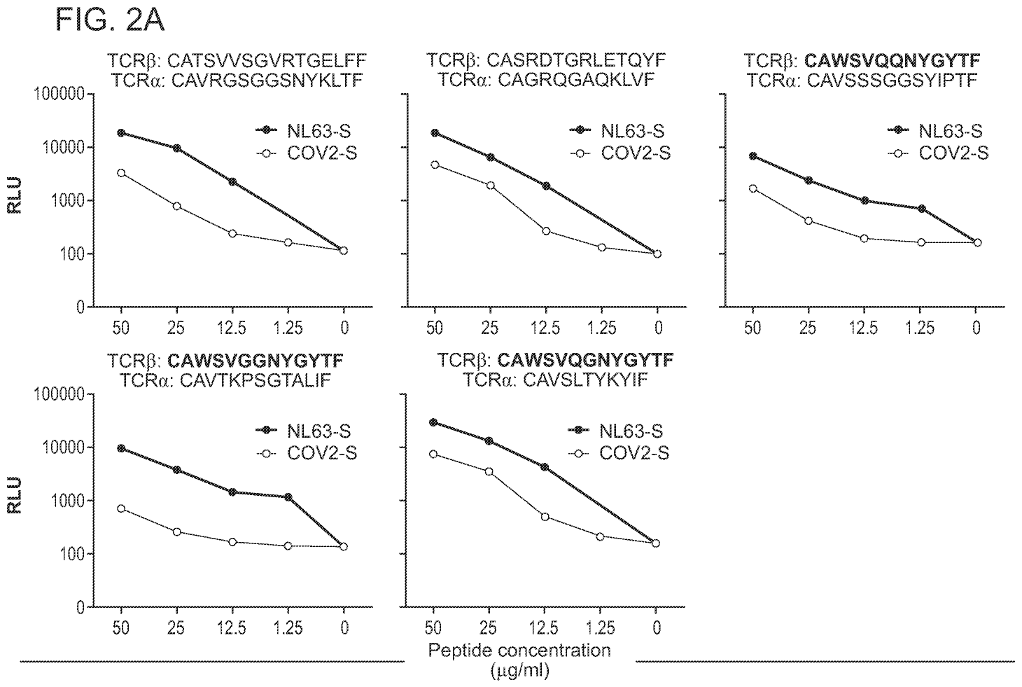

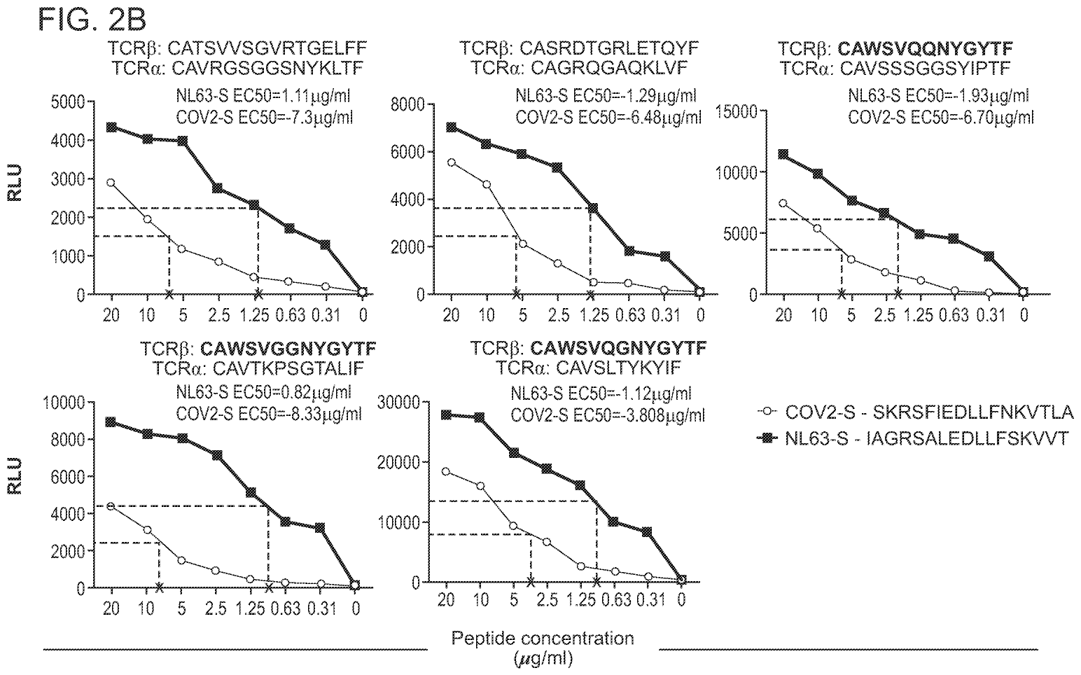

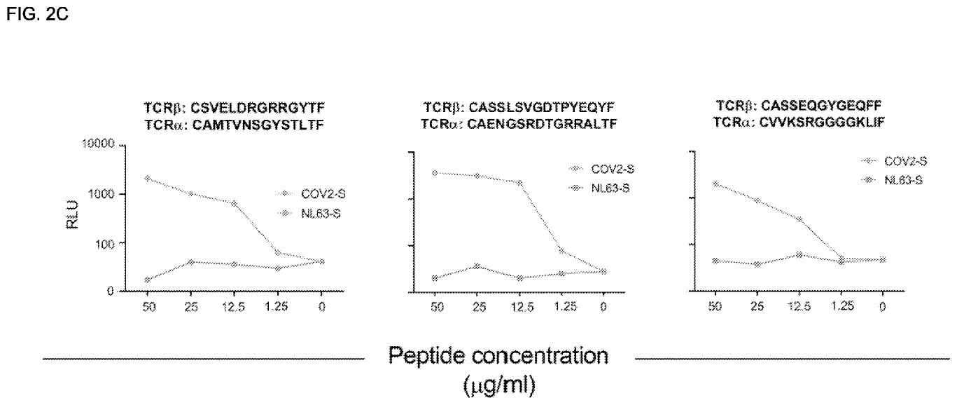

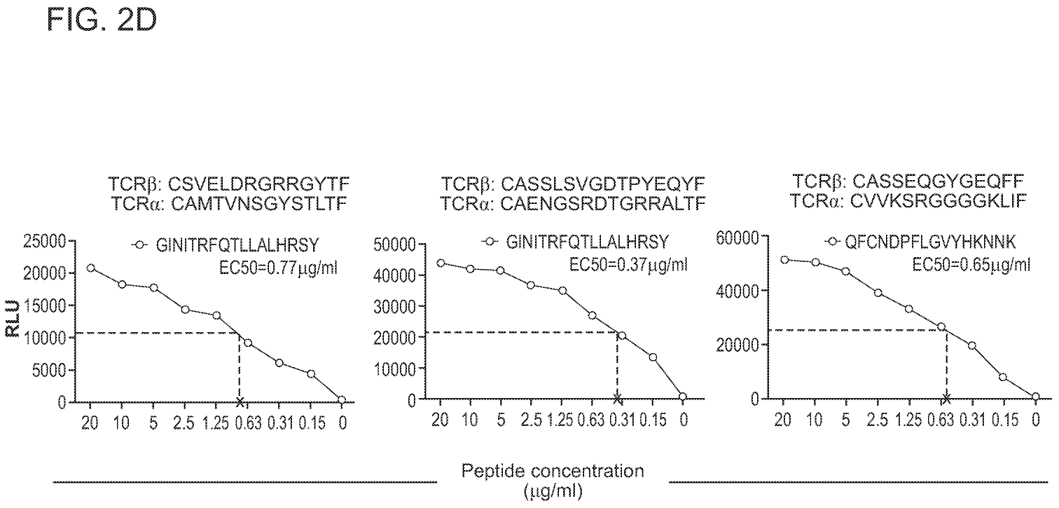

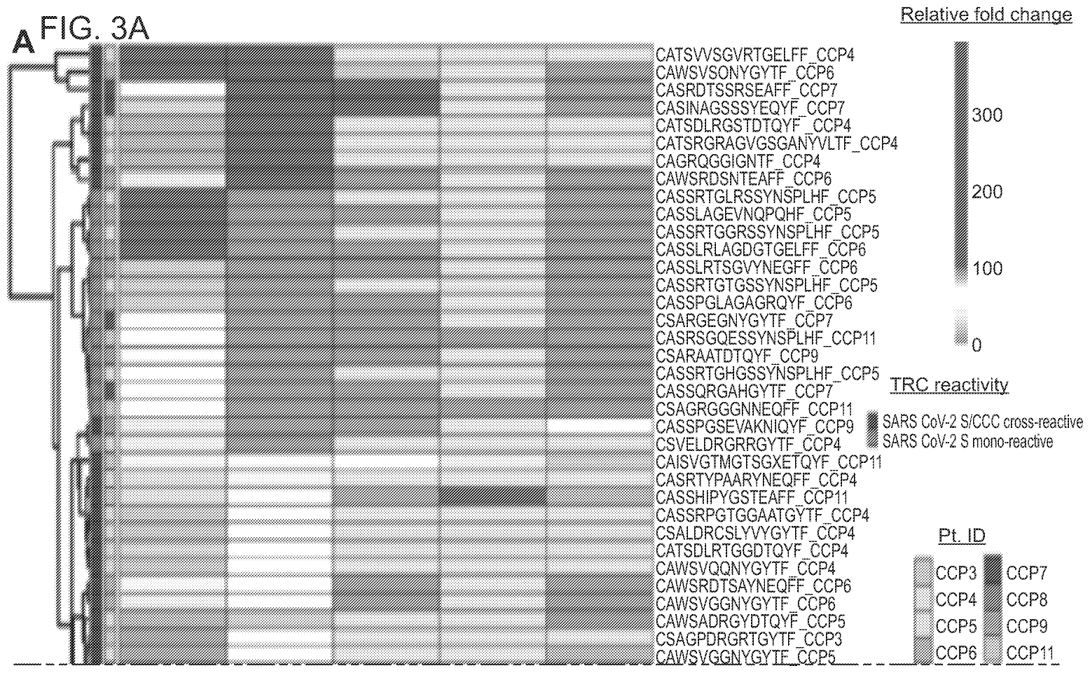

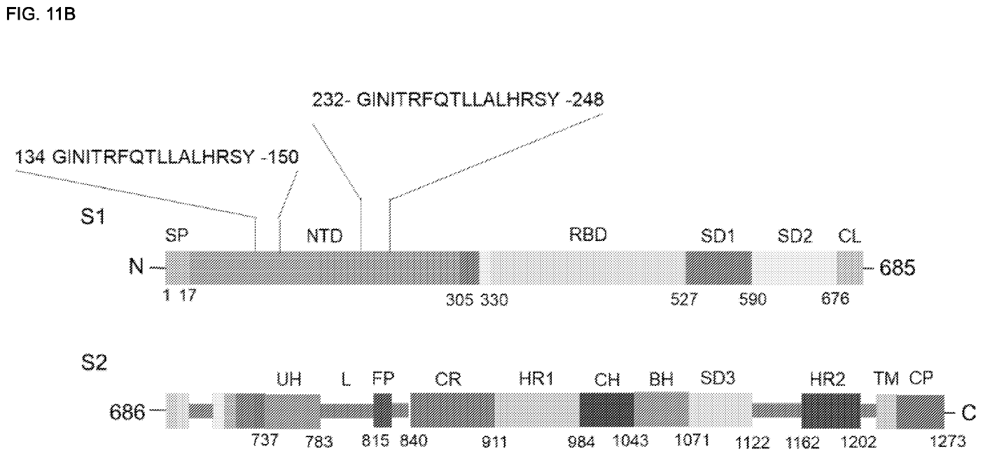

A recent study demonstrated reduced avidity of the SARS-CoV-2/CCC cross-reactive T cell response (30), however this has not been evaluated at the individual clonotype level. To determine the avidity of individual SARS-CoV-2 reactive TCRs, the cognate alpha chain for three mono-reactive (recognizing only SARS-CoV-2 S) and five cross-reactive TCRs detected in CCP4 (FIGS. 1B and 7D) were detected, including the 3 TCRs that were detected in multiple patients. The entire TCR was cloned into a CD4-overexpressing Jurkat NFAT-luciferase reporter system, which specifically reads out the quantitative strength of TCR engagement, commonly referred to as “functional avidity”. Cross-reactivity was confirmed for all 5 cross-reactive TCRs (FIG. 2A), with lower NFAT activity induced by SARS-CoV-2 relative to HCoV-NL63 S protein. Mono-reactive responses of three SARS-CoV-2 S-specific TCRs were also confirmed using this TCR cloning system (FIGS. 2C and 11A-11B).

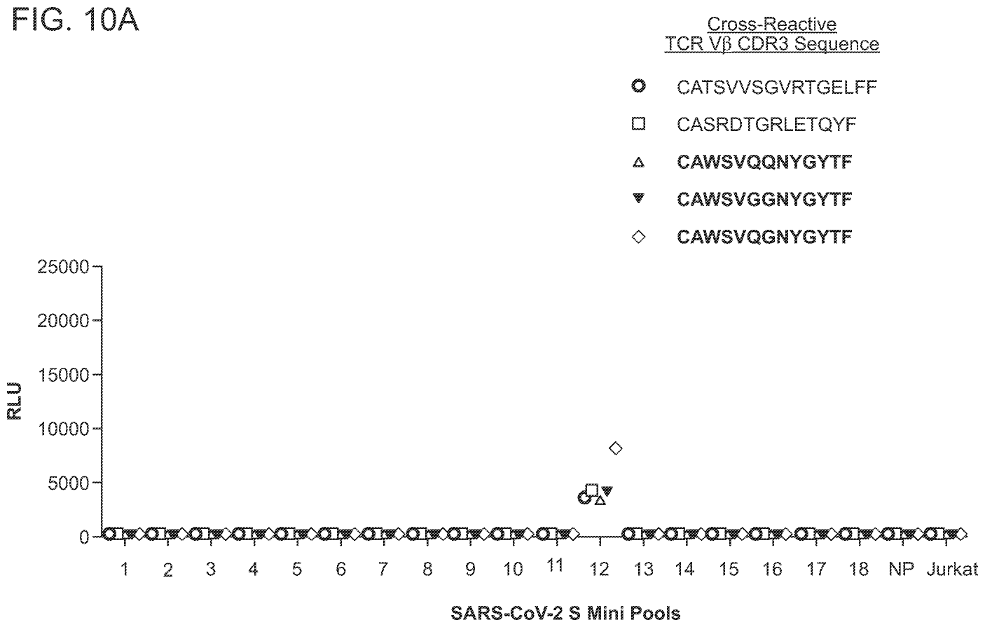

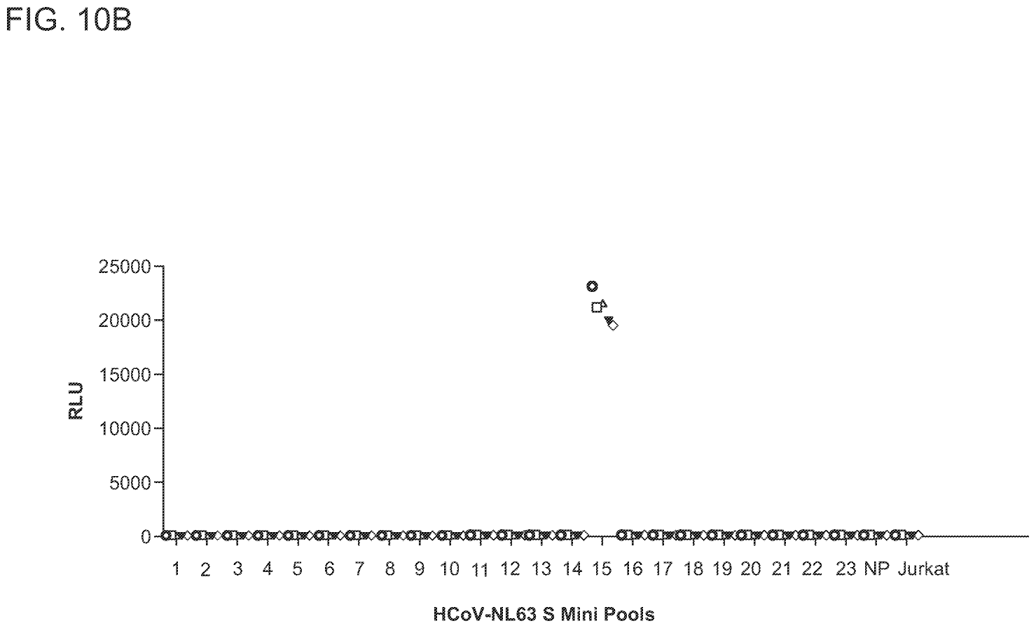

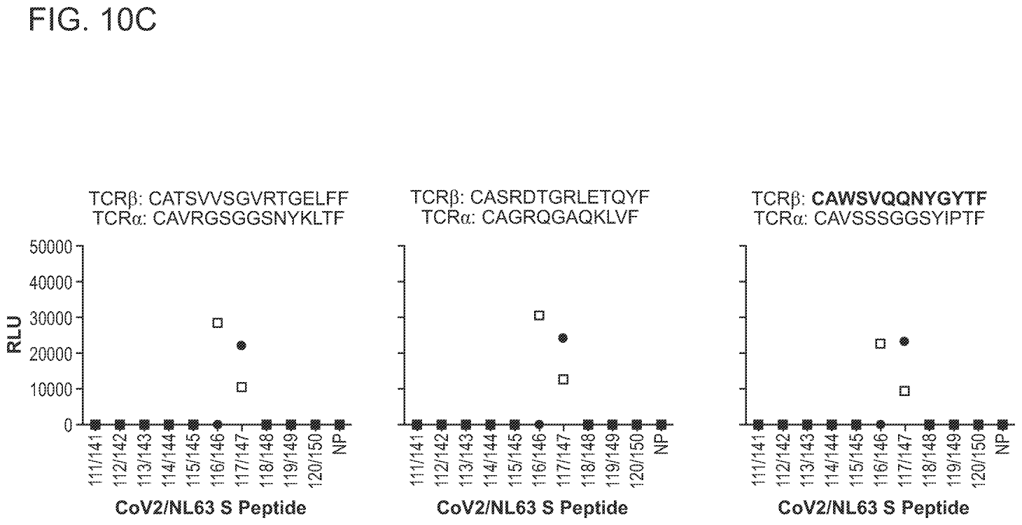

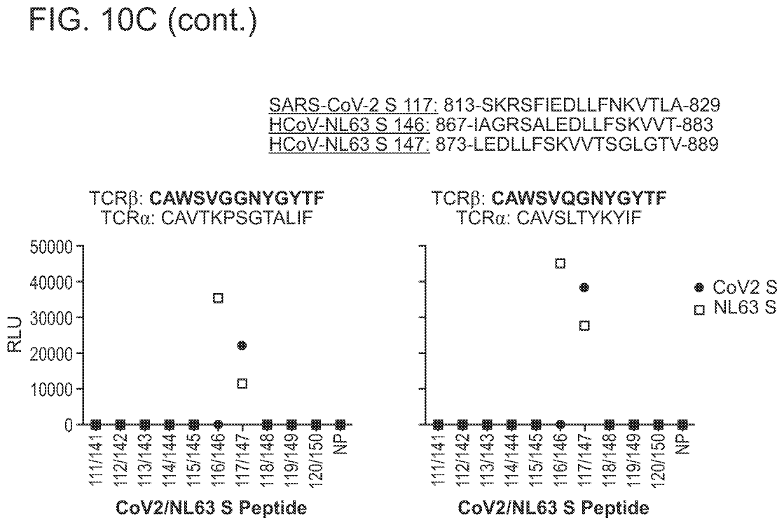

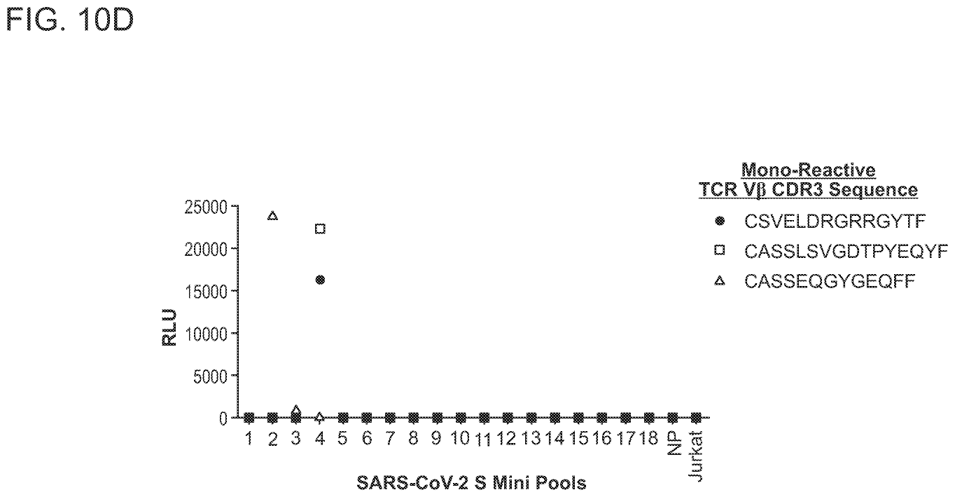

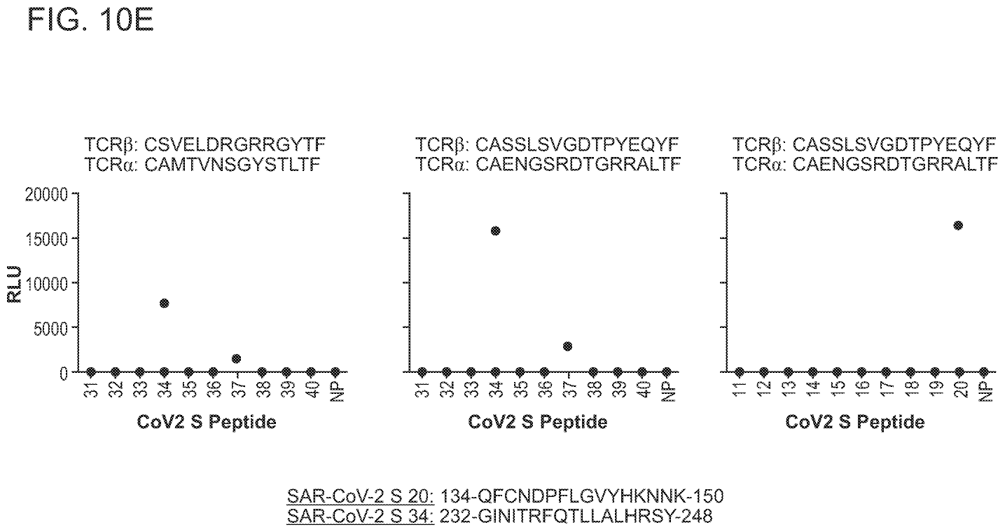

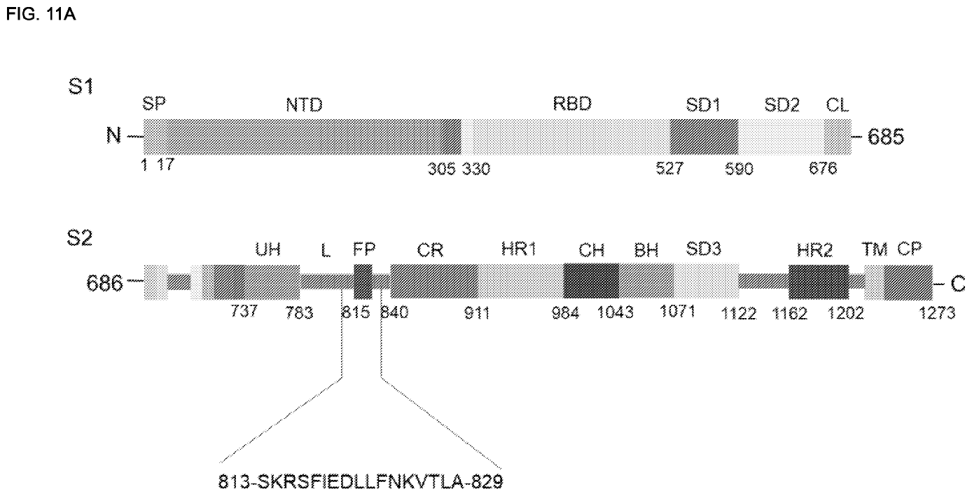

To then map the precise SARS-CoV-2 and HCoV-NL63 S protein region(s) eliciting these responses, the TCR-transfected Jurkat cells were tested for reactivity against mini pools of 10 peptides each. Cross-reactive TCRs were cloned into the Jurkat reporter cell line and stimulated them with each pool (FIGS. 10A and 10B) and subsequently with individual 15-mer peptides from the positive pools (SARS-CoV-2 pool 12 and HCoV-NL63 pool 15 and Table 2,below. All cross-reactive TCRs recognized SARS-CoV-2—SKRSFIEDLLFNKVTLA (amino acids 813-829), which is in the S2 linker region (31) (FIGS. 11A-11B) and cross-recognized the IAGRSALEDLLFSKVVT (867-883) region from HCoV-NL63 S (FIG. 10C). The SARS-CoV-2 sequence, EDLLFNKV, within this 17-mer, differs from the cognate HCoV-NL63 sequence by only one amino acid substitution, an asparagine to serine at position 6 (EDLLFSKV), which likely does not alter the polarity of the peptide and could represent the core 8-mer responsible for TCR contact and/or MHC class II binding region.

TABLE 2

| | Peptide | | | |
|---|---|---|---|---|
| | Mini | Peptide | | |
| Virus | Pool | No. | Length | Sequence |
| HCoV-NL63 | 15 | 141 of 226 | 17 | 837-SNAFSLANVTSFGDYNL-853 |
| HCoV-NL63 | 15 | 142 of 226 | 17 | 843-ANVTSFGDYNLSSVLPQ-859 |
| HCoV-NL63 | 15 | 143 of 226 | 17 | 849-GDYNLSSVLPQRNIRSS-865 |
| HCoV-NL63 | 15 | 144 of 226 | 17 | 855-SVLPQRNIRSSRIAGRS-871 |
| HCoV-NL63 | 15 | 145 of 226 | 17 | 861-NIRSSRIAGRSALEDLL-877 |
| HCoV-NL63 | 15 | 146 of 226 | 17 | 867-IAGRSALEDLLFSKVVT-883 |
| HCoV-NL63 | 15 | 147 of 226 | 17 | 873-LEDLLFSKVVTSGLGTV-889 |
| HCoV-NL63 | 15 | 148 of 226 | 17 | 879-SKVVTSGLGTVDVDYKS-895 |
| HCoV-NL63 | 15 | 149 of 226 | 17 | 885-GLGTVDVDYKSCTKGLS-901 |
| HCoV-NL63 | 15 | 150 of 226 | 17 | 891-VDYKSCTKGLSIADLAC-907 |
| SARS-CoV2 | 2 | 11 of 181 | 17 | 71-SGTNGTKRFDNPVLPFN-87 |
| SARS-CoV2 | 2 | 12 of 181 | 17 | 78-RFDNPVLPFNDGVYFAS-94 |
| SARS-CoV2 | 2 | 13 of 181 | 17 | 85-PFNDGVYFASTEKSNII-101 |
| SARS-CoV2 | 2 | 14 of 181 | 17 | 92-FASTEKSNIIRGWIFGT-108 |
| SARS-CoV2 | 2 | 15 of 181 | 17 | 99-NIIRGWIFGTTLDSKTQ-115 |
| SARS-CoV2 | 2 | 16 of 181 | 17 | 106-FGTTLDSKTQSLLIVNN-122 |
| SARS-CoV2 | 2 | 17 of 181 | 17 | 113-KTQSLLIVNNATNVVIK-129 |
| SARS-CoV2 | 2 | 18 of 181 | 17 | 120-VNNATNVVIKVCEFQFC-136 |
| SARS-CoV2 | 2 | 19 of 181 | 17 | 127-VIKVCEFQFCNDPFLGV-143 |
| SARS-CoV2 | 2 | 20 of 181 | 17 | 134-QFCNDPFLGVYYHKNNK-150 |
| SARS-CoV2 | 4 | 31 of 181 | 17 | 211-NLVRDLPQGFSALEPLV-227 |
| SARS-CoV2 | 4 | 32 of 181 | 17 | 218-QGFSALEPLVDLPIGIN-234 |
| SARS-CoV2 | 4 | 33 of 181 | 17 | 225-PLVDLPIGINITRFQTL-241 |
| SARS-CoV2 | 4 | 34 of 181 | 17 | 232-GINITRFQTLLALHRSY-248 |
| SARS-CoV2 | 4 | 35 of 181 | 17 | 239-QTLLALHRSYLTPGDSS-255 |
| SARS-CoV2 | 4 | 36 of 181 | 17 | 246-RSYLTPGDSSSGWTAGA-262 |
| SARS-CoV2 | 4 | 37 of 181 | 17 | 253-DSSSGWTAGAAAYYVGY-269 |
| SARS-CoV2 | 4 | 38 of 181 | 17 | 260-AGAAAYYVGYLQPRTFL-276 |
| SARS-CoV2 | 4 | 39 of 181 | 17 | 267-VGYLQPRTFLLKYNENG-283 |
| SARS-CoV2 | 4 | 40 of 181 | 17 | 274-TFLLKYNENGTITDAVD-290 |
| SARS-CoV2 | 12 | 111 of 181 | 17 | 771-AVEQDKNTQEVFAQVKQ-787 |
| SARS-CoV2 | 12 | 112 of 181 | 17 | 778-TQEVFAQVKQIYKTPPI-794 |
| SARS-CoV2 | 12 | 113 of 181 | 17 | 785-VKQIYKTPPIKDFGGFN-801 |
| SARS-CoV2 | 12 | 114 of 181 | 17 | 792-PPIKDFGGFNFSQILPD-808 |
| SARS-CoV2 | 12 | 115 of 181 | 17 | 799-GFNFSQILPDPSKPSKR-815 |
| SARS-CoV2 | 12 | 116 of 181 | 17 | 806-LPDPSKPSKRSFIEDLL-822 |
| SARS-CoV2 | 12 | 117 of 181 | 17 | 813-SKRSFIEDLLFNKVTLA-829 |
| SARS-CoV2 | 12 | 118 of 181 | 17 | 820-DLLFNKVTLADAGFIKQ-836 |
| SARS-CoV2 | 12 | 119 of 181 | 17 | 827-TLADAGFIKQYGDCLGD-843 |
| SARS-CoV2 | 12 | 120 of 181 | 17 | 834-IKQYGDCLGDIAARDLI-850 |

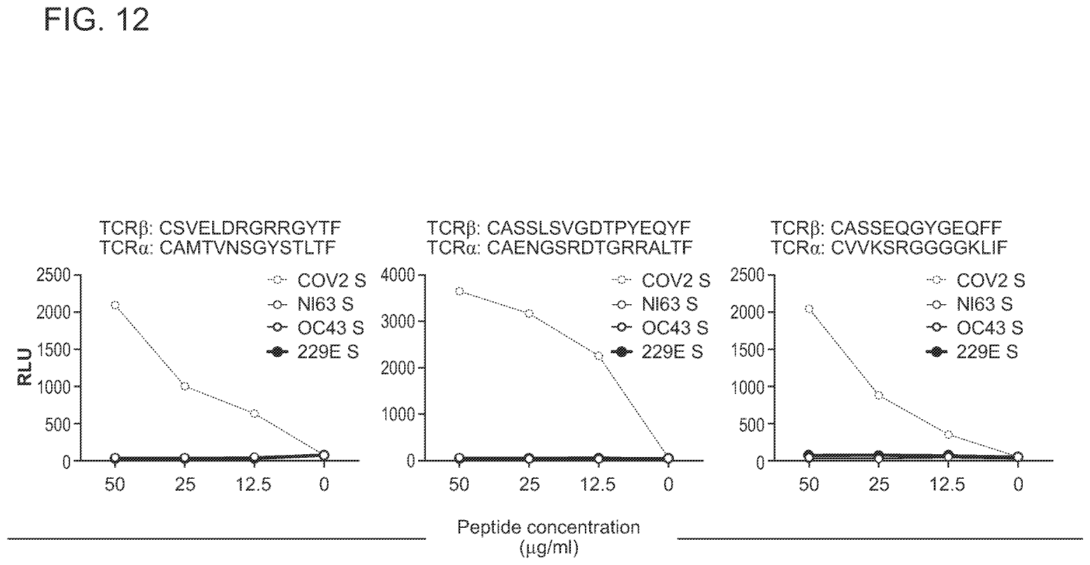

Peptide titration experiments defined a maximum RLU range of 4,360 to 27,900 for HCoV-NL63-IAGRSALEDLLFSKVVT. The maximum RLU for SARS-CoV-2-SKRSFIEDLLFNKVTLA was not reached at 20 ug/ml but ranged from 2,890 to 18,300. The cross-reactive TCR functional avidity (EC50; peptide concentration required to reach ½ maximum RLU) for HCoV-NL63 S-IAGRSALEDLLFSKVVT ranged from 0.82 ug/ml to 1.93 ug/ml with average 1.25 ug/ml (FIG. 2B). To estimate the EC50 for SARS-CoV-2, the maximum RLU reached after peptide stimulation was used. The SARS-CoV-2 S-SKRSFIEDLLFNKVTLA functional avidity ranged from 3.80 ug/ml to 8.33 ug/ml with average 6.54 ug/ml; roughly 5-fold lower than the functional avidity for HCoV-NL63 S peptide. Using the jurkat transfer approach, the minimal 17-mers for the 3 mono-reactive TCRs were identified and confirmed that they do not cross-recognize HCoV-NL63 S protein, even at high peptide concentrations (FIG. 2C and FIG. 12). Two TCRs recognized SARS-CoV-2—GINITRFQTLLALHRSY (232-248) and one TCR recognized SARS-CoV-2—QFCNDPFLGVYHKNNK (134-150) (FIGS. 10D and 10E), which are both present in the S1 N-terminal domain (31) (FIGS. 11A-11B). Interestingly, the EC50 for mono-reactive TCRs ranged from 0.37 ug/ml to 0.77 ug/ml with an average 0.6 ug/ml. (FIG. 2D). These data demonstrate significantly reduced functional avidity of the cross-reactive SARS-CoV-2 T cell response relative to the CCC response (p=0.0011), compatible with an antigenic imprinting mechanism.

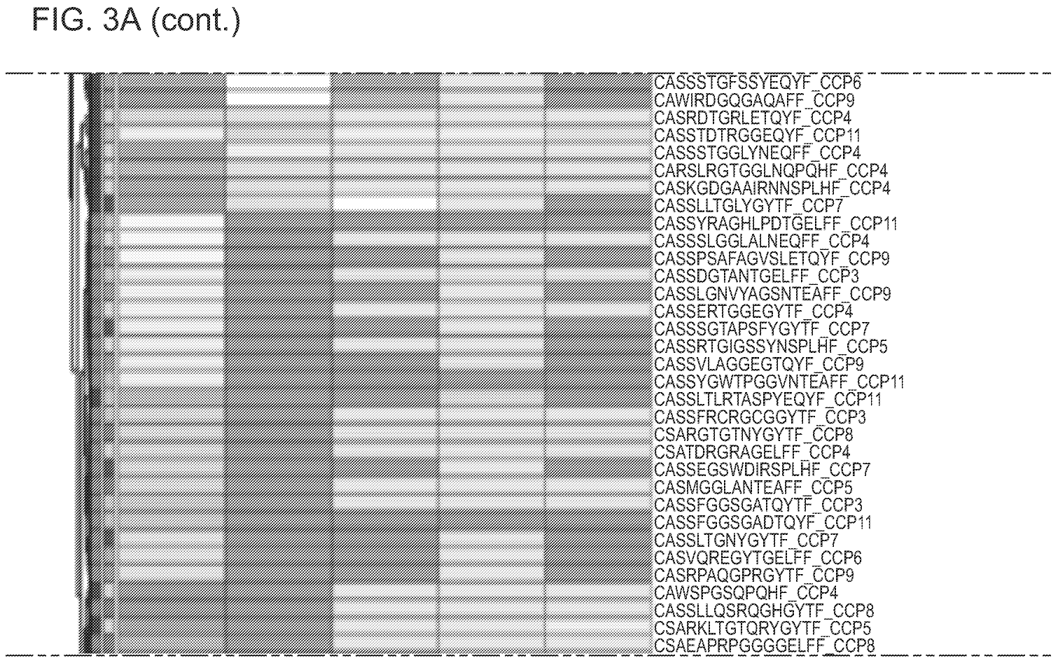

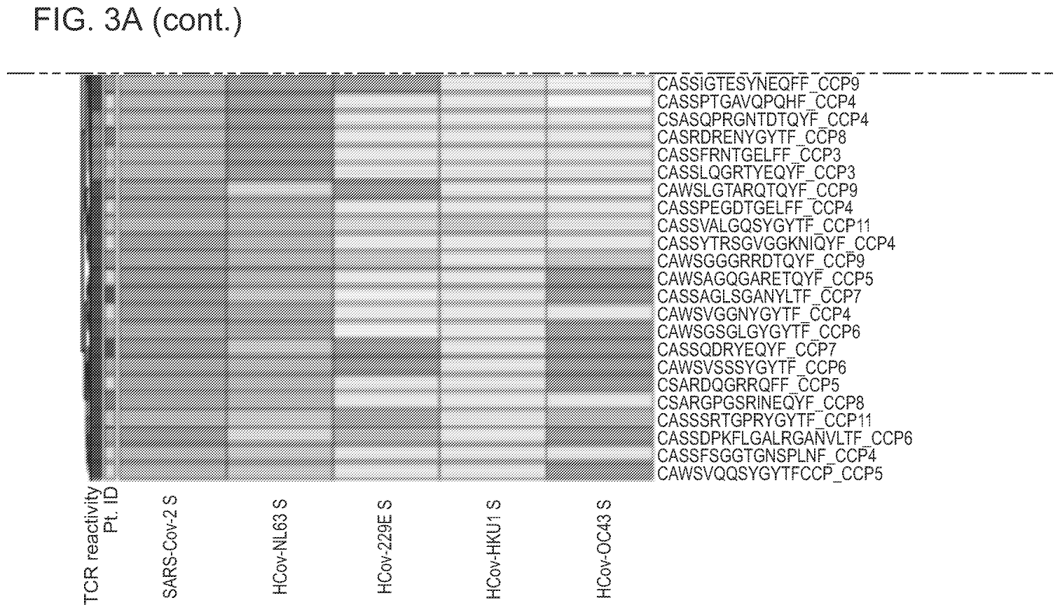

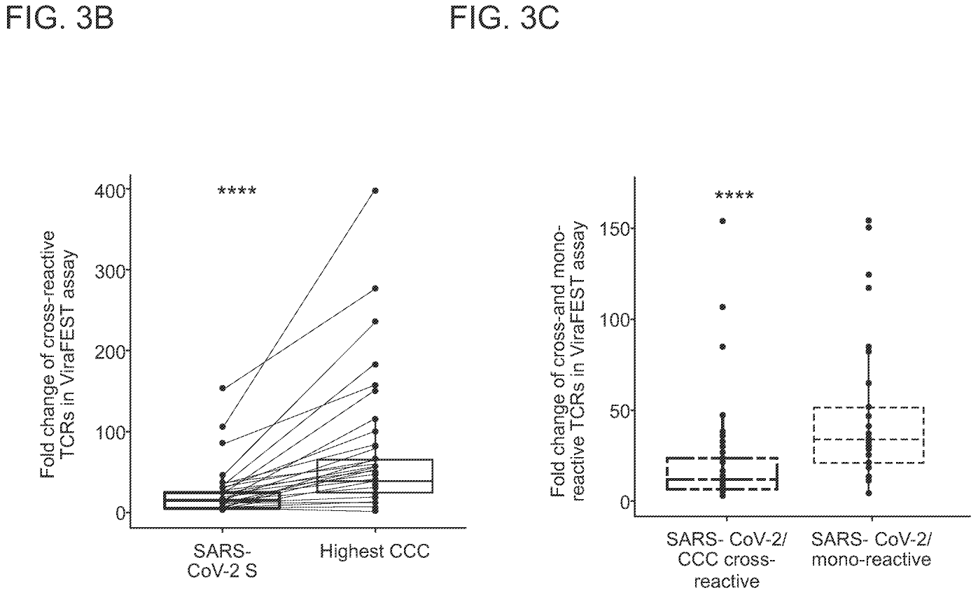

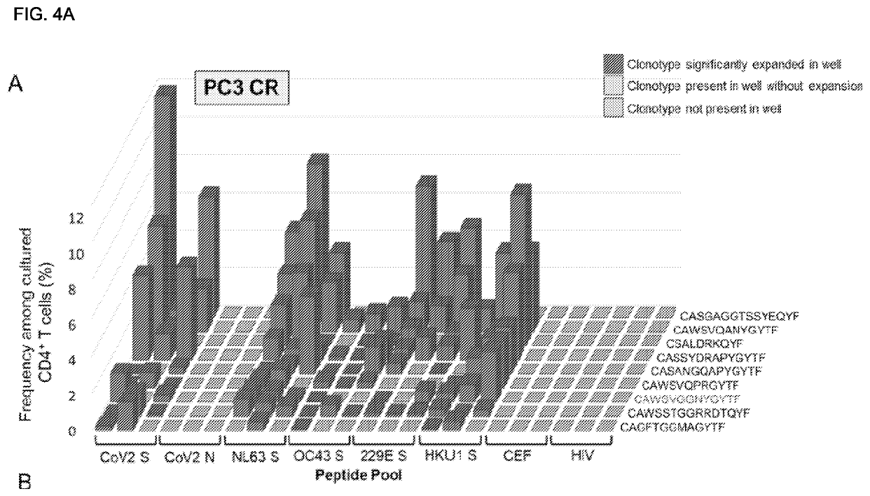

To further assess the quality of the functional T cell response, the magnitude of SARS-CoV-2/CCC cross-reactive T cell expansion was compared with mono-reactive T cell expansion in the ViraFEST assay (FIG. 3A). An unsupervised clustering approach was used to visualize the fold change of in vitro clonotypic expansion of mono-reactive clones, as well as the clonotypic expansion of cross-reactive TCRs in response to SARS-CoV-2 S or the CCC that elicited the highest fold change. Cross-reactive clones clustered together indicating similar in vitro expansion patterns (FIG. 3A). Quantitatively, the cross-reactive clones had a significantly lower level of antigen-specific expansion in response to the SARS-CoV-2 S protein relative to CCC S protein (p=3.0E−10; FIG. 3B). Additionally, SARS-CoV-2 cross-reactive clonotypic expansions were significantly lower than SARS-CoV-2 mono-reactive expansions (p=6.7E−7; FIG. 3C), thus demonstrating reduced expansion of clones with cross-reactive TCRs for SARS-CoV-2 and supporting the hypothesis that a subset of observed T cell responses to SARS-CoV-2 likely resulted from pre-existing cross-reactive clones rather than de novo priming by SARS-CoV-2 infection.

### Example 3: Detection of Recall SARS-CoV-2/CCC Cross-Reactive CD4+ T Cells in SARS-CoV-2-Unexposed Donors

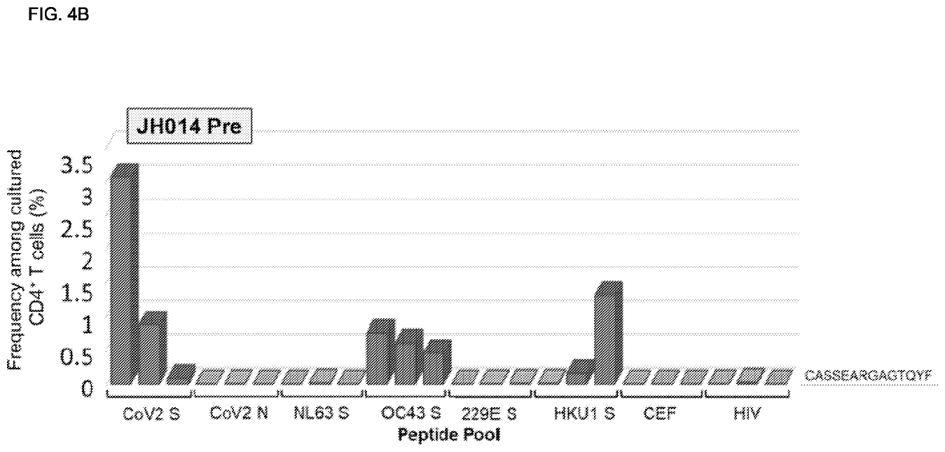

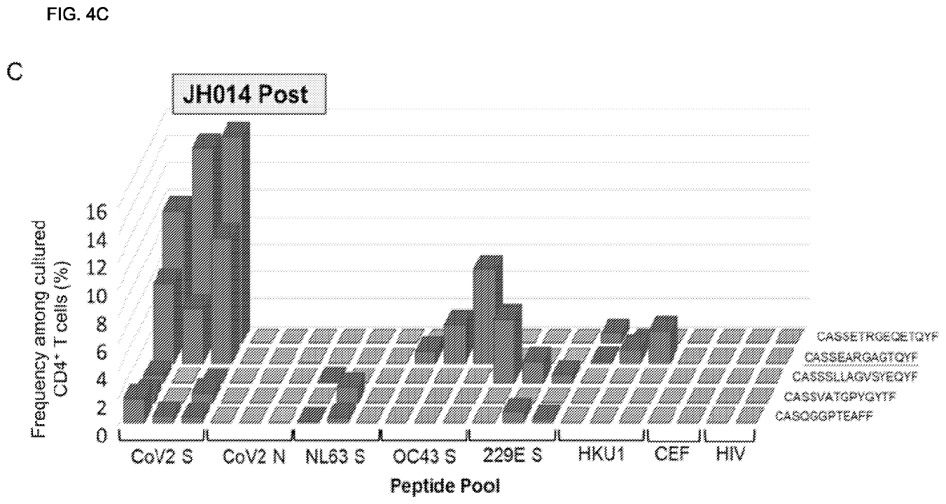

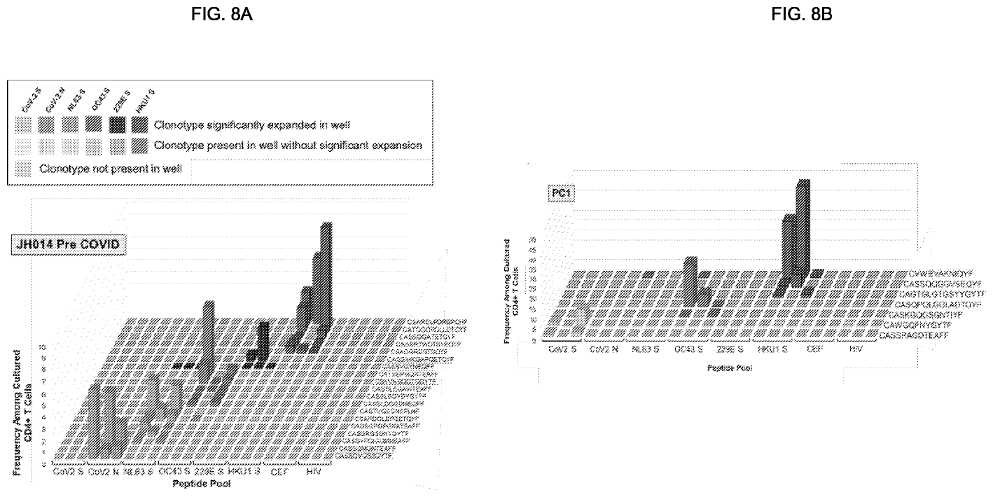

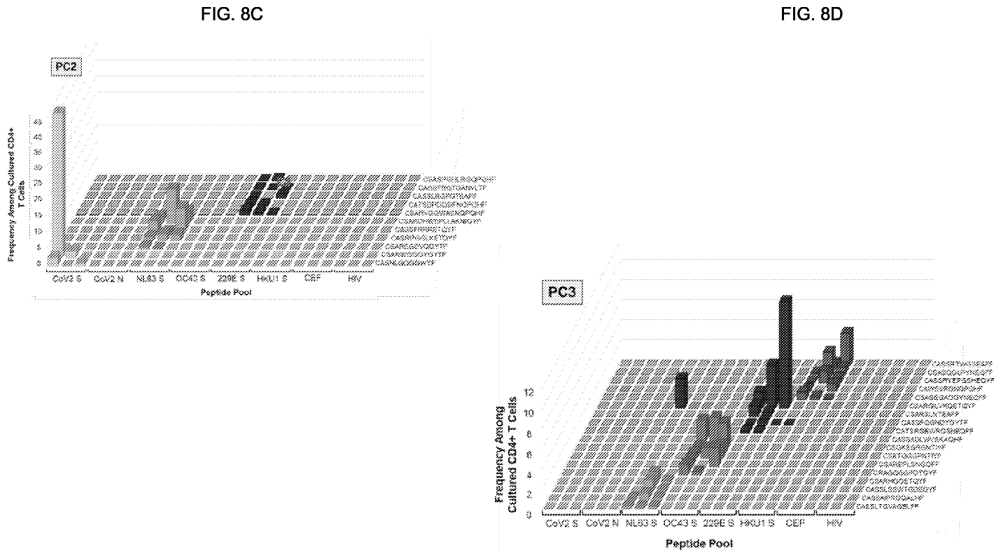

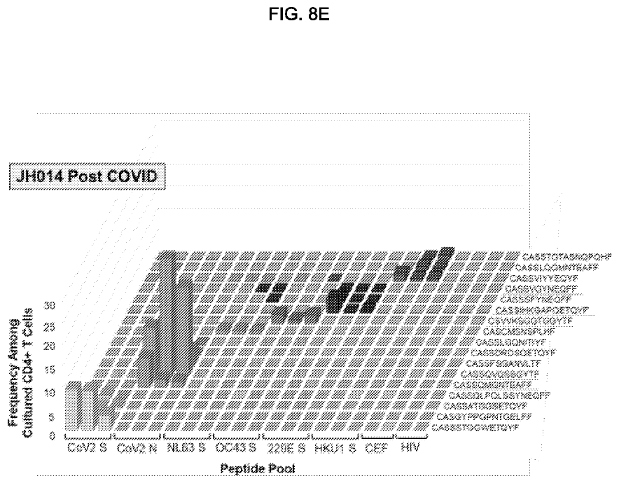

The cross-reactivity detected in CCPs could have been generated by recent infection with SARS-CoV-2 or primed by past infection with a CCC. To explore the possibility that memory CD4+ T cells against SARS-CoV-2 resulted from prior CCC exposure, the ViraFEST assay was used to test CD4+ T cells obtained from four healthy donors between 2011-2018, before the COVID-19 pandemic (PC1, PC2, PC3, and JH014-pre; Table 1 and FIG. 8A-8D). SARS-CoV-2/CCC cross-reactivity was detected in PC3 and JH014 (FIGS. 4A and 4B). Notably, the CAWSVGGNYGTYF clone, identified as being SARS-CoV-2/CCC cross-reactive in convalescent patients CCP4, CCP5, and CCP6, also functionally expanded in the PC3 ViraFEST assay (red font, FIG. 4A), confirming the existence of cross-reactive memory CD4+ T cell responses at the clonal level prior to SARS-CoV-2 exposure. Of note, PC1 and PC2 also demonstrated mono-reactive SARS-CoV-2 S specific CD4+ clonal expansion. Neither donor had any response to SARS-CoV-2 N (FIGS. 8B and 8C).

T cell responses were evaluated in peripheral blood samples obtained from JH014 during the COVID-19 pandemic (July 2020; FIG. 8E). JH014 and his household contacts had symptoms consistent with COVID-19 in April of 2020, however none of the household members were tested for infection and JH014 is seronegative for SARS-CoV-2-specific antibodies. Prior to the pandemic, a cross-reactive memory response (clone CASSEARGAGTQYF) specific for SARS-CoV2 S, HCoV-OC43 S and HCoV-HKU1 S was detected using the ViraFEST assay (red underline, FIG. 4B). Several additional cross-reactive clones were detected in July 2020, in addition to the same clone identified prior to the pandemic (red underline, FIG. 4C). SARS-CoV-2 monoreactive clones were identified in the July 2020 assay (FIG. 8A). Taken together, these support the idea that true clonal cross-reactive memory CD4+ T cell responses existed prior to the COVID-19 pandemic, that these clonotypes maintain their antigen-specific in vitro expansive ability, and can be identified by re-stimulation of CCC and SARS-CoV-2 S protein antigens.

### Example 4: SARS-CoV-2/CCC Cross-Reactive TCRs Share Strong Sequence Homology Within and Between Patients

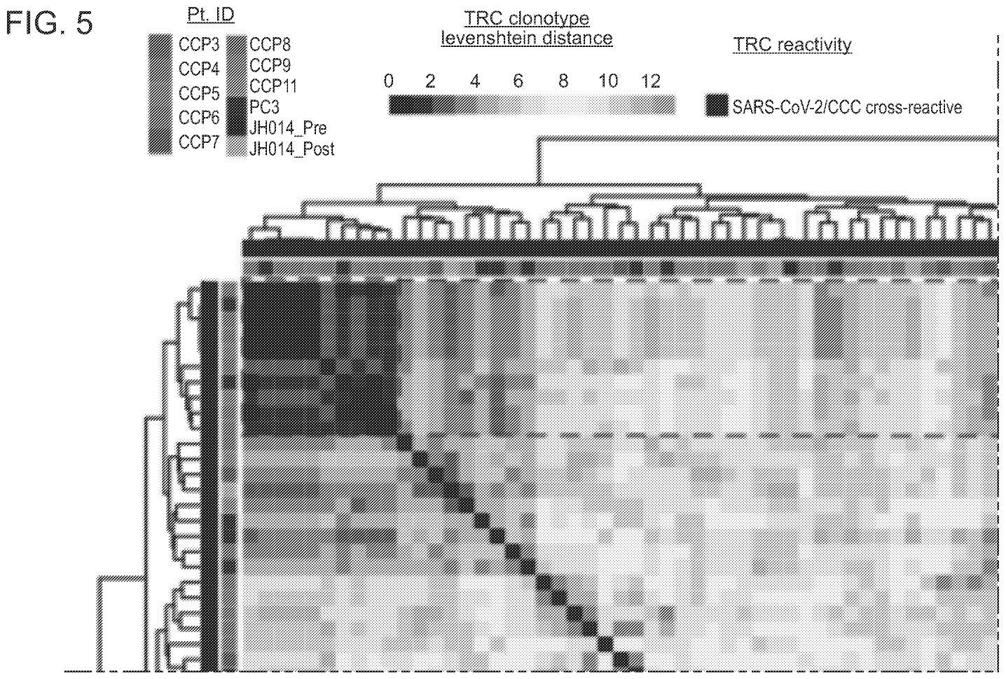

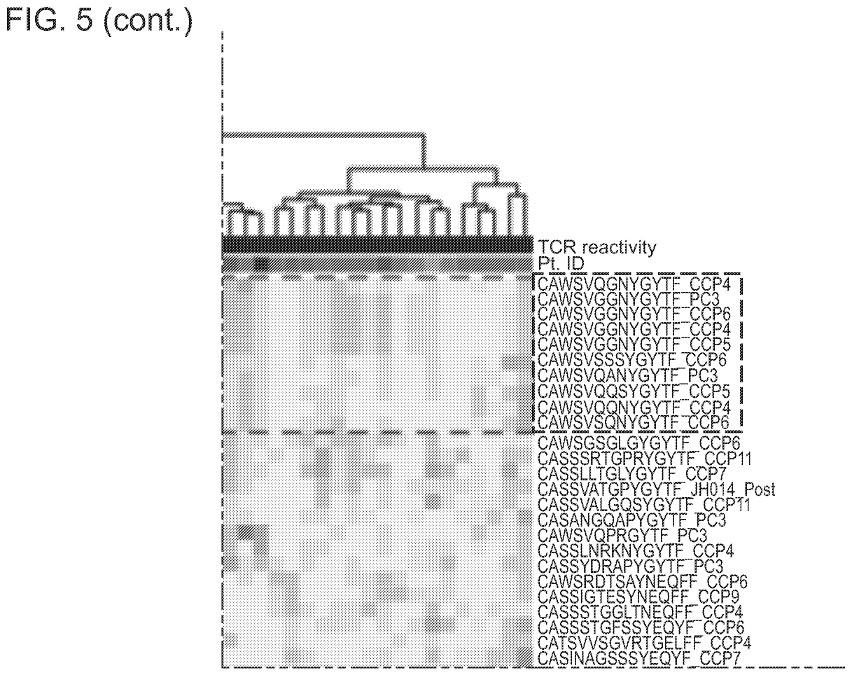

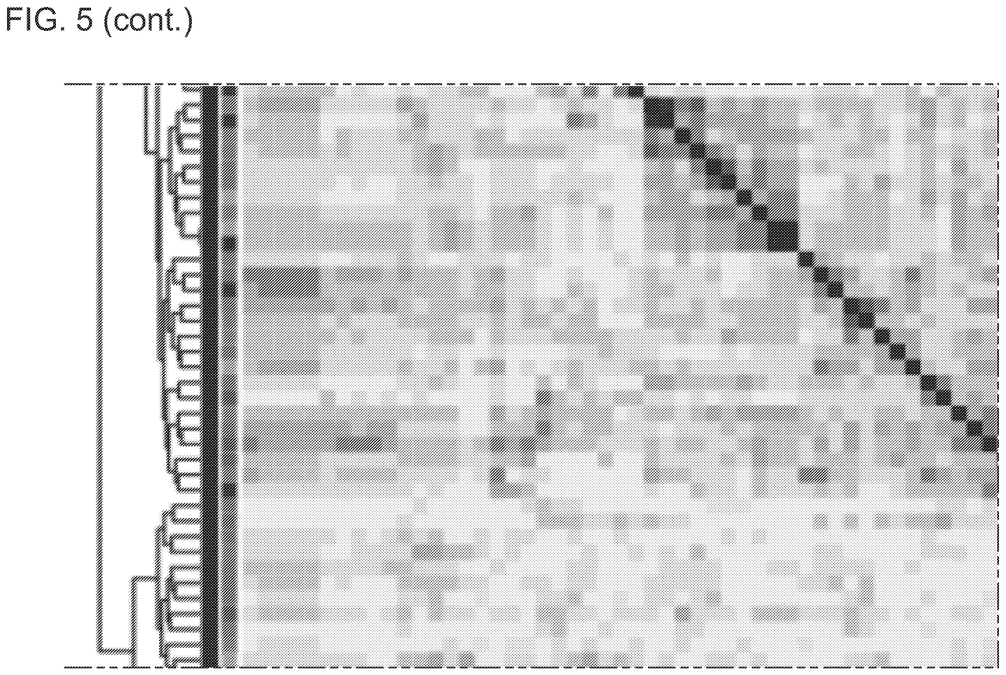

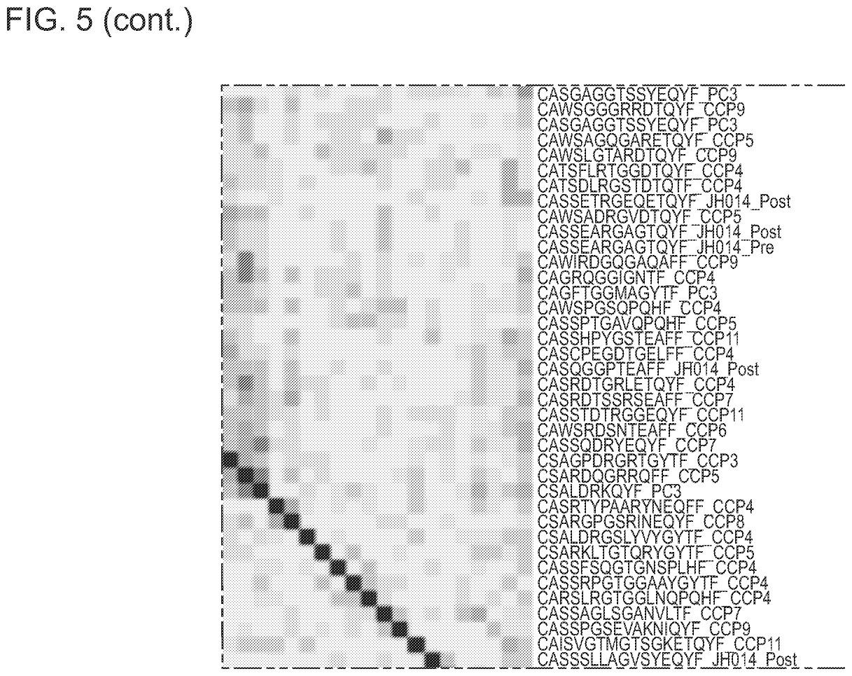

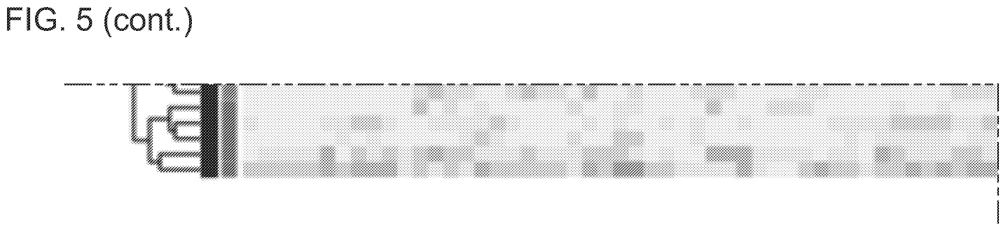

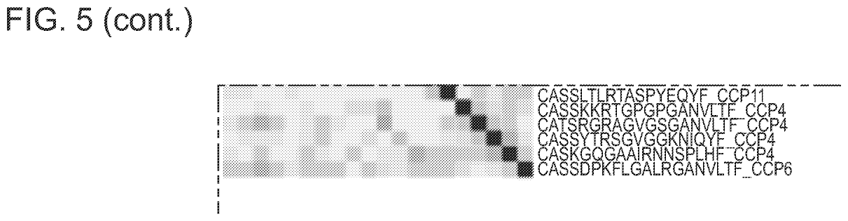

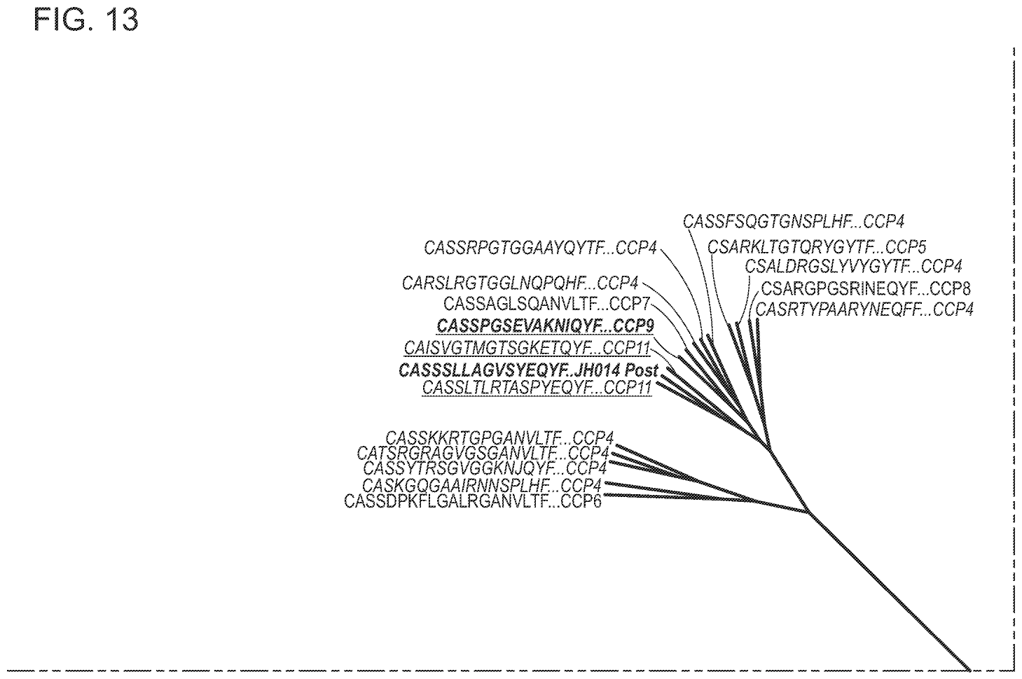

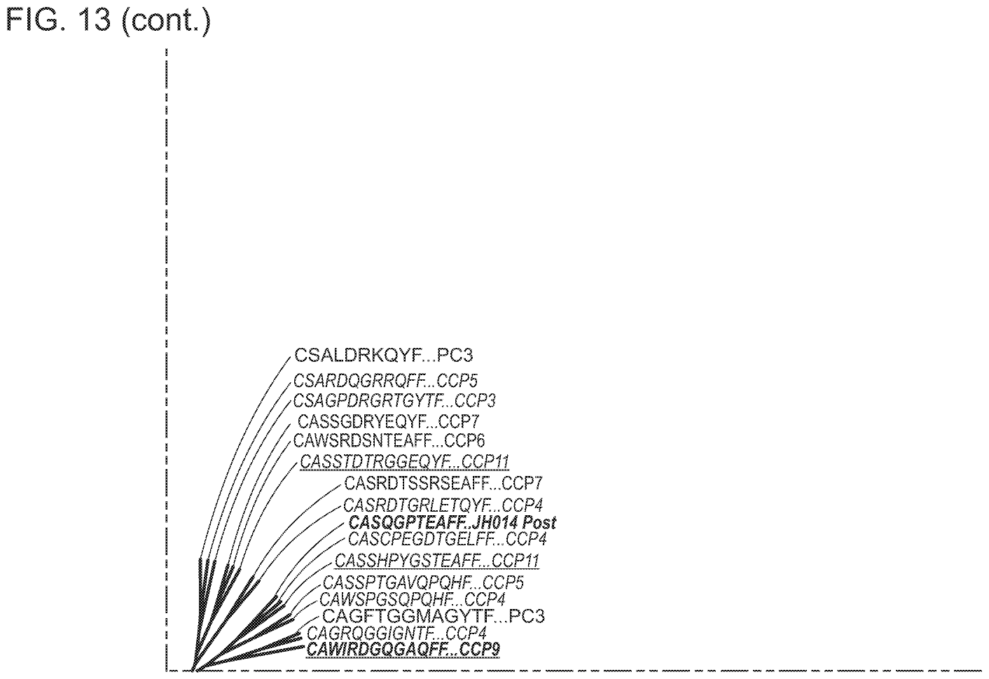

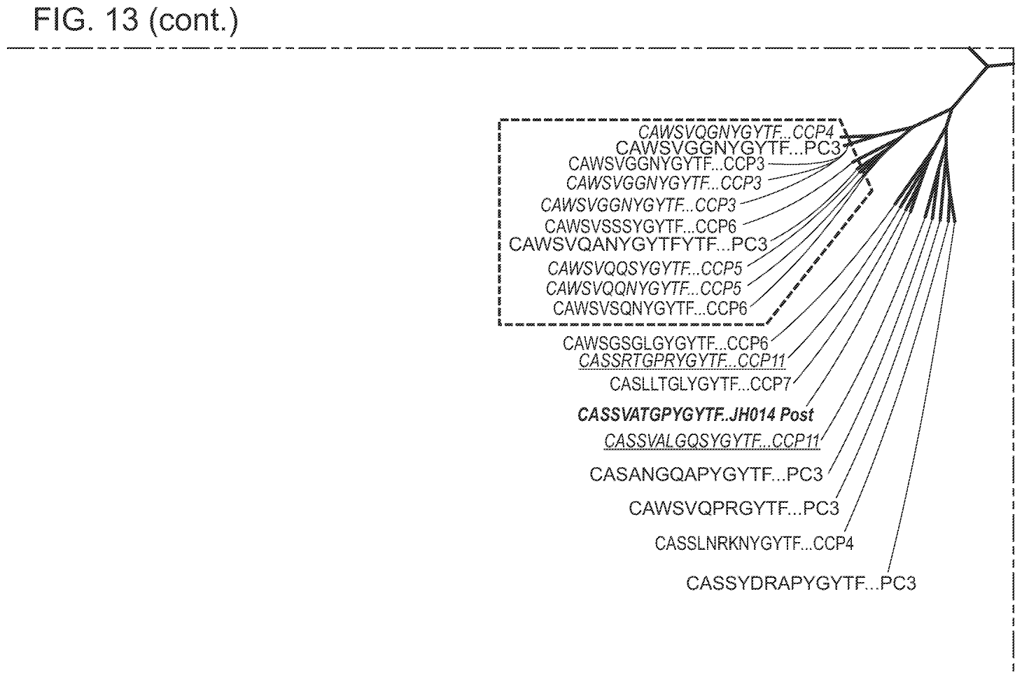

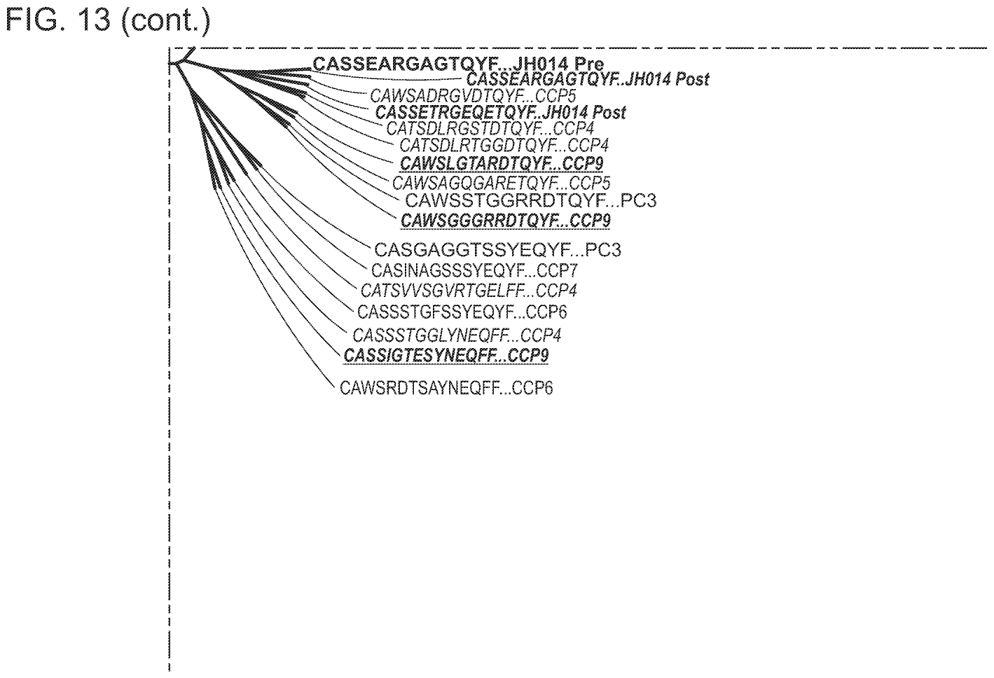

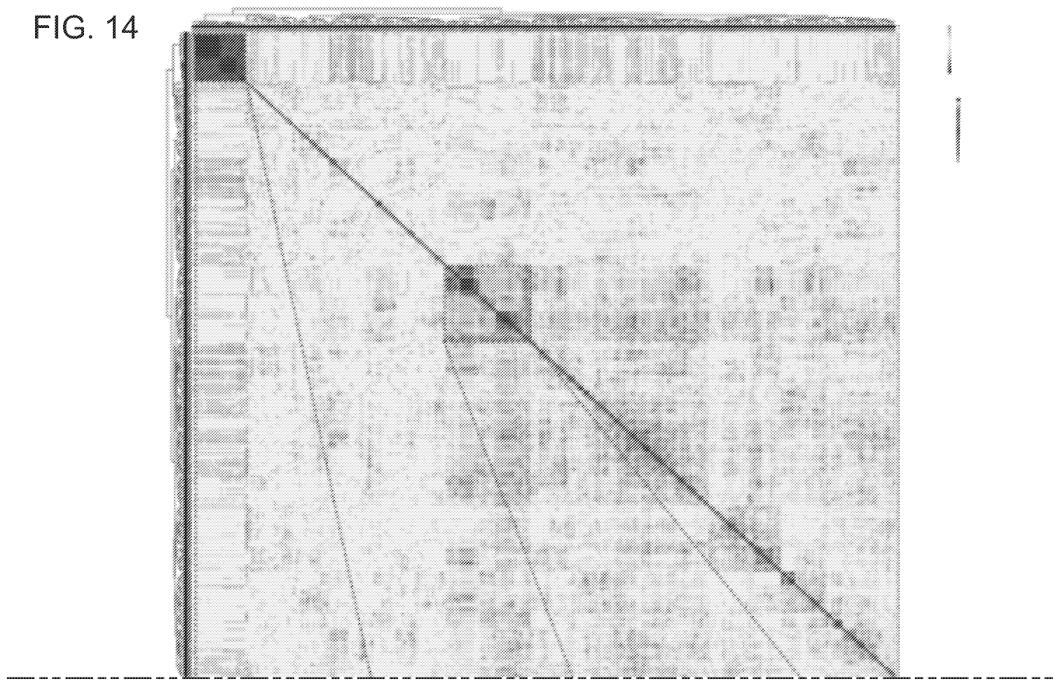

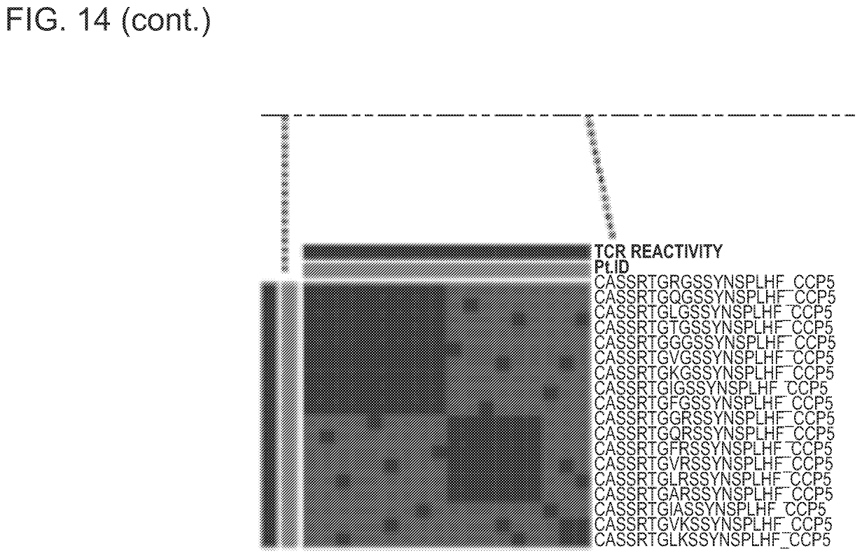

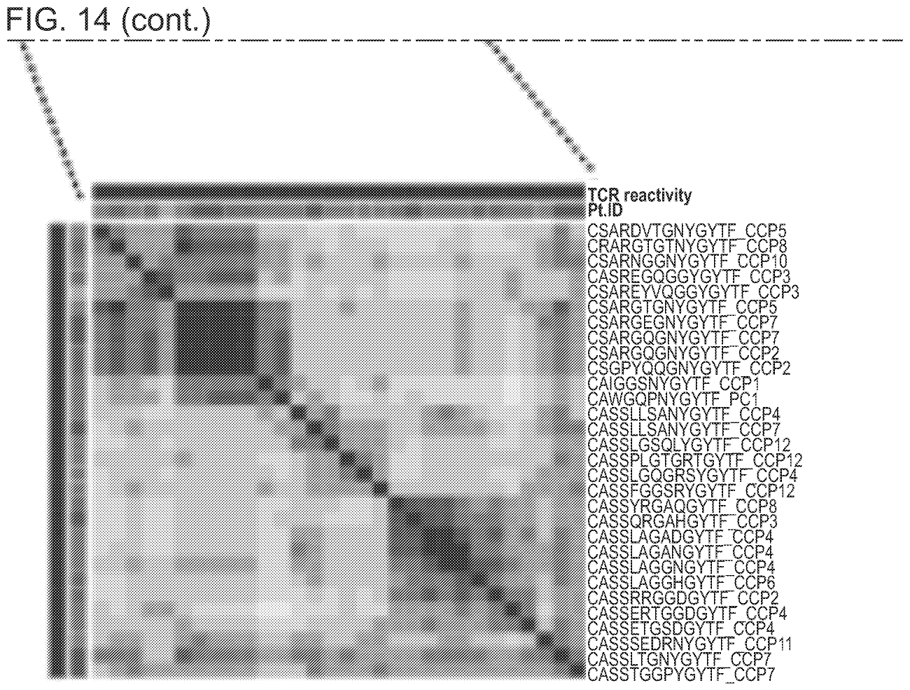

It has been shown that TCRs with shared viral antigen specificity may converge toward biased distribution of variable gene usage or CDR3 sequence identity (32). This is supported by TCR Vβ CDR3 sequence homology studies and may result from immunodominant epitopes (33), HLA super-families (34), and/or repeated stimulation by epitopes to which there is cross-recognition (35). TCR Vβ CDR3 sequence homology of the SARS-CoV-2 mono- and cross-reactive TCRs identified in the study. The Levenshtein distance was calculated between each cross-reactive TCR. Seven different cross-reactive TCR Vβ CDR3 sequences with high sequence homology (mutual Levenshtein distance <=3) were found in four patients (PC3, CCP4, CCP5, and CCP6; FIG. 5 and FIG. 13). Similarly, homology between SARS-CoV-2 S reactive TCR Vβ CDR3s was observed both within (CCP5) and between multiple patients (CCP1-8, 10-12, PC1; FIG. 14).

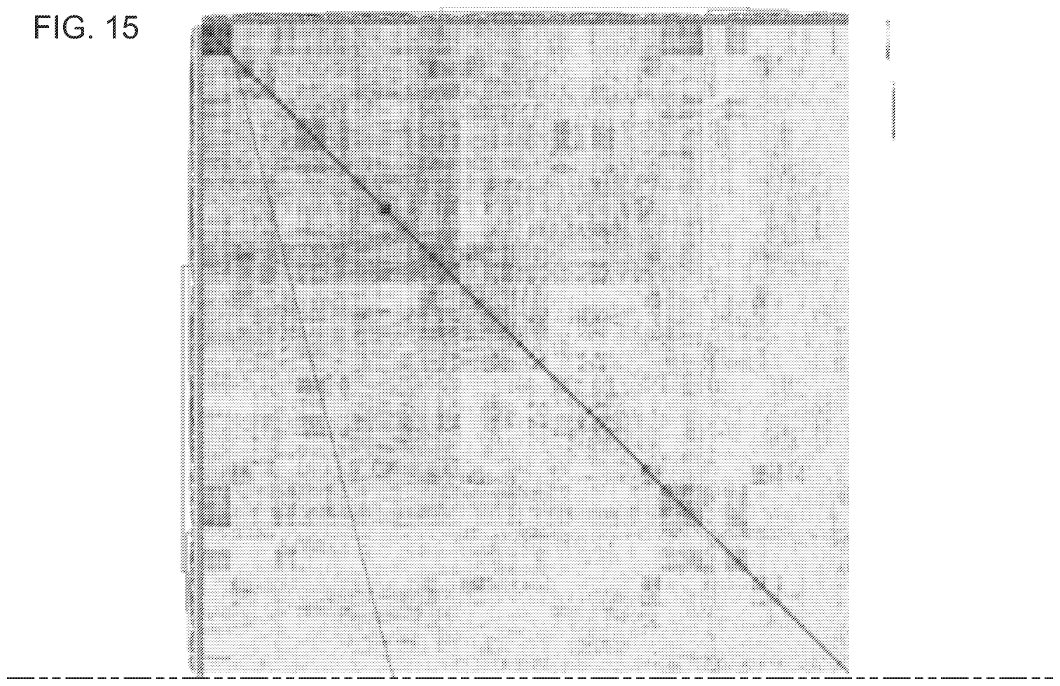

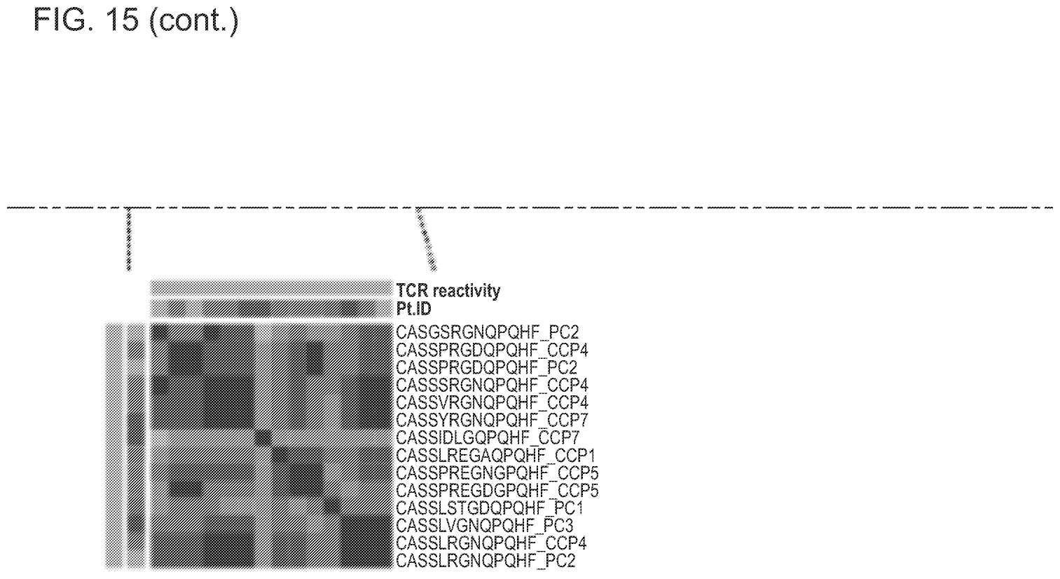

This phenomenon is not unexpected and is often seen in response to pathogens (36). Indeed, TCR homology was observed in clones reactive to the CMV, EBV, Flu (CEF) pool a positive control was used (FIG. 15). However, when combined with the data demonstrating multiple cross-reactive TCRs converging to recognize a single 17-mer SARS-CoV-2 epitope, the data suggest significant TCR convergent evolution towards recognition of immunodominant epitopes in a TCR Vβ CDR3 sequence-dependent manner.

### Example 5: Prevalence of TCR Clonotypes Cross-Reactive for SARS-CoV-2 and CCCs

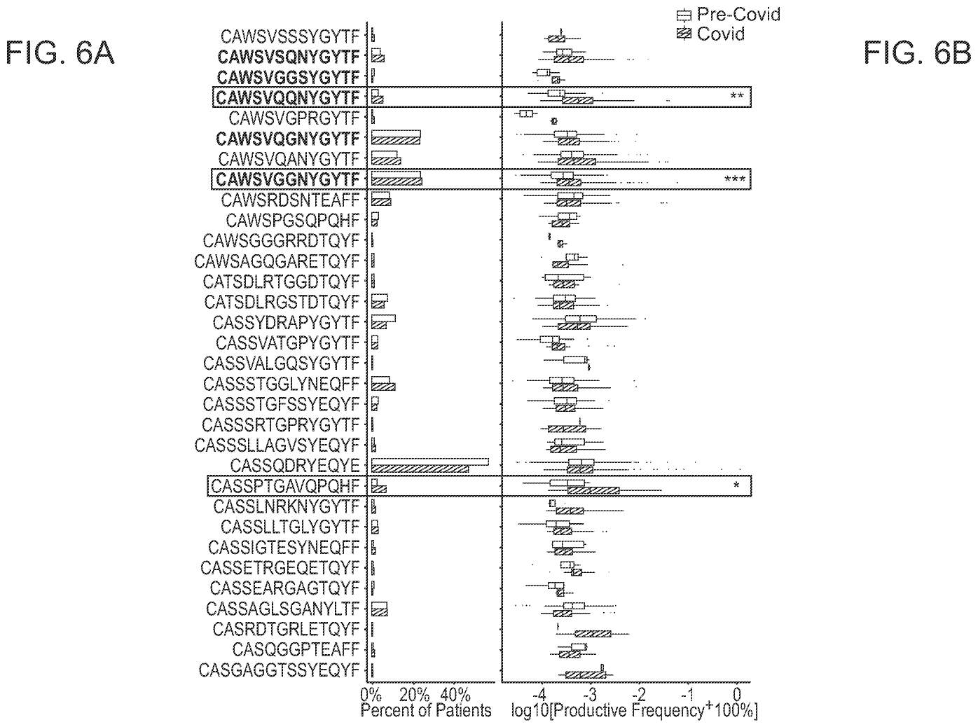

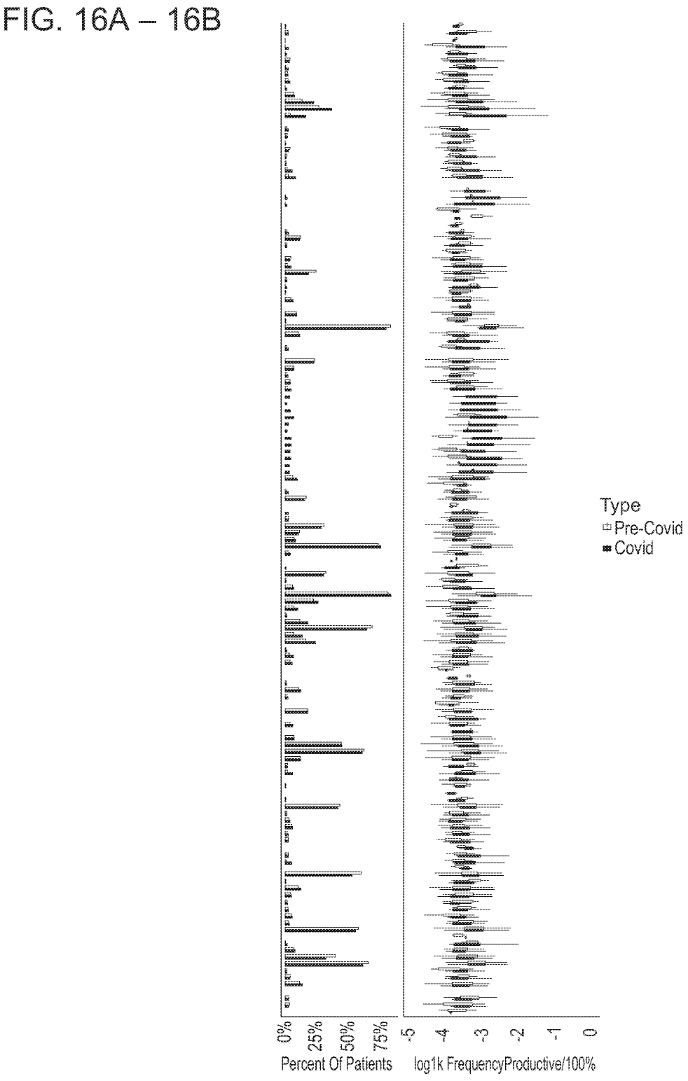

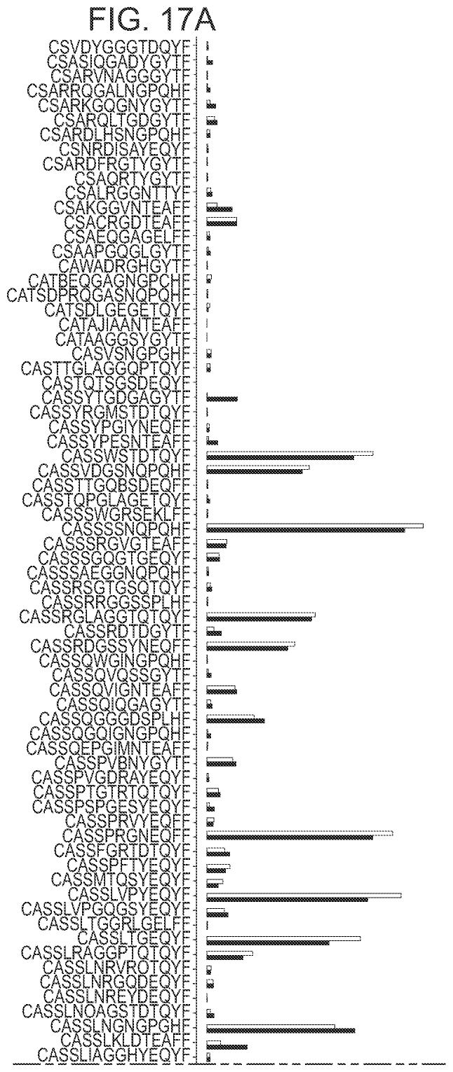

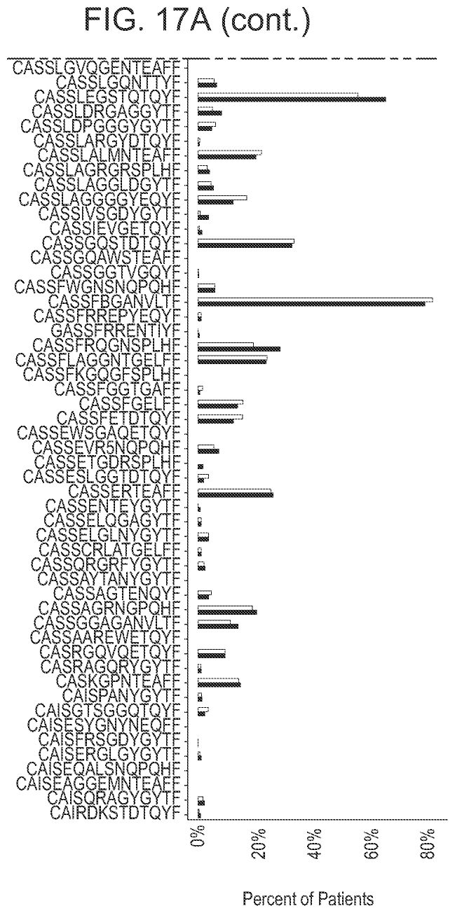

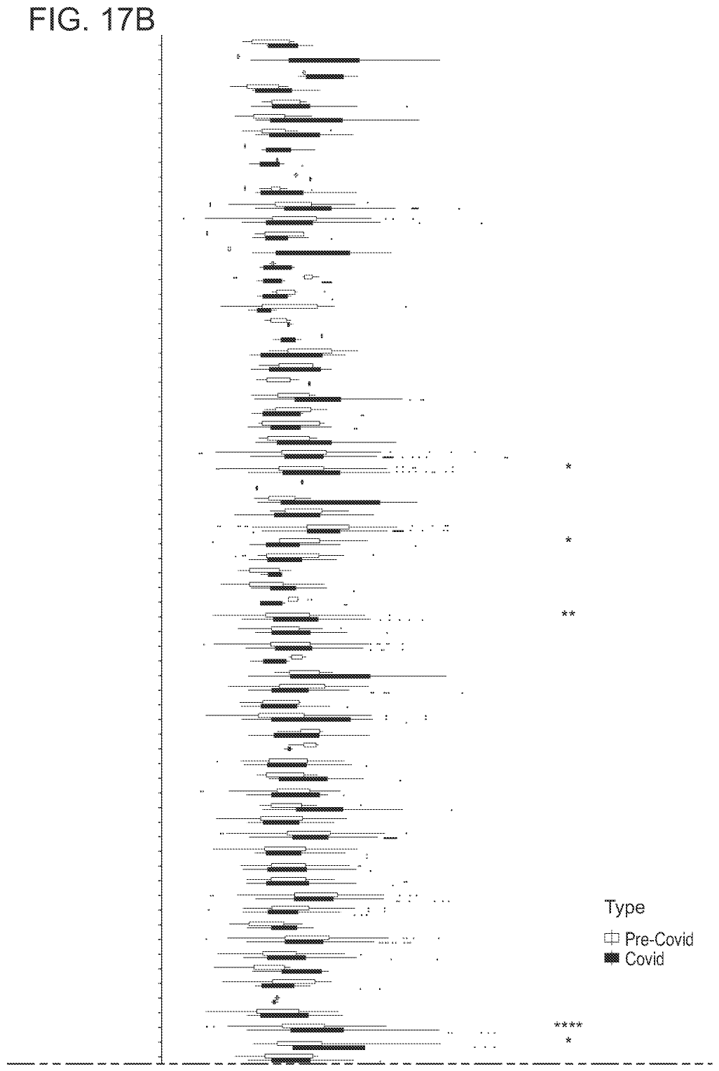

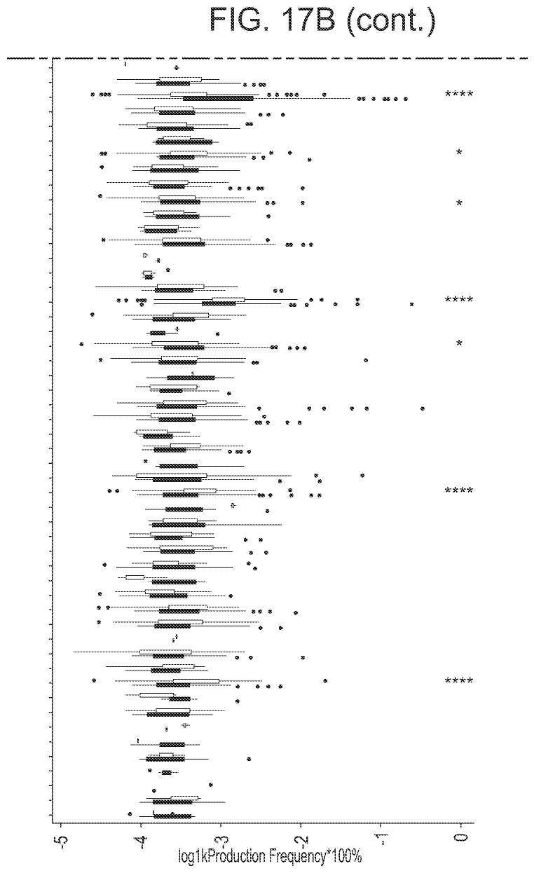

To explore whether the TCR Vβ CDR3 sequences identified by ViraFEST assay are present in the general population, the TCRs of interest from two publicly-available TCR-sequencing datasets were queried, one collected prior to the COVID-19 pandemic (21) and the second from patients with documented SARS-CoV-2 infection (22, 23). Many SARS-CoV-2 S and N mono-reactive TCRs were detected in the pre-COVID and COVID datasets, with some found in >75% of patients queried (FIG. 16A-16B and FIG. 17A-17B). Of the cross-reactive clonotypes identified in the study, 47.8% were found in patients enrolled in these unrelated studies. CASSQDRYEQYF (SEQ ID NO: 310) was the most-prevalent cross-reactive clone, found in 56.2% of pre-COVID patients and 46.5% of COVID patients (FIG. 6A).

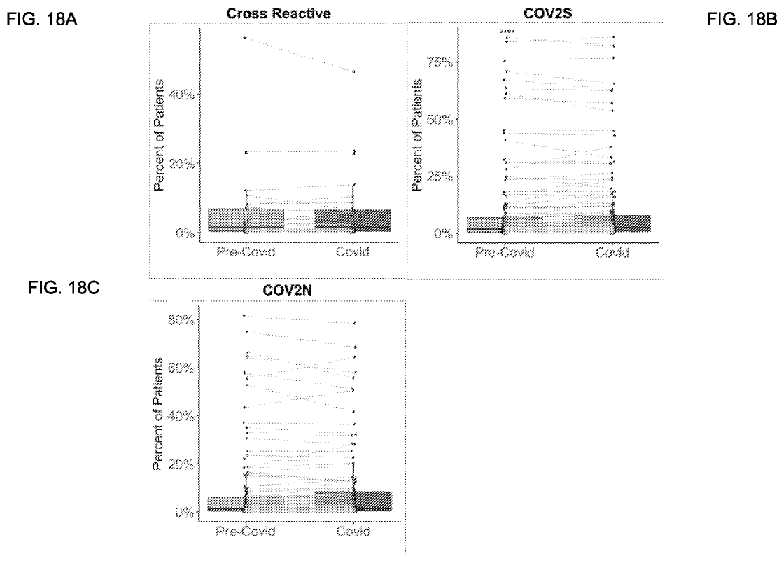

CAWSVQQNYGYTF (SEQ ID NO: 5 and SEQ ID NO: 291) and CAWSVGGNYGYTF (SEQ ID NO: 6 and SEQ ID NO: 289), which were identified in several of the donors as TCRs corresponding to cross-reactive memory CD4+ T cells, were found in 2.8% and 23.2% of pre-COVID and 4.8% and 23.6% of COVID patients, respectively (FIG. 6A). Overall, there was no difference in the percent of patients harboring cross-reactive clones before vs. during the pandemic (FIG. 6A and FIG. 18A), despite detecting a significantly higher proportion of patients with SARS-CoV-2 S, but not N, mono-reactive clonotypes (FIGS. 18B and 18C). diverse trends for each clonotype. Three cross-reactive clonotypes (CAWSVQQNYGYTF (SEQ ID NO: 291 and SEQ ID NO: 5), CAWSVGGNYGYTF (SEQ ID NO: 289 and SEQ ID NO: 6), and CASSPTGAVQPQHF (SEQ ID NO: 290 and SEQ ID NO: 8)), including two clones shared between the CCPs, were significantly higher in frequency in COVID patients compared to pre-COVID patients (p=8.86E−05, 6.12E−06, and 8.61E−04, respectively; FIG. 6B).

### Methods

### Study Participants, Biospecimens, and HLA Haplotying

This study was conducted according the Declaration of Helsinki principles. COVID-19 convalescent patients (CCPs) refer to patients who tested positive for SARS-CoV-2 by nasal swab PCR test, were symptomatic but not hospitalized, and have since recovered from COVID-19. Pre-COVID healthy donors (PCs) refer to peripheral blood mononuclear cell (PBMC) donors whose blood was drawn and processed prior to the SARS-CoV-2 epidemic (between 2011 and 2018) (43). The median age of the 12 CCPs was 38.5 (range 21 to 72). There were 8 males and 4 females. 2 of the subjects were Hispanic. There were 8 Caucasians, 2 African Americans, 1 Asian, and 1 multiracial individual. All the subjects except CCP2 had mild disease and were not hospitalized. CCP2 has well controlled HIV on antiretroviral therapy and developed severe disease. Leukapheresis product was commercially purchased for all unexposed donors between 2011-2018. CCPs were enrolled to protocols approved by the Johns Hopkins University IRB and provided written informed consent. Peripheral Blood Mononuclear Cells (PBMCs) from each study participant and unexposed donor were isolated from leukapheresis product or whole blood via Ficoll-Paque Plus gradient centrifugation and were viably cryopreserved at −140 C or were used immediately in FEST assay. CCP4 lymphoblastoid cell lines (LCL) were conducted via EBV transformation of peripheral blood B cells was at the Genetic Resources Core Facility, Johns Hopkins Institute of Genetic Medicine, Baltimore, MD. Low resolution MHC class I and II haplotyping was performed on DNA from each subject at the Johns Hopkins Hospital Immunogenetics Laboratory. High resolution was done for CCP4, CCP5, CCP6, and PC3 at the Johns Hopkins Hospital Immunogenetics Laboratory.

### Identification of Human Coronavirus-Specific T Cells

Overlapping peptide pools spanning the S protein of three common human coronaviruses (HCoV-NL63, HCoV-OC43, and HCoV-229E; BEI and jpt and HCoV-HKU1; jpt), as well as overlapping peptide pools spanning the S and N proteins of SARS-CoV-2 (BEI and jpt) were used to stimulate CD4+ T cells in the ViraFEST assay as described previously (24), with minor modifications. Briefly, 2×10⁶ PBMC were plated in culture medium (IMDM, 5% human AB serum, 10) IU/ml IL-2, 50 μg/mL gentamicin) with 10 ug/ml of individual HCoV and CoV-2 peptide pools, a positive control CEFX Ultra SuperStim consisting of pooled CMV. EBV, and Flu MHC-II restricted epitopes (jpt, PM-CEFX-3), a negative control HIV-1 Nef peptide pool (NIH AIDS Reagents), or without peptide. Each assay condition was performed in triplicate unless otherwise noted. On day 3, half the media was replaced with fresh culture media containing IL-2 (final concentration of 10 IU/mL IL-2). On day 7, half the media was replaced with fresh culture media containing IL-2 (final concentration of 10 IU/ml IL-2). On day 10, cells were harvested and CD4+ T cells were isolated using the Easy Sep CD4+ T cell isolation kit (STEMCELL, 17952). DNA was extracted from cultured CD4+ T cells using the Qiamp micro-DNA kit according to the manufacturer's instructions. TCRseq of DNA extracted from cultured CD4⁺ T cells was performed by the Johns Hopkins FEST and TCR Immunogenomics Core Facility (FTIC) using the Oncomine TCR Beta short-read assay (Illumina, Inc). Samples were pooled and sequenced on an Illumina iSeq 100 using unique dual indexes.

Data pre-processing was performed to eliminate non-productive TCR sequences and to align and trim the nucleotide sequences to obtain only the CDR3 region. Sequences not beginning with “C” or ending with “F” or “W” and having less than 7 amino acids in the CDR3 were eliminated. Resultant processed data files were uploaded to our publicly available MANAFEST analysis web app (stat-apps.onc.jhmi.edu) to bioinformatically identify antigen-specific T cell clonotypes. Clones were considered positive based on the following criteria: 1) significantly expanded in the culture of interest (in two of three replicate wells) compared to the reference culture (PBMC cultured with 10 IU/ml IL-2 and HIV-1 Nef pool or media without peptide for HIV+ donor CCP2) at an FDR less than the specified threshold (<0.05; default value), 2) significantly expanded in the culture wells of interest compared to every other culture well performed in tandem (FDR<0.05; default value), and 3) have an odds ratio >5 (default value)). To identify cross-reactive responses, statistical criteria established previously was used (29).

Identification of the Cognate TCRα for SARS-CoV-2 and HCoV-CCC specific Vβ CDR3s

PBMC from CCP4 were cultured for 10 days with SARS-CoV-2 S, SARS-CoV-2 N, and HCoV-NL63 S peptide pools as described above. On day 10, live CD4+ T cells were FACS-sorted and subjected to single cell 5′ VDJ sequencing to identify phased TCRα and TCRB chain sequences at single cell resolution using the 10× Genomics Chromium Single Cell 5′ VDJ sequencing platform on a Chromium Controller (10× Genomics) to achieve a target cell capture rate of 10,000 individual cells per sample. All samples were processed simultaneously, and the resulting libraries were prepared in a single batch following the manufacturer's instructions for VDJ library preparation. The resulting 5′ VDJ libraries were subjected to next generation sequencing at the Sidney Kimmel Comprehensive Cancer Center Experimental and Computational Genomics Core. Resulting data were pre-processed and analyzed using cellranger VDJ software (10× Genomics) and visualized using Vloupe browser (10× Genomics) to identify the paired TCR Va chain for the cognate CDR3 Vb chains identified by ViraFEST. IMGT Repertoire was used to identify the full amino acid sequence for each V and J gene for both the TCRα and TCRβ chains.

### Generation of a Jurkat Reporter Cell Line

A gBlock was created with the full TCRα and TCRβ chains separately and human constant regions and was synthesized (Integrated DNA Technologies, IDT). To generate a Jurkat reporter cell in which the TCRs of interest were transferred, the endogenous T cell receptor (TCR) α and β chains were knocked out of a specific Jurkat line that contains a luciferase reporter driven by an NFAT-response element (Promega) using the Alt-R CRISPR system (Integrated DNA Technologies, IDT). Two sequential rounds of CRISPR knockout were performed using crDNA targeting the TCRα constant region (AGAGTCTCTCAGCTGGTACA) and the TCRβ constant region (AGAAGGTGGCCGAGACCCTC). crDNA and tracrRNA (IDT) were resuspended at 100 uM with Nuclear-Free Duplex Buffer. They were duplexed at a 1:1 molar ratio according to the manufacturer's instructions. The duplexed RNA was cooled to room temperature before mixing with Cas9 Nuclease at a 1.2:1 molar ratio for 15 minutes. 40 pmols of Cas9 RNP complexed with gRNA were mixed with 500,000 cells in 20 ul of OptiMEM, loaded into a 0.1 cm cuvette (Bio-Rad) and electroporated at 90V and 15 ms using an ECM 2001 (BTX, Holliston, MA). Cells were transferred to complete growth medium and expanded for 7 days. Limiting dilution was used to acquire single cell clones and gDNA was harvested using the Quick-DNATM96 Kit (Zymo Research, Irvine, CA). The regions flanking the CRISPIR cut sites were PCR amplified (TCRα forward primer: GCCTAAGTTGGGGAGACCAC, reverse primer: GAAGCAAGGAAACAGCCTGC; TCR13 forward primer: TCGCTGTGTTTGAGCCATCAGA, reverse primer: ATGAACCACAGGTGCCCAATTC) and Sanger Sequenced. Only TCRα⁻/13⁻ clones were selected. Complete knockout was confirmed by failure to restore CD3 expression on electroporation with only a TCRα or TCR13 chain, and successful CD3 expression on electroporation with both TCR chains.

CD8 was transduced into the TCRα⁻/13⁻ Jurkat reporter cells using the MSCV retroviral expression system (Clontech). gBlocks (IDT) encoding CD8α and CD813 chains separated by a T2A self-cleaving peptide was cloned into the pMSCVpuro retroviral vector by HiFi DNA assembly (New England Biolabs). The plasmid was then co-transfected with a pVSV-G envelope vector into the GP2-293 packaging cell line per the manufacturer's instructions. Viral supernatant was harvested 48 hours after transfection and concentrated 20-fold using Retro-X Concentrator (Clontech). For transduction, non-tissue cultured treated 48-well plates were coated with 150 μL retronectin (Clontech) in PBS at 10 ug/mL overnight at 4° C. Plates were then blocked with 10% FBS for 1 hr at RT followed by washing once with PBS. After removing PBS, viral particles and 2×10⁵ of TCRα⁻/13⁻ Jurkat reporter cells were added to each well in a total volume of 500 μL cell culture media. Plates were spun at 2000 g for 1 hr at 20° C. then incubated at 37° C. Selection with 1 μg/mL puromycin (Thermo Fisher Scientific) began three days later. Single cell clones were established by limiting dilution and clones were subsequently screened for CD8 expression by flow cytometry. To generate a Jurkat reporter line that expresses both CD4 and CD8, CD4 viral particles were produced and transduced into the CD8-expressing Jurkat reporter cells using similar procedures.

### Jurkat TCR Transfer

TCRs of interest were introduced into the CD4/CD8 TCRα⁻/β⁻ Jurkat reporter line by cloning the TCRα and TCRβ chains separately into the pCI vector (Promega) by HiFi DNA assembly (New England Biolabs). The two plasmids were co-electroporated into the TCRα⁻/β⁻ Jurkat reporter line using 4 mm cuvettes (Bio-Rad) and 275V for 10 ms for 3 pulses at 0.1 interval between pulses. Cells were rested in RPMI 10% FBS at 37° C. for 24 hours. TCR expression efficiency was assessed by CD3 expression using flow cytometry. After rest live Jurkat cells were counted and plated at 1:1 ratio with a patient matched lymphoblastoid cell line (LCL) and peptide pools. Peptide titrations were carried out from 50 ug/ml to 1.25 ug/ml to assess TCR reactivity to peptide pools. Cells and peptide were co-cultured for 24 hours. TCR activity was assessed by NFAT-luciferase reporter readout using Bio-Glo™ Luciferase Assay System (Promega).

### Epitope Identification and Avidity Analysis

Cross-reactive and mono-reactive TCRs were cloned into the Jurkat reporter cell line and plated at a 1:1 ratio with patient-derived lymphoblastic cell lines (LCL) and first with mini pools consisting of 10 peptides all together comprising the entirety of the SARS-CoV-2 or HCoV-NL63 S protein. Once the stimulating mini pool was identified, the same TCRs were again transfected into Jurkats and plated with LCL and the individual SARS-CoV-2 and HCoV-NL63 peptides representing the stimulating mini pool. Once the specific peptide was identified peptide titrations were performed from 20 ug/ml to 0.15 ug/ml to assess TCR avidity for each stimulating peptide. TCR activity was again assessed by NFAT-luciferase reporter readout using Bio-Glo™ Luciferase Assay System (Promega). TCR EC50 was calculated by identifying the peptide concentration (ug/ml) required to reach ½ plateaued RLU. If 20 ug/ml of peptide was insufficient to maximize Jurkat-TCR activation, then EC₅₀ was estimated by calculating the peptide concentration (ug/ml) required to read ½ maximum RLU reached in our assay. TCR EC₅₀ was then used as a metric to estimate TCR relative avidity for individual 17-mer peptides. A two-tailed student's t test was performed using the mean of the EC₅₀ of cross-reactive TCRs for SARS-CoV-2 and the mean of the EC50 of mono-reactive TCRs for SARS-CoV-2.

### Statistical Comparisons of Functional Avidity

One-sided Wilcoxon signed-rank test was performed to compare the fold change of cross-reactive clones in response to CCC S vs. SARS-CoV-2 S. One-sided Mann-Whitney U test was used to compare the fold change of SARS-CoV-2 S mono-reactive vs. cross-reactive clones. P<0.05 was considered statistically significant.

### Heatmaps and Unrooted Phylogenetic Trees

The non-redundant TCR sequences were defined by excluding the first 3 and last 3 amino acids of the TCR V13 CDR3 region due to significant sequence overlap at the beginning and end of the CDR3 sequence (36, 44). The levenshtein distance between each pair of TCR sequences was calculated based on non-redundant TCRs, using the ‘stringdist’ R package (45). The TCR sequence homology pattern was visualized in a heatmap and an unrooted phylogenetic tree, where each row of the heatmap and each leaf of the unrooted phylogenetic tree represented a TCR V13 CDR3 sequence from a sample. The heatmap and unrooted phylogenetic tree were generated using the ‘pheatmap’ and ‘ape’ R packages respectively. All analyses were performed using R software, version 3.6.1.

### Analysis of TCR Vβ CDR3 Sequences in Public TCR Sequencing Datasets

Adaptive Biotechnologies has two large datasets that were used in this study. The first is a repository of TCR V13 sequencing files from 786 subjects acquired prior to the COVID-19 pandemic in a study of T cell receptor repertoire characteristics associated with CMV exposure (21). The second dataset consists of 1,414 subjects who were exposed to, acutely infected with, or recovered from COVID-19 in the ImmunoCODE database (22, 23). These datasets were downloaded from the immuneACCESS data portal with “ImmunoCODERelease” tag value “002”. One subject was excluded from our subsequent analysis due to unreadable format of the raw data. Of the remaining 1,413 samples, the TCR V13 CDR3 sequences identified from our MANAFEST assay were queried, and the number of samples and productive frequency aggregating at TCR amino acid level was summarized. Roughly 90 million unique TCR CDR3 sequences were generated. To quantify the difference of productive frequency in pre-COVID and COVID Adaptive datasets at clonotype level, the Mann-Whitney U test was used with multiple testing correction by Benjamini-Hochberg procedure to control false discovery rate (FDR<0.05).

### REFERENCES

1. Huang C, Wang Y, Li X, Ren L, Zhao J, Hu Y, Zhang L, Fan G, Xu J, Gu X, Cheng Z, Yu T, Xia J, Wei Y, Wu W, Xie X, Yin W, Li H, Liu M, Xiao Y, Gao H, Guo L, Xie J, Wang G, Jiang R, Gao Z, Jin Q, Wang J CB. Clinical features of patients infected with 2019 novel coronavirus in Wuhan, China. Lancet.
2. Richardson S et al. Presenting Characteristics, Comorbidities, and Outcomes among 5700 Patients Hospitalized with COVID-19 in the New York City Area. JAMA—J. Am. Med. Assoc. 2020; 323 (20):2052-2059.
3. van de Ven K, de Heij F, van Dijken H, Ferreira JA, de Jonge J. Systemic and respiratory T-cells induced by seasonal H1N1 influenza protect against pandemic H2N2 in ferrets [Internet]. Commun. Biol. 2020; 3 (1). doi:10.1038/s42003-020-01278-5.
4. Sridhar S et al. Cellular immune correlates of protection against symptomatic pandemic influenza. Nat. Med. 2013; 19 (10):1305-1312.
5. Todryk SM et al. Correlation of memory T cell responses against TRAP with protection from clinical malaria, and CD4+ CD25high T cells with susceptibility in Kenyans. PLOS One 2008; 3 (4). doi:10.1371/journal.pone.0002027.
6. Walsh EE, Shin JH, Falsey AR. Clinical impact of human coronaviruses 229E and OC43 infection in diverse adult populations. J. Infect. Dis. 2013; 208 (10):1634-1642.
7. Sagar M, White L, Mizgerd JP. Hcov Covid Disease2020.
8. Killerby ME et al. Human coronavirus circulation in the United States 2014-2017 [Internet]. J. Clin. Virol. 2018; 101:52-56.
9. Edridge AWD et al. Seasonal coronavirus protective immunity is short-lasting [Internet]. Nat. Med. 2020; 26 (November). doi:10.1038/s41591-020-1083-1
10. Galanti M, Shaman J. Direct Observation of Repeated Infections With Endemic Coronaviruses. J. Infect. Dis. 2020; (1):1-7.
11. Le Bert N et al. SARS-CoV-2-specific T cell immunity in cases of COVID-19 and SARS, and uninfected controls [Internet]. Nature 2020; 584 (7821):457-462.
12. Grifoni A et al. Targets of T Cell Responses to SARS-CoV-2 Coronavirus in Humans with COVID-19 Disease and Unexposed Individuals [Internet]. Cell 2020; 181 (7):1489-1501.e15.
13. Sekine T et al. Robust T Cell Immunity in Convalescent Individuals with Asymptomatic or Mild COVID-19. Cell 2020; 183 (1):158-168.e14.
14. Braun J et al. SARS-CoV-2-reactive T cells in healthy donors and patients with COVID-1. Nature [published online ahead of print: 2020]; (April). doi:10.1038/s41586-020-2598-9.
15. Ng K et al. Pre-existing and de novo humoral immunity to SARS-CoV-2 in humans [published online ahead of print: 2020]; doi:10.1101/2020.05.14.095414.
16. Woldemeskel BA et al. Healthy donor T-cell responses to common cold coronaviruses and SARS-CoV-2. J. Clin. Invest. [published online ahead of print: 2020]; doi:10.1172/jci143120.
17. Mateus J et al. Selective and cross-reactive SARS-CoV-2 T cell epitopes in unexposed humans. Science 2020; 370 (6512):89-94.
18. Nelde A et al. SARS-CoV-2-derived peptides define heterologous and COVID-19-induced T cell recognition. Nat. Immunol. [published online ahead of print: 2020]; doi:10.1038/s41590-020-00808-x
19. Weiskopf D et al. Phenotype and kinetics of SARS-CoV-2-specific T cells in COVID-19 patients with acute respiratory distress syndrome [Internet]. Sci. Immunol. 2020; 5 (48):eabd2071.
20. Meckiff BJ et al. Single-cell transcriptomic analysis of SARS-CoV-2 reactive CD4+ T cells [Internet]. bioRxiv 2020;2020.06.12.148916.
21. Emerson RO et al. Immunosequencing identifies signatures of cytomegalovirus exposure history and HLA-mediated effects on the T cell repertoire. Nat. Genet. 2017; 49 (5):659-665.
22. Snyder TM et al. Magnitude and Dynamics of the T-Cell Response to SARS-CoV-2 Infection at Both Individual and Population Levels [Internet]. medRxiv Prepr. Serv. Heal. Sci. 2020; 2020.07.31.20165647.
23. Nolan S et al. A large-scale database of T-cell receptor beta (TCR β) sequences and binding associations from natural and synthetic exposure to SARS-CoV-2.2020; 1-28.
24. Danilova L et al. The Mutation-Associated Neoantigen Functional Expansion of Specific T cells (MANAFEST) assay: a sensitive platform for monitoring antitumor immunity [Internet]. Cancer Immunol. Res. [published online ahead of print: January 1, 2018]; http://cancerimmunolres.aacrjournals.org/content/early/2018/06/12/2326-6066.CIR-18-0129 abstract. cited
25. Forde PM et al. Neoadjuvant PD-1 Blockade in Resectable Lung Cancer [Internet]. N. Engl. J. Med. 2018; 378 (21):1976-1986.
26. Smith KN et al. Persistent mutant oncogene specific T cells in two patients benefitting from anti-PD-1 [Internet]. J. Immunother. Cancer 2019; 7 (1):40.
27. Anagnostou V et al. Evolution of neoantigen landscape during immune checkpoint blockade in non-small cell lung cancer. Cancer Discov. 2017; 7 (3):264-276.
28. Le DT et al. Mismatch repair deficiency predicts response of solid tumors to PD-1 blockade. [Internet]. Science 2017; 357 (6349):409-413.
29. Chan HY et al. A T Cell Receptor Sequencing-Based Assay Identifies Cross-Reactive Recall CD8+T Cell Clonotypes Against Autologous HIV-1 Epitope Variants. Front. Immunol. 2020; 11 (April):1-9.
30. Bacher P et al. Low-Avidity CD4 (+) T Cell Responses to SARS-CoV-2 in Unexposed Individuals and Humans with Severe COVID-19. Immunity [published online ahead of print: November 2020]; doi:10.1016/j.immuni.2020.11.016.
31. Wintjens R, Bifani AM, Bifani P. Impact of glycan cloud on the B-cell epitope prediction of SARS-CoV-2 Spike protein [Internet]. npj Vaccines 2020; 5 (1):1-8.
32. Gil A et al. Epstein-barr virus epitope-major histocompatibility complex interaction combined with convergent recombination drives selection of diverse T cell receptor α and β repertoires. MBio 2020; 11 (2). doi:10.1128/mBio.00250-20.
33. Kedzierska K. Turner SJ, Doherty PC. Conserved T cell receptor usage in primary and recall responses to an immunodominant influenza virus nucleoprotein epitope. Proc. Natl. Acad. Sci. U.S.A. 2004; 101 (14):4942-4947.
34. Shen WJ, Zhang X, Zhang S, Liu C, Cui W. The utility of supertype clustering in prediction for class II MHC-peptide binding. Molecules 2018; 23 (11):1-18.
35. Li H, Ye C, Ji G, Han J. Determinants of public T cell responses. Cell Res. 2012; 22 (1):33-42.
36. Glanville J et al. Identifying specificity groups in the T cell receptor repertoire [Internet]. Nature 2017; 547 (7661):94-98.
37. Chandrashekar A et al. SARS-CoV-2 infection protects against rechallenge in rhesus macaques [Internet]. Science (80-.). 2020; 369 (6505):812 LP-817.
38. Robson B. COVID-19 Coronavirus spike protein analysis for synthetic vaccines, a peptidomimetic antagonist, and therapeutic drugs, and analysis of a proposed achilles' heel conserved region to minimize probability of escape mutations and drug resistance. Comput. Biol. Med. 2020; 121:103749.
39. Mongkolsapaya J et al. T cell responses in dengue hemorrhagic fever: are cross-reactive T cells suboptimal?. J. Immunol. 2006; 176 (6):3821-3829.
40. Nelson SA, Sant AJ. Imprinting and Editing of the Human CD4 T Cell Response to Influenza Virus. Front. Immunol. 2019; 10:932.
41. Nienen M et al. The Role of Pre-existing Cross-Reactive Central Memory CD4 T-Cells in Vaccination With Previously Unseen Influenza Strains. Front. Immunol. 2019; 10:593.
42. Saletti G et al. Older adults lack SARS CoV-2 cross-reactive T lymphocytes directed to human coronaviruses OC43 and NL63. Sci. Rep. 2020; 10 (1):21447.
43. Cox AL et al. Prospective evaluation of community-acquired acute-phase hepatitis C virus infection. Clin. Infect. Dis. an Off. Publ. Infect. Dis. Soc. Am. 2005; 40 (7):951-958.
44. Hou X et al. Analysis of the Repertoire Features of TCR Beta Chain CDR3 in Human by High-Throughput Sequencing [Internet]. Cell. Physiol. Biochem. 2016; 39 (2):651-667.
45. van der Loo MPJ. The stringdist package for approximate string matching. RJ. 2014; 6(1):111-122.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A T cell receptor (TCR) comprising an alpha and/or beta chain, wherein said alpha and/or beta chain comprises at least one
complementarity-determining region (CDR3)
comprising at least one sequence of SEQ IDs 1-547.

2. The TCR of claim 1, wherein the TCR comprises an alpha chain, wherein the alpha chain comprises at least one sequence of SEQ ID NO: 1-13 or 545-547.

3. The TCR of claim 1, wherein the TCR comprises a peptide comprises a cross-reactive peptide comprising at least one sequence of CAWSVQGNYGYTF, CAWSVGGNYGYTF, CAWSVSSSYGYTF, CAWSVQANYGYTF, CAWSVQQSYGYTF, CAWSVQQNYGYTF or CAWSVSQNYGYTF.

4. The TCR of claim 1, wherein the TCR comprises a mono-reactive peptide comprising at least one sequence of CSARDVTGNYGYTF, CSARGTGTNYGYTF, CSVENGGNYGYTF, CASREGQGGYGYTF, CSAREWGGGYGYTF, CSARGTGNYGYTF, CSARGEGNYGYTF, CSARGWGNYGYTF, CSARGQGNYGYTF, CSGRGQGNYGYTF, CAIGGDSNYGYTF, CAWGWFNYGYTF, CASSLLSANYGYTF, CASSLGPFYGYTF, CASSLGWQLYGYTF, CASSPLGTGRYGYTF, CASSLGDGRSYGYTF, CASSFGGSRYGYTF, CASSYRGAQGYTF, CASSQRGAHGYTF, CASSLRGADGYTF, CASSLRGANGYTF, CASSLRGGNGYTF, CASSLAGGHGYTF, CASSRRGGDGYTF, or CASSERTGGEGYTF.

5. The TCR of claim 1, further comprising a variable domain, a constant domain, or fragments thereof.

6. The TCR of claim 1, further comprising the ability to bind to a tumor antigen/HLA complex.

7. A nucleic acid molecule encoding the TCR of claim 1.

8. A vector comprising a nucleic acid molecule according to claim 7.

9. A host cell containing or expressing a vector according to claim 8.

10. A human T cell containing or expressing a TCR according to claim 1.

11. A non-human T cell containing or expressing a TCR according to claim 1.

12. A composition of claim 1 for use in the treatment of COVID-19.

13. The composition of claim 12, wherein the treatment comprises application of antiviral therapy.

14. The composition of claim 12, wherein the treatment comprises immunotherapy.

15. (canceled)

16. A method for treating a subject having a COVID-19 infection, exhibiting symptoms of a COVID-19 infection, or having suspected exposure to COVID-19,
the method comprising:
administering to a subject in need thereof an effective amount of a composition of claim 1.

17. The method of claim 16 the composition is co-administered with one or more distinct anti-viral agents.

18. The method of claim 16 wherein the composition is co-administered with one or more of hyperimmune globulins, remdesivir, oseltamivir, Galidesivir (BCX4430, Immucillin-A), 3-Deazaneplanocin A (DZNep, C-c3 Ado), Favipiravir (T-705, Avigan), lopinavir;
ritonavir, lopinavir/ritonavir (e.g. KALETRA), ribavirin, or lopinavir/ritonavir/ribavirin, Recombinant human interferon α1β, Huaier (including Huaier Granule), Eculizumab (Soliris), Recombinant human angiotensin-converting enzyme 2 (rhACE2), Carrimycin, Umifenivir (Arbidol), chloroquine phosphate, T89 (Dantonic), Fingolimod (including Fingolimod 0.5 mg), N-acetylcysteine+ Fuzheng Huayu Tablet, YinHu QingWen Decoction, LV-SMENP-DC vaccine and/or antigen-specific CTLs; a steroid (including dexamethasone); antibodies (including monoclonal antibodies such as amlanivimab (LY-CoV555) and REGN-COV2 (Regeneron) and AZD7442 (antibody combination, AstraZenca); and/or an IL-6 receptor agonist (including for example tocilizumab and sarilumab).

19-20. (canceled)

21. A method of treating cells infected by or exposed to COVID-19, the method comprising an effective amount of a composition of claim 1.

22-26. (canceled)

27. A T cell receptor (TCR) comprising an alpha and beta chain, wherein the alpha and beta chain SEQ ID NOS: 1-13 or 545-547,
or
An engineered cell expressing a T cell receptor (TCR) comprising an alpha and beta chain, wherein the alpha and beta chain comprises SEQ ID NOS: 1-13 or 545-547.

28-34. (canceled)

35. A T cell receptor (TCR) alpha chain comprising amino acid sequences of: CAVRGSGGSNYKLTF, CAFMKPRGSQGNLIF, CALSEGNYGGSQGNLIF, CAGRQGAQKLVF, CAVSSSGGSYIPTF, CAVTKPSGTALIF, CIESNTGKLIF, CAVSLTYKYIF, CAMTVNSGYSTLTF, CAENSGTYKYIF, CAENGSRDTGRRALTF, CIVRDNAGNMLTF, CVVKSRGGGGKLIF, CAVRTYGQNFVF, CAGFNYGGSQGNLIF or CAGRVYNNNDMRF.
or
A T cell receptor (TCR) comprising an alpha chain comprising an amino acid sequence of: CAVRGSGGSNYKLTF. CAFMKPRGSOGNLIF, CALSEGNYGGSOGNLIF. CAGROGAOKLVF. CAVSSSGGSYIPTE. CAVTKPSGTALIF, CIESNTGKLIF, CAVSLTYKYIF, CAMTVNSGYSTLTF. CAENSGTYKYIF. CAENGSRDTGRRALTE. CIVRDNAGNMLTF. CVVKSRGGGGKLIF, CAVRTYGONFVE. CAGFNYGGSOGNLIF
or CAGRVYNNNDMRF and a beta chain comprising SEQ ID NOS: 1-546 or 547,
or
A composition comprising at least one sequence of SEQ ID NOs: 1-547 to diagnose, treat or monitor COVID-19 disease.

36. (canceled)


SEQUENCE LISTING (943 sequences)

SEQ ID NO: 1 - protein, 16 aa, Artificial Sequence
  Description of Artificial Sequence Synthetic peptide
CATSVVSGVRTGELFF

SEQ ID NO: 2 - protein, 17 aa, Artificial Sequence
  Description of Artificial Sequence Synthetic peptide
CASSRPGTGGAAYGYTF

SEQ ID NO: 3 - protein, 16 aa, Artificial Sequence
  Description of Artificial Sequence Synthetic peptide
CASRTYPAARYNEQFF

SEQ ID NO: 4 - protein, 14 aa, Artificial Sequence
  Description of Artificial Sequence Synthetic peptide
CASRDTGRLETQYF

SEQ ID NO: 5 - protein, 13 aa, Artificial Sequence
  Description of Artificial Sequence Synthetic peptide
CAWSVQQNYGYTF

SEQ ID NO: 6 - protein, 13 aa, Artificial Sequence
  Description of Artificial Sequence Synthetic peptide
CAWSVGGNYGYTF

SEQ ID NO: 7 - protein, 17 aa, Artificial Sequence
  Description of Artificial Sequence Synthetic peptide
CASSKKRTGPGANVLTF

SEQ ID NO: 8 - protein, 13 aa, Artificial Sequence
  Description of Artificial Sequence Synthetic peptide
CAWSVQGNYGYTF

SEQ ID NO: 9 - protein, 14 aa, Artificial Sequence
  Description of Artificial Sequence Synthetic peptide
CSVELDRGRRGYTF

SEQ ID NO: 10 - protein, 14 aa, Artificial Sequence
  Description of Artificial Sequence Synthetic peptide
CASSQLGRTGELFF

SEQ ID NO: 11 - protein, 16 aa, Artificial Sequence
  Description of Artificial Sequence Synthetic peptide
CASSLSVGDTPYEQYF

SEQ ID NO: 12 - protein, 15 aa, Artificial Sequence
  Description of Artificial Sequence Synthetic peptide
CSAGRATDGHLEQYF

SEQ ID NO: 13 - protein, 13 aa, Artificial Sequence
  Description of Artificial Sequence Synthetic peptide
CASSEQGYGEQFF

SEQ ID NO: 14 - protein, 13 aa, Artificial Sequence
  Description of Artificial Sequence Synthetic peptide
CAIGGDSNYGYTF

SEQ ID NO: 15 - protein, 13 aa, Artificial Sequence
  Description of Artificial Sequence Synthetic peptide
CASKRDSINGYTF

SEQ ID NO: 16 - protein, 12 aa, Artificial Sequence
  Description of Artificial Sequence Synthetic peptide
CASSDAYYGYTF

SEQ ID NO: 17 - protein, 14 aa, Artificial Sequence
  Description of Artificial Sequence Synthetic peptide
CASSRGGFDTEAFF

SEQ ID NO: 18 - protein, 15 aa, Artificial Sequence
  Description of Artificial Sequence Synthetic peptide
CASTETPGAFRGYTF

SEQ ID NO: 19 - protein, 16 aa, Artificial Sequence
  Description of Artificial Sequence Synthetic peptide
CSARATGTSVYNEQFF

SEQ ID NO: 20 - protein, 14 aa, Artificial Sequence
  Description of Artificial Sequence Synthetic peptide
CSARSTDRAGGYTF

SEQ ID NO: 21 - protein, 12 aa, Artificial Sequence
  Description of Artificial Sequence Synthetic peptide
CSVTGSGEKLFF

SEQ ID NO: 22 - protein, 11 aa, Artificial Sequence
  Description of Artificial Sequence Synthetic peptide
CASSERETQYF

SEQ ID NO: 23 - protein, 17 aa, Artificial Sequence
  Description of Artificial Sequence Synthetic peptide
CASSLRRGDRGSYGYTF

SEQ ID NO: 24 - protein, 15 aa, Artificial Sequence
  Description of Artificial Sequence Synthetic peptide
CASSLVGGGAETQYF

SEQ ID NO: 25 - protein, 13 aa, Artificial Sequence
  Description of Artificial Sequence Synthetic peptide
CASSPLRGQKAFF

SEQ ID NO: 26 - protein, 15 aa, Artificial Sequence
  Description of Artificial Sequence Synthetic peptide
CASSSRQGALEGYTF

SEQ ID NO: 27 - protein, 15 aa, Artificial Sequence
  Description of Artificial Sequence Synthetic peptide
CATSSRTGGGNTIYF

SEQ ID NO: 28 - protein, 13 aa, Artificial Sequence
  Description of Artificial Sequence Synthetic peptide
CSVENGGNYGYTF

SEQ ID NO: 29 - protein, 15 aa, Artificial Sequence
  Description of Artificial Sequence Synthetic peptide
CAISEGQGSTDTQYF

SEQ ID NO: 30 - protein, 16 aa, Artificial Sequence
  Description of Artificial Sequence Synthetic peptide
CASRGTREGLTDTQYF

SEQ ID NO: 31 - protein, 17 aa, Artificial Sequence
  Description of Artificial Sequence Synthetic peptide
CASRSGQESSYNSPLHF

SEQ ID NO: 32 - protein, 12 aa, Artificial Sequence
  Description of Artificial Sequence Synthetic peptide
CASRTSGQPQHF

SEQ ID NO: 33 - protein, 15 aa, Artificial Sequence
  Description of Artificial Sequence Synthetic peptide
CASSFGGSGADTQYF

SEQ ID NO: 34 - protein, 16 aa, Artificial Sequence
  Description of Artificial Sequence Synthetic peptide
CASSLYVRWAHNEQFF

SEQ ID NO: 35 - protein, 14 aa, Artificial Sequence
  Description of Artificial Sequence Synthetic peptide
CASSSEGRNYGYTF

SEQ ID NO: 36 - protein, 15 aa, Artificial Sequence
  Description of Artificial Sequence Synthetic peptide
CASSSFGGGTDTQYF

SEQ ID NO: 37 - protein, 15 aa, Artificial Sequence
  Description of Artificial Sequence Synthetic peptide
CASSYALERADTQYF

SEQ ID NO: 38 - protein, 18 aa, Artificial Sequence
  Description of Artificial Sequence Synthetic peptide
CASSYGWTPGGVNTEAFF

SEQ ID NO: 39 - protein, 18 aa, Artificial Sequence
  Description of Artificial Sequence Synthetic peptide
CASSYRAGHLPDTGELFF

SEQ ID NO: 40 - protein, 14 aa, Artificial Sequence
  Description of Artificial Sequence Synthetic peptide
CSAGRGGGNNEQFF

SEQ ID NO: 41 - protein, 15 aa, Artificial Sequence
  Description of Artificial Sequence Synthetic peptide
CSAHGPWTEQETQYF

SEQ ID NO: 42 - protein, 12 aa, Artificial Sequence
  Description of Artificial Sequence Synthetic peptide
CSASPTTDTQYF

SEQ ID NO: 43 - protein, 17 aa, Artificial Sequence
  Description of Artificial Sequence Synthetic peptide
CSVPFRGGGDINQPQHF

SEQ ID NO: 44 - protein, 10 aa, Artificial Sequence
  Description of Artificial Sequence Synthetic peptide
CASGDRGAFF

SEQ ID NO: 45 - protein, 16 aa, Artificial Sequence
  Description of Artificial Sequence Synthetic peptide
CASKEGVKGSNQPQHF

SEQ ID NO: 46 - protein, 16 aa, Artificial Sequence
  Description of Artificial Sequence Synthetic peptide
CASLLSAGGGRYGYTF

SEQ ID NO: 47 - protein, 15 aa, Artificial Sequence
  Description of Artificial Sequence Synthetic peptide
CASRGLAGARDTQYF

SEQ ID NO: 48 - protein, 14 aa, Artificial Sequence
  Description of Artificial Sequence Synthetic peptide
CASRPRGGNQPQHF

SEQ ID NO: 49 - protein, 15 aa, Artificial Sequence
  Description of Artificial Sequence Synthetic peptide
CASSDRDRGYYEQYF

SEQ ID NO: 50 - protein, 16 aa, Artificial Sequence
  Description of Artificial Sequence Synthetic peptide
CASSDSKFSTDTEAFF

SEQ ID NO: 51 - protein, 14 aa, Artificial Sequence
  Description of Artificial Sequence Synthetic peptide
CASSDSRSNQPQHF

SEQ ID NO: 52 - protein, 15 aa, Artificial Sequence
  Description of Artificial Sequence Synthetic peptide
CASSEKASGYNEQFF

SEQ ID NO: 53 - protein, 14 aa, Artificial Sequence
  Description of Artificial Sequence Synthetic peptide
CASSFGGSRYGYTF

SEQ ID NO: 54 - protein, 15 aa, Artificial Sequence
  Description of Artificial Sequence Synthetic peptide
CASSFGLAGFYEQYF

SEQ ID NO: 55 - protein, 16 aa, Artificial Sequence
  Description of Artificial Sequence Synthetic peptide
CASSGGLAGATYEQYF

SEQ ID NO: 56 - protein, 15 aa, Artificial Sequence
  Description of Artificial Sequence Synthetic peptide
CASSIGIDRPYGYTF

SEQ ID NO: 57 - protein, 15 aa, Artificial Sequence
  Description of Artificial Sequence Synthetic peptide
CASSIGTDRPYGYTF

SEQ ID NO: 58 - protein, 15 aa, Artificial Sequence
  Description of Artificial Sequence Synthetic peptide
CASSIKGSSNQPQHF

SEQ ID NO: 59 - protein, 14 aa, Artificial Sequence
  Description of Artificial Sequence Synthetic peptide
CASSIQGLQQTQYF

SEQ ID NO: 60 - protein, 17 aa, Artificial Sequence
  Description of Artificial Sequence Synthetic peptide
CASSLAPGTGFGYGYTF

SEQ ID NO: 61 - protein, 15 aa, Artificial Sequence
  Description of Artificial Sequence Synthetic peptide
CASSLGKARIYEQYF

SEQ ID NO: 62 - protein, 15 aa, Artificial Sequence
  Description of Artificial Sequence Synthetic peptide
CASSLGLAAFYEQYF

SEQ ID NO: 63 - protein, 14 aa, Artificial Sequence
  Description of Artificial Sequence Synthetic peptide
CASSLGSQLYGYTF

SEQ ID NO: 64 - protein, 16 aa, Artificial Sequence
  Description of Artificial Sequence Synthetic peptide
CASSPGDRGSGQPQHF

SEQ ID NO: 65 - protein, 15 aa, Artificial Sequence
  Description of Artificial Sequence Synthetic peptide
CASSPLGTGRYGYTF

SEQ ID NO: 66 - protein, 12 aa, Artificial Sequence
  Description of Artificial Sequence Synthetic peptide
CASSPSGNTIYF

SEQ ID NO: 67 - protein, 15 aa, Artificial Sequence
  Description of Artificial Sequence Synthetic peptide
CASSPTDRGNQPQHF

SEQ ID NO: 68 - protein, 15 aa, Artificial Sequence
  Description of Artificial Sequence Synthetic peptide
CASSQGLAGFYEQYF

SEQ ID NO: 69 - protein, 13 aa, Artificial Sequence
  Description of Artificial Sequence Synthetic peptide
CASSRLTGREAFF

SEQ ID NO: 70 - protein, 12 aa, Artificial Sequence
  Description of Artificial Sequence Synthetic peptide
CASSSQFWEQYF

SEQ ID NO: 71 - protein, 13 aa, Artificial Sequence
  Description of Artificial Sequence Synthetic peptide
CASSTDGRQPQHF

SEQ ID NO: 72 - protein, 16 aa, Artificial Sequence
  Description of Artificial Sequence Synthetic peptide
CASSWGKRGRAQPQHF

SEQ ID NO: 73 - protein, 13 aa, Artificial Sequence
  Description of Artificial Sequence Synthetic peptide
CATRDSSYNEQFF

SEQ ID NO: 74 - protein, 16 aa, Artificial Sequence
  Description of Artificial Sequence Synthetic peptide
CATSDGDLPNNQPQHF

SEQ ID NO: 75 - protein, 15 aa, Artificial Sequence
  Description of Artificial Sequence Synthetic peptide
CATSDPLGPTDTQYF

SEQ ID NO: 76 - protein, 14 aa, Artificial Sequence
  Description of Artificial Sequence Synthetic peptide
CATSDPRQGMKAFF

SEQ ID NO: 77 - protein, 11 aa, Artificial Sequence
  Description of Artificial Sequence Synthetic peptide
CATSEKGEQYF

SEQ ID NO: 78 - protein, 16 aa, Artificial Sequence
  Description of Artificial Sequence Synthetic peptide
CSAIQEGRNSNQPQHF

SEQ ID NO: 79 - protein, 14 aa, Artificial Sequence
  Description of Artificial Sequence Synthetic peptide
CSALDRRSGNTIYF

SEQ ID NO: 80 - protein, 13 aa, Artificial Sequence
  Description of Artificial Sequence Synthetic peptide
CSAPARGYSPLHF

SEQ ID NO: 81 - protein, 14 aa, Artificial Sequence
  Description of Artificial Sequence Synthetic peptide
CSARLPGRAQPQHF

SEQ ID NO: 82 - protein, 14 aa, Artificial Sequence
  Description of Artificial Sequence Synthetic peptide
CSARPGLDNQPQHF

SEQ ID NO: 83 - protein, 15 aa, Artificial Sequence
  Description of Artificial Sequence Synthetic peptide
CSARVGQGGSYEQYF

SEQ ID NO: 84 - protein, 15 aa, Artificial Sequence
  Description of Artificial Sequence Synthetic peptide
CSASIGRDSSYEQYF

SEQ ID NO: 85 - protein, 15 aa, Artificial Sequence
  Description of Artificial Sequence Synthetic peptide
CASGPHGTENSPLHF

SEQ ID NO: 86 - protein, 15 aa, Artificial Sequence
  Description of Artificial Sequence Synthetic peptide
CASRPRQSMNTEAFF

SEQ ID NO: 87 - protein, 15 aa, Artificial Sequence
  Description of Artificial Sequence Synthetic peptide
CASSLAGGTYNEQFF

SEQ ID NO: 88 - protein, 13 aa, Artificial Sequence
  Description of Artificial Sequence Synthetic peptide
CASSLAGPDTQYF

SEQ ID NO: 89 - protein, 17 aa, Artificial Sequence
  Description of Artificial Sequence Synthetic peptide
CASSLGVGGLANYGYTF

SEQ ID NO: 90 - protein, 13 aa, Artificial Sequence
  Description of Artificial Sequence Synthetic peptide
CASSRRGGDGYTF

SEQ ID NO: 91 - protein, 11 aa, Artificial Sequence
  Description of Artificial Sequence Synthetic peptide
CASSSTYEQYF

SEQ ID NO: 92 - protein, 13 aa, Artificial Sequence
  Description of Artificial Sequence Synthetic peptide
CSARGQGNYGYTF

SEQ ID NO: 93 - protein, 13 aa, Artificial Sequence
  Description of Artificial Sequence Synthetic peptide
CSGRGQGNYGYTF

SEQ ID NO: 94 - protein, 14 aa, Artificial Sequence
  Description of Artificial Sequence Synthetic peptide
CASREGQGGYGYTF

SEQ ID NO: 95 - protein, 15 aa, Artificial Sequence
  Description of Artificial Sequence Synthetic peptide
CASRLTSGSENIQYF

SEQ ID NO: 96 - protein, 15 aa, Artificial Sequence
  Description of Artificial Sequence Synthetic peptide
CASSFRDRGQGGYTF

SEQ ID NO: 97 - protein, 13 aa, Artificial Sequence
  Description of Artificial Sequence Synthetic peptide
CASSFRNTGELFF

SEQ ID NO: 98 - protein, 12 aa, Artificial Sequence
  Description of Artificial Sequence Synthetic peptide
CASSLARKGYTF

SEQ ID NO: 99 - protein, 15 aa, Artificial Sequence
  Description of Artificial Sequence Synthetic peptide
CASSQGTANTGELFF

SEQ ID NO: 100 - protein, 12 aa, Artificial Sequence
  Description of Artificial Sequence Synthetic peptide
CASSSGRDTQYF

SEQ ID NO: 101 - protein, 17 aa, Artificial Sequence
  Description of Artificial Sequence Synthetic peptide
CASSSSLLTGSGNTIYF

SEQ ID NO: 102 - protein, 13 aa, Artificial Sequence
  Description of Artificial Sequence Synthetic peptide
CAGRAGEQYTQYF

SEQ ID NO: 103 - protein, 17 aa, Artificial Sequence
  Description of Artificial Sequence Synthetic peptide
CAISESPSGGPTNEQFF

SEQ ID NO: 104 - protein, 17 aa, Artificial Sequence
  Description of Artificial Sequence Synthetic peptide
CARTELKTSGENNEQFF

SEQ ID NO: 105 - protein, 15 aa, Artificial Sequence
  Description of Artificial Sequence Synthetic peptide
CASALAGEAKNIQYF

SEQ ID NO: 106 - protein, 12 aa, Artificial Sequence
  Description of Artificial Sequence Synthetic peptide
CASRTGNQPQHF

SEQ ID NO: 107 - protein, 17 aa, Artificial Sequence
  Description of Artificial Sequence Synthetic peptide
CASSDIGLAGDTDTQYF

SEQ ID NO: 108 - protein, 16 aa, Artificial Sequence
  Description of Artificial Sequence Synthetic peptide
CASSDRGGQGPDTQYF

SEQ ID NO: 109 - protein, 14 aa, Artificial Sequence
  Description of Artificial Sequence Synthetic peptide
CASSERTGGEGYTF

SEQ ID NO: 110 - protein, 11 aa, Artificial Sequence
  Description of Artificial Sequence Synthetic peptide
CASSESIGYTF

SEQ ID NO: 111 - protein, 13 aa, Artificial Sequence
  Description of Artificial Sequence Synthetic peptide
CASSETGSDGYTF

SEQ ID NO: 112 - protein, 15 aa, Artificial Sequence
  Description of Artificial Sequence Synthetic peptide
CASSFRDRGLTEAFF

SEQ ID NO: 113 - protein, 14 aa, Artificial Sequence
  Description of Artificial Sequence Synthetic peptide
CASSGSGALSPLHF

SEQ ID NO: 114 - protein, 14 aa, Artificial Sequence
  Description of Artificial Sequence Synthetic peptide
CASSGSGNLGAQYF

SEQ ID NO: 115 - protein, 16 aa, Artificial Sequence
  Description of Artificial Sequence Synthetic peptide
CASSLARQGNTGELFF

SEQ ID NO: 116 - protein, 15 aa, Artificial Sequence
  Description of Artificial Sequence Synthetic peptide
CASSLERGGQSPLHF

SEQ ID NO: 117 - protein, 15 aa, Artificial Sequence
  Description of Artificial Sequence Synthetic peptide
CASSLGDGRSYGYTF

SEQ ID NO: 118 - protein, 14 aa, Artificial Sequence
  Description of Artificial Sequence Synthetic peptide
CASSLLSANYGYTF

SEQ ID NO: 119 - protein, 15 aa, Artificial Sequence
  Description of Artificial Sequence Synthetic peptide
CASSLPGTGVTEAFF

SEQ ID NO: 120 - protein, 13 aa, Artificial Sequence
  Description of Artificial Sequence Synthetic peptide
CASSLRGADGYTF

SEQ ID NO: 121 - protein, 13 aa, Artificial Sequence
  Description of Artificial Sequence Synthetic peptide
CASSLRGANGYTF

SEQ ID NO: 122 - protein, 13 aa, Artificial Sequence
  Description of Artificial Sequence Synthetic peptide
CASSLRGGNGYTF

SEQ ID NO: 123 - protein, 16 aa, Artificial Sequence
  Description of Artificial Sequence Synthetic peptide
CASSLRGQAALGELFF

SEQ ID NO: 124 - protein, 17 aa, Artificial Sequence
  Description of Artificial Sequence Synthetic peptide
CASSLVGGTRSTDTQYF

SEQ ID NO: 125 - protein, 14 aa, Artificial Sequence
  Description of Artificial Sequence Synthetic peptide
CASSQLGRTGELFF

SEQ ID NO: 126 - protein, 15 aa, Artificial Sequence
  Description of Artificial Sequence Synthetic peptide
CASSQTGQGVEGYTF

SEQ ID NO: 127 - protein, 17 aa, Artificial Sequence
  Description of Artificial Sequence Synthetic peptide
CASSSETSGSITGTQYF

SEQ ID NO: 128 - protein, 11 aa, Artificial Sequence
  Description of Artificial Sequence Synthetic peptide
CASSSGKPQHF

SEQ ID NO: 129 - protein, 11 aa, Artificial Sequence
  Description of Artificial Sequence Synthetic peptide
CASSSGKTQYF

SEQ ID NO: 130 - protein, 16 aa, Artificial Sequence
  Description of Artificial Sequence Synthetic peptide
CASSSLGGLALNEQFF

SEQ ID NO: 131 - protein, 15 aa, Artificial Sequence
  Description of Artificial Sequence Synthetic peptide
CASSYSTTSTDTQYF

SEQ ID NO: 132 - protein, 15 aa, Artificial Sequence
  Description of Artificial Sequence Synthetic peptide
CASTDRGLFAGELFF

SEQ ID NO: 133 - protein, 12 aa, Artificial Sequence
  Description of Artificial Sequence Synthetic peptide
CATSDFKGGYTF

SEQ ID NO: 134 - protein, 15 aa, Artificial Sequence
  Description of Artificial Sequence Synthetic peptide
CATSSRTGSIYGYTF

SEQ ID NO: 135 - protein, 13 aa, Artificial Sequence
  Description of Artificial Sequence Synthetic peptide
CSAPGRDRQPQHF

SEQ ID NO: 136 - protein, 15 aa, Artificial Sequence
  Description of Artificial Sequence Synthetic peptide
CSARDARGGNTEAFF

SEQ ID NO: 137 - protein, 14 aa, Artificial Sequence
  Description of Artificial Sequence Synthetic peptide
CSARNGPSNQPQHF

SEQ ID NO: 138 - protein, 15 aa, Artificial Sequence
  Description of Artificial Sequence Synthetic peptide
CSASQPRGNTDTQYF

SEQ ID NO: 139 - protein, 14 aa, Artificial Sequence
  Description of Artificial Sequence Synthetic peptide
CSATDRGRAGELFF

SEQ ID NO: 140 - protein, 11 aa, Artificial Sequence
  Description of Artificial Sequence Synthetic peptide
CSGSTVGEQYF

SEQ ID NO: 141 - protein, 14 aa, Artificial Sequence
  Description of Artificial Sequence Synthetic peptide
CSVELDRGRRGYTF

SEQ ID NO: 142 - protein, 13 aa, Artificial Sequence
  Description of Artificial Sequence Synthetic peptide
CSVRRGRDTEAFF

SEQ ID NO: 143 - protein, 14 aa, Artificial Sequence
  Description of Artificial Sequence Synthetic peptide
CASNQGQGSKTQYF

SEQ ID NO: 144 - protein, 16 aa, Artificial Sequence
  Description of Artificial Sequence Synthetic peptide
CASSLLGIRGTDTQYF

SEQ ID NO: 145 - protein, 15 aa, Artificial Sequence
  Description of Artificial Sequence Synthetic peptide
CASSLVTGIRYEQYF

SEQ ID NO: 146 - protein, 18 aa, Artificial Sequence
  Description of Artificial Sequence Synthetic peptide
CASSRTGARSSYNSPLHF

SEQ ID NO: 147 - protein, 18 aa, Artificial Sequence
  Description of Artificial Sequence Synthetic peptide
CASSRTGFGSSYNSPLHF

SEQ ID NO: 148 - protein, 18 aa, Artificial Sequence
  Description of Artificial Sequence Synthetic peptide
CASSRTGFRSSYNSPLHF

SEQ ID NO: 149 - protein, 18 aa, Artificial Sequence
  Description of Artificial Sequence Synthetic peptide
CASSRTGGGSSYNSPLHF

SEQ ID NO: 150 - protein, 18 aa, Artificial Sequence
  Description of Artificial Sequence Synthetic peptide
CASSRTGGRSSYNSPLHF

SEQ ID NO: 151 - protein, 18 aa, Artificial Sequence
  Description of Artificial Sequence Synthetic peptide
CASSRTGIASSYNSPLHF

SEQ ID NO: 152 - protein, 18 aa, Artificial Sequence
  Description of Artificial Sequence Synthetic peptide
CASSRTGIGSSYNSPLHF

SEQ ID NO: 153 - protein, 18 aa, Artificial Sequence
  Description of Artificial Sequence Synthetic peptide
CASSRTGKGSSYNSPLHF

SEQ ID NO: 154 - protein, 18 aa, Artificial Sequence
  Description of Artificial Sequence Synthetic peptide
CASSRTGLGSSYNSPLHF

SEQ ID NO: 155 - protein, 18 aa, Artificial Sequence
  Description of Artificial Sequence Synthetic peptide
CASSRTGLKSSYNSPLHF

SEQ ID NO: 156 - protein, 18 aa, Artificial Sequence
  Description of Artificial Sequence Synthetic peptide
CASSRTGLRSSYNSPLHF

SEQ ID NO: 157 - protein, 18 aa, Artificial Sequence
  Description of Artificial Sequence Synthetic peptide
CASSRTGQGSSYNSPLHF

SEQ ID NO: 158 - protein, 18 aa, Artificial Sequence
  Description of Artificial Sequence Synthetic peptide
CASSRTGQRSSYNSPLHF

SEQ ID NO: 159 - protein, 18 aa, Artificial Sequence
  Description of Artificial Sequence Synthetic peptide
CASSRTGRGSSYNSPLHF

SEQ ID NO: 160 - protein, 18 aa, Artificial Sequence
  Description of Artificial Sequence Synthetic peptide
CASSRTGTGSSYNSPLHF

SEQ ID NO: 161 - protein, 18 aa, Artificial Sequence
  Description of Artificial Sequence Synthetic peptide
CASSRTGVGSSYNSPLHF

SEQ ID NO: 162 - protein, 18 aa, Artificial Sequence
  Description of Artificial Sequence Synthetic peptide
CASSRTGVKSSYNSPLHF

SEQ ID NO: 163 - protein, 18 aa, Artificial Sequence
  Description of Artificial Sequence Synthetic peptide
CASSRTGVRSSYNSPLHF

SEQ ID NO: 164 - protein, 16 aa, Artificial Sequence
  Description of Artificial Sequence Synthetic peptide
CASSSPATGRNQPQHF

SEQ ID NO: 165 - protein, 19 aa, Artificial Sequence
  Description of Artificial Sequence Synthetic peptide
CSALRYGPGQGMENTEAFF

SEQ ID NO: 166 - protein, 14 aa, Artificial Sequence
  Description of Artificial Sequence Synthetic peptide
CSARDVTGNYGYTF

SEQ ID NO: 167 - protein, 13 aa, Artificial Sequence
  Description of Artificial Sequence Synthetic peptide
CSARGTGNYGYTF

SEQ ID NO: 168 - protein, 13 aa, Artificial Sequence
  Description of Artificial Sequence Synthetic peptide
CAGSDGLLSPLHF

SEQ ID NO: 169 - protein, 17 aa, Artificial Sequence
  Description of Artificial Sequence Synthetic peptide
CAISEIVFVGSYNEQFF

SEQ ID NO: 170 - protein, 15 aa, Artificial Sequence
  Description of Artificial Sequence Synthetic peptide
CASNPGGAVYNEQFF

SEQ ID NO: 171 - protein, 14 aa, Artificial Sequence
  Description of Artificial Sequence Synthetic peptide
CASRLAGGSYEQYF

SEQ ID NO: 172 - protein, 15 aa, Artificial Sequence
  Description of Artificial Sequence Synthetic peptide
CASSERWTGVTEAFF

SEQ ID NO: 173 - protein, 16 aa, Artificial Sequence
  Description of Artificial Sequence Synthetic peptide
CASSFRTGSLDYGYTF

SEQ ID NO: 174 - protein, 15 aa, Artificial Sequence
  Description of Artificial Sequence Synthetic peptide
CASSLAGEVNQPQHF

SEQ ID NO: 175 - protein, 12 aa, Artificial Sequence
  Description of Artificial Sequence Synthetic peptide
CASSLRGDTQYF

SEQ ID NO: 176 - protein, 17 aa, Artificial Sequence
  Description of Artificial Sequence Synthetic peptide
CASSLRLAGDGTGELFF

SEQ ID NO: 177 - protein, 16 aa, Artificial Sequence
  Description of Artificial Sequence Synthetic peptide
CASSLRTSGVYNEQFF

SEQ ID NO: 178 - protein, 16 aa, Artificial Sequence
  Description of Artificial Sequence Synthetic peptide
CASSPARSANTGELFF

SEQ ID NO: 179 - protein, 14 aa, Artificial Sequence
  Description of Artificial Sequence Synthetic peptide
CASSPDLHSNEQFF

SEQ ID NO: 180 - protein, 15 aa, Artificial Sequence
  Description of Artificial Sequence Synthetic peptide
CASSPGLAGAETQYF

SEQ ID NO: 181 - protein, 15 aa, Artificial Sequence
  Description of Artificial Sequence Synthetic peptide
CASSPGLAGAGRQYF

SEQ ID NO: 182 - protein, 14 aa, Artificial Sequence
  Description of Artificial Sequence Synthetic peptide
CASSPPAASDEQYF

SEQ ID NO: 183 - protein, 14 aa, Artificial Sequence
  Description of Artificial Sequence Synthetic peptide
CASSVSGILGEQYF

SEQ ID NO: 184 - protein, 16 aa, Artificial Sequence
  Description of Artificial Sequence Synthetic peptide
CASSYMTSGGTDTQYF

SEQ ID NO: 185 - protein, 13 aa, Artificial Sequence
  Description of Artificial Sequence Synthetic peptide
CASSYRGAQGYTF

SEQ ID NO: 186 - protein, 15 aa, Artificial Sequence
  Description of Artificial Sequence Synthetic peptide
CASSYSSGGIYGYTF

SEQ ID NO: 187 - protein, 15 aa, Artificial Sequence
  Description of Artificial Sequence Synthetic peptide
CASSYSSGGSYEQYF

SEQ ID NO: 188 - protein, 15 aa, Artificial Sequence
  Description of Artificial Sequence Synthetic peptide
CASSYSTGGIYEQYF

SEQ ID NO: 189 - protein, 12 aa, Artificial Sequence
  Description of Artificial Sequence Synthetic peptide
CASTESTGHGTF

SEQ ID NO: 190 - protein, 13 aa, Artificial Sequence
  Description of Artificial Sequence Synthetic peptide
CASTTGQGEGYTF

SEQ ID NO: 191 - protein, 15 aa, Artificial Sequence
  Description of Artificial Sequence Synthetic peptide
CASVQREGYTGELFF

SEQ ID NO: 192 - protein, 11 aa, Artificial Sequence
  Description of Artificial Sequence Synthetic peptide
CATGTGLGYTF

SEQ ID NO: 193 - protein, 13 aa, Artificial Sequence
  Description of Artificial Sequence Synthetic peptide
CSVGATGLGGYTF

SEQ ID NO: 194 - protein, 15 aa, Artificial Sequence
  Description of Artificial Sequence Synthetic peptide
CASSEFRMSGDTQYF

SEQ ID NO: 195 - protein, 16 aa, Artificial Sequence
  Description of Artificial Sequence Synthetic peptide
CASSEGSWDIRSPLHF

SEQ ID NO: 196 - protein, 13 aa, Artificial Sequence
  Description of Artificial Sequence Synthetic peptide
CASSLANTYEQYF

SEQ ID NO: 197 - protein, 16 aa, Artificial Sequence
  Description of Artificial Sequence Synthetic peptide
CASSLATGAATDTQYF

SEQ ID NO: 198 - protein, 14 aa, Artificial Sequence
  Description of Artificial Sequence Synthetic peptide
CASSLDRLNQPQHF

SEQ ID NO: 199 - protein, 13 aa, Artificial Sequence
  Description of Artificial Sequence Synthetic peptide
CASSLGPFYGYTF

SEQ ID NO: 200 - protein, 14 aa, Artificial Sequence
  Description of Artificial Sequence Synthetic peptide
CASSLSGPQETQYF

SEQ ID NO: 201 - protein, 13 aa, Artificial Sequence
  Description of Artificial Sequence Synthetic peptide
CASSLTGNYGYTF

SEQ ID NO: 202 - protein, 14 aa, Artificial Sequence
  Description of Artificial Sequence Synthetic peptide
CASSPPANTDTQYF

SEQ ID NO: 203 - protein, 15 aa, Artificial Sequence
  Description of Artificial Sequence Synthetic peptide
CASSPSATQQETQYF

SEQ ID NO: 204 - protein, 13 aa, Artificial Sequence
  Description of Artificial Sequence Synthetic peptide
CASSQRGAHGYTF

SEQ ID NO: 205 - protein, 16 aa, Artificial Sequence
  Description of Artificial Sequence Synthetic peptide
CASSSGTAPSFYGYTF

SEQ ID NO: 206 - protein, 13 aa, Artificial Sequence
  Description of Artificial Sequence Synthetic peptide
CASSTGGPYGYTF

SEQ ID NO: 207 - protein, 15 aa, Artificial Sequence
  Description of Artificial Sequence Synthetic peptide
CASSTLDRRGGPLHF

SEQ ID NO: 208 - protein, 14 aa, Artificial Sequence
  Description of Artificial Sequence Synthetic peptide
CASSYDRVGQPQHF

SEQ ID NO: 209 - protein, 12 aa, Artificial Sequence
  Description of Artificial Sequence Synthetic peptide
CAWSVDRYEQYF

SEQ ID NO: 210 - protein, 13 aa, Artificial Sequence
  Description of Artificial Sequence Synthetic peptide
CSAPGVGSYEQYF

SEQ ID NO: 211 - protein, 13 aa, Artificial Sequence
  Description of Artificial Sequence Synthetic peptide
CSARGEGNYGYTF

SEQ ID NO: 212 - protein, 15 aa, Artificial Sequence
  Description of Artificial Sequence Synthetic peptide
CAISESITGGGRAFF

SEQ ID NO: 213 - protein, 14 aa, Artificial Sequence
  Description of Artificial Sequence Synthetic peptide
CASMGGLANTEAFF

SEQ ID NO: 214 - protein, 14 aa, Artificial Sequence
  Description of Artificial Sequence Synthetic peptide
CASRDNGQGVMWTF

SEQ ID NO: 215 - protein, 13 aa, Artificial Sequence
  Description of Artificial Sequence Synthetic peptide
CASRDRENYGYTF

SEQ ID NO: 216 - protein, 13 aa, Artificial Sequence
  Description of Artificial Sequence Synthetic peptide
CASRGQGNQPQHF

SEQ ID NO: 217 - protein, 14 aa, Artificial Sequence
  Description of Artificial Sequence Synthetic peptide
CASSEGQGAGGYTF

SEQ ID NO: 218 - protein, 14 aa, Artificial Sequence
  Description of Artificial Sequence Synthetic peptide
CASSKPRTGDGYTF

SEQ ID NO: 219 - protein, 13 aa, Artificial Sequence
  Description of Artificial Sequence Synthetic peptide
CASSLAGGHGYTF

SEQ ID NO: 220 - protein, 17 aa, Artificial Sequence
  Description of Artificial Sequence Synthetic peptide
CASSLAGGPVNGGELFF

SEQ ID NO: 221 - protein, 11 aa, Artificial Sequence
  Description of Artificial Sequence Synthetic peptide
CASSLGTVHTF

SEQ ID NO: 222 - protein, 16 aa, Artificial Sequence
  Description of Artificial Sequence Synthetic peptide
CASSLLQSRQGHGYTF

SEQ ID NO: 223 - protein, 13 aa, Artificial Sequence
  Description of Artificial Sequence Synthetic peptide
CASSLVVQDSLHF

SEQ ID NO: 224 - protein, 15 aa, Artificial Sequence
  Description of Artificial Sequence Synthetic peptide
CASSRDSSDGQPQHF

SEQ ID NO: 225 - protein, 16 aa, Artificial Sequence
  Description of Artificial Sequence Synthetic peptide
CASSSWDRGINSPLHF

SEQ ID NO: 226 - protein, 15 aa, Artificial Sequence
  Description of Artificial Sequence Synthetic peptide
CATSRAGGSADTQYF

SEQ ID NO: 227 - protein, 14 aa, Artificial Sequence
  Description of Artificial Sequence Synthetic peptide
CATSSDRVGSPLHF

SEQ ID NO: 228 - protein, 16 aa, Artificial Sequence
  Description of Artificial Sequence Synthetic peptide
CSAEAPRPGGGGELFF

SEQ ID NO: 229 - protein, 14 aa, Artificial Sequence
  Description of Artificial Sequence Synthetic peptide
CSAILAGGRNEQFF

SEQ ID NO: 230 - protein, 16 aa, Artificial Sequence
  Description of Artificial Sequence Synthetic peptide
CSAPLQGGGTGNEQFF

SEQ ID NO: 231 - protein, 14 aa, Artificial Sequence
  Description of Artificial Sequence Synthetic peptide
CSARGTGTNYGYTF

SEQ ID NO: 232 - protein, 14 aa, Artificial Sequence
  Description of Artificial Sequence Synthetic peptide
CSARPGLHYYEQYF

SEQ ID NO: 233 - protein, 12 aa, Artificial Sequence
  Description of Artificial Sequence Synthetic peptide
CSARTGADGYTF

SEQ ID NO: 234 - protein, 14 aa, Artificial Sequence
  Description of Artificial Sequence Synthetic peptide
CASRPAQGPRGYTF

SEQ ID NO: 235 - protein, 13 aa, Artificial Sequence
  Description of Artificial Sequence Synthetic peptide
CASSGTGVWEQYF

SEQ ID NO: 236 - protein, 17 aa, Artificial Sequence
  Description of Artificial Sequence Synthetic peptide
CASSLGNWAGSNTEAFF

SEQ ID NO: 237 - protein, 13 aa, Artificial Sequence
  Description of Artificial Sequence Synthetic peptide
CASSLQDATEAFF

SEQ ID NO: 238 - protein, 18 aa, Artificial Sequence
  Description of Artificial Sequence Synthetic peptide
CASSLVAGTGIGFQPQHF

SEQ ID NO: 239 - protein, 17 aa, Artificial Sequence
  Description of Artificial Sequence Synthetic peptide
CASSPLGQGRDLYGYTF

SEQ ID NO: 240 - protein, 18 aa, Artificial Sequence
  Description of Artificial Sequence Synthetic peptide
CASSPSAFAGVSLETQYF

SEQ ID NO: 241 - protein, 16 aa, Artificial Sequence
  Description of Artificial Sequence Synthetic peptide
CASSSQTSGGQETQYF

SEQ ID NO: 242 - protein, 15 aa, Artificial Sequence
  Description of Artificial Sequence Synthetic peptide
CASSVLAGGEGTQYF

SEQ ID NO: 243 - protein, 13 aa, Artificial Sequence
  Description of Artificial Sequence Synthetic peptide
CATSRGPLEKLFF

SEQ ID NO: 244 - protein, 14 aa, Artificial Sequence
  Description of Artificial Sequence Synthetic peptide
CATYPGGEAAEQYF

SEQ ID NO: 245 - protein, 14 aa, Artificial Sequence
  Description of Artificial Sequence Synthetic peptide
CAWSPDGKAYEQYF

SEQ ID NO: 246 - protein, 12 aa, Artificial Sequence
  Description of Artificial Sequence Synthetic peptide
CSARAATDTQYF

SEQ ID NO: 247 - protein, 13 aa, Artificial Sequence
  Description of Artificial Sequence Synthetic peptide
CSARDHGTDTQYF

SEQ ID NO: 248 - protein, 17 aa, Artificial Sequence
  Description of Artificial Sequence Synthetic peptide
CSARGQVGLAIEYTQYF

SEQ ID NO: 249 - protein, 16 aa, Artificial Sequence
  Description of Artificial Sequence Synthetic peptide
CSARRENGGSYNEQFF

SEQ ID NO: 250 - protein, 11 aa, Artificial Sequence
  Description of Artificial Sequence Synthetic peptide
CSARRMTGQAF

SEQ ID NO: 251 - protein, 15 aa, Artificial Sequence
  Description of Artificial Sequence Synthetic peptide
CSARVPVGRNTEAFF

SEQ ID NO: 252 - protein, 16 aa, Artificial Sequence
  Description of Artificial Sequence Synthetic peptide
CASGYPPGPNTGELFF

SEQ ID NO: 253 - protein, 14 aa, Artificial Sequence
  Description of Artificial Sequence Synthetic peptide
CASSATGGSETQYF

SEQ ID NO: 254 - protein, 17 aa, Artificial Sequence
  Description of Artificial Sequence Synthetic peptide
CASSEARVSEATGELFF

SEQ ID NO: 255 - protein, 17 aa, Artificial Sequence
  Description of Artificial Sequence Synthetic peptide
CASSEGSGRPSRDEQFF

SEQ ID NO: 256 - protein, 15 aa, Artificial Sequence
  Description of Artificial Sequence Synthetic peptide
CASSETRGEQETQYF

SEQ ID NO: 257 - protein, 17 aa, Artificial Sequence
  Description of Artificial Sequence Synthetic peptide
CASSQLPQLSSYNEQFF

SEQ ID NO: 258 - protein, 14 aa, Artificial Sequence
  Description of Artificial Sequence Synthetic peptide
CASSSTGGWETQYF

SEQ ID NO: 259 - protein, 16 aa, Artificial Sequence
  Description of Artificial Sequence Synthetic peptide
CSARSPSGSSYNEQFF

SEQ ID NO: 260 - protein, 13 aa, Artificial Sequence
  Description of Artificial Sequence Synthetic peptide
CASSRAGDTEAFF

SEQ ID NO: 261 - protein, 12 aa, Artificial Sequence
  Description of Artificial Sequence Synthetic peptide
CAWGQFNYGYTF

SEQ ID NO: 262 - protein, 12 aa, Artificial Sequence
  Description of Artificial Sequence Synthetic peptide
CASNLGQGGWYF

SEQ ID NO: 263 - protein, 17 aa, Artificial Sequence
  Description of Artificial Sequence Synthetic peptide
CASSWETSGSFPSEQYF

SEQ ID NO: 264 - protein, 14 aa, Artificial Sequence
  Description of Artificial Sequence Synthetic peptide
CSAREWGGGYGYTF

SEQ ID NO: 265 - protein, 18 aa, Artificial Sequence
  Description of Artificial Sequence Synthetic peptide
CAISVGTMGTSGKETQYF

SEQ ID NO: 266 - protein, 14 aa, Artificial Sequence
  Description of Artificial Sequence Synthetic peptide
CASSHPYGSTEAFF

SEQ ID NO: 267 - protein, 17 aa, Artificial Sequence
  Description of Artificial Sequence Synthetic peptide
CASSLTLRTASPYEQYF

SEQ ID NO: 268 - protein, 15 aa, Artificial Sequence
  Description of Artificial Sequence Synthetic peptide
CASSSRTGPRYGYTF

SEQ ID NO: 269 - protein, 14 aa, Artificial Sequence
  Description of Artificial Sequence Synthetic peptide
CASSTDTRGGEQYF

SEQ ID NO: 270 - protein, 15 aa, Artificial Sequence
  Description of Artificial Sequence Synthetic peptide
CASSVALGQSYGYTF

SEQ ID NO: 271 - protein, 14 aa, Artificial Sequence
  Description of Artificial Sequence Synthetic peptide
CSAGPDRGRTGYTF

SEQ ID NO: 272 - protein, 12 aa, Artificial Sequence
  Description of Artificial Sequence Synthetic peptide
CAGRQGGIGNTF

SEQ ID NO: 273 - protein, 17 aa, Artificial Sequence
  Description of Artificial Sequence Synthetic peptide
CARSLRGTGGLNQPQHF

SEQ ID NO: 274 - protein, 14 aa, Artificial Sequence
  Description of Artificial Sequence Synthetic peptide
CASCPEGDTGELFF

SEQ ID NO: 275 - protein, 18 aa, Artificial Sequence
  Description of Artificial Sequence Synthetic peptide
CASKGQGAAIRNNSPLHF

SEQ ID NO: 276 - protein, 14 aa, Artificial Sequence
  Description of Artificial Sequence Synthetic peptide
CASRDTGRLETQYF

SEQ ID NO: 277 - protein, 16 aa, Artificial Sequence
  Description of Artificial Sequence Synthetic peptide
CASRTYPAARYNEQFF

SEQ ID NO: 278 - protein, 16 aa, Artificial Sequence
  Description of Artificial Sequence Synthetic peptide
CASSFSQGTGNSPLHF

SEQ ID NO: 279 - protein, 17 aa, Artificial Sequence
  Description of Artificial Sequence Synthetic peptide
CASSKKRTGPGANVLTF

SEQ ID NO: 280 - protein, 14 aa, Artificial Sequence
  Description of Artificial Sequence Synthetic peptide
CASSLNRKNYGYTF

SEQ ID NO: 281 - protein, 17 aa, Artificial Sequence
  Description of Artificial Sequence Synthetic peptide
CASSRPGTGGAAYGYTF

SEQ ID NO: 282 - protein, 15 aa, Artificial Sequence
  Description of Artificial Sequence Synthetic peptide
CASSSTGGLYNEQFF

SEQ ID NO: 283 - protein, 18 aa, Artificial Sequence
  Description of Artificial Sequence Synthetic peptide
CASSYTRSGVGGKNIQYF

SEQ ID NO: 284 - protein, 15 aa, Artificial Sequence
  Description of Artificial Sequence Synthetic peptide
CATSDLRGSTDTQYF

SEQ ID NO: 285 - protein, 15 aa, Artificial Sequence
  Description of Artificial Sequence Synthetic peptide
CATSDLRTGGDTQYF

SEQ ID NO: 286 - protein, 19 aa, Artificial Sequence
  Description of Artificial Sequence Synthetic peptide
CATSRGRAGVGSGANVLTF

SEQ ID NO: 287 - protein, 16 aa, Artificial Sequence
  Description of Artificial Sequence Synthetic peptide
CATSVVSGVRTGELFF

SEQ ID NO: 288 - protein, 12 aa, Artificial Sequence
  Description of Artificial Sequence Synthetic peptide
CAWSPGSQPQHF

SEQ ID NO: 289 - protein, 13 aa, Artificial Sequence
  Description of Artificial Sequence Synthetic peptide
CAWSVGGNYGYTF

SEQ ID NO: 290 - protein, 13 aa, Artificial Sequence
  Description of Artificial Sequence Synthetic peptide
CAWSVQGNYGYTF

SEQ ID NO: 291 - protein, 13 aa, Artificial Sequence
  Description of Artificial Sequence Synthetic peptide
CAWSVQQNYGYTF

SEQ ID NO: 292 - protein, 16 aa, Artificial Sequence
  Description of Artificial Sequence Synthetic peptide
CSALDRGSLYVYGYTF

SEQ ID NO: 293 - protein, 14 aa, Artificial Sequence
  Description of Artificial Sequence Synthetic peptide
CASSPTGAVQPQHF

SEQ ID NO: 294 - protein, 14 aa, Artificial Sequence
  Description of Artificial Sequence Synthetic peptide
CAWSADRGVDTQYF

SEQ ID NO: 295 - protein, 15 aa, Artificial Sequence
  Description of Artificial Sequence Synthetic peptide
CAWSAGQGARETQYF

SEQ ID NO: 296 - protein, 13 aa, Artificial Sequence
  Description of Artificial Sequence Synthetic peptide
CAWSVQQSYGYTF

SEQ ID NO: 297 - protein, 12 aa, Artificial Sequence
  Description of Artificial Sequence Synthetic peptide
CSARDQGRRQFF

SEQ ID NO: 298 - protein, 16 aa, Artificial Sequence
  Description of Artificial Sequence Synthetic peptide
CSARKLTGTQRYGYTF

SEQ ID NO: 299 - protein, 20 aa, Artificial Sequence
  Description of Artificial Sequence Synthetic peptide
CASSDPKFLGALRGANVLTF

SEQ ID NO: 300 - protein, 15 aa, Artificial Sequence
  Description of Artificial Sequence Synthetic peptide
CASSSTGFSSYEQYF

SEQ ID NO: 301 - protein, 14 aa, Artificial Sequence
  Description of Artificial Sequence Synthetic peptide
CAWSGSGLGYGYTF

SEQ ID NO: 302 - protein, 13 aa, Artificial Sequence
  Description of Artificial Sequence Synthetic peptide
CAWSRDSNTEAFF

SEQ ID NO: 303 - protein, 15 aa, Artificial Sequence
  Description of Artificial Sequence Synthetic peptide
CAWSRDTSAYNEQFF

SEQ ID NO: 304 - protein, 13 aa, Artificial Sequence
  Description of Artificial Sequence Synthetic peptide
CAWSVSQNYGYTF

SEQ ID NO: 305 - protein, 13 aa, Artificial Sequence
  Description of Artificial Sequence Synthetic peptide
CAWSVSSSYGYTF

SEQ ID NO: 306 - protein, 15 aa, Artificial Sequence
  Description of Artificial Sequence Synthetic peptide
CASINAGSSSYEQYF

SEQ ID NO: 307 - protein, 14 aa, Artificial Sequence
  Description of Artificial Sequence Synthetic peptide
CASRDTSSRSEAFF

SEQ ID NO: 308 - protein, 15 aa, Artificial Sequence
  Description of Artificial Sequence Synthetic peptide
CASSAGLSGANVLTF

SEQ ID NO: 309 - protein, 14 aa, Artificial Sequence
  Description of Artificial Sequence Synthetic peptide
CASSLLTGLYGYTF

SEQ ID NO: 310 - protein, 12 aa, Artificial Sequence
  Description of Artificial Sequence Synthetic peptide
CASSQDRYEQYF

SEQ ID NO: 311 - protein, 15 aa, Artificial Sequence
  Description of Artificial Sequence Synthetic peptide
CSARGPGSRINEQYF

SEQ ID NO: 312 - protein, 15 aa, Artificial Sequence
  Description of Artificial Sequence Synthetic peptide
CASSIGTESYNEQFF

SEQ ID NO: 313 - protein, 16 aa, Artificial Sequence
  Description of Artificial Sequence Synthetic peptide
CASSPGSEVAKNIQYF

SEQ ID NO: 314 - protein, 14 aa, Artificial Sequence
  Description of Artificial Sequence Synthetic peptide
CAWIRDGQGAQAFF

SEQ ID NO: 315 - protein, 14 aa, Artificial Sequence
  Description of Artificial Sequence Synthetic peptide
CAWSGGGRRDTQYF

SEQ ID NO: 316 - protein, 14 aa, Artificial Sequence
  Description of Artificial Sequence Synthetic peptide
CAWSLGTARDTQYF

SEQ ID NO: 317 - protein, 12 aa, Artificial Sequence
  Description of Artificial Sequence Synthetic peptide
CASQGGPTEAFF

SEQ ID NO: 318 - protein, 14 aa, Artificial Sequence
  Description of Artificial Sequence Synthetic peptide
CASSEARGAGTQYF

SEQ ID NO: 319 - protein, 15 aa, Artificial Sequence
  Description of Artificial Sequence Synthetic peptide
CASSETRGEQETQYF

SEQ ID NO: 320 - protein, 16 aa, Artificial Sequence
  Description of Artificial Sequence Synthetic peptide
CASSSLLAGVSYEQYF

SEQ ID NO: 321 - protein, 14 aa, Artificial Sequence
  Description of Artificial Sequence Synthetic peptide
CASSVATGPYGYTF

SEQ ID NO: 322 - protein, 13 aa, Artificial Sequence
  Description of Artificial Sequence Synthetic peptide
CAGFTGGMAGYTF

SEQ ID NO: 323 - protein, 14 aa, Artificial Sequence
  Description of Artificial Sequence Synthetic peptide
CASANGQAPYGYTF

SEQ ID NO: 324 - protein, 15 aa, Artificial Sequence
  Description of Artificial Sequence Synthetic peptide
CASGAGGTSSYEQYF

SEQ ID NO: 325 - protein, 14 aa, Artificial Sequence
  Description of Artificial Sequence Synthetic peptide
CASSYDRAPYGYTF

SEQ ID NO: 326 - protein, 15 aa, Artificial Sequence
  Description of Artificial Sequence Synthetic peptide
CAWSSTGGRRDTQYF

SEQ ID NO: 327 - protein, 13 aa, Artificial Sequence
  Description of Artificial Sequence Synthetic peptide
CAWSVQANYGYTF

SEQ ID NO: 328 - protein, 12 aa, Artificial Sequence
  Description of Artificial Sequence Synthetic peptide
CAWSVQPRGYTF

SEQ ID NO: 329 - protein, 10 aa, Artificial Sequence
  Description of Artificial Sequence Synthetic peptide
CSALDRKQYF

SEQ ID NO: 330 - protein, 13 aa, Artificial Sequence
  Description of Artificial Sequence Synthetic peptide
CASRGQVQETQYF

SEQ ID NO: 331 - protein, 14 aa, Artificial Sequence
  Description of Artificial Sequence Synthetic peptide
CASSLEFQAGELFF

SEQ ID NO: 332 - protein, 15 aa, Artificial Sequence
  Description of Artificial Sequence Synthetic peptide
CASSLRQDSTSPLHF

SEQ ID NO: 333 - protein, 16 aa, Artificial Sequence
  Description of Artificial Sequence Synthetic peptide
CASSLVSGVGFYGYTF

SEQ ID NO: 334 - protein, 15 aa, Artificial Sequence
  Description of Artificial Sequence Synthetic peptide
CASSPTGTRTDTQYF

SEQ ID NO: 335 - protein, 15 aa, Artificial Sequence
  Description of Artificial Sequence Synthetic peptide
CATSTGTGRVDTQYF

SEQ ID NO: 336 - protein, 13 aa, Artificial Sequence
  Description of Artificial Sequence Synthetic peptide
CAIRDKSTDTQYF

SEQ ID NO: 337 - protein, 12 aa, Artificial Sequence
  Description of Artificial Sequence Synthetic peptide
CAISDISDEQYF

SEQ ID NO: 338 - protein, 15 aa, Artificial Sequence
  Description of Artificial Sequence Synthetic peptide
CAISEQALSNQPQHF

SEQ ID NO: 339 - protein, 14 aa, Artificial Sequence
  Description of Artificial Sequence Synthetic peptide
CAISGTSGGDTQYF

SEQ ID NO: 340 - protein, 12 aa, Artificial Sequence
  Description of Artificial Sequence Synthetic peptide
CAISPANYGYTF

SEQ ID NO: 341 - protein, 15 aa, Artificial Sequence
  Description of Artificial Sequence Synthetic peptide
CASRLKPRSTGELFF

SEQ ID NO: 342 - protein, 15 aa, Artificial Sequence
  Description of Artificial Sequence Synthetic peptide
CASSESLGGTDTQYF

SEQ ID NO: 343 - protein, 14 aa, Artificial Sequence
  Description of Artificial Sequence Synthetic peptide
CASSGQAWSTEAFF

SEQ ID NO: 344 - protein, 14 aa, Artificial Sequence
  Description of Artificial Sequence Synthetic peptide
CASSLALMNTEAFF

SEQ ID NO: 345 - protein, 18 aa, Artificial Sequence
  Description of Artificial Sequence Synthetic peptide
CASSLPMEPLEGDTEAFF

SEQ ID NO: 346 - protein, 16 aa, Artificial Sequence
  Description of Artificial Sequence Synthetic peptide
CASSLRGAVRTGELFF

SEQ ID NO: 347 - protein, 12 aa, Artificial Sequence
  Description of Artificial Sequence Synthetic peptide
CASSPFTYEQYF

SEQ ID NO: 348 - protein, 16 aa, Artificial Sequence
  Description of Artificial Sequence Synthetic peptide
CASSPQGGSLAYEQYF

SEQ ID NO: 349 - protein, 14 aa, Artificial Sequence
  Description of Artificial Sequence Synthetic peptide
CASSPTTVKYGYTF

SEQ ID NO: 350 - protein, 16 aa, Artificial Sequence
  Description of Artificial Sequence Synthetic peptide
CASSQDETGDQETQYF

SEQ ID NO: 351 - protein, 14 aa, Artificial Sequence
  Description of Artificial Sequence Synthetic peptide
CASSVDGSNQPQHF

SEQ ID NO: 352 - protein, 14 aa, Artificial Sequence
  Description of Artificial Sequence Synthetic peptide
CASSYPEGQSLAFF

SEQ ID NO: 353 - protein, 15 aa, Artificial Sequence
  Description of Artificial Sequence Synthetic peptide
CASTLSGYNTGELFF

SEQ ID NO: 354 - protein, 15 aa, Artificial Sequence
  Description of Artificial Sequence Synthetic peptide
CASTMGQGNTDTQYF

SEQ ID NO: 355 - protein, 16 aa, Artificial Sequence
  Description of Artificial Sequence Synthetic peptide
CASTTGLAGGQETQYF

SEQ ID NO: 356 - protein, 17 aa, Artificial Sequence
  Description of Artificial Sequence Synthetic peptide
CAWSVRSSGALNNEQFF

SEQ ID NO: 357 - protein, 12 aa, Artificial Sequence
  Description of Artificial Sequence Synthetic peptide
CAWWDRGHGYTF

SEQ ID NO: 358 - protein, 14 aa, Artificial Sequence
  Description of Artificial Sequence Synthetic peptide
CSARKGQGNYGYTF

SEQ ID NO: 359 - protein, 12 aa, Artificial Sequence
  Description of Artificial Sequence Synthetic peptide
CSASGLAHSQYF

SEQ ID NO: 360 - protein, 14 aa, Artificial Sequence
  Description of Artificial Sequence Synthetic peptide
CSVDYGGGTDTQYF

SEQ ID NO: 361 - protein, 15 aa, Artificial Sequence
  Description of Artificial Sequence Synthetic peptide
CAISESVGNYNEQFF

SEQ ID NO: 362 - protein, 12 aa, Artificial Sequence
  Description of Artificial Sequence Synthetic peptide
CAISPDMDGYTF

SEQ ID NO: 363 - protein, 13 aa, Artificial Sequence
  Description of Artificial Sequence Synthetic peptide
CASAWRTSGELFF

SEQ ID NO: 364 - protein, 11 aa, Artificial Sequence
  Description of Artificial Sequence Synthetic peptide
CASGTRHEQYF

SEQ ID NO: 365 - protein, 12 aa, Artificial Sequence
  Description of Artificial Sequence Synthetic peptide
CASKGPNTEAFF

SEQ ID NO: 366 - protein, 17 aa, Artificial Sequence
  Description of Artificial Sequence Synthetic peptide
CASLPGLGFGLQETQYF

SEQ ID NO: 367 - protein, 17 aa, Artificial Sequence
  Description of Artificial Sequence Synthetic peptide
CASSDYGAGTGLKGYTF

SEQ ID NO: 368 - protein, 14 aa, Artificial Sequence
  Description of Artificial Sequence Synthetic peptide
CASSEVRSNQPQHF

SEQ ID NO: 369 - protein, 13 aa, Artificial Sequence
  Description of Artificial Sequence Synthetic peptide
CASSFRRENTIYF

SEQ ID NO: 370 - protein, 15 aa, Artificial Sequence
  Description of Artificial Sequence Synthetic peptide
CASSIDLRQGATQYF

SEQ ID NO: 371 - protein, 11 aa, Artificial Sequence
  Description of Artificial Sequence Synthetic peptide
CASSLTGEQYF

SEQ ID NO: 372 - protein, 12 aa, Artificial Sequence
  Description of Artificial Sequence Synthetic peptide
CASSPRGNEQFF

SEQ ID NO: 373 - protein, 12 aa, Artificial Sequence
  Description of Artificial Sequence Synthetic peptide
CASSRDTDGYTF

SEQ ID NO: 374 - protein, 15 aa, Artificial Sequence
  Description of Artificial Sequence Synthetic peptide
CASSSAEGGNQPQHF

SEQ ID NO: 375 - protein, 13 aa, Artificial Sequence
  Description of Artificial Sequence Synthetic peptide
CASSSNGPSGYTF

SEQ ID NO: 376 - protein, 14 aa, Artificial Sequence
  Description of Artificial Sequence Synthetic peptide
CASKMDSRNQPQHF

SEQ ID NO: 377 - protein, 14 aa, Artificial Sequence
  Description of Artificial Sequence Synthetic peptide
CASKPTGAAGAQHF

SEQ ID NO: 378 - protein, 15 aa, Artificial Sequence
  Description of Artificial Sequence Synthetic peptide
CASSAGGAGANVLTF

SEQ ID NO: 379 - protein, 18 aa, Artificial Sequence
  Description of Artificial Sequence Synthetic peptide
CASSEATFGTSGTDTQYF

SEQ ID NO: 380 - protein, 15 aa, Artificial Sequence
  Description of Artificial Sequence Synthetic peptide
CASSEWSGAQETQYF

SEQ ID NO: 381 - protein, 18 aa, Artificial Sequence
  Description of Artificial Sequence Synthetic peptide
CASSFGVPGQVTGQPQHF

SEQ ID NO: 382 - protein, 12 aa, Artificial Sequence
  Description of Artificial Sequence Synthetic peptide
CASSSSNQPQHF

SEQ ID NO: 383 - protein, 18 aa, Artificial Sequence
  Description of Artificial Sequence Synthetic peptide
CASSTDRLGTYSNQPQHF

SEQ ID NO: 384 - protein, 12 aa, Artificial Sequence
  Description of Artificial Sequence Synthetic peptide
CASSWTVPPLHF

SEQ ID NO: 385 - protein, 13 aa, Artificial Sequence
  Description of Artificial Sequence Synthetic peptide
CATARAANTEAFF

SEQ ID NO: 386 - protein, 17 aa, Artificial Sequence
  Description of Artificial Sequence Synthetic peptide
CATSDPRQGASNQPQHF

SEQ ID NO: 387 - protein, 15 aa, Artificial Sequence
  Description of Artificial Sequence Synthetic peptide
CATSEQGAGNQPQHF

SEQ ID NO: 388 - protein, 15 aa, Artificial Sequence
  Description of Artificial Sequence Synthetic peptide
CATSRGERGGTEAFF

SEQ ID NO: 389 - protein, 13 aa, Artificial Sequence
  Description of Artificial Sequence Synthetic peptide
CRFSALGNTEAFF

SEQ ID NO: 390 - protein, 12 aa, Artificial Sequence
  Description of Artificial Sequence Synthetic peptide
CSAGRGDTEAFF

SEQ ID NO: 391 - protein, 15 aa, Artificial Sequence
  Description of Artificial Sequence Synthetic peptide
CSAGSRGGANTEAFF

SEQ ID NO: 392 - protein, 13 aa, Artificial Sequence
  Description of Artificial Sequence Synthetic peptide
CSAKGGVNTEAFF

SEQ ID NO: 393 - protein, 12 aa, Artificial Sequence
  Description of Artificial Sequence Synthetic peptide
CSALRGGNTIYF

SEQ ID NO: 394 - protein, 14 aa, Artificial Sequence
  Description of Artificial Sequence Synthetic peptide
CSAPPRGAPGTQYF

SEQ ID NO: 395 - protein, 15 aa, Artificial Sequence
  Description of Artificial Sequence Synthetic peptide
CSAPQNGGGRETQYF

SEQ ID NO: 396 - protein, 13 aa, Artificial Sequence
  Description of Artificial Sequence Synthetic peptide
CSARNRLDGSYTF

SEQ ID NO: 397 - protein, 19 aa, Artificial Sequence
  Description of Artificial Sequence Synthetic peptide
CSARVYRAGASSGANVLTF

SEQ ID NO: 398 - protein, 19 aa, Artificial Sequence
  Description of Artificial Sequence Synthetic peptide
CSASNFRTSVSSGANVLTF

SEQ ID NO: 399 - protein, 13 aa, Artificial Sequence
  Description of Artificial Sequence Synthetic peptide
CSASSRHTGELFF

SEQ ID NO: 400 - protein, 14 aa, Artificial Sequence
  Description of Artificial Sequence Synthetic peptide
CSATWAFSNQPQHF

SEQ ID NO: 401 - protein, 13 aa, Artificial Sequence
  Description of Artificial Sequence Synthetic peptide
CASSELQGAGYTF

SEQ ID NO: 402 - protein, 14 aa, Artificial Sequence
  Description of Artificial Sequence Synthetic peptide
CASSETGDRSPLHF

SEQ ID NO: 403 - protein, 14 aa, Artificial Sequence
  Description of Artificial Sequence Synthetic peptide
CASSIVSGDYGYTF

SEQ ID NO: 404 - protein, 15 aa, Artificial Sequence
  Description of Artificial Sequence Synthetic peptide
CASSPSPGESYEQYF

SEQ ID NO: 405 - protein, 13 aa, Artificial Sequence
  Description of Artificial Sequence Synthetic peptide
CASSQIQGAGYTF

SEQ ID NO: 406 - protein, 15 aa, Artificial Sequence
  Description of Artificial Sequence Synthetic peptide
CASSRSGTGSDTQYF

SEQ ID NO: 407 - protein, 16 aa, Artificial Sequence
  Description of Artificial Sequence Synthetic peptide
CASSTQPGLAGETQYF

SEQ ID NO: 408 - protein, 15 aa, Artificial Sequence
  Description of Artificial Sequence Synthetic peptide
CASSYRGMSTDTQYF

SEQ ID NO: 409 - protein, 13 aa, Artificial Sequence
  Description of Artificial Sequence Synthetic peptide
CATMRGGSAEAFF

SEQ ID NO: 410 - protein, 13 aa, Artificial Sequence
  Description of Artificial Sequence Synthetic peptide
CAIRDYSADTQYF

SEQ ID NO: 411 - protein, 13 aa, Artificial Sequence
  Description of Artificial Sequence Synthetic peptide
CAISDRAGYGYTF

SEQ ID NO: 412 - protein, 16 aa, Artificial Sequence
  Description of Artificial Sequence Synthetic peptide
CAISEAGGFMNTEAFF

SEQ ID NO: 413 - protein, 14 aa, Artificial Sequence
  Description of Artificial Sequence Synthetic peptide
CAISGWDGHYGYTF

SEQ ID NO: 414 - protein, 13 aa, Artificial Sequence
  Description of Artificial Sequence Synthetic peptide
CASRAGQRYGYTF

SEQ ID NO: 415 - protein, 14 aa, Artificial Sequence
  Description of Artificial Sequence Synthetic peptide
CASSDRGRFYGYTF

SEQ ID NO: 416 - protein, 13 aa, Artificial Sequence
  Description of Artificial Sequence Synthetic peptide
CASSEGSHSTQHF

SEQ ID NO: 417 - protein, 15 aa, Artificial Sequence
  Description of Artificial Sequence Synthetic peptide
CASSLAGRGRSPLHF

SEQ ID NO: 418 - protein, 13 aa, Artificial Sequence
  Description of Artificial Sequence Synthetic peptide
CASSLDYSQIQHF

SEQ ID NO: 419 - protein, 16 aa, Artificial Sequence
  Description of Artificial Sequence Synthetic peptide
CASSLKGAGWTGELFF

SEQ ID NO: 420 - protein, 13 aa, Artificial Sequence
  Description of Artificial Sequence Synthetic peptide
CASSMTDSYEQYF

SEQ ID NO: 421 - protein, 13 aa, Artificial Sequence
  Description of Artificial Sequence Synthetic peptide
CASSPVSNYGYTF

SEQ ID NO: 422 - protein, 14 aa, Artificial Sequence
  Description of Artificial Sequence Synthetic peptide
CASSTDRRKGGYTF

SEQ ID NO: 423 - protein, 16 aa, Artificial Sequence
  Description of Artificial Sequence Synthetic peptide
CATHIGQGSSNQPQHF

SEQ ID NO: 424 - protein, 12 aa, Artificial Sequence
  Description of Artificial Sequence Synthetic peptide
CSAEQGAGELFF

SEQ ID NO: 425 - protein, 11 aa, Artificial Sequence
  Description of Artificial Sequence Synthetic peptide
CSAQRTYGYTF

SEQ ID NO: 426 - protein, 15 aa, Artificial Sequence
  Description of Artificial Sequence Synthetic peptide
CSARNRGLNNQPQHF

SEQ ID NO: 427 - protein, 15 aa, Artificial Sequence
  Description of Artificial Sequence Synthetic peptide
CASKRPGRMNTEAFF

SEQ ID NO: 428 - protein, 19 aa, Artificial Sequence
  Description of Artificial Sequence Synthetic peptide
CASRHREKTGGEQNQPQHF

SEQ ID NO: 429 - protein, 16 aa, Artificial Sequence
  Description of Artificial Sequence Synthetic peptide
CASRQREGATIYGYTF

SEQ ID NO: 430 - protein, 10 aa, Artificial Sequence
  Description of Artificial Sequence Synthetic peptide
CASSFGELFF

SEQ ID NO: 431 - protein, 18 aa, Artificial Sequence
  Description of Artificial Sequence Synthetic peptide
CASSFRIKFAGSNNEQFF

SEQ ID NO: 432 - protein, 19 aa, Artificial Sequence
  Description of Artificial Sequence Synthetic peptide
CASSFRIKLQGGPAYEQYF

SEQ ID NO: 433 - protein, 14 aa, Artificial Sequence
  Description of Artificial Sequence Synthetic peptide
CASSFRQGNSPLHF

SEQ ID NO: 434 - protein, 13 aa, Artificial Sequence
  Description of Artificial Sequence Synthetic peptide
CASSLKLDTEAFF

SEQ ID NO: 435 - protein, 13 aa, Artificial Sequence
  Description of Artificial Sequence Synthetic peptide
CASSLNGNQPQHF

SEQ ID NO: 436 - protein, 15 aa, Artificial Sequence
  Description of Artificial Sequence Synthetic peptide
CASSLPSGDSNEQFF

SEQ ID NO: 437 - protein, 16 aa, Artificial Sequence
  Description of Artificial Sequence Synthetic peptide
CASSLRAGGPTDTQYF

SEQ ID NO: 438 - protein, 14 aa, Artificial Sequence
  Description of Artificial Sequence Synthetic peptide
CASSSGQGTGEQYF

SEQ ID NO: 439 - protein, 14 aa, Artificial Sequence
  Description of Artificial Sequence Synthetic peptide
CASSYPGIYNEQFF

SEQ ID NO: 440 - protein, 18 aa, Artificial Sequence
  Description of Artificial Sequence Synthetic peptide
CASSYRVKYQGGNQPQHF

SEQ ID NO: 441 - protein, 13 aa, Artificial Sequence
  Description of Artificial Sequence Synthetic peptide
CSARAYQNYGYTF

SEQ ID NO: 442 - protein, 13 aa, Artificial Sequence
  Description of Artificial Sequence Synthetic peptide
CAEGGLAGREQYF

SEQ ID NO: 443 - protein, 14 aa, Artificial Sequence
  Description of Artificial Sequence Synthetic peptide
CASELAGVVGELFF

SEQ ID NO: 444 - protein, 14 aa, Artificial Sequence
  Description of Artificial Sequence Synthetic peptide
CASSELGLNYGYTF

SEQ ID NO: 445 - protein, 11 aa, Artificial Sequence
  Description of Artificial Sequence Synthetic peptide
CASSERTEAFF

SEQ ID NO: 446 - protein, 15 aa, Artificial Sequence
  Description of Artificial Sequence Synthetic peptide
CASSESGNGADTQYF

SEQ ID NO: 447 - protein, 15 aa, Artificial Sequence
  Description of Artificial Sequence Synthetic peptide
CASSFWGNSNQPQHF

SEQ ID NO: 448 - protein, 12 aa, Artificial Sequence
  Description of Artificial Sequence Synthetic peptide
CASSGGTVGQYF

SEQ ID NO: 449 - protein, 14 aa, Artificial Sequence
  Description of Artificial Sequence Synthetic peptide
CASSIDGGSGEAFF

SEQ ID NO: 450 - protein, 14 aa, Artificial Sequence
  Description of Artificial Sequence Synthetic peptide
CASSKGPSLETQYF

SEQ ID NO: 451 - protein, 15 aa, Artificial Sequence
  Description of Artificial Sequence Synthetic peptide
CASSLAGGGGYEQYF

SEQ ID NO: 452 - protein, 14 aa, Artificial Sequence
  Description of Artificial Sequence Synthetic peptide
CASSLARGYDTQYF

SEQ ID NO: 453 - protein, 15 aa, Artificial Sequence
  Description of Artificial Sequence Synthetic peptide
CASSLDPGGGYGYTF

SEQ ID NO: 454 - protein, 14 aa, Artificial Sequence
  Description of Artificial Sequence Synthetic peptide
CASSLDRGAGGYTF

SEQ ID NO: 455 - protein, 16 aa, Artificial Sequence
  Description of Artificial Sequence Synthetic peptide
CASSLGVQGENTEAFF

SEQ ID NO: 456 - protein, 14 aa, Artificial Sequence
  Description of Artificial Sequence Synthetic peptide
CASSLNREYDEQYF

SEQ ID NO: 457 - protein, 14 aa, Artificial Sequence
  Description of Artificial Sequence Synthetic peptide
CASSLNRGQDEQYF

SEQ ID NO: 458 - protein, 14 aa, Artificial Sequence
  Description of Artificial Sequence Synthetic peptide
CASSLNRVRDTQYF

SEQ ID NO: 459 - protein, 15 aa, Artificial Sequence
  Description of Artificial Sequence Synthetic peptide
CASSQDWGKVQPQHF

SEQ ID NO: 460 - protein, 14 aa, Artificial Sequence
  Description of Artificial Sequence Synthetic peptide
CASSSWGRGEKLFF

SEQ ID NO: 461 - protein, 19 aa, Artificial Sequence
  Description of Artificial Sequence Synthetic peptide
CASSVTSGGATSSTDTQYF

SEQ ID NO: 462 - protein, 12 aa, Artificial Sequence
  Description of Artificial Sequence Synthetic peptide
CASSWGTDTQYF

SEQ ID NO: 463 - protein, 13 aa, Artificial Sequence
  Description of Artificial Sequence Synthetic peptide
CSAAPGQGLGYTF

SEQ ID NO: 464 - protein, 16 aa, Artificial Sequence
  Description of Artificial Sequence Synthetic peptide
CSARASTGTGGGKLFF

SEQ ID NO: 465 - protein, 15 aa, Artificial Sequence
  Description of Artificial Sequence Synthetic peptide
CSASHTGGVSDTQYF

SEQ ID NO: 466 - protein, 13 aa, Artificial Sequence
  Description of Artificial Sequence Synthetic peptide
CSNRASGFSPLHF

SEQ ID NO: 467 - protein, 13 aa, Artificial Sequence
  Description of Artificial Sequence Synthetic peptide
CASNRIDPSEQFF

SEQ ID NO: 468 - protein, 15 aa, Artificial Sequence
  Description of Artificial Sequence Synthetic peptide
CASRANRDLNYGYTF

SEQ ID NO: 469 - protein, 14 aa, Artificial Sequence
  Description of Artificial Sequence Synthetic peptide
CASSAAREWETQYF

SEQ ID NO: 470 - protein, 14 aa, Artificial Sequence
  Description of Artificial Sequence Synthetic peptide
CASSAYTANYGYTF

SEQ ID NO: 471 - protein, 14 aa, Artificial Sequence
  Description of Artificial Sequence Synthetic peptide
CASSDRLATGELFF

SEQ ID NO: 472 - protein, 14 aa, Artificial Sequence
  Description of Artificial Sequence Synthetic peptide
CASSENRDGHEQFF

SEQ ID NO: 473 - protein, 13 aa, Artificial Sequence
  Description of Artificial Sequence Synthetic peptide
CASSENTEYGYTF

SEQ ID NO: 474 - protein, 15 aa, Artificial Sequence
  Description of Artificial Sequence Synthetic peptide
CASSEVASGERAQYF

SEQ ID NO: 475 - protein, 12 aa, Artificial Sequence
  Description of Artificial Sequence Synthetic peptide
CASSFGGTGAFF

SEQ ID NO: 476 - protein, 16 aa, Artificial Sequence
  Description of Artificial Sequence Synthetic peptide
CASSFLAGGNTGELFF

SEQ ID NO: 477 - protein, 14 aa, Artificial Sequence
  Description of Artificial Sequence Synthetic peptide
CASSFVAYLNEQFF

SEQ ID NO: 478 - protein, 16 aa, Artificial Sequence
  Description of Artificial Sequence Synthetic peptide
CASSLRAARGAYGYTF

SEQ ID NO: 479 - protein, 15 aa, Artificial Sequence
  Description of Artificial Sequence Synthetic peptide
CASSPVGDRAYEQYF

SEQ ID NO: 480 - protein, 15 aa, Artificial Sequence
  Description of Artificial Sequence Synthetic peptide
CASSRDGSSYNEQFF

SEQ ID NO: 481 - protein, 18 aa, Artificial Sequence
  Description of Artificial Sequence Synthetic peptide
CSARGIWTSGGDSYEQYF

SEQ ID NO: 482 - protein, 13 aa, Artificial Sequence
  Description of Artificial Sequence Synthetic peptide
CASRGDGRSPLHF

SEQ ID NO: 483 - protein, 14 aa, Artificial Sequence
  Description of Artificial Sequence Synthetic peptide
CASSAEKGRSGYTF

SEQ ID NO: 484 - protein, 13 aa, Artificial Sequence
  Description of Artificial Sequence Synthetic peptide
CASSAGTENIQYF

SEQ ID NO: 485 - protein, 15 aa, Artificial Sequence
  Description of Artificial Sequence Synthetic peptide
CASSESGRAMETQYF

SEQ ID NO: 486 - protein, 16 aa, Artificial Sequence
  Description of Artificial Sequence Synthetic peptide
CASSKVVITGPYGYTF

SEQ ID NO: 487 - protein, 14 aa, Artificial Sequence
  Description of Artificial Sequence Synthetic peptide
CASSLAGGLDGYTF

SEQ ID NO: 488 - protein, 18 aa, Artificial Sequence
  Description of Artificial Sequence Synthetic peptide
CASSLETGTGGDNQPQHF

SEQ ID NO: 489 - protein, 16 aa, Artificial Sequence
  Description of Artificial Sequence Synthetic peptide
CASSLGSSGEKDTQYF

SEQ ID NO: 490 - protein, 15 aa, Artificial Sequence
  Description of Artificial Sequence Synthetic peptide
CASSLIAGGHYEQYF

SEQ ID NO: 491 - protein, 16 aa, Artificial Sequence
  Description of Artificial Sequence Synthetic peptide
CASSLNPAGSTDTQYF

SEQ ID NO: 492 - protein, 16 aa, Artificial Sequence
  Description of Artificial Sequence Synthetic peptide
CASSLVPGQGSYEQYF

SEQ ID NO: 493 - protein, 12 aa, Artificial Sequence
  Description of Artificial Sequence Synthetic peptide
CASSLVPYEQYF

SEQ ID NO: 494 - protein, 12 aa, Artificial Sequence
  Description of Artificial Sequence Synthetic peptide
CASSPRVYEQFF

SEQ ID NO: 495 - protein, 15 aa, Artificial Sequence
  Description of Artificial Sequence Synthetic peptide
CASSQGDIGNQPQHF

SEQ ID NO: 496 - protein, 14 aa, Artificial Sequence
  Description of Artificial Sequence Synthetic peptide
CASSQGQGDSPLHF

SEQ ID NO: 497 - protein, 16 aa, Artificial Sequence
  Description of Artificial Sequence Synthetic peptide
CASSRGLAGGTDTQYF

SEQ ID NO: 498 - protein, 14 aa, Artificial Sequence
  Description of Artificial Sequence Synthetic peptide
CASSRRGGSSPLHF

SEQ ID NO: 499 - protein, 14 aa, Artificial Sequence
  Description of Artificial Sequence Synthetic peptide
CASSRWQGRTEAFF

SEQ ID NO: 500 - protein, 14 aa, Artificial Sequence
  Description of Artificial Sequence Synthetic peptide
CASSSRGVGTEAFF

SEQ ID NO: 501 - protein, 15 aa, Artificial Sequence
  Description of Artificial Sequence Synthetic peptide
CASSTTGQGSDEQFF

SEQ ID NO: 502 - protein, 11 aa, Artificial Sequence
  Description of Artificial Sequence Synthetic peptide
CASVSNQPQHF

SEQ ID NO: 503 - protein, 14 aa, Artificial Sequence
  Description of Artificial Sequence Synthetic peptide
CATSDLGEGETQYF

SEQ ID NO: 504 - protein, 13 aa, Artificial Sequence
  Description of Artificial Sequence Synthetic peptide
CSARDLTGDGYTF

SEQ ID NO: 505 - protein, 14 aa, Artificial Sequence
  Description of Artificial Sequence Synthetic peptide
CSASDNRRNYGYTF

SEQ ID NO: 506 - protein, 13 aa, Artificial Sequence
  Description of Artificial Sequence Synthetic peptide
CAISDRGASEAFF

SEQ ID NO: 507 - protein, 14 aa, Artificial Sequence
  Description of Artificial Sequence Synthetic peptide
CAISERSGDYGYTF

SEQ ID NO: 508 - protein, 13 aa, Artificial Sequence
  Description of Artificial Sequence Synthetic peptide
CASSFESTDTQYF

SEQ ID NO: 509 - protein, 15 aa, Artificial Sequence
  Description of Artificial Sequence Synthetic peptide
CASSFKGQGFSPLHF

SEQ ID NO: 510 - protein, 14 aa, Artificial Sequence
  Description of Artificial Sequence Synthetic peptide
CASSGQGSTDTQYF

SEQ ID NO: 511 - protein, 14 aa, Artificial Sequence
  Description of Artificial Sequence Synthetic peptide
CASSLEGSTDTQYF

SEQ ID NO: 512 - protein, 13 aa, Artificial Sequence
  Description of Artificial Sequence Synthetic peptide
CASSTSNEYGYTF

SEQ ID NO: 513 - protein, 18 aa, Artificial Sequence
  Description of Artificial Sequence Synthetic peptide
CASSVTGTGGLGNSPLHF

SEQ ID NO: 514 - protein, 14 aa, Artificial Sequence
  Description of Artificial Sequence Synthetic peptide
CASSYPESNTEAFF

SEQ ID NO: 515 - protein, 14 aa, Artificial Sequence
  Description of Artificial Sequence Synthetic peptide
CASSYTGQGAGYTF

SEQ ID NO: 516 - protein, 14 aa, Artificial Sequence
  Description of Artificial Sequence Synthetic peptide
CASTLEYSTDTQYF

SEQ ID NO: 517 - protein, 14 aa, Artificial Sequence
  Description of Artificial Sequence Synthetic peptide
CASTQTSGSDEQYF

SEQ ID NO: 518 - protein, 13 aa, Artificial Sequence
  Description of Artificial Sequence Synthetic peptide
CATAAGGSYGYTF

SEQ ID NO: 519 - protein, 15 aa, Artificial Sequence
  Description of Artificial Sequence Synthetic peptide
CATSDGQGFADTQYF

SEQ ID NO: 520 - protein, 13 aa, Artificial Sequence
  Description of Artificial Sequence Synthetic peptide
CSARDISAYEQYF

SEQ ID NO: 521 - protein, 15 aa, Artificial Sequence
  Description of Artificial Sequence Synthetic peptide
CSARRQGALNQPQHF

SEQ ID NO: 522 - protein, 13 aa, Artificial Sequence
  Description of Artificial Sequence Synthetic peptide
CSARVNAGGGYTF

SEQ ID NO: 523 - protein, 14 aa, Artificial Sequence
  Description of Artificial Sequence Synthetic peptide
CSASIQGADYGYTF

SEQ ID NO: 524 - protein, 14 aa, Artificial Sequence
  Description of Artificial Sequence Synthetic peptide
CSASLARANTEAFF

SEQ ID NO: 525 - protein, 14 aa, Artificial Sequence
  Description of Artificial Sequence Synthetic peptide
CAIKTGSTNYGYTF

SEQ ID NO: 526 - protein, 14 aa, Artificial Sequence
  Description of Artificial Sequence Synthetic peptide
CAISERGLGYGYTF

SEQ ID NO: 527 - protein, 13 aa, Artificial Sequence
  Description of Artificial Sequence Synthetic peptide
CASSAGRNQPQHF

SEQ ID NO: 528 - protein, 14 aa, Artificial Sequence
  Description of Artificial Sequence Synthetic peptide
CASSFRREPYEQYF

SEQ ID NO: 529 - protein, 13 aa, Artificial Sequence
  Description of Artificial Sequence Synthetic peptide
CASSIEVGETQYF

SEQ ID NO: 530 - protein, 16 aa, Artificial Sequence
  Description of Artificial Sequence Synthetic peptide
CASSLTGAVFGTEAFF

SEQ ID NO: 531 - protein, 15 aa, Artificial Sequence
  Description of Artificial Sequence Synthetic peptide
CASSLTGGRLGELFF

SEQ ID NO: 532 - protein, 14 aa, Artificial Sequence
  Description of Artificial Sequence Synthetic peptide
CASSPGRITDTQYF

SEQ ID NO: 533 - protein, 16 aa, Artificial Sequence
  Description of Artificial Sequence Synthetic peptide
CASSQEPGIMNTEAFF

SEQ ID NO: 534 - protein, 14 aa, Artificial Sequence
  Description of Artificial Sequence Synthetic peptide
CASSQWGINQPQHF

SEQ ID NO: 535 - protein, 16 aa, Artificial Sequence
  Description of Artificial Sequence Synthetic peptide
CASSRRGGGASYGYTF

SEQ ID NO: 536 - protein, 14 aa, Artificial Sequence
  Description of Artificial Sequence Synthetic peptide
CSAGWGGGNAGGTF

SEQ ID NO: 537 - protein, 15 aa, Artificial Sequence
  Description of Artificial Sequence Synthetic peptide
CSAPKGQLLQETQYF

SEQ ID NO: 538 - protein, 14 aa, Artificial Sequence
  Description of Artificial Sequence Synthetic peptide
CSARDFRGTYGYTF

SEQ ID NO: 539 - protein, 14 aa, Artificial Sequence
  Description of Artificial Sequence Synthetic peptide
CSARDLHSNQPQHF

SEQ ID NO: 540 - protein, 13 aa, Artificial Sequence
  Description of Artificial Sequence Synthetic peptide
CASSFSGANVLTF

SEQ ID NO: 541 - protein, 12 aa, Artificial Sequence
  Description of Artificial Sequence Synthetic peptide
CASSLGQNTIYF

SEQ ID NO: 542 - protein, 13 aa, Artificial Sequence
  Description of Artificial Sequence Synthetic peptide
CASSQIVTGGAFF

SEQ ID NO: 543 - protein, 13 aa, Artificial Sequence
  Description of Artificial Sequence Synthetic peptide
CASSQMGNTEAFF

SEQ ID NO: 544 - protein, 13 aa, Artificial Sequence
  Description of Artificial Sequence Synthetic peptide
CASSQVQSSGYTF

SEQ ID NO: 545 - protein, 13 aa, Artificial Sequence
  Description of Artificial Sequence Synthetic peptide
CSARGWGNYGYTF

SEQ ID NO: 546 - protein, 12 aa, Artificial Sequence
  Description of Artificial Sequence Synthetic peptide
CAWGWFNYGYTF

SEQ ID NO: 547 - protein, 14 aa, Artificial Sequence
  Description of Artificial Sequence Synthetic peptide
CASSLGWQLYGYTF

SEQ ID NO: 548 - protein, 15 aa, Artificial Sequence
  Description of Artificial Sequence Synthetic peptide
CAVRGSGGSNYKLTF

SEQ ID NO: 549 - protein, 15 aa, Artificial Sequence
  Description of Artificial Sequence Synthetic peptide
CAFMKPRGSQGNLIF

SEQ ID NO: 550 - protein, 17 aa, Artificial Sequence
  Description of Artificial Sequence Synthetic peptide
CALSEGNYGGSQGNLIF

SEQ ID NO: 551 - protein, 12 aa, Artificial Sequence
  Description of Artificial Sequence Synthetic peptide
CAGRQGAQKLVF

SEQ ID NO: 552 - protein, 14 aa, Artificial Sequence
  Description of Artificial Sequence Synthetic peptide
CAVSSSGGSYIPTF

SEQ ID NO: 553 - protein, 13 aa, Artificial Sequence
  Description of Artificial Sequence Synthetic peptide
CAVTKPSGTALIF

SEQ ID NO: 554 - protein, 11 aa, Artificial Sequence
  Description of Artificial Sequence Synthetic peptide
CIESNTGKLIF

SEQ ID NO: 555 - protein, 11 aa, Artificial Sequence
  Description of Artificial Sequence Synthetic peptide
CAVSLTYKYIF

SEQ ID NO: 556 - protein, 14 aa, Artificial Sequence
  Description of Artificial Sequence Synthetic peptide
CAMTVNSGYSTLTF

SEQ ID NO: 557 - protein, 12 aa, Artificial Sequence
  Description of Artificial Sequence Synthetic peptide
CAENSGTYKYIF

SEQ ID NO: 558 - protein, 16 aa, Artificial Sequence
  Description of Artificial Sequence Synthetic peptide
CAENGSRDTGRRALTF

SEQ ID NO: 559 - protein, 13 aa, Artificial Sequence
  Description of Artificial Sequence Synthetic peptide
CIVRDNAGNMLTF

SEQ ID NO: 560 - protein, 14 aa, Artificial Sequence
  Description of Artificial Sequence Synthetic peptide
CVVKSRGGGGKLIF

SEQ ID NO: 561 - protein, 17 aa, Artificial Sequence
  Description of Artificial Sequence Synthetic peptide
SKRSFIEDLLFNKVTLA

SEQ ID NO: 562 - protein, 17 aa, Artificial Sequence
  Description of Artificial Sequence Synthetic peptide
GINITRFQTLLALHRSY

SEQ ID NO: 563 - protein, 16 aa, Artificial Sequence
  Description of Artificial Sequence Synthetic peptide
QFCNDPFLGVYHKNNK

SEQ ID NO: 564 - protein, 17 aa, Artificial Sequence
  Description of Artificial Sequence Synthetic peptide
IAGRSALEDLLFSKVVT

SEQ ID NO: 565 - protein, 8 aa, Artificial Sequence
  Description of Artificial Sequence Synthetic peptide
EDLLFNKV

SEQ ID NO: 566 - protein, 8 aa, Artificial Sequence
  Description of Artificial Sequence Synthetic peptide
EDLLFSKV

SEQ ID NO: 567 - protein, 17 aa, Artificial Sequence
  Description of Artificial Sequence Synthetic peptide
SNAFSLANVTSFGDYNL

SEQ ID NO: 568 - protein, 17 aa, Artificial Sequence
  Description of Artificial Sequence Synthetic peptide
ANVTSFGDYNLSSVLPQ

SEQ ID NO: 569 - protein, 17 aa, Artificial Sequence
  Description of Artificial Sequence Synthetic peptide
GDYNLSSVLPQRNIRSS

SEQ ID NO: 570 - protein, 17 aa, Artificial Sequence
  Description of Artificial Sequence Synthetic peptide
SVLPQRNIRSSRIAGRS

SEQ ID NO: 571 - protein, 17 aa, Artificial Sequence
  Description of Artificial Sequence Synthetic peptide
NIRSSRIAGRSALEDLL

SEQ ID NO: 572 - protein, 17 aa, Artificial Sequence
  Description of Artificial Sequence Synthetic peptide
LEDLLFSKVVTSGLGTV

SEQ ID NO: 573 - protein, 17 aa, Artificial Sequence
  Description of Artificial Sequence Synthetic peptide
SKVVTSGLGTVDVDYKS

SEQ ID NO: 574 - protein, 17 aa, Artificial Sequence
  Description of Artificial Sequence Synthetic peptide
GLGTVDVDYKSCTKGLS

SEQ ID NO: 575 - protein, 17 aa, Artificial Sequence
  Description of Artificial Sequence Synthetic peptide
VDYKSCTKGLSIADLAC

SEQ ID NO: 576 - protein, 17 aa, Artificial Sequence
  Description of Artificial Sequence Synthetic peptide
SGTNGTKRFDNPVLPFN

SEQ ID NO: 577 - protein, 17 aa, Artificial Sequence
  Description of Artificial Sequence Synthetic peptide
RFDNPVLPFNDGVYFAS

SEQ ID NO: 578 - protein, 17 aa, Artificial Sequence
  Description of Artificial Sequence Synthetic peptide
PFNDGVYFASTEKSNII

SEQ ID NO: 579 - protein, 17 aa, Artificial Sequence
  Description of Artificial Sequence Synthetic peptide
FASTEKSNIIRGWIFGT

SEQ ID NO: 580 - protein, 17 aa, Artificial Sequence
  Description of Artificial Sequence Synthetic peptide
NIIRGWIFGTTLDSKTQ

SEQ ID NO: 581 - protein, 17 aa, Artificial Sequence
  Description of Artificial Sequence Synthetic peptide
FGTTLDSKTQSLLIVNN

SEQ ID NO: 582 - protein, 17 aa, Artificial Sequence
  Description of Artificial Sequence Synthetic peptide
KTQSLLIVNNATNVVIK

SEQ ID NO: 583 - protein, 17 aa, Artificial Sequence
  Description of Artificial Sequence Synthetic peptide
VNNATNVVIKVCEFQFC

SEQ ID NO: 584 - protein, 17 aa, Artificial Sequence
  Description of Artificial Sequence Synthetic peptide
VIKVCEFQFCNDPFLGV

SEQ ID NO: 585 - protein, 17 aa, Artificial Sequence
  Description of Artificial Sequence Synthetic peptide
QFCNDPFLGVYYHKNNK

SEQ ID NO: 586 - protein, 17 aa, Artificial Sequence
  Description of Artificial Sequence Synthetic peptide
NLVRDLPQGFSALEPLV

SEQ ID NO: 587 - protein, 17 aa, Artificial Sequence
  Description of Artificial Sequence Synthetic peptide
QGFSALEPLVDLPIGIN

SEQ ID NO: 588 - protein, 17 aa, Artificial Sequence
  Description of Artificial Sequence Synthetic peptide
PLVDLPIGINITRFQTL

SEQ ID NO: 589 - protein, 17 aa, Artificial Sequence
  Description of Artificial Sequence Synthetic peptide
QTLLALHRSYLTPGDSS

SEQ ID NO: 590 - protein, 17 aa, Artificial Sequence
  Description of Artificial Sequence Synthetic peptide
RSYLTPGDSSSGWTAGA

SEQ ID NO: 591 - protein, 17 aa, Artificial Sequence
  Description of Artificial Sequence Synthetic peptide
DSSSGWTAGAAAYYVGY

SEQ ID NO: 592 - protein, 17 aa, Artificial Sequence
  Description of Artificial Sequence Synthetic peptide
AGAAAYYVGYLQPRTFL

SEQ ID NO: 593 - protein, 17 aa, Artificial Sequence
  Description of Artificial Sequence Synthetic peptide
VGYLQPRTFLLKYNENG

SEQ ID NO: 594 - protein, 17 aa, Artificial Sequence
  Description of Artificial Sequence Synthetic peptide
TFLLKYNENGTITDAVD

SEQ ID NO: 595 - protein, 17 aa, Artificial Sequence
  Description of Artificial Sequence Synthetic peptide
AVEQDKNTQEVFAQVKQ

SEQ ID NO: 596 - protein, 17 aa, Artificial Sequence
  Description of Artificial Sequence Synthetic peptide
TQEVFAQVKQIYKTPPI

SEQ ID NO: 597 - protein, 17 aa, Artificial Sequence
  Description of Artificial Sequence Synthetic peptide
VKQIYKTPPIKDFGGFN

SEQ ID NO: 598 - protein, 17 aa, Artificial Sequence
  Description of Artificial Sequence Synthetic peptide
PPIKDFGGFNFSQILPD

SEQ ID NO: 599 - protein, 17 aa, Artificial Sequence
  Description of Artificial Sequence Synthetic peptide
GFNFSQILPDPSKPSKR

SEQ ID NO: 600 - protein, 17 aa, Artificial Sequence
  Description of Artificial Sequence Synthetic peptide
LPDPSKPSKRSFIEDLL

SEQ ID NO: 601 - protein, 17 aa, Artificial Sequence
  Description of Artificial Sequence Synthetic peptide
DLLFNKVTLADAGFIKQ

SEQ ID NO: 602 - protein, 17 aa, Artificial Sequence
  Description of Artificial Sequence Synthetic peptide
TLADAGFIKQYGDCLGD

SEQ ID NO: 603 - protein, 17 aa, Artificial Sequence
  Description of Artificial Sequence Synthetic peptide
IKQYGDCLGDIAARDLI

SEQ ID NO: 604 - protein, 13 aa, Artificial Sequence
  Description of Artificial Sequence Synthetic peptide
CAWSVGGNYGTYF

SEQ ID NO: 605 - DNA, 20 bp, Artificial Sequence
  Description of Artificial Sequence Synthetic oligonucleotide
agagtctctcagctggtaca

SEQ ID NO: 606 - DNA, 20 bp, Artificial Sequence
  Description of Artificial Sequence Synthetic oligonucleotide
agaaggtggccgagaccctc

SEQ ID NO: 607 - DNA, 20 bp, Artificial Sequence
  Description of Artificial Sequence Synthetic primer
gcctaagttggggagaccac

SEQ ID NO: 608 - DNA, 20 bp, Artificial Sequence
  Description of Artificial Sequence Synthetic primer
gaagcaaggaaacagcctgc

SEQ ID NO: 609 - DNA, 22 bp, Artificial Sequence
  Description of Artificial Sequence Synthetic primer
tcgctgtgtttgagccatcaga

SEQ ID NO: 610 - DNA, 22 bp, Artificial Sequence
  Description of Artificial Sequence Synthetic primer
atgaaccacaggtgcccaattc

SEQ ID NO: 611 - protein, 12 aa, Artificial Sequence
  Description of Artificial Sequence Synthetic peptide
CAGFQGGIGNTF

SEQ ID NO: 612 - protein, 14 aa, Artificial Sequence
  Description of Artificial Sequence Synthetic peptide
CATSDLRGSTTQYF

SEQ ID NO: 613 - protein, 18 aa, Artificial Sequence
  Description of Artificial Sequence Synthetic peptide
CARSLRGTGGNLNQPQHF

SEQ ID NO: 614 - protein, 15 aa, Artificial Sequence
  Description of Artificial Sequence Synthetic peptide
CASSSTGFSSYEQYT

SEQ ID NO: 615 - protein, 12 aa, Artificial Sequence
  Description of Artificial Sequence Synthetic peptide
CAWSVSSYGYTF

SEQ ID NO: 616 - protein, 14 aa, Artificial Sequence
  Description of Artificial Sequence Synthetic peptide
CSAGPGSRINEQYF

SEQ ID NO: 617 - protein, 13 aa, Artificial Sequence
  Description of Artificial Sequence Synthetic peptide
CAVSSSGGSYIPT

SEQ ID NO: 618 - protein, 15 aa, Artificial Sequence
  Description of Artificial Sequence Synthetic peptide
CASSFRQRGQGGYTF

SEQ ID NO: 619 - protein, 14 aa, Artificial Sequence
  Description of Artificial Sequence Synthetic peptide
CASSLQGRTYEQYF

SEQ ID NO: 620 - protein, 15 aa, Artificial Sequence
  Description of Artificial Sequence Synthetic peptide
CASSYGGGGGETQYF

SEQ ID NO: 621 - protein, 13 aa, Artificial Sequence
  Description of Artificial Sequence Synthetic peptide
CSAQGDTGNTTYF

SEQ ID NO: 622 - protein, 15 aa, Artificial Sequence
  Description of Artificial Sequence Synthetic peptide
CASSLGGPQGHGYTF

SEQ ID NO: 623 - protein, 17 aa, Artificial Sequence
  Description of Artificial Sequence Synthetic peptide
CASSKASSGGAGDTQYF

SEQ ID NO: 624 - protein, 14 aa, Artificial Sequence
  Description of Artificial Sequence Synthetic peptide
CASSIVGRNTEAFF

SEQ ID NO: 625 - protein, 15 aa, Artificial Sequence
  Description of Artificial Sequence Synthetic peptide
CSARDPRGASAGYTF

SEQ ID NO: 626 - protein, 12 aa, Artificial Sequence
  Description of Artificial Sequence Synthetic peptide
CAGGDSNYGYTF

SEQ ID NO: 627 - protein, 14 aa, Artificial Sequence
  Description of Artificial Sequence Synthetic peptide
CASSLGHSMQPQHF

SEQ ID NO: 628 - protein, 15 aa, Artificial Sequence
  Description of Artificial Sequence Synthetic peptide
CASRYTGGSTDTQYF

SEQ ID NO: 629 - protein, 18 aa, Artificial Sequence
  Description of Artificial Sequence Synthetic peptide
CSARDGGSSTTSKMEQFF

SEQ ID NO: 630 - protein, 16 aa, Artificial Sequence
  Description of Artificial Sequence Synthetic peptide
CATPTPSDASSWEQYF

SEQ ID NO: 631 - protein, 13 aa, Artificial Sequence
  Description of Artificial Sequence Synthetic peptide
CASSLLGDYEQYF

SEQ ID NO: 632 - protein, 15 aa, Artificial Sequence
  Description of Artificial Sequence Synthetic peptide
CASSESRGGDAEAFF

SEQ ID NO: 633 - protein, 15 aa, Artificial Sequence
  Description of Artificial Sequence Synthetic peptide
CASSETGGDSGELFF

SEQ ID NO: 634 - protein, 13 aa, Artificial Sequence
  Description of Artificial Sequence Synthetic peptide
CASTQGLNTEAFF

SEQ ID NO: 635 - protein, 15 aa, Artificial Sequence
  Description of Artificial Sequence Synthetic peptide
CASSDGRGEQETQYF

SEQ ID NO: 636 - protein, 14 aa, Artificial Sequence
  Description of Artificial Sequence Synthetic peptide
CASRELAGLDTQYF

SEQ ID NO: 637 - protein, 13 aa, Artificial Sequence
  Description of Artificial Sequence Synthetic peptide
CSALPGLANTQYF

SEQ ID NO: 638 - protein, 12 aa, Artificial Sequence
  Description of Artificial Sequence Synthetic peptide
CASSLGPGTQYF

SEQ ID NO: 639 - protein, 14 aa, Artificial Sequence
  Description of Artificial Sequence Synthetic peptide
CASSVSVGTYEQYF

SEQ ID NO: 640 - protein, 11 aa, Artificial Sequence
  Description of Artificial Sequence Synthetic peptide
CASSSLDTQYF

SEQ ID NO: 641 - protein, 15 aa, Artificial Sequence
  Description of Artificial Sequence Synthetic peptide
CASNHREGHVGGHTF

SEQ ID NO: 642 - protein, 13 aa, Artificial Sequence
  Description of Artificial Sequence Synthetic peptide
CASSELGGAGHTF

SEQ ID NO: 643 - protein, 15 aa, Artificial Sequence
  Description of Artificial Sequence Synthetic peptide
CASSYRSMSTDTGYF

SEQ ID NO: 644 - protein, 13 aa, Artificial Sequence
  Description of Artificial Sequence Synthetic peptide
CATMRSGSAEAFF

SEQ ID NO: 645 - protein, 12 aa, Artificial Sequence
  Description of Artificial Sequence Synthetic peptide
CASSQQGAGHTF

SEQ ID NO: 646 - protein, 17 aa, Artificial Sequence
  Description of Artificial Sequence Synthetic peptide
CASSLGVGGLANYQYTF

SEQ ID NO: 647 - protein, 13 aa, Artificial Sequence
  Description of Artificial Sequence Synthetic peptide
CSGRGQGNYGHTF

SEQ ID NO: 648 - protein, 14 aa, Artificial Sequence
  Description of Artificial Sequence Synthetic peptide
CASSLGGTYNEQFF

SEQ ID NO: 649 - protein, 14 aa, Artificial Sequence
  Description of Artificial Sequence Synthetic peptide
CASSLSPGNYGYTF

SEQ ID NO: 650 - protein, 15 aa, Artificial Sequence
  Description of Artificial Sequence Synthetic peptide
CSASFPGVMNTEAFF

SEQ ID NO: 651 - protein, 12 aa, Artificial Sequence
  Description of Artificial Sequence Synthetic peptide
CASSSSRTSYTF

SEQ ID NO: 652 - protein, 13 aa, Artificial Sequence
  Description of Artificial Sequence Synthetic peptide
CASSSMNTGELFF

SEQ ID NO: 653 - protein, 14 aa, Artificial Sequence
  Description of Artificial Sequence Synthetic peptide
CASSLVPGNYGYTF

SEQ ID NO: 654 - protein, 12 aa, Artificial Sequence
  Description of Artificial Sequence Synthetic peptide
CASSLDYSQQHF

SEQ ID NO: 655 - protein, 13 aa, Artificial Sequence
  Description of Artificial Sequence Synthetic peptide
CASSSYLTSGYTF

SEQ ID NO: 656 - protein, 15 aa, Artificial Sequence
  Description of Artificial Sequence Synthetic peptide
CASSLIGNSAETQYF

SEQ ID NO: 657 - protein, 16 aa, Artificial Sequence
  Description of Artificial Sequence Synthetic peptide
CASSLGGQGNTGELFF

SEQ ID NO: 658 - protein, 15 aa, Artificial Sequence
  Description of Artificial Sequence Synthetic peptide
CASRRGLAGNNEQFF

SEQ ID NO: 659 - protein, 16 aa, Artificial Sequence
  Description of Artificial Sequence Synthetic peptide
CATYPTSGTSTGELFF

SEQ ID NO: 660 - protein, 15 aa, Artificial Sequence
  Description of Artificial Sequence Synthetic peptide
CASSADSTRNQPQHF

SEQ ID NO: 661 - protein, 14 aa, Artificial Sequence
  Description of Artificial Sequence Synthetic peptide
CATSDTGVSGELFF

SEQ ID NO: 662 - protein, 12 aa, Artificial Sequence
  Description of Artificial Sequence Synthetic peptide
CASSPRTGGAFF

SEQ ID NO: 663 - protein, 15 aa, Artificial Sequence
  Description of Artificial Sequence Synthetic peptide
CASSGDSTRNQPQHF

SEQ ID NO: 664 - protein, 12 aa, Artificial Sequence
  Description of Artificial Sequence Synthetic peptide
CASSPRGGGYTF

SEQ ID NO: 665 - protein, 12 aa, Artificial Sequence
  Description of Artificial Sequence Synthetic peptide
CASGTGYTEAFF

SEQ ID NO: 666 - protein, 14 aa, Artificial Sequence
  Description of Artificial Sequence Synthetic peptide
CASSADRAPYGYTF

SEQ ID NO: 667 - protein, 17 aa, Artificial Sequence
  Description of Artificial Sequence Synthetic peptide
CASSRTGGSSYNSPLHF

SEQ ID NO: 668 - protein, 10 aa, Artificial Sequence
  Description of Artificial Sequence Synthetic peptide
CSADRETQYF

SEQ ID NO: 669 - protein, 14 aa, Artificial Sequence
  Description of Artificial Sequence Synthetic peptide
CASSLAGGDGTDTI

SEQ ID NO: 670 - protein, 12 aa, Artificial Sequence
  Description of Artificial Sequence Synthetic peptide
CATSDNLDTQYF

SEQ ID NO: 671 - protein, 14 aa, Artificial Sequence
  Description of Artificial Sequence Synthetic peptide
CASSLASGNYGYTF

SEQ ID NO: 672 - protein, 15 aa, Artificial Sequence
  Description of Artificial Sequence Synthetic peptide
CSARPRQGKVDTQYF

SEQ ID NO: 673 - protein, 14 aa, Artificial Sequence
  Description of Artificial Sequence Synthetic peptide
CASSLSGMRDEAFF

SEQ ID NO: 674 - protein, 14 aa, Artificial Sequence
  Description of Artificial Sequence Synthetic peptide
CASSLTFASGELFF

SEQ ID NO: 675 - protein, 13 aa, Artificial Sequence
  Description of Artificial Sequence Synthetic peptide
CASSVDRGYGYTF

SEQ ID NO: 676 - protein, 14 aa, Artificial Sequence
  Description of Artificial Sequence Synthetic peptide
CASSLDAGTDTQYF

SEQ ID NO: 677 - protein, 14 aa, Artificial Sequence
  Description of Artificial Sequence Synthetic peptide
CSATSGGFQETQYF

SEQ ID NO: 678 - protein, 19 aa, Artificial Sequence
  Description of Artificial Sequence Synthetic peptide
CSATSPQEHGGAFTDTGYF

SEQ ID NO: 679 - protein, 12 aa, Artificial Sequence
  Description of Artificial Sequence Synthetic peptide
CASSFGVYGYTF

SEQ ID NO: 680 - protein, 13 aa, Artificial Sequence
  Description of Artificial Sequence Synthetic peptide
CASSVVNTGELFF

SEQ ID NO: 681 - protein, 12 aa, Artificial Sequence
  Description of Artificial Sequence Synthetic peptide
CASRWLGGELFF

SEQ ID NO: 682 - protein, 14 aa, Artificial Sequence
  Description of Artificial Sequence Synthetic peptide
CASSYGLTISSYTF

SEQ ID NO: 683 - protein, 14 aa, Artificial Sequence
  Description of Artificial Sequence Synthetic peptide
CASSAYTANNSYTF

SEQ ID NO: 684 - protein, 13 aa, Artificial Sequence
  Description of Artificial Sequence Synthetic peptide
CASSFNTEYGYTF

SEQ ID NO: 685 - protein, 16 aa, Artificial Sequence
  Description of Artificial Sequence Synthetic peptide
CASSLRVVRGAYGYTF

SEQ ID NO: 686 - protein, 15 aa, Artificial Sequence
  Description of Artificial Sequence Synthetic peptide
CASSLASEWMQPQHF

SEQ ID NO: 687 - protein, 15 aa, Artificial Sequence
  Description of Artificial Sequence Synthetic peptide
CASSPGLASASAQYF

SEQ ID NO: 688 - protein, 15 aa, Artificial Sequence
  Description of Artificial Sequence Synthetic peptide
CASTRPGILKSPLHF

SEQ ID NO: 689 - protein, 14 aa, Artificial Sequence
  Description of Artificial Sequence Synthetic peptide
CASGLVSSQARWFF

SEQ ID NO: 690 - protein, 13 aa, Artificial Sequence
  Description of Artificial Sequence Synthetic peptide
CASSLGMNYGYTF

SEQ ID NO: 691 - protein, 14 aa, Artificial Sequence
  Description of Artificial Sequence Synthetic peptide
CASSYLGQNTEAFF

SEQ ID NO: 692 - protein, 12 aa, Artificial Sequence
  Description of Artificial Sequence Synthetic peptide
CANRGNNYGYTF

SEQ ID NO: 693 - protein, 12 aa, Artificial Sequence
  Description of Artificial Sequence Synthetic peptide
CASSPTGCGYTF

SEQ ID NO: 694 - protein, 15 aa, Artificial Sequence
  Description of Artificial Sequence Synthetic peptide
CSATGALSYNSPLHF

SEQ ID NO: 695 - protein, 13 aa, Artificial Sequence
  Description of Artificial Sequence Synthetic peptide
CATSREGNQPQHF

SEQ ID NO: 696 - protein, 13 aa, Artificial Sequence
  Description of Artificial Sequence Synthetic peptide
CASSLDRATEAFF

SEQ ID NO: 697 - protein, 14 aa, Artificial Sequence
  Description of Artificial Sequence Synthetic peptide
CASMSGRSSYEQYF

SEQ ID NO: 698 - protein, 17 aa, Artificial Sequence
  Description of Artificial Sequence Synthetic peptide
CASSLAGLAPNTGELFF

SEQ ID NO: 699 - protein, 16 aa, Artificial Sequence
  Description of Artificial Sequence Synthetic peptide
CASTNASTLGNQPQHF

SEQ ID NO: 700 - protein, 13 aa, Artificial Sequence
  Description of Artificial Sequence Synthetic peptide
CASRDRAHYGYTF

SEQ ID NO: 701 - protein, 15 aa, Artificial Sequence
  Description of Artificial Sequence Synthetic peptide
CASSKVVTGPYGYTF

SEQ ID NO: 702 - protein, 14 aa, Artificial Sequence
  Description of Artificial Sequence Synthetic peptide
CASSQGDGNGPQHF

SEQ ID NO: 703 - protein, 11 aa, Artificial Sequence
  Description of Artificial Sequence Synthetic peptide
CASWGNQPCHF

SEQ ID NO: 704 - protein, 15 aa, Artificial Sequence
  Description of Artificial Sequence Synthetic peptide
CASSEGSADHSPLHF

SEQ ID NO: 705 - protein, 16 aa, Artificial Sequence
  Description of Artificial Sequence Synthetic peptide
CASSSGTAPQFYQYTF

SEQ ID NO: 706 - protein, 13 aa, Artificial Sequence
  Description of Artificial Sequence Synthetic peptide
CASSQMGAHGHTF

SEQ ID NO: 707 - protein, 14 aa, Artificial Sequence
  Description of Artificial Sequence Synthetic peptide
CASSLARRNTEAFF

SEQ ID NO: 708 - protein, 15 aa, Artificial Sequence
  Description of Artificial Sequence Synthetic peptide
CSARGRGPNINEQFF

SEQ ID NO: 709 - protein, 17 aa, Artificial Sequence
  Description of Artificial Sequence Synthetic peptide
CSARDPARGQGKSPLHF

SEQ ID NO: 710 - protein, 13 aa, Artificial Sequence
  Description of Artificial Sequence Synthetic peptide
CASNQQRNYEQYF

SEQ ID NO: 711 - protein, 15 aa, Artificial Sequence
  Description of Artificial Sequence Synthetic peptide
CASSSRQGGTGELFF

SEQ ID NO: 712 - protein, 14 aa, Artificial Sequence
  Description of Artificial Sequence Synthetic peptide
CASSFGESNQPQHF

SEQ ID NO: 713 - protein, 16 aa, Artificial Sequence
  Description of Artificial Sequence Synthetic peptide
CASSRAGKVAKNIQYF

SEQ ID NO: 714 - protein, 15 aa, Artificial Sequence
  Description of Artificial Sequence Synthetic peptide
CASNHRGPANYGYTF

SEQ ID NO: 715 - protein, 13 aa, Artificial Sequence
  Description of Artificial Sequence Synthetic peptide
CASSYRVNTEAFF

SEQ ID NO: 716 - protein, 16 aa, Artificial Sequence
  Description of Artificial Sequence Synthetic peptide
CASSLDREGYTDTQYF

SEQ ID NO: 717 - protein, 15 aa, Artificial Sequence
  Description of Artificial Sequence Synthetic peptide
CATPRDGSSYNEQFF

SEQ ID NO: 718 - protein, 13 aa, Artificial Sequence
  Description of Artificial Sequence Synthetic peptide
CSARKGRGYGYTF

SEQ ID NO: 719 - protein, 13 aa, Artificial Sequence
  Description of Artificial Sequence Synthetic peptide
CASKRDSNQPQHF

SEQ ID NO: 720 - protein, 14 aa, Artificial Sequence
  Description of Artificial Sequence Synthetic peptide
CASSPTGGDSPLHF

SEQ ID NO: 721 - protein, 16 aa, Artificial Sequence
  Description of Artificial Sequence Synthetic peptide
CASSEFSSGGRDTQYF

SEQ ID NO: 722 - protein, 13 aa, Artificial Sequence
  Description of Artificial Sequence Synthetic peptide
CASSPLAGPRQYF

SEQ ID NO: 723 - protein, 17 aa, Artificial Sequence
  Description of Artificial Sequence Synthetic peptide
CASSLRRGDNGSYGYTF

SEQ ID NO: 724 - protein, 15 aa, Artificial Sequence
  Description of Artificial Sequence Synthetic peptide
CASSSRQGALEQYTF

SEQ ID NO: 725 - protein, 13 aa, Artificial Sequence
  Description of Artificial Sequence Synthetic peptide
CASSQQWDQAGHF

SEQ ID NO: 726 - protein, 14 aa, Artificial Sequence
  Description of Artificial Sequence Synthetic peptide
CASVVGRAPDTQYF

SEQ ID NO: 727 - protein, 11 aa, Artificial Sequence
  Description of Artificial Sequence Synthetic peptide
CASSVGQAGHF

SEQ ID NO: 728 - protein, 20 aa, Artificial Sequence
  Description of Artificial Sequence Synthetic peptide
CASSAQAQGSLASGTGCLFF

SEQ ID NO: 729 - protein, 16 aa, Artificial Sequence
  Description of Artificial Sequence Synthetic peptide
CASNPQAQNITDFQYF

SEQ ID NO: 730 - protein, 14 aa, Artificial Sequence
  Description of Artificial Sequence Synthetic peptide
CSATAGLNTGELFF

SEQ ID NO: 731 - protein, 12 aa, Artificial Sequence
  Description of Artificial Sequence Synthetic peptide
CASSISEEYQYF

SEQ ID NO: 732 - protein, 11 aa, Artificial Sequence
  Description of Artificial Sequence Synthetic peptide
CAGGTQETQYF

SEQ ID NO: 733 - protein, 15 aa, Artificial Sequence
  Description of Artificial Sequence Synthetic peptide
CASSESGTTNTEAFF

SEQ ID NO: 734 - protein, 15 aa, Artificial Sequence
  Description of Artificial Sequence Synthetic peptide
CASQNQLIQTQTQYF

SEQ ID NO: 735 - protein, 13 aa, Artificial Sequence
  Description of Artificial Sequence Synthetic peptide
CASSLFQGETQYF

SEQ ID NO: 736 - protein, 15 aa, Artificial Sequence
  Description of Artificial Sequence Synthetic peptide
CSASSGGGAANTQYF

SEQ ID NO: 737 - protein, 13 aa, Artificial Sequence
  Description of Artificial Sequence Synthetic peptide
CASAGQYQCTQYF

SEQ ID NO: 738 - protein, 17 aa, Artificial Sequence
  Description of Artificial Sequence Synthetic peptide
CSARSSGQGFSTDTQYF

SEQ ID NO: 739 - protein, 16 aa, Artificial Sequence
  Description of Artificial Sequence Synthetic peptide
CSSSSGGRLSASMTYF

SEQ ID NO: 740 - protein, 14 aa, Artificial Sequence
  Description of Artificial Sequence Synthetic peptide
CATSLLVILMEQFF

SEQ ID NO: 741 - protein, 16 aa, Artificial Sequence
  Description of Artificial Sequence Synthetic peptide
CASSQPPLTTRYEQYF

SEQ ID NO: 742 - protein, 13 aa, Artificial Sequence
  Description of Artificial Sequence Synthetic peptide
CSAALGAKDTQYF

SEQ ID NO: 743 - protein, 13 aa, Artificial Sequence
  Description of Artificial Sequence Synthetic peptide
CASSCRQGTEAFF

SEQ ID NO: 744 - protein, 17 aa, Artificial Sequence
  Description of Artificial Sequence Synthetic peptide
CASLPQLGFGLQEYQYF

SEQ ID NO: 745 - protein, 13 aa, Artificial Sequence
  Description of Artificial Sequence Synthetic peptide
CASSYTNQITIYF

SEQ ID NO: 746 - protein, 18 aa, Artificial Sequence
  Description of Artificial Sequence Synthetic peptide
CASSQAPCASTGLKGYTF

SEQ ID NO: 747 - protein, 16 aa, Artificial Sequence
  Description of Artificial Sequence Synthetic peptide
CAQQQANQTTPGPQHF

SEQ ID NO: 748 - protein, 12 aa, Artificial Sequence
  Description of Artificial Sequence Synthetic peptide
CAEPCAIQGYTF

SEQ ID NO: 749 - protein, 18 aa, Artificial Sequence
  Description of Artificial Sequence Synthetic peptide
CASSYQISHLPGTGILFF

SEQ ID NO: 750 - protein, 17 aa, Artificial Sequence
  Description of Artificial Sequence Synthetic peptide
CASASQQESSYHGHLHF

SEQ ID NO: 751 - protein, 15 aa, Artificial Sequence
  Description of Artificial Sequence Synthetic peptide
CASSFQQSGAQTQYF

SEQ ID NO: 752 - protein, 14 aa, Artificial Sequence
  Description of Artificial Sequence Synthetic peptide
CSASGGGGMNEQFF

SEQ ID NO: 753 - protein, 18 aa, Artificial Sequence
  Description of Artificial Sequence Synthetic peptide
CASSYGMTPQGINTEAFF

SEQ ID NO: 754 - protein, 14 aa, Artificial Sequence
  Description of Artificial Sequence Synthetic peptide
CSASQKGLGNTIYF

SEQ ID NO: 755 - protein, 16 aa, Artificial Sequence
  Description of Artificial Sequence Synthetic peptide
CASSLAVGVIFYGYTF

SEQ ID NO: 756 - protein, 14 aa, Artificial Sequence
  Description of Artificial Sequence Synthetic peptide
CASSSSFSLGTQYF

SEQ ID NO: 757 - protein, 14 aa, Artificial Sequence
  Description of Artificial Sequence Synthetic peptide
CASSYQGAHTEAFF

SEQ ID NO: 758 - protein, 15 aa, Artificial Sequence
  Description of Artificial Sequence Synthetic peptide
CASNRPQQGNYGYTF

SEQ ID NO: 759 - protein, 14 aa, Artificial Sequence
  Description of Artificial Sequence Synthetic peptide
CSANDFLISLEAFF

SEQ ID NO: 760 - protein, 14 aa, Artificial Sequence
  Description of Artificial Sequence Synthetic peptide
CSANGITIGLEAFF

SEQ ID NO: 761 - protein, 12 aa, Artificial Sequence
  Description of Artificial Sequence Synthetic peptide
CSAIGTLYLQYF

SEQ ID NO: 762 - protein, 13 aa, Artificial Sequence
  Description of Artificial Sequence Synthetic peptide
CSAHDMAALEAFF

SEQ ID NO: 763 - protein, 17 aa, Artificial Sequence
  Description of Artificial Sequence Synthetic peptide
CASSPTSTEQANYGYTF

SEQ ID NO: 764 - protein, 16 aa, Artificial Sequence
  Description of Artificial Sequence Synthetic peptide
CASAITQSVSNTEAFF

SEQ ID NO: 765 - protein, 14 aa, Artificial Sequence
  Description of Artificial Sequence Synthetic peptide
CASTETGLNYGYTF

SEQ ID NO: 766 - protein, 14 aa, Artificial Sequence
  Description of Artificial Sequence Synthetic peptide
CASPQQTFPYGYTF

SEQ ID NO: 767 - protein, 13 aa, Artificial Sequence
  Description of Artificial Sequence Synthetic peptide
CASVGTPQEQQYF

SEQ ID NO: 768 - protein, 14 aa, Artificial Sequence
  Description of Artificial Sequence Synthetic peptide
CASLYSGSGYGYTF

SEQ ID NO: 769 - protein, 13 aa, Artificial Sequence
  Description of Artificial Sequence Synthetic peptide
CSAAGGNNTEAFF

SEQ ID NO: 770 - protein, 15 aa, Artificial Sequence
  Description of Artificial Sequence Synthetic peptide
CSAFQNQGGRETQYF

SEQ ID NO: 771 - protein, 12 aa, Artificial Sequence
  Description of Artificial Sequence Synthetic peptide
CASSSSNQFQYF

SEQ ID NO: 772 - protein, 17 aa, Artificial Sequence
  Description of Artificial Sequence Synthetic peptide
CASSLATGTSGTDTQYF

SEQ ID NO: 773 - protein, 15 aa, Artificial Sequence
  Description of Artificial Sequence Synthetic peptide
CATSLQGAGNQPQHF

SEQ ID NO: 774 - protein, 14 aa, Artificial Sequence
  Description of Artificial Sequence Synthetic peptide
CSARPGLDMQPQHF

SEQ ID NO: 775 - protein, 15 aa, Artificial Sequence
  Description of Artificial Sequence Synthetic peptide
CSAQEQRNGNQPQHF

SEQ ID NO: 776 - protein, 15 aa, Artificial Sequence
  Description of Artificial Sequence Synthetic peptide
CASSLGLAAFYDQYF

SEQ ID NO: 777 - protein, 14 aa, Artificial Sequence
  Description of Artificial Sequence Synthetic peptide
CSARDLPDREPQHF

SEQ ID NO: 778 - protein, 15 aa, Artificial Sequence
  Description of Artificial Sequence Synthetic peptide
CATGGDRGLLDTQYF

SEQ ID NO: 779 - protein, 13 aa, Artificial Sequence
  Description of Artificial Sequence Synthetic peptide
CASSQGATETQYF

SEQ ID NO: 780 - protein, 15 aa, Artificial Sequence
  Description of Artificial Sequence Synthetic peptide
CASSRTWDTSYEQYF

SEQ ID NO: 781 - protein, 13 aa, Artificial Sequence
  Description of Artificial Sequence Synthetic peptide
CSADGRDSTDQYF

SEQ ID NO: 782 - protein, 16 aa, Artificial Sequence
  Description of Artificial Sequence Synthetic peptide
CASSIHKGAPQETQYF

SEQ ID NO: 783 - protein, 12 aa, Artificial Sequence
  Description of Artificial Sequence Synthetic peptide
CASSVGYNEQFF

SEQ ID NO: 784 - protein, 14 aa, Artificial Sequence
  Description of Artificial Sequence Synthetic peptide
CATSEPGQRTEAFF

SEQ ID NO: 785 - protein, 14 aa, Artificial Sequence
  Description of Artificial Sequence Synthetic peptide
CSVVKSGGTGGYTF

SEQ ID NO: 786 - protein, 14 aa, Artificial Sequence
  Description of Artificial Sequence Synthetic peptide
CASSLEGAWTEAFF

SEQ ID NO: 787 - protein, 14 aa, Artificial Sequence
  Description of Artificial Sequence Synthetic peptide
CASSLSGYDYGYTF

SEQ ID NO: 788 - protein, 14 aa, Artificial Sequence
  Description of Artificial Sequence Synthetic peptide
CASSLDGGDNEQFF

SEQ ID NO: 789 - protein, 14 aa, Artificial Sequence
  Description of Artificial Sequence Synthetic peptide
CASTVGSGNSPLHF

SEQ ID NO: 790 - protein, 15 aa, Artificial Sequence
  Description of Artificial Sequence Synthetic peptide
CSARDGLSPQETQYF

SEQ ID NO: 791 - protein, 16 aa, Artificial Sequence
  Description of Artificial Sequence Synthetic peptide
CASSQPGPGKATEAFF

SEQ ID NO: 792 - protein, 14 aa, Artificial Sequence
  Description of Artificial Sequence Synthetic peptide
CASSRGSGNYGYTF

SEQ ID NO: 793 - protein, 16 aa, Artificial Sequence
  Description of Artificial Sequence Synthetic peptide
CASSYFGIGQMNEAFF

SEQ ID NO: 794 - protein, 13 aa, Artificial Sequence
  Description of Artificial Sequence Synthetic peptide
CASSQVQSSQYTF

SEQ ID NO: 795 - protein, 12 aa, Artificial Sequence
  Description of Artificial Sequence Synthetic peptide
CVWEVAKNIQYF

SEQ ID NO: 796 - protein, 14 aa, Artificial Sequence
  Description of Artificial Sequence Synthetic peptide
CASSQQGGVSEQYF

SEQ ID NO: 797 - protein, 16 aa, Artificial Sequence
  Description of Artificial Sequence Synthetic peptide
CAGTGLGTGSYYGYTF

SEQ ID NO: 798 - protein, 16 aa, Artificial Sequence
  Description of Artificial Sequence Synthetic peptide
CASQPQLGGLADTQYF

SEQ ID NO: 799 - protein, 14 aa, Artificial Sequence
  Description of Artificial Sequence Synthetic peptide
CASKGQGSGNTIYF

SEQ ID NO: 800 - protein, 15 aa, Artificial Sequence
  Description of Artificial Sequence Synthetic peptide
CSASPGHLRGQPQHF

SEQ ID NO: 801 - protein, 15 aa, Artificial Sequence
  Description of Artificial Sequence Synthetic peptide
CASSTRGTGANVLTF

SEQ ID NO: 802 - protein, 14 aa, Artificial Sequence
  Description of Artificial Sequence Synthetic peptide
CASSLRGFGTEAFF

SEQ ID NO: 803 - protein, 16 aa, Artificial Sequence
  Description of Artificial Sequence Synthetic peptide
CATSDPGQDFNQPQHF

SEQ ID NO: 804 - protein, 16 aa, Artificial Sequence
  Description of Artificial Sequence Synthetic peptide
CSARVGGWASNQPQHF

SEQ ID NO: 805 - protein, 18 aa, Artificial Sequence
  Description of Artificial Sequence Synthetic peptide
CSARDHWEPLLAKNIQYF

SEQ ID NO: 806 - protein, 13 aa, Artificial Sequence
  Description of Artificial Sequence Synthetic peptide
CASSFRRRETQYF

SEQ ID NO: 807 - protein, 14 aa, Artificial Sequence
  Description of Artificial Sequence Synthetic peptide
CASRPGGLKETQYF

SEQ ID NO: 808 - protein, 13 aa, Artificial Sequence
  Description of Artificial Sequence Synthetic peptide
CSAREGDVQGYTF

SEQ ID NO: 809 - protein, 13 aa, Artificial Sequence
  Description of Artificial Sequence Synthetic peptide
CSARWGGGYGYTF

SEQ ID NO: 810 - protein, 13 aa, Artificial Sequence
  Description of Artificial Sequence Synthetic peptide
CASSRTWGSEAFF

SEQ ID NO: 811 - protein, 14 aa, Artificial Sequence
  Description of Artificial Sequence Synthetic peptide
CSASQGLPYNEQFF

SEQ ID NO: 812 - protein, 15 aa, Artificial Sequence
  Description of Artificial Sequence Synthetic peptide
CASSRYEPGSHEQYF

SEQ ID NO: 813 - protein, 13 aa, Artificial Sequence
  Description of Artificial Sequence Synthetic peptide
CAWSVRGNQPQHF

SEQ ID NO: 814 - protein, 15 aa, Artificial Sequence
  Description of Artificial Sequence Synthetic peptide
CSAGEGAGGYNEQFF

SEQ ID NO: 815 - protein, 15 aa, Artificial Sequence
  Description of Artificial Sequence Synthetic peptide
CSARGILVKQETQYF

SEQ ID NO: 816 - protein, 12 aa, Artificial Sequence
  Description of Artificial Sequence Synthetic peptide
CSARSLNTEAFF

SEQ ID NO: 817 - protein, 14 aa, Artificial Sequence
  Description of Artificial Sequence Synthetic peptide
CASSFQGNDYGYTF

SEQ ID NO: 818 - protein, 16 aa, Artificial Sequence
  Description of Artificial Sequence Synthetic peptide
CATSRGEWRGSNEQFF

SEQ ID NO: 819 - protein, 15 aa, Artificial Sequence
  Description of Artificial Sequence Synthetic peptide
CASSADLWVSKAQHF

SEQ ID NO: 820 - protein, 13 aa, Artificial Sequence
  Description of Artificial Sequence Synthetic peptide
CSGKEGRGNTIYF

SEQ ID NO: 821 - protein, 13 aa, Artificial Sequence
  Description of Artificial Sequence Synthetic peptide
CSATQGGPNTIYF

SEQ ID NO: 822 - protein, 13 aa, Artificial Sequence
  Description of Artificial Sequence Synthetic peptide
CSAREPLSNGQFF

SEQ ID NO: 823 - protein, 13 aa, Artificial Sequence
  Description of Artificial Sequence Synthetic peptide
CRAGQGGPDTQYF

SEQ ID NO: 824 - protein, 12 aa, Artificial Sequence
  Description of Artificial Sequence Synthetic peptide
CSARHGQETQYF

SEQ ID NO: 825 - protein, 15 aa, Artificial Sequence
  Description of Artificial Sequence Synthetic peptide
CASSLSSWTGDEQYF

SEQ ID NO: 826 - protein, 14 aa, Artificial Sequence
  Description of Artificial Sequence Synthetic peptide
CASSAIPRGQALHF

SEQ ID NO: 827 - protein, 14 aa, Artificial Sequence
  Description of Artificial Sequence Synthetic peptide
CASSLTGVAGELFF

SEQ ID NO: 828 - protein, 15 aa, Artificial Sequence
  Description of Artificial Sequence Synthetic peptide
CASSTGTASNQPQHF

SEQ ID NO: 829 - protein, 14 aa, Artificial Sequence
  Description of Artificial Sequence Synthetic peptide
CASSLQGMNTEAFF

SEQ ID NO: 830 - protein, 12 aa, Artificial Sequence
  Description of Artificial Sequence Synthetic peptide
CASSVIYYEQYF

SEQ ID NO: 831 - protein, 12 aa, Artificial Sequence
  Description of Artificial Sequence Synthetic peptide
CASSSFYNEQFF

SEQ ID NO: 832 - protein, 12 aa, Artificial Sequence
  Description of Artificial Sequence Synthetic peptide
CASCMSNSPLHF

SEQ ID NO: 833 - protein, 13 aa, Artificial Sequence
  Description of Artificial Sequence Synthetic peptide
CASSLGQNITIYF

SEQ ID NO: 834 - protein, 14 aa, Artificial Sequence
  Description of Artificial Sequence Synthetic peptide
CASSDRDSQETQYF

SEQ ID NO: 835 - protein, 14 aa, Artificial Sequence
  Description of Artificial Sequence Synthetic peptide
CASSKGSGNYGYTF

SEQ ID NO: 836 - protein, 15 aa, Artificial Sequence
  Description of Artificial Sequence Synthetic peptide
CASRKGLAGEETQYF

SEQ ID NO: 837 - protein, 14 aa, Artificial Sequence
  Description of Artificial Sequence Synthetic peptide
CASSLVSGNYGYTF

SEQ ID NO: 838 - protein, 14 aa, Artificial Sequence
  Description of Artificial Sequence Synthetic peptide
CASGQGSGNSPLHF

SEQ ID NO: 839 - protein, 14 aa, Artificial Sequence
  Description of Artificial Sequence Synthetic peptide
CASSPGDHTDTQYF

SEQ ID NO: 840 - protein, 14 aa, Artificial Sequence
  Description of Artificial Sequence Synthetic peptide
CASSAASANYGFTF

SEQ ID NO: 841 - protein, 14 aa, Artificial Sequence
  Description of Artificial Sequence Synthetic peptide
CASSFSVDNYGFTF

SEQ ID NO: 842 - protein, 13 aa, Artificial Sequence
  Description of Artificial Sequence Synthetic peptide
CASSAGANYGYTF

SEQ ID NO: 843 - protein, 13 aa, Artificial Sequence
  Description of Artificial Sequence Synthetic peptide
CASSQGANYGYTF

SEQ ID NO: 844 - protein, 14 aa, Artificial Sequence
  Description of Artificial Sequence Synthetic peptide
CASSPGSGNSPLIF

SEQ ID NO: 845 - protein, 14 aa, Artificial Sequence
  Description of Artificial Sequence Synthetic peptide
CACKTGTMNTEAFF

SEQ ID NO: 846 - protein, 14 aa, Artificial Sequence
  Description of Artificial Sequence Synthetic peptide
CASSLGPGDSPLHF

SEQ ID NO: 847 - protein, 13 aa, Artificial Sequence
  Description of Artificial Sequence Synthetic peptide
CASGQSNTGELFF

SEQ ID NO: 848 - protein, 16 aa, Artificial Sequence
  Description of Artificial Sequence Synthetic peptide
CSARDEGGESNQPQHF

SEQ ID NO: 849 - protein, 13 aa, Artificial Sequence
  Description of Artificial Sequence Synthetic peptide
CASSSQSNTEAFF

SEQ ID NO: 850 - protein, 15 aa, Artificial Sequence
  Description of Artificial Sequence Synthetic peptide
CSARTPAGGQDTQYF

SEQ ID NO: 851 - protein, 16 aa, Artificial Sequence
  Description of Artificial Sequence Synthetic peptide
CSARDPFGSGLYGFTF

SEQ ID NO: 852 - protein, 13 aa, Artificial Sequence
  Description of Artificial Sequence Synthetic peptide
CASFASSTDTQYF

SEQ ID NO: 853 - protein, 15 aa, Artificial Sequence
  Description of Artificial Sequence Synthetic peptide
CASTLAGDAQKTQAT

SEQ ID NO: 854 - protein, 14 aa, Artificial Sequence
  Description of Artificial Sequence Synthetic peptide
CASSQMAGAGFQYF

SEQ ID NO: 855 - protein, 10 aa, Artificial Sequence
  Description of Artificial Sequence Synthetic peptide
CAKEEGTGFF

SEQ ID NO: 856 - protein, 14 aa, Artificial Sequence
  Description of Artificial Sequence Synthetic peptide
CASSAAPSGAMATF

SEQ ID NO: 857 - protein, 13 aa, Artificial Sequence
  Description of Artificial Sequence Synthetic peptide
CASSLDGGGEQFP

SEQ ID NO: 858 - protein, 14 aa, Artificial Sequence
  Description of Artificial Sequence Synthetic peptide
CASSPTSGKLTQYF

SEQ ID NO: 859 - protein, 13 aa, Artificial Sequence
  Description of Artificial Sequence Synthetic peptide
CSAFDPVQGTQYF

SEQ ID NO: 860 - protein, 13 aa, Artificial Sequence
  Description of Artificial Sequence Synthetic peptide
CANNLAAQFTQYF

SEQ ID NO: 861 - protein, 14 aa, Artificial Sequence
  Description of Artificial Sequence Synthetic peptide
CASSEGVGTDTQNF

SEQ ID NO: 862 - protein, 14 aa, Artificial Sequence
  Description of Artificial Sequence Synthetic peptide
CASSASQQNYGFTF

SEQ ID NO: 863 - protein, 16 aa, Artificial Sequence
  Description of Artificial Sequence Synthetic peptide
CASTANTQTGPDTQYF

SEQ ID NO: 864 - protein, 16 aa, Artificial Sequence
  Description of Artificial Sequence Synthetic peptide
CASSRRMTSGTDTQYF

SEQ ID NO: 865 - protein, 13 aa, Artificial Sequence
  Description of Artificial Sequence Synthetic peptide
CASTTYSQETQTF

SEQ ID NO: 866 - protein, 14 aa, Artificial Sequence
  Description of Artificial Sequence Synthetic peptide
CSVRLRSYNSPLHF

SEQ ID NO: 867 - protein, 14 aa, Artificial Sequence
  Description of Artificial Sequence Synthetic peptide
CARKGQSANQPQHF

SEQ ID NO: 868 - protein, 14 aa, Artificial Sequence
  Description of Artificial Sequence Synthetic peptide
CASSTYDRAPEAFF

SEQ ID NO: 869 - protein, 14 aa, Artificial Sequence
  Description of Artificial Sequence Synthetic peptide
CASSFGLAGYTQYF

SEQ ID NO: 870 - protein, 15 aa, Artificial Sequence
  Description of Artificial Sequence Synthetic peptide
CASSLGTHKGNEQFF

SEQ ID NO: 871 - protein, 14 aa, Artificial Sequence
  Description of Artificial Sequence Synthetic peptide
CASSLGLAGYTQYF

SEQ ID NO: 872 - protein, 15 aa, Artificial Sequence
  Description of Artificial Sequence Synthetic peptide
CASSLGVGLGYEQYF

SEQ ID NO: 873 - protein, 12 aa, Artificial Sequence
  Description of Artificial Sequence Synthetic peptide
CASSTYLDTQYF

SEQ ID NO: 874 - protein, 14 aa, Artificial Sequence
  Description of Artificial Sequence Synthetic peptide
CASSFNTGNQPGHF

SEQ ID NO: 875 - protein, 15 aa, Artificial Sequence
  Description of Artificial Sequence Synthetic peptide
CASSAGGASNQPQHF

SEQ ID NO: 876 - protein, 15 aa, Artificial Sequence
  Description of Artificial Sequence Synthetic peptide
CASSELRGEDLTQYF

SEQ ID NO: 877 - protein, 14 aa, Artificial Sequence
  Description of Artificial Sequence Synthetic peptide
CASSPGSGNSPLHF

SEQ ID NO: 878 - protein, 14 aa, Artificial Sequence
  Description of Artificial Sequence Synthetic peptide
CSARGGQSSYEQYF

SEQ ID NO: 879 - protein, 15 aa, Artificial Sequence
  Description of Artificial Sequence Synthetic peptide
CSARVGPSNTGELFF

SEQ ID NO: 880 - protein, 13 aa, Artificial Sequence
  Description of Artificial Sequence Synthetic peptide
CASSPYLNYGYTF

SEQ ID NO: 881 - protein, 14 aa, Artificial Sequence
  Description of Artificial Sequence Synthetic peptide
CASSQYFDTGNTYF

SEQ ID NO: 882 - protein, 14 aa, Artificial Sequence
  Description of Artificial Sequence Synthetic peptide
CASANGQNFYGYTF

SEQ ID NO: 883 - protein, 15 aa, Artificial Sequence
  Description of Artificial Sequence Synthetic peptide
CASSSYQFGVGGYTF

SEQ ID NO: 884 - protein, 12 aa, Artificial Sequence
  Description of Artificial Sequence Synthetic peptide
CASSLYSQTGYF

SEQ ID NO: 885 - protein, 12 aa, Artificial Sequence
  Description of Artificial Sequence Synthetic peptide
CASSCRDSPLYF

SEQ ID NO: 886 - protein, 13 aa, Artificial Sequence
  Description of Artificial Sequence Synthetic peptide
CASTTYLADTQYF

SEQ ID NO: 887 - protein, 13 aa, Artificial Sequence
  Description of Artificial Sequence Synthetic peptide
CASSPYLNTEAFF

SEQ ID NO: 888 - protein, 12 aa, Artificial Sequence
  Description of Artificial Sequence Synthetic peptide
CASSTTPPEAFF

SEQ ID NO: 889 - protein, 14 aa, Artificial Sequence
  Description of Artificial Sequence Synthetic peptide
CASSFNIRGTPLHF

SEQ ID NO: 890 - protein, 12 aa, Artificial Sequence
  Description of Artificial Sequence Synthetic peptide
CASSGDRYEQYF

SEQ ID NO: 891 - protein, 13 aa, Artificial Sequence
  Description of Artificial Sequence Synthetic peptide
CASSQWGNTEAFF

SEQ ID NO: 892 - protein, 12 aa, Artificial Sequence
  Description of Artificial Sequence Synthetic peptide
CASSQQGAGYTF

SEQ ID NO: 893 - protein, 14 aa, Artificial Sequence
  Description of Artificial Sequence Synthetic peptide
CASSQGQGQSPLHF

SEQ ID NO: 894 - protein, 14 aa, Artificial Sequence
  Description of Artificial Sequence Synthetic peptide
CASSQGQGNQPQHF

SEQ ID NO: 895 - protein, 15 aa, Artificial Sequence
  Description of Artificial Sequence Synthetic peptide
CASSQEFGMNTEAFF

SEQ ID NO: 896 - protein, 13 aa, Artificial Sequence
  Description of Artificial Sequence Synthetic peptide
CASSPVSNYSYTF

SEQ ID NO: 897 - protein, 15 aa, Artificial Sequence
  Description of Artificial Sequence Synthetic peptide
CASSPVGQRAYEQYF

SEQ ID NO: 898 - protein, 13 aa, Artificial Sequence
  Description of Artificial Sequence Synthetic peptide
CASSPGRTDTQYF

SEQ ID NO: 899 - protein, 13 aa, Artificial Sequence
  Description of Artificial Sequence Synthetic peptide
CASSMTQSYEQYF

SEQ ID NO: 900 - protein, 16 aa, Artificial Sequence
  Description of Artificial Sequence Synthetic peptide
CASSLRAGGPTQTQYF

SEQ ID NO: 901 - protein, 14 aa, Artificial Sequence
  Description of Artificial Sequence Synthetic peptide
CASSLNRNRDTQYF

SEQ ID NO: 902 - protein, 16 aa, Artificial Sequence
  Description of Artificial Sequence Synthetic peptide
CASSLNFAGSTQTQYF

SEQ ID NO: 903 - protein, 15 aa, Artificial Sequence
  Description of Artificial Sequence Synthetic peptide
CASSLLAGGHYEQYF

SEQ ID NO: 904 - protein, 12 aa, Artificial Sequence
  Description of Artificial Sequence Synthetic peptide
CASSLGQNTTYF

SEQ ID NO: 905 - protein, 15 aa, Artificial Sequence
  Description of Artificial Sequence Synthetic peptide
CASSLGPGGGYGYTF

SEQ ID NO: 906 - protein, 14 aa, Artificial Sequence
  Description of Artificial Sequence Synthetic peptide
CASSIMSGDYGYTF

SEQ ID NO: 907 - protein, 14 aa, Artificial Sequence
  Description of Artificial Sequence Synthetic peptide
CASSGQWWSTEAFF

SEQ ID NO: 908 - protein, 12 aa, Artificial Sequence
  Description of Artificial Sequence Synthetic peptide
CASSGGTNGQYF

SEQ ID NO: 909 - protein, 12 aa, Artificial Sequence
  Description of Artificial Sequence Synthetic peptide
CASSFRRENTWF

SEQ ID NO: 910 - protein, 14 aa, Artificial Sequence
  Description of Artificial Sequence Synthetic peptide
CASSPRQGNSPLHF

SEQ ID NO: 911 - protein, 16 aa, Artificial Sequence
  Description of Artificial Sequence Synthetic peptide
CASSFLAGGNTGPLFF

SEQ ID NO: 912 - protein, 15 aa, Artificial Sequence
  Description of Artificial Sequence Synthetic peptide
CASSEWSGWQETQYF

SEQ ID NO: 913 - protein, 14 aa, Artificial Sequence
  Description of Artificial Sequence Synthetic peptide
CASSEWISNGPQHF

SEQ ID NO: 914 - protein, 12 aa, Artificial Sequence
  Description of Artificial Sequence Synthetic peptide
CASSAGTFNQYF

SEQ ID NO: 915 - protein, 14 aa, Artificial Sequence
  Description of Artificial Sequence Synthetic peptide
CAISGTSSGDTQYF

SEQ ID NO: 916 - protein, 15 aa, Artificial Sequence
  Description of Artificial Sequence Synthetic peptide
CASSESWGNYNEQFF

SEQ ID NO: 917 - protein, 14 aa, Artificial Sequence
  Description of Artificial Sequence Synthetic peptide
CAISERSQDYGYTF

SEQ ID NO: 918 - protein, 15 aa, Artificial Sequence
  Description of Artificial Sequence Synthetic peptide
CAISEQALSNGPQHF

SEQ ID NO: 919 - protein, 13 aa, Artificial Sequence
  Description of Artificial Sequence Synthetic peptide
CAISQRAGYGYTF

SEQ ID NO: 920 - protein, 12 aa, Artificial Sequence
  Description of Artificial Sequence Synthetic peptide
CARDKSTDTQYF

SEQ ID NO: 921 - protein, 13 aa, Artificial Sequence
  Description of Artificial Sequence Synthetic peptide
CASGSRGNQPQHF

SEQ ID NO: 922 - protein, 13 aa, Artificial Sequence
  Description of Artificial Sequence Synthetic peptide
CASSPRGDQPQHF

SEQ ID NO: 923 - protein, 13 aa, Artificial Sequence
  Description of Artificial Sequence Synthetic peptide
CASSSRGNQPQHF

SEQ ID NO: 924 - protein, 13 aa, Artificial Sequence
  Description of Artificial Sequence Synthetic peptide
CASSVRGNQPQHF

SEQ ID NO: 925 - protein, 13 aa, Artificial Sequence
  Description of Artificial Sequence Synthetic peptide
CASSYRGNQPQHF

SEQ ID NO: 926 - protein, 13 aa, Artificial Sequence
  Description of Artificial Sequence Synthetic peptide
CASSIDLGQPQHF

SEQ ID NO: 927 - protein, 14 aa, Artificial Sequence
  Description of Artificial Sequence Synthetic peptide
CASSLREGAQPQHF

SEQ ID NO: 928 - protein, 14 aa, Artificial Sequence
  Description of Artificial Sequence Synthetic peptide
CASSPREGNQPQHF

SEQ ID NO: 929 - protein, 14 aa, Artificial Sequence
  Description of Artificial Sequence Synthetic peptide
CASSPREGDQPQHF

SEQ ID NO: 930 - protein, 14 aa, Artificial Sequence
  Description of Artificial Sequence Synthetic peptide
CASSLSTGDQPQHF

SEQ ID NO: 931 - protein, 13 aa, Artificial Sequence
  Description of Artificial Sequence Synthetic peptide
CASSLVGNQPQHF

SEQ ID NO: 932 - protein, 13 aa, Artificial Sequence
  Description of Artificial Sequence Synthetic peptide
CASSLRGNQPQHF

SEQ ID NO: 933 - protein, 13 aa, Artificial Sequence
  Description of Artificial Sequence Synthetic peptide
CSASQGADYGYTF

SEQ ID NO: 934 - protein, 12 aa, Artificial Sequence
  Description of Artificial Sequence Synthetic peptide
CSARDSAYEQYF

SEQ ID NO: 935 - protein, 12 aa, Artificial Sequence
  Description of Artificial Sequence Synthetic peptide
CSALRGGNTTYF

SEQ ID NO: 936 - protein, 12 aa, Artificial Sequence
  Description of Artificial Sequence Synthetic peptide
CSAGKGDTEAFF

SEQ ID NO: 937 - protein, 13 aa, Artificial Sequence
  Description of Artificial Sequence Synthetic peptide
CSAAFGQGLGYTF

SEQ ID NO: 938 - protein, 13 aa, Artificial Sequence
  Description of Artificial Sequence Synthetic peptide
CATANAANTEAFF

SEQ ID NO: 939 - protein, 15 aa, Artificial Sequence
  Description of Artificial Sequence Synthetic peptide
CASSYRGMSTQTQYF

SEQ ID NO: 940 - protein, 14 aa, Artificial Sequence
  Description of Artificial Sequence Synthetic peptide
CASSWDGSNQPQHF

SEQ ID NO: 941 - protein, 16 aa, Artificial Sequence
  Description of Artificial Sequence Synthetic peptide
CASSRGLAGGTQTQYF

SEQ ID NO: 942 - protein, 12 aa, Artificial Sequence
  Description of Artificial Sequence Synthetic peptide
CASSNDTDGYTF

SEQ ID NO: 943 - protein, 14 aa, Artificial Sequence
  Description of Artificial Sequence Synthetic peptide
CASSQWGINGPQHF